(19) United States
(12) Patent Application Publication
(10) Pub. No.: US 2025/0288659 A1
(43) Pub. Date: Sep. 18, 2025

(54) NUCLEIC ACID CONSTRUCT AND USE THEREOF

(71) Applicant: SHANGHAI REGENELEAD THERAPIES CO., LTD., Shanghai (CN)

(72) Inventors: Zhanying HU, Shanghai (CN); Xin MING, Shanghai (CN); Lu GAO, Shanghai (CN); Li WANG, Shanghai (CN); Qingyuan ZHU, Shanghai (CN); Qihui ZHU, Shanghai (CN); Haitao WAN, Shanghai (CN); Qingxin CHEN, Shanghai (CN); Yujie YANG, Shanghai (CN)

(21) Appl. No.: 18/859,960

(22) PCT Filed: Apr. 27, 2023
(86) PCT No.: PCT/CN2023/091194

§ 371 Date: Oct. 24, 2024

(30) Foreign Application Priority Data
Apr. 27, 2022 (CN) 202210456261.9

(51) Int. Cl. A61K 39/145; A61K 39/00; A61K 39/215; A61K 39/385; C07K 14/005; C07K 14/47

(52) U.S. Cl. CPC A61K 39/145; A61K 39/215; A61K 39/385; C07K 14/005; C07K 14/4703; A61K 2039/53; C12N 2760/16122; C12N 2760/16134; C12N 2760/16222; C12N 2760/16234; C12N 2770/20022; C12N 2770/20034

(57) ABSTRACT

The present disclosure relates to a nucleic acid construct and use thereof. Specifically, the present disclosure relates to a nucleic acid construct comprising an engineered UTR, and use thereof in the prevention and treatment of a disease (for example, prevention of virus infection). The UTR can significantly improve the expression efficiency of a target gene in the nucleic acid construct.

The present disclosure claims priority to Chinese Patent Application No. CN202210456261.9, filed on Apr. 27, 2022 and entitled “NUCLEIC ACID CONSTRUCT AND USE THEREOF”, which is incorporated herein by reference in its entirety.

### TECHNICAL FIELD

The present disclosure belongs to the field of nucleic acid drugs and particularly relates to a nucleic acid construct comprising engineered UTRs and use thereof for preventing or treating a disease (e.g., viral infection).

### BACKGROUND

Vaccines are an important area of medical research. Existing vaccines include inactivated vaccines, adenoviral vector vaccines, attenuated influenza virus vector vaccines, recombinant protein vaccines, and nucleic acid vaccines (including RNA vaccines and DNA vaccines). In RNA vaccines, mRNA is the active ingredient, and it mainly contains a cap structure (Cap), a 5′ untranslated region (UTR), an open reading frame (ORF) that encodes an antigen protein, a 3′UTR, and a poly(A) tail structure. The 5′UTR is essential for recruiting ribosomes to the mRNA and initiating codon selection, and it plays an important role in regulating translation efficiency and shaping the cellular proteome (Ivanov, et al. Science. 2016, 352(6292): 1413-1416). Eukaryotic 3′UTRs have various regulatory motifs that can be recognized by microRNAs (miRNAs) and rbp to control mRNA stability, localization, and translation (Mazumder et al., 2003; Mayr, 2017). The polyA tail (located at the 3′ end of the mRNA) is another element that determines mRNA stability and protein levels. Optional 3′UTRs not only affect mRNA stability and translation but also control mRNA localization (Tushev et al., 2018). The design and optimization of mRNA, particularly the selection and use of its UTRs, are critical steps in the entire preparation process of mRNA vaccine products. Therefore, developing efficient UTRs remains an urgent need in the field of mRNA drugs.

The present disclosure provides mRNA vaccines with new UTR structures that offer the advantage of enabling stable and efficient expression of the target gene. The vaccines can be generally applied to mRNA vaccines (e.g., vaccines for influenza or COVID-19) to regulate the expression of the target gene, showcasing excellent prospects for clinical pharmaceutical applications.

### SUMMARY

The present disclosure provides a nucleic acid element capable of regulating the expression of a target gene, and a nucleic acid construct. The nucleotide construct comprises at least one nucleic acid element capable of regulating the expression of a target gene.

### Nucleic Acid Element

In some embodiments, the nucleic acid element is a 5′ untranslated region element (5′UTR).

In some embodiments, the 5′UTR is selected from the group consisting of or is a 5′UTR derived from any one of the genes Rho GTPase activating protein (ARHGAP), heat shock 27 kDa protein 1 (HSPB1), hemoglobin subunit beta (HBB), C—C motif chemokine ligand 13 (CCL13), and the like, or a derivative sequence thereof.

In some embodiments, the 5′UTR in the nucleic acid construct is derived from or is a 5′UTR sequence of an ARHGAP gene or a derivative sequence thereof. In some embodiments, the ARHGAP includes ARHGAP1, ARHGAP2, ARHGAP3, ARHGAP4, ARHGAP5, ARHGAP6, ARHGAP7 (DLC1), ARHGAP8, ARHGAP9, ARHGAP10, ARHGAP12, ARHGAP13 (SRGAP1), ARHGAP14 (SRGAP2), ARHGAP15, ARHGAP17 (RICH1), ARHGAP18, ARHGAP19, ARHGAP20, ARHGAP21, ARHGAP22, ARHGAP23, ARHGAP24, ARHGAP25, and ARHGAP26.

In some embodiments, the 5′UTR is derived from or is a 5′UTR sequence of an ARHGAP15 gene or a derivative sequence thereof. In some embodiments, the ARHGAP15 gene is derived from any species, such as human ARHGAP15, baboon ARHGAP15, monkey ARHGAP15, or mouse ARHGAP15.

In some embodiments, the 5′UTR is derived from or is a 5′UTR sequence of the human ARHGAP15 gene or a derivative sequence thereof. In some embodiments, the 5′UTR of the ARHGAP15 gene comprises or is the nucleotide sequence set forth in SEQ ID NO: 1 or a nucleotide sequence having at least 80%, 85%, 90%, 95%, 96%, 97%, 98%, 99%, or 100% identity thereto.

In some embodiments, the 5′UTR comprises at least one point mutation capable of being used to inhibit an ATG within the UTR from initiating translation. In some embodiments, the point mutation is selected from the group consisting of a mutation at any one or more sites of A, T, or G of the ATG sequence in the 5′UTR. In some embodiments, the point mutation is a mutation at site A of the ATG sequence in the 5′UTR. In some embodiments, the mutation is to mutate A into G, C, or T. In some embodiments, the mutation is to mutate the ATG into a GTG, CTG, or TTG. Further, the mutation is capable of being used to inhibit the ATG within the UTR from initiating translation.

In some embodiments, the aforementioned 5′UTR sequence derived from the ARHGAP15 gene comprises at least one point mutation capable of being used to inhibit an ATG within the UTR from initiating translation. In some embodiments, the point mutation is selected from the group consisting of a mutation at any one or more sites of A, T, or G of the ATG sequence in the 5′UTR. In some embodiments, the point mutation is a mutation at site A of the ATG sequence in the 5′UTR. In some embodiments, the mutation is to mutate A into G, C, or T.

In some embodiments, the aforementioned 5′UTR sequence derived from the ARHGAP15 gene comprises the nucleotide sequence set forth in SEQ ID NO: 44, or, the 5′UTR comprises a nucleotide sequence having at least 80%, 85%, 90%, 95%, 96%, 97%, 98%, or 99% identity to SEQ ID NO: 44, wherein N₁ is selected from the group consisting of A, G, C, and T.

In some embodiments, the aforementioned 5′UTR sequence derived from the ARHGAP15 gene comprises a nucleotide sequence in which an ATG is mutated into a GTG. In some embodiments, the 5′UTR of the ARHGAP15 gene comprises or is the nucleotide sequence set forth in SEQ ID NO: 2 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an ATG is mutated into a CTG. In some embodiments, the 5′UTR of the ARHGAP15 gene comprises or is the nucleotide sequence set forth in SEQ ID NO: 42 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an ATG is mutated into a TTG. In some embodiments, the 5′UTR of the ARHGAP15 gene comprises or is the nucleotide sequence set forth in SEQ ID NO: 43 or a nucleotide sequence having at least 80%, 85%, 90%, 95%, 96%, 97%, 98%, 99%, or 100% identity thereto.

In some embodiments, the 5′UTR is a 5′UTR truncation. In some embodiments, the 5′UTR truncation still maintains active functions similar to those of a natural 5′UTR, e.g., still maintains a function of regulating expression of a protein from an encoding target gene of the ORF. In some embodiments, the 5′UTR truncation has enhanced active functions, e.g., an enhanced function of regulating expression of a protein from an encoding target gene of the ORF, as compared to a natural 5′UTR.

In some embodiments, a truncation mode for the 5′UTR truncation comprises: in the sequence direction from the 5′ end to the 3′ end, deleting a sequence of contiguous nucleotides from the 5′ end; and/or, in the sequence direction from the 3′ end to the 5′ end, deleting a sequence of contiguous nucleotides from the 3′ end. In some embodiments, a truncation mode for the 5′UTR truncation comprises, in the sequence direction from the 5′ end to the 3′ end, deleting a sequence of contiguous nucleotides from the 5′ end, and keeping a nucleotide sequence of the 3′ end. In some embodiments, a truncation mode for the 5′UTR truncation comprises, in the sequence direction from the 3′ end to the 5′ end, deleting a sequence of contiguous nucleotides from the 3′ end, and keeping a nucleotide sequence of the 5′ end. In some embodiments, a truncation mode for the 5′UTR truncation comprises, in the sequence direction from the 5′ end to the 3′ end, deleting a sequence of contiguous nucleotides from the 5′ end; and the truncation mode for the 5′UTR truncation comprises, in the sequence direction from the 3′ end to the 5′ end, deleting a sequence of contiguous nucleotides from the 3′ end. In some embodiments, the 5′UTR truncation also comprises the point mutation according to any of the preceding embodiments. In some embodiments, the 5′UTR truncation does not comprise the point mutation according to any of the preceding embodiments. In some embodiments, the 5′UTR truncation retains a sequence the length of which is at least 90%, 80%, 70%, 60%, 50%, 40%, 30%, 20%, 15%, 10%, 5%, or 1% of the length of a natural 5′UTR sequence.

In some embodiments, the aforementioned 5′UTR sequence derived from the ARHGAP15 gene comprises or is an ARHGAP15 5′UTR truncation. In some embodiments, a truncation mode for the truncation comprises, in the direction from the 5′ end to the 3′ end of the ARHGAP15 5′UTR, deleting a sequence of contiguous nucleotides from the 5′ end; and/or, in the sequence direction from the 3′ end to the 5′ end, deleting a sequence of contiguous nucleotides from the 3′ end.

In some embodiments, a truncation mode for the truncation comprises, in the direction from the 5′ end to the 3′ end of the ARHGAP15 5′UTR, deleting a sequence of contiguous nucleotides from the 5′ end, and keeping a nucleotide sequence of the 3′ end. In some embodiments, a truncation mode for the 5′UTR truncation comprises, in the direction from the 3′ end to the 5′ end of the ARHGAP15 5′UTR, deleting a sequence of contiguous nucleotides from the 3′ end, and keeping a nucleotide sequence of the 5′ end. In some embodiments, a truncation mode for the 5′UTR truncation is, in the direction from the 5′ end to the 3′ end of the ARHGAP15 5′UTR, to delete a sequence of contiguous nucleotides from the 5′ end; and, in the sequence direction from the 3′ end to the 5′ end, to delete a sequence of contiguous nucleotides from the 3′ end.

In some embodiments, the truncation retains a sequence the length of which is at least 90%, 80%, 70%, 60%, 50%, 40%, 30%, 20%, 15%, 10%, 5%, or 1% of the length of the natural ARHGAP15 5′UTR (SEQ ID NO: 1).

In some embodiments, the 5′UTR truncation comprises a sequence of at least 5 contiguous nucleotides from the sequence set forth in SEQ ID NO: 2 or 44; in some embodiments, the 5′UTR truncation comprises a sequence of 5-62 contiguous nucleotides from the sequence set forth in SEQ ID NO: 2 or 44; in some embodiments, the 5′UTR truncation comprises a sequence of 7-62 contiguous nucleotides from the sequence set forth in SEQ ID NO: 2 or 44; in some embodiments, the 5′UTR truncation comprises a sequence of 7-59 contiguous nucleotides from the sequence set forth in SEQ ID NO: 2; illustratively, the 5′UTR truncation comprises a sequence of 5, 7, 8, 9, 10, 11, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 contiguous nucleotides from the sequence set forth in SEQ ID NO: 2 or 44.

In some embodiments, the nucleotide at the 5′ end of the 5′UTR truncation (i.e., the starting nucleotide of the 5′UTR truncation) is the nucleotide at any one of positions 1-57, according to natural counting, of the sequence set forth in SEQ ID NO: 2 or 44. In some embodiments, the nucleotide at the 5′ end of the 5′UTR truncation is the nucleotide at any one of positions 1-13 or 17-29, according to natural counting, of the sequence set forth in SEQ ID NO: 2 or 44, for example, the nucleotide at any one of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and 57.

Illustratively, the nucleotide at the 5′ end of the 5′UTR truncation, and the length of the sequence of contiguous nucleotides from the sequence set forth in SEQ ID NO: 2 that the 5′UTR truncation comprises are shown in the table below:

| Starting | The length of the sequence of contiguous nucleotides |
|---|---|
| nucleotide | from the sequence set forth in SEQ ID NO: 2 |
| Position 1, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, 61, or 62 |
| Position 2, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, 61, or 62 |
| Position 3, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, or 61 |
| Position 4, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60 |
| Position 5, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59 |
| Position 9, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55 |
| Position 13, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51 |
| Position 17, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, 47 |
| Position 21, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43 |
| Position 23, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41 |
| Position 25, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39 |
| Position 29, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35 |
| Position 33, G | 5, 6, 7, 8, 9, 10, 11, 12, 13, 14, 15, 16, 17, 18, 19, 20, 21, 22, 23, 24, 25, 26, |
| | 27, 28, 29, 30, 31 |
| Position 37, T | 5, 6, 7, 8, 9, 10, 11, 12, 13, 14, 15, 16, 17, 18, 19, 20, 21, 22, 23, 24, 25, 26, 27 |
| Position 41, C | 5, 6, 7, 8, 9, 10, 11, 12, 13, 14, 15, 16, 17, 18, 19, 20, 21, 22, 23 |
| Position 45, A | 5, 6, 7, 8, 9, 10, 11, 12, 13, 14, 15, 16, 17, 18, 19 |
| Position 49, G | 5, 6, 7, 8, 9, 10, 11, 12, 13, 14, 15 |
| Position 53, A | 5, 6, 7, 8, 9, 10, 11 |
| Position 57, C | 5, 6, 7 |

In some embodiments, the 5′UTR truncation is 59 bp, 55 bp, 51 bp, 47 bp, 43 bp, 39 bp, 35 bp, 31 bp, 27 bp, 23 bp, 19 bp, 15 bp, 11 bp, or 7 bp in length.

In some embodiments, the ARHGAP15 5′UTR truncation also comprises the point mutation according to any of the preceding embodiments. In some embodiments, the ARHGAP15 5′UTR truncation also comprises a nucleotide sequence in which an ATG is mutated into a GTG. In some embodiments, the aforementioned 5′UTR derived from the ARHGAP15 gene comprises or is the nucleotide sequence set forth in any one of SEQ ID NOs: 28-30 or a nucleotide sequence having at least 80%, 85%, 90%, 95%, 96%, 97%, 98%, 99%, or 100% identity thereto.

In some embodiments, the ARHGAP15 5′UTR truncation does not comprise the point mutation according to any of the preceding embodiments. In some embodiments, the aforementioned 5′UTR of the ARHGAP15 gene comprises or is the nucleotide sequences set forth in SEQ ID NOs: 31-40 and comprises CTATAAT; or a nucleotide sequence having at least 80%, 85%, 90%, 95%, 96%, 97%, 98%, 99%, or 100% identity to any one of the aforementioned sequences.

In some embodiments, the 5′UTR truncation comprises at least CTATAAT or the nucleotide sequence set forth in SEQ ID NO: 193.

In some embodiments, the ARHGAP15 5′UTR truncation comprises or is the nucleotide sequence set forth in any one of SEQ ID NOs: 41, 76, and 137-196 or a nucleotide sequence having at least 80%, 85%, 90%, 95%, 96%, 97%, 98%, 99%, or 100% identity thereto.

In some embodiments, the aforementioned 5′UTR derived from the HSPB1 gene comprises or is the nucleotide sequence set forth in any one of SEQ ID NOs: 3-5 or a nucleotide sequence having at least 80%, 85%, 90%, 95%, 96%, 97%, 98%, 99%, or 100% identity to any one of SEQ ID NOs: 3-5.

In some embodiments, the 5′UTR comprises or is the nucleotide sequence set forth in any one of SEQ ID NOs: 1-2, 3-5, 28-43, 76, and 137-196, or the following sequence: CTATAAT, or a nucleotide sequence having at least 80%, 85%, 90%, 95%, 96%, 97%, 98%, 99%, or 100% identity to any one of the aforementioned sequences.

### Nucleic Acid Construct

The present disclosure provides a nucleic acid construct comprising:
(a) an open reading frame (ORF), and
(b) a 5′ untranslated region element (5′UTR).

In some embodiments, the ORF is a polynucleotide sequence encoding a target gene protein.

In some embodiments, the target gene is heterologous. In some other embodiments, the target gene is endogenous.

In some embodiments, the 5′UTR in the nucleic acid construct is located upstream of the open reading frame. In some embodiments, the 5′UTR in the nucleic acid construct is located at the 5′ end of the open reading frame.

In some embodiments, the 5′UTR in the nucleic acid construct is selected from the group consisting of or is a 5′UTR derived from any one of the genes Rho GTPase activating protein (ARHGAP), heat shock 27 kDa protein 1 (HSPB1), hemoglobin subunit beta (HBB), C—C motif chemokine ligand 13 (CCL13), and the like, or a derivative sequence thereof.

In some embodiments, the 5′UTR in the nucleic acid construct is derived from or is a 5′UTR sequence of an ARHGAP gene or a derivative sequence thereof. In some embodiments, the ARHGAP includes ARHGAP1, ARHGAP2, ARHGAP3, ARHGAP4, ARHGAP5, ARHGAP6, ARHGAP7 (DLC1), ARHGAP8, ARHGAP9, ARHGAP10, ARHGAP12, ARHGAP13 (SRGAP1), ARHGAP14 (SRGAP2), ARHGAP15, ARHGAP17 (RICH1), ARHGAP18, ARHGAP19, ARHGAP20, ARHGAP21, ARHGAP22, ARHGAP23, ARHGAP24, ARHGAP25, and ARHGAP26.

In some embodiments, the 5′UTR in the nucleic acid construct is derived from or is a 5′UTR sequence of an ARHGAP15 gene or a derivative sequence thereof. In some embodiments, the ARHGAP15 gene is derived from any species, such as human ARHGAP15, baboon ARHGAP15, monkey ARHGAP15, or mouse ARHGAP15.

In some embodiments, the 5′UTR in the nucleic acid construct is derived from or is a 5′UTR sequence of the human ARHGAP15 gene or a derivative sequence thereof. In some embodiments, the 5′UTR of the ARHGAP15 gene comprises or is the nucleotide sequence set forth in SEQ ID NO: 1 or a nucleotide sequence having at least 80%, 85%, 90%, 95%, 96%, 97%, 98%, 99%, or 100% identity thereto.

In some embodiments, the 5′UTR in the nucleic acid construct comprises at least one point mutation capable of being used to inhibit an ATG within the UTR from initiating translation. In some embodiments, the point mutation is selected from the group consisting of a mutation at any one or more sites of A, T, or G of the ATG sequence in the 5′UTR. In some embodiments, the point mutation is a mutation at site A of the ATG sequence in the 5′UTR. In some embodiments, the mutation is to mutate A into G, C, or T. In some embodiments, the mutation is to mutate the ATG into a GTG, CTG, or TTG. Further, the mutation is capable of being used to inhibit the ATG within the UTR from initiating translation.

In some embodiments, the aforementioned 5′UTR sequence derived from the ARHGAP15 gene comprises at least one point mutation capable of being used to inhibit an ATG within the UTR from initiating translation. In some embodiments, the point mutation is selected from the group consisting of a mutation at any one or more sites of A, T, or G of the ATG sequence in the 5′UTR. In some embodiments, the point mutation is a mutation at site A of the ATG sequence in the 5′UTR. In some embodiments, the mutation is to mutate A into G, C, or T.

In some embodiments, the aforementioned 5′UTR sequence derived from the ARHGAP15 gene comprises the nucleotide sequence set forth in SEQ ID NO: 44, or, the 5′UTR comprises a nucleotide sequence having at least 80%, 85%, 90%, 95%, 96%, 97%, 98%, or 99% identity to SEQ ID NO: 44, wherein N₁ is selected from the group consisting of A, G, C, and T.

In some embodiments, the aforementioned 5′UTR sequence derived from the ARHGAP15 gene comprises a nucleotide sequence in which an ATG is mutated into a GTG. In some embodiments, the 5′UTR of the ARHGAP15 gene comprises or is the nucleotide sequence set forth in SEQ ID NO: 2 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an ATG is mutated into a CTG. In some embodiments, the 5′UTR of the ARHGAP15 gene comprises or is the nucleotide sequence set forth in SEQ ID NO: 42 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an ATG is mutated into a TTG. In some embodiments, the 5′UTR of the ARHGAP15 gene comprises or is the nucleotide sequence set forth in SEQ ID NO: 43 or a nucleotide sequence having at least 80%, 85%, 90%, 95%, 96%, 97%, 98%, 99%, or 100% identity thereto.

In some embodiments, the 5′UTR in the nucleic acid construct of the present disclosure is a 5′UTR truncation. In some embodiments, the 5′UTR truncation still maintains active functions similar to those of a natural 5′UTR, e.g., still maintains a function of regulating expression of a protein from an encoding target gene of the ORF. In some embodiments, the 5′UTR truncation has enhanced active functions, e.g., an enhanced function of regulating expression of a protein from an encoding target gene of the ORF, as compared to a natural 5′UTR.

In some embodiments, a truncation mode for the 5′UTR truncation comprises: in the sequence direction from the 5′ end to the 3′ end, deleting a sequence of contiguous nucleotides from the 5′ end; and/or, in the sequence direction from the 3′ end to the 5′ end, deleting a sequence of contiguous nucleotides from the 3′ end. In some embodiments, a truncation mode for the 5′UTR truncation comprises, in the sequence direction from the 5′ end to the 3′ end, deleting a sequence of contiguous nucleotides from the 5′ end, and keeping a nucleotide sequence of the 3′ end. In some embodiments, a truncation mode for the 5′UTR truncation comprises, in the sequence direction from the 3′ end to the 5′ end, deleting a sequence of contiguous nucleotides from the 3′ end, and keeping a nucleotide sequence of the 5′ end. In some embodiments, a truncation mode for the 5′UTR truncation comprises, in the sequence direction from the 5′ end to the 3′ end, deleting a sequence of contiguous nucleotides from the 5′ end; and the truncation mode for the 5′UTR truncation comprises, in the sequence direction from the 3′ end to the 5′ end, deleting a sequence of contiguous nucleotides from the 3′ end.

In some embodiments, the 5′UTR truncation also comprises the point mutation according to any of the preceding embodiments. In some embodiments, the 5′UTR truncation does not comprise the point mutation according to any of the preceding embodiments. In some embodiments, the 5′UTR truncation retains a sequence the length of which is at least 90%, 80%, 70%, 60%, 50%, 40%, 30%, 20%, 15%, 10%, 5%, or 1% of the length of a natural 5′UTR sequence.

In some embodiments, the aforementioned 5′UTR sequence derived from the ARHGAP15 gene comprises or is an ARHGAP15 5′UTR truncation. In some embodiments, a truncation mode for the truncation comprises, in the direction from the 5′ end to the 3′ end of the ARHGAP15 5′UTR, deleting a sequence of contiguous nucleotides from the 5′ end; and/or, in the sequence direction from the 3′ end to the 5′ end, deleting a sequence of contiguous nucleotides from the 3′ end.

In some embodiments, a truncation mode for the truncation comprises, in the direction from the 5′ end to the 3′ end of the ARHGAP15 5′UTR, deleting a sequence of contiguous nucleotides from the 5′ end, and keeping a nucleotide sequence of the 3′ end. In some embodiments, a truncation mode for the 5′UTR truncation comprises, in the direction from the 3′ end to the 5′ end of the ARHGAP15 5′UTR, deleting a sequence of contiguous nucleotides from the 3′ end, and keeping a nucleotide sequence of the 5′ end. In some embodiments, a truncation mode for the 5′UTR truncation is, in the direction from the 5′ end to the 3′ end of the ARHGAP15 5′UTR, to delete a sequence of contiguous nucleotides from the 5′ end; and, in the sequence direction from the 3′ end to the 5′ end, to delete a sequence of contiguous nucleotides from the 3′ end.

In some embodiments, the truncation retains a sequence the length of which is at least 90%, 80%, 70%, 60%, 50%, 40%, 30%, 20%, 15%, 10%, 5%, or 1% of the length of the natural ARHGAP15 5′UTR (SEQ ID NO: 1).

In some embodiments, the 5′UTR truncation comprises a sequence of at least 5 contiguous nucleotides from the nucleotide sequence set forth in SEQ ID NO: 2 or 44; in some embodiments, the 5′UTR truncation comprises a sequence of 5-62 contiguous nucleotides from the sequence set forth in SEQ ID NO: 2 or 44; in some embodiments, the 5′UTR truncation comprises a sequence of 7-62 contiguous nucleotides from the sequence set forth in SEQ ID NO: 2 or 44; in some embodiments, the 5′UTR truncation comprises a sequence of 7-59 contiguous nucleotides from the sequence set forth in SEQ ID NO: 2; illustratively, the 5′UTR truncation comprises a sequence of 5, 7, 8, 9, 10, 11, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 contiguous nucleotides from the sequence set forth in SEQ ID NO: 2.

In some embodiments, the nucleotide at the 5′ end of the 5′UTR truncation (i.e., the starting nucleotide of the 5′UTR truncation) is the nucleotide at any one of positions 1-57, according to natural counting, of the sequence set forth in SEQ ID NO: 2 or 44. In some embodiments, the nucleotide at the 5′ end of the 5′UTR truncation is the nucleotide at any one of positions 1-13 or 17-29, according to natural counting, of the sequence set forth in SEQ ID NO: 2 or 44, for example, the nucleotide at any one of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and 57.

Illustratively, the nucleotide at the 5′ end of the 5′UTR truncation, and the length of the sequence of contiguous nucleotides from the sequence set forth in SEQ ID NO: 2 that the 5′UTR truncation comprises are shown in the table below:

| Starting | The length of the sequence of contiguous nucleotides |
|---|---|
| nucleotide | from the sequence set forth in SEQ ID NO: 2 |
| Position 1, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, 61, or 62 |
| Position 2, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, 61, or 62 |
| Position 3, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60, or 61 |
| Position 4, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59, 60 |
| Position 5, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55, 56, 57, 58, 59 |
| Position 9, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51, 52, 53, 54, 55 |
| Position 13, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, |
| | 47, 48, 49, 50, 51 |
| Position 17, G | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43, 44, 45, 46, 47 |
| Position 21, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41, 42, 43 |
| Position 23, T | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39, 40, 41 |
| Position 25, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35, 36, 37, 38, 39 |
| Position 29, A | 5, 6, 7, 8, 9, 10, 11, 12, 13, 14, 15, 16, 17, 18, 19, 20, 21, 22, 23, 24, 25, 26, |
| | 27, 28, 29, 30, 31, 32, 33, 34, 35 |
| Position 33, G | 5, 6, 7, 8, 9, 10, 11, 12, 13, 14, 15, 16, 17, 18, 19, 20, 21, 22, 23, 24, 25, 26, |
| | 27, 28, 29, 30, 31 |
| Position 37, T | 5, 6, 7, 8, 9, 10, 11, 12, 13, 14, 15, 16, 17, 18, 19, 20, 21, 22, 23, 24, 25, 26, 27 |
| Position 41, C | 5, 6, 7, 8, 9, 10, 11, 12, 13, 14, 15, 16, 17, 18, 19, 20, 21, 22, 23 |
| Position 45, A | 5, 6, 7, 8, 9, 10, 11, 12, 13, 14, 15, 16, 17, 18, 19 |
| Position 49, G | 5, 6, 7, 8, 9, 10, 11, 12, 13, 14, 15 |
| Position 53, A | 5, 6, 7, 8, 9, 10, 11 |
| Position 57, C | 5, 6, 7 |

In some embodiments, the 5′UTR truncation is 59 bp, 55 bp, 51 bp, 47 bp, 43 bp, 39 bp, 35 bp, 31 bp, 27 bp, 23 bp, 19 bp, 15 bp, 11 bp, or 7 bp in length.

In some embodiments, the ARHGAP15 5′UTR truncation also comprises the point mutation according to any of the preceding embodiments. In some embodiments, the ARHGAP15 5′UTR truncation also comprises a nucleotide sequence in which an ATG is mutated into a GTG. In some embodiments, the aforementioned 5′UTR derived from the ARHGAP15 gene comprises or is the nucleotide sequence set forth in any one of SEQ ID NOs: 28-30 or a nucleotide sequence having at least 80%, 85%, 90%, 95%, 96%, 97%, 98%, 99%, or 100% identity thereto.

In some embodiments, the ARHGAP15 5′UTR truncation does not comprise the point mutation according to any of the preceding embodiments. In some embodiments, the aforementioned 5′UTR of the ARHGAP15 gene comprises or is the nucleotide sequences set forth in SEQ ID NOs: 31-40, CTATAAT, or a nucleotide sequence having at least 80%, 85%, 90%, 95%, 96%, 97%, 98%, 99%, or 100% identity thereto.

In some embodiments, the 5′UTR truncation comprises at least CTATAAT or the nucleotide sequence set forth in SEQ ID NO: 193.

In some embodiments, the ARHGAP15 5′UTR truncation comprises or is the nucleotide sequence set forth in any one of SEQ ID NOs: 41, 76, and 137-196 or a nucleotide sequence having at least 80%, 85%, 90%, 95%, 96%, 97%, 98%, 99%, or 100% identity thereto.

In some embodiments, the aforementioned 5′UTR derived from the HSPB1 gene comprises or is the nucleotide sequence set forth in any one of SEQ ID NOs: 3-5 or a nucleotide sequence having at least 80%, 85%, 90%, 95%, 96%, 97%, 98%, 99%, or 100% identity to any one of SEQ ID NOs: 3-5.

In some embodiments, the 5′UTR in the nucleic acid constructs of the present disclosure comprises or is the nucleotide sequence set forth in any one of SEQ ID NOs: 1-2, 3-5, 28-43, 76, and 137-196, or the following sequence: CTATAAT; or a nucleotide sequence having at least 80%, 85%, 90%, 95%, 96%, 97%, 98%, 99%, or 100% identity thereto.

In some specific embodiments, the 5′UTR in the nucleic acid construct according to any one of the above comprises or is the nucleotide sequence set forth in any one of SEQ ID NOs: 1-5, 28-43, 76, and 137-196, or the following sequence: CTATAAT.

In some embodiments, the nucleic acid construct of the present disclosure also further comprises: (c) a 3′ untranslated region element (3′UTR).

In some embodiments, the open reading frame of the present disclosure is derived from a different gene than the 5′UTR and/or the 3′UTR. In some embodiments, the nucleic acid construct of the present disclosure comprises at least one open reading frame, at least one 5′UTR, or at least one 3′UTR. In some embodiments, the 5′UTR and 3′UTR in the nucleic acid construct of the present disclosure are from the same source or different sources, e.g., derived from the same gene or different genes. In some embodiments, the 5′UTR and 3′UTR in the nucleic acid construct of the present disclosure are derived from the same species or different species.

In some embodiments, the 3′UTR in the nucleic acid construct of the present disclosure is located downstream of the open reading frame. In some embodiments, the 3′UTR in the nucleic acid construct is located at the 3′ end of the open reading frame.

In some embodiments, the 3′UTR in the nucleic acid construct of the present disclosure is selected from the group consisting of or is a 3′UTR derived from any one of the genes hemoglobin subunit beta (HBB), ARHGAP15, coronin 1A (CORO1A), hemopexin (HPX), and the like, or a derivative sequence thereof.

In some embodiments, the 3′UTR is derived from or is a 3′UTR sequence of the HBB or ARHGAP15 gene or a derivative sequence thereof.

In some embodiments, the aforementioned 3′UTR derived from the HBB gene comprises or is the nucleotide sequence set forth in SEQ ID NO: 7 or a nucleotide sequence having at least 80%, 85%, 90%, 95%, 96%, 97%, 98%, 99%, or 100% identity thereto.

In some embodiments, the aforementioned 3′UTR derived from the ARHGAP15 gene comprises or is the nucleotide sequence set forth in SEQ ID NO: 8 or a nucleotide sequence having at least 80%, 85%, 90%, 95%, 96%, 97%, 98%, 99%, or 100% identity thereto.

In some embodiments, the aforementioned 3′UTR derived from the CORO1A gene comprises or is the nucleotide sequence set forth in SEQ ID NO: 9 or a nucleotide sequence having at least 80%, 85%, 90%, 95%, 96%, 97%, 98%, 99%, or 100% identity thereto.

In some embodiments, the aforementioned 3′UTR derived from the HPX gene comprises or is the nucleotide sequence set forth in SEQ ID NO: 10 or a nucleotide sequence having at least 80%, 85%, 90%, 95%, 96%, 97%, 98%, 99%, or 100% identity thereto.

In some embodiments, the 3′UTR in the nucleic acid construct of the present disclosure comprises or is the nucleotide sequence set forth in any one of SEQ ID NOs: 7-10 or a nucleotide sequence having at least 80%, 85%, 90%, 95%, 96%, 97%, 98%, 99%, or 100% identity thereto. In some specific embodiments, the 3′UTR comprises or is the nucleotide sequence set forth in SEQ ID NO: 7 or 8.

In some embodiments, the nucleic acid construct comprises a 5′UTR and a 3′UTR, wherein:
the 5′UTR is selected from the group consisting of or is a 5′UTR derived from any one of the genes ARHGAP (e.g., ARHGAP15), HSPB1, HBB, CCL13, and the like, or a derivative sequence thereof, and the 3′UTR is selected from the group consisting of or is a 3′UTR derived from any one of the genes HBB, ARHGAP15, CORO1A, HPX, and the like, or a derivative sequence thereof.

In some embodiments, the nucleic acid construct comprises a 5′UTR and a 3′UTR, the 5′UTR and the 3′UTR being selected from the group consisting of any one of the following:
the 5′UTR is derived from or is a 5′UTR of the ARHGAP15 gene or a derivative sequence thereof, and the 3′UTR is derived from or is a 3′UTR of the HBB gene or a derivative sequence thereof;
the 5′UTR is derived from or is a 5′UTR of the ARHGAP15 gene or a derivative sequence thereof, and the 3′UTR is derived from or is a 3′UTR of the ARHGAP15 gene or a derivative sequence thereof;
the 5′UTR is derived from or is a 5′UTR of the ARHGAP15 gene or a derivative sequence thereof, and the 3′UTR is derived from or is a 3′UTR of the CORO1A gene or a derivative sequence thereof;
the 5′UTR is derived from or is a 5′UTR of the ARHGAP15 gene or a derivative sequence thereof, and the 3′UTR is derived from or is a 3′UTR of the HPX gene or a derivative sequence thereof;
the 5′UTR is derived from or is a 5′UTR of the HBB gene or a derivative sequence thereof, and the 3′UTR is derived from or is a 3′UTR of the HBB gene or a derivative sequence thereof;
the 5′UTR is derived from or is a 5′UTR of the HBB gene or a derivative sequence thereof, and the 3′UTR is derived from or is a 3′UTR of the ARHGAP15 gene or a derivative sequence thereof;
the 5′UTR is derived from or is a 5′UTR of the HBB gene or a derivative sequence thereof, and the 3′UTR is derived from or is a 3′UTR of the CORO1A gene or a derivative sequence thereof;
the 5′UTR is derived from or is a 5′UTR of the HBB gene or a derivative sequence thereof, and the 3′UTR is derived from or is a 3′UTR of the HPX gene or a derivative sequence thereof;
the 5′UTR is derived from or is a 5′UTR of the HSPB1 gene or a derivative sequence thereof, and the 3′UTR is derived from or is a 3′UTR of the HBB gene or a derivative sequence thereof;
the 5′UTR is derived from or is a 5′UTR of the HSPB1 gene or a derivative sequence thereof, and the 3′UTR is derived from or is a 3′UTR of the ARHGAP15 gene or a derivative sequence thereof;
the 5′UTR is derived from or is a 5′UTR of the HSPB1 gene or a derivative sequence thereof, and the 3′UTR is derived from or is a 3′UTR of the CORO1A gene or a derivative sequence thereof;
the 5′UTR is derived from or is a 5′UTR of the HSPB1 gene or a derivative sequence thereof, and the 3′UTR is derived from or is a 3′UTR of the HPX gene or a derivative sequence thereof; or
the 5′UTR is derived from or is a 5′UTR of the CCL13 gene or a derivative sequence thereof, and the 3′UTR is derived from or is a 3′UTR of the HBB gene or a derivative sequence thereof;
the 5′UTR is derived from or is a 5′UTR of the CCL13 gene or a derivative sequence thereof, and the 3′UTR is derived from or is a 3′UTR of the ARHGAP15 gene or a derivative sequence thereof;
the 5′UTR is derived from or is a 5′UTR of the CCL13 gene or a derivative sequence thereof, and the 3′UTR is derived from or is a 3′UTR of the CORO1A gene or a derivative sequence thereof; or
the 5′UTR is derived from or is a 5′UTR of the CCL13 gene or a derivative sequence thereof, and the 3′UTR is derived from or is a 3′UTR of the HPX gene or a derivative sequence thereof.

In some embodiments, the nucleic acid construct of the present disclosure comprises a 5′UTR and a 3′UTR, the 5′UTR and the 3′UTR being selected from the group consisting of any one of the following:
the 5′UTR comprises or is the nucleotide sequence set forth in any one of SEQ ID NOs: 1-2, 28-43, 76, and 137-196 and CTATAAT or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 7 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 1-2, 28-43, 76, and 137-196 and CTATAAT or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 8 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 1-2, 28-43, 76, and 137-196 and CTATAAT or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 9 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 1-2, 28-43, 76, and 137-196 and CTATAAT or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 10 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 3 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 7 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 3 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 8 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 3 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 9 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 3 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 10 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 4 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 7 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 4 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 8 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 4 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 9 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 4 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 10 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 5 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 7 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 5 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 8 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 5 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 9 or a nucleotide sequence having at least 80%, 85%, 90%, 95%, 96%, 97%, 98%, 99%, or 100% identity thereto; and
the 5′UTR comprises or is the nucleotide sequence set forth in SEQ ID NO: 5 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 10 or a nucleotide sequence having at least 80%, 85%, 90%, 95%, 96%, 97%, 98%, 99%, or 100% identity thereto.

In some embodiments, the nucleic acid construct comprises a 5′UTR and a 3′UTR, wherein:
the 5′UTR comprises or is the nucleotide sequence set forth in any one of SEQ ID NOs: 1-2, 28-43, 76, and 137-196 and CTATAAT, and the 3′UTR is derived from or is a 3′UTR derived from the HBB, ARHGAP15, CORO1A, or HPX gene or a derivative sequence thereof.

In some specific embodiments, the nucleic acid construct comprises a 5′UTR and a 3′UTR, wherein:
the 5′UTR comprises or is the nucleotide sequence set forth in any one of SEQ ID NOs: 1-2, 28-43, 76, and 137-196 and CTATAAT, and the 3′UTR comprises or is the nucleotide sequence set forth in any one of SEQ ID NOs: 7-10.

In some embodiments, the nucleic acid construct of the present disclosure also further comprises: (d) a polyadenylic acid (poly-A) tail.

In some embodiments, the poly-A tail in the nucleic acid construct is located downstream of the 3′UTR. In some embodiments, the poly-A tail in the nucleic acid construct is located at the 3′ end of the 3′UTR. In some embodiments, the poly-A tail is at the 3′ end of the nucleic acid construct. In some embodiments, the poly-A tail is at least about 50, 100, 150, 200, 300, 400, or 500 nucleotides in length.

In some embodiments, the poly-A tail includes, but is not limited to, HGH polyA, SV40polyA, BGH polyA, rbGlob polyA, or SV40late polyA.

In some specific embodiments, the poly-A tail comprises or is the nucleotide sequence set forth in SEQ ID NO: 16 or 135.

In some embodiments, in the 5′-to-3′ direction, the nucleic acid construct according to any one of the above comprises any one of i)-iv):
i) a 5′UTR, and an open reading frame (ORF);
ii) an open reading frame (ORF), and a 3′UTR;
iii) a 5′UTR, an open reading frame (ORF), and a 3′UTR; and
iv) a 5′UTR, an open reading frame (ORF), a 3′UTR, and a poly-A tail;
wherein the ORF is derived from a different gene than the 5′UTR and/or the 3′UTR.

In some embodiments, the 5′UTR in i) and iii) to iv) is selected from the group consisting of a 5′UTR derived from any one of the genes ARHGAP, HSPB1, HBB, CCL13, and the like, or a derivative sequence thereof (e.g., a 5′UTR of ARHGAP15 or a derivative sequence thereof).

In some embodiments, the 3′UTR in ii) to iv) is selected from the group consisting of a 3′UTR derived from any one of the genes HBB, ARHGAP15, CORO1A, HPX, and the like, or a derivative sequence thereof (e.g., a 3′UTR of HBB or ARHGAP15 or a derivative sequence thereof).

In some embodiments, the poly-A tail in iv) to iv) comprises, for example, the nucleotide sequence set forth in SEQ ID NO: 16 or 135.

In some embodiments, the 5′UTR in i) comprises or is the nucleotide sequence set forth in any one of SEQ ID NOs: 1-5, 28-43, 76, and 137-196 and CTATAAT.

In some embodiments, the 3′UTR in ii) comprises or is the nucleotide sequence set forth in any one of SEQ ID NOs: 7-10.

In some embodiments, the 5′UTR in iii) comprises or is the nucleotide sequence set forth in any one of SEQ ID NOs: 1-5, 28-43, 76, and 137-196 and CTATAAT, and the 3′UTR comprises or is the nucleotide sequence set forth in any one of SEQ ID NOs: 7-10.

In some embodiments, the 5′UTR in iv) comprises or is the nucleotide sequence set forth in any one of SEQ ID NOs: 1-5, 28-43, 76, and 137-196 and CTATAAT, the 3′UTR comprises or is the nucleotide sequence set forth in any one of SEQ ID NOs: 7-10, and the poly-A tail comprises or is the nucleotide sequence set forth in SEQ ID NO: 16 or 135.

In the present disclosure, in the nucleic acid construct according to any one of the above, the ORF comprises a nucleotide sequence encoding at least one polypeptide or protein. In some embodiments, the nucleotide sequence may be a codon-optimized nucleotide sequence.

In some embodiments, the polypeptide or protein encoded by the ORF is a fluorescent protein or a luciferase.

In some embodiments, the polypeptide or protein encoded by the ORF is a viral antigen. Illustratively, the viral antigen includes, but is not limited to, antigens of influenza viruses, coronaviruses, respiratory syncytial virus, human immunodeficiency viruses, herpes simplex virus, rabies virus, or EB virus, among others.

In some embodiments, the viral antigen is a coronavirus antigen. In some embodiments, the coronavirus is a human-infecting coronavirus, such as SARS-CoV-2 (COVID-19), SARS-CoV, HCoV-229E, HCoV-OC43, HCoV-NL63, HCoV-HKU1, or MERS-CoV. In some embodiments, the coronavirus is SARS-COV-2. In some embodiments, the coronavirus antigen is a structural protein. In some embodiments, the structural protein is selected from the group consisting of a spike protein (S protein), an envelope protein (E protein), a membrane protein (M protein), and a nucleocapsid protein (N protein). In some embodiments, the structural protein is a spike protein. In some embodiments, the spike protein is a SARS-COV-2 spike protein. In some embodiments, the SARS-COV-2 spike protein is selected from the group consisting of a spike protein of any one of the viral strains SARS-COV-2 (e.g., wild-type SARS-COV-2), SARS-COV-2 Alpha(B.1.1.7), SARS-COV-2 Beta(B.1.351), SARS-COV-2 Gamma(P.1), SARS-COV-2 Kappa(B.1.617.1), SARS-COV-2 Delta(B.1.617.2), SARS-COV-2 Omicron(B.1.1.529), SARS-COV-2 Omicron(B.A.4), and the like.

In some embodiments, the SARS-COV-2 spike protein comprises or is the amino acid sequence set forth in any one of SEQ ID NOs: 12, 14, 21, 23, 25, 80, and 136 or a sequence having at least 80%, 85%, 90%, 95%, 96%, 97%, 98%, 99%, or 100% amino acid identity thereto.

In some embodiments, the ORF comprises a codon-optimized nucleotide sequence comprising a sequence having at least 80%, 85%, 90%, 95%, 96%, 97%, 98%, 99%, or 100% identity to a wild-type nucleotide sequence encoding a SARS-COV-2 antigen (e.g., wild-type SARS-COV-2, SEQ ID NO: 81).

In some embodiments, the ORF encoding the polypeptide or protein comprises or is the nucleotide sequence set forth in any one of SEQ ID NOs: 13, 15, 20, 22, 24, 81, and 97 or a nucleotide sequence having at least 80%, 85%, 90%, 95%, 96%, 97%, 98%, 99%, or 100% identity thereto.

In some embodiments, the ORF encodes an influenza virus antigen. In some embodiments, the influenza virus is selected from the group consisting of influenza A virus and influenza B virus; illustratively, the influenza virus is influenza A virus H1N1, influenza A virus H3N2, influenza A virus H3N8, influenza A virus H2N2, influenza A virus H5N1, influenza A virus H9N2, influenza A virus H7N7, influenza B virus/Victoria (e.g., influenza B virus/Washington/02/2019), influenza B virus/Yamagata (e.g., influenza B/Phuket/3073/2013), or the like. In some embodiments, the influenza virus antigen is a structural protein of an influenza virus, e.g., hemagglutinin (HA), neuraminidase (NA), M2 ion channel, matrix protein M1, or nucleoprotein (NP). In some specific embodiments, the influenza virus antigen is an HA protein of an influenza virus, e.g., an HA protein of influenza A virus H1N1, influenza A virus H3N2, influenza B virus Victoria (e.g., influenza B/Washington/02/2019), or influenza B virus Yamagata (e.g., influenza B/Phuket/3073/2013). In some specific embodiments, the influenza virus antigen is an NA protein of an influenza virus, e.g., an NA protein of influenza A virus H1N1, influenza A virus H3N2, influenza B virus Victoria (e.g., influenza B/Washington/02/2019), or influenza B virus Yamagata (e.g., influenza B/Phuket/3073/2013).

In some embodiments, the HA protein comprises or is the amino acid sequence set forth in any one of SEQ ID NOs: 83-86 and 98-101 or a sequence having at least 80%, 85%, 90%, 95%, 96%, 97%, 98%, 99%, or 100% amino acid identity thereto.

In some embodiments, the ORF comprises a codon-optimized nucleotide sequence comprising a sequence having at least 80%, 85%, 90%, 95%, 96%, 97%, 98%, 99%, or 100% identity to a wild-type nucleotide sequence encoding an HA antigen.

In some embodiments, the ORF comprises or is the nucleotide sequence set forth in any one of SEQ ID NOs: 87-90 and 102-105 or a sequence having at least 80%, 85%, 90%, 95%, 96%, 97%, 98%, 99%, or 100% identity thereto.

In some embodiments, the ORF comprises or is the nucleotide sequence set forth in any one of SEQ ID NOs: 93-96 or a sequence having at least 80%, 85%, 90%, 95%, 96%, 97%, 98%, 99%, or 100% identity thereto.

In some embodiments, the present disclosure provides a nucleic acid construct comprising, in the 5′-to-3′ direction, a 5′UTR, an ORF, a 3′UTR, and a poly-A tail in sequence. In some embodiments, the 5′UTR comprises or is the nucleotide sequence set forth in any one of SEQ ID NOs: 1-5, 28-43, 76, and 137-196 and CTATAAT; the ORF comprises or is the nucleotide sequence set forth in any one of SEQ ID NOs: 13, 15, 20, 22, 24, 81, 97, 87-90, 93-96, and 102-105, the 3′UTR comprises or is the nucleotide sequence set forth in any one of SEQ ID NOs: 7-10, and the poly-A tail comprises or is the nucleotide sequence set forth in SEQ ID NO: 16 or 135.

Illustratively, the nucleic acid construct comprises the nucleotide sequences set forth in SEQ ID NOs: 18-19.

In the present disclosure, including any embodiment thereof, the nucleic acid construct is a nucleic acid molecule, e.g., a cDNA.

### RNA Molecule

The present disclosure provides an RNA molecule comprising:
(a) an open reading frame (ORF), and
(b) a 5′ untranslated region element (5′UTR).

In some embodiments, the ORF is a polynucleotide sequence encoding a target gene protein.

In some embodiments, the target gene is heterologous. In some other embodiments, the target gene is endogenous.

In some embodiments, the 5′UTR in the RNA molecule is located upstream of the open reading frame. In some embodiments, the 5′UTR in the RNA molecule is located at the 5′ end of the open reading frame.

In some embodiments, the 5′UTR in the RNA molecule is selected from the group consisting of or is a 5′UTR derived from any one of the genes Rho GTPase activating protein (ARHGAP), heat shock 27 kDa protein 1 (HSPB1), hemoglobin subunit beta (HBB), C—C motif chemokine ligand 13 (CCL13), and the like, or a derivative sequence thereof.

In some embodiments, the 5′UTR in the RNA molecule is derived from or is a 5′UTR sequence of an ARHGAP gene or a derivative sequence thereof. In some embodiments, the ARHGAP includes ARHGAP1, ARHGAP2, ARHGAP3, ARHGAP4, ARHGAP5, ARHGAP6, ARHGAP7 (DLC1), ARHGAP8, ARHGAP9, ARHGAP10, ARHGAP12, ARHGAP13 (SRGAP1), ARHGAP14 (SRGAP2), ARHGAP15, ARHGAP17 (RICH1), ARHGAP18, ARHGAP19, ARHGAP20, ARHGAP21, ARHGAP22, ARHGAP23, ARHGAP24, ARHGAP25, and ARHGAP26.

In some embodiments, the 5′UTR is derived from or is a 5′UTR sequence of an ARHGAP15 gene or a derivative sequence thereof. In some embodiments, the ARHGAP15 gene is derived from any species, such as human ARHGAP15, baboon ARHGAP15, or mouse ARHGAP15.

In some embodiments, the 5′UTR is derived from or is a 5′UTR sequence of the human ARHGAP15 gene or a derivative sequence thereof. In some embodiments, the 5′UTR of the ARHGAP15 gene comprises or is the nucleotide sequence set forth in SEQ ID NO: 45 or a nucleotide sequence having at least 80%, 85%, 90%, 95%, 96%, 97%, 98%, 99%, or 100% identity thereto.

In some embodiments, the 5′UTR in the RNA molecule of the present disclosure comprises at least one point mutation, the point mutation including the point mutation in any embodiment of the aforementioned nucleic acid construct. In some embodiments, the mutation is to mutate A into G, C, or U.

In some embodiments, the aforementioned 5′UTR sequence derived from the ARHGAP15 gene comprises the nucleotide sequence set forth in SEQ ID NO: 79, or, the 5′UTR comprises a nucleotide sequence having at least 80%, 85%, 90%, 95%, 96%, 97%, 98%, or 99% identity to SEQ ID NO: 79, wherein N2 is selected from the group consisting of A, G, C, and U.

In some embodiments, the aforementioned 5′UTR sequence derived from the ARHGAP15 gene comprises a nucleotide sequence in which an AUG is mutated into a GUG. In some embodiments, the 5′UTR derived from the ARHGAP15 gene comprises or is the nucleotide sequence set forth in SEQ ID NO: 46 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an AUG is mutated into a CUG. In some embodiments, the 5′UTR derived from the ARHGAP15 gene comprises or is the nucleotide sequence set forth in SEQ ID NO: 77 or a nucleotide sequence having at least 80%, 85%, 90%, 95%, 96%, 97%, 98%, 99%, or 100% identity thereto.

In some embodiments, the aforementioned 5′UTR sequence derived from the ARHGAP15 gene comprises a nucleotide sequence in which an AUG is mutated into a UUG. In some embodiments, the 5′UTR derived from the ARHGAP15 gene comprises or is the nucleotide sequence set forth in SEQ ID NO: 78 or a nucleotide sequence having at least 80%, 85%, 90%, 95%, 96%, 97%, 98%, 99%, or 100% identity thereto.

In some embodiments, the 5′UTR in the RNA molecule of the present disclosure is a 5′UTR truncation. In some embodiments, the 5′UTR truncation still maintains active functions similar to those of a natural 5′UTR, e.g., still maintains a function of regulating expression of a protein from an encoding target gene of the ORF.

In some embodiments, a truncation mode for the 5′UTR truncation comprises: in the sequence direction from the 5′ end to the 3′ end, deleting a sequence of contiguous nucleotides from the 5′ end; and/or, in the sequence direction from the 3′ end to the 5′ end, deleting a sequence of contiguous nucleotides from the 3′ end. In some embodiments, a truncation mode for the 5′UTR truncation comprises, in the sequence direction from the 5′ end to the 3′ end, deleting a sequence of contiguous nucleotides from the 5′ end, and keeping a nucleotide sequence of the 3′ end. In some embodiments, a truncation mode for the 5′UTR truncation comprises, in the sequence direction from the 3′ end to the 5′ end, deleting a sequence of contiguous nucleotides from the 3′ end, and keeping a nucleotide sequence of the 5′ end. In some embodiments, a truncation mode for the 5′UTR truncation comprises, in the sequence direction from the 5′ end to the 3′ end, deleting a sequence of contiguous nucleotides from the 5′ end; and the truncation mode for the 5′UTR truncation comprises, in the sequence direction from the 3′ end to the 5′ end, deleting a sequence of contiguous nucleotides from the 3′ end. In some embodiments, the 5′UTR truncation also comprises the point mutation according to any of the preceding embodiments. In some embodiments, the 5′UTR truncation does not comprise the point mutation according to any of the preceding embodiments. In some embodiments, the 5′UTR truncation retains a sequence the length of which is at least 90%, 80%, 70%, 60%, 50%, 40%, 30%, 20%, 15%, 10%, 5%, or 1% of the length of a natural 5′UTR sequence.

In some embodiments, the aforementioned 5′UTR sequence derived from the ARHGAP15 gene comprises or is an ARHGAP15 5′UTR truncation. In some embodiments, a truncation mode for the truncation comprises, in the direction from the 5′ end to the 3′ end of the ARHGAP15 5′UTR, deleting a sequence of contiguous nucleotides from the 5′ end; and/or, in the sequence direction from the 3′ end to the 5′ end, deleting a sequence of contiguous nucleotides from the 3′ end.

In some embodiments, a truncation mode for the truncation comprises, in the direction from the 5′ end to the 3′ end of the ARHGAP15 5′UTR, deleting a sequence of contiguous nucleotides from the 5′ end, and keeping a nucleotide sequence of the 3′ end. In some embodiments, a truncation mode for the 5′UTR truncation comprises, in the direction from the 3′ end to the 5′ end of the ARHGAP15 5′UTR, deleting a sequence of contiguous nucleotides from the 3′ end, and keeping a nucleotide sequence of the 5′ end. In some embodiments, a truncation mode for the 5′UTR truncation is, in the direction from the 5′ end to the 3′ end of the ARHGAP15 5′UTR, to delete a sequence of contiguous nucleotides from the 5′ end; and, in the sequence direction from the 3′ end to the 5′ end, to delete a sequence of contiguous nucleotides from the 3′ end.

In some embodiments, the truncation retains a sequence the length of which is at least 90%, 80%, 70%, 60%, 50%, 40%, 30%, 20%, 15%, 10%, 5%, or 1% of the length of the natural ARHGAP15 5′UTR (SEQ ID NO: 45).

In some embodiments, the 5′UTR truncation comprises a sequence of at least 5 contiguous nucleotides from the nucleotide sequence set forth in SEQ ID NO: 46 or 79; in some embodiments, the 5′UTR truncation comprises a sequence of 5-62 contiguous nucleotides from the sequence set forth in SEQ ID NO: 46 or 79; in some embodiments, the 5′UTR truncation comprises a sequence of 7-62 contiguous nucleotides from the sequence set forth in SEQ ID NO: 46 or 79; in some embodiments, the 5′UTR truncation comprises a sequence of 7-59 contiguous nucleotides from the sequence set forth in SEQ ID NO: 46 or 79; illustratively, the 5′UTR truncation comprises a sequence of 5, 7, 8, 9, 10, 11, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 contiguous nucleotides from the sequence set forth in SEQ ID NO: 2.

In some embodiments, the nucleotide at the 5′ end of the 5′UTR truncation (i.e., the starting nucleotide of the 5′UTR truncation) is the nucleotide at any one of positions 1-57, according to natural counting, of the sequence set forth in SEQ ID NO: 46 or 79. In some embodiments, the nucleotide at the 5′ end of the 5′UTR truncation is the nucleotide at any one of positions 1-13 or 17-29, according to natural counting, of the sequence set forth in SEQ ID NO: 46 or 79, for example, the nucleotide at any one of positions 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, and 57.

In some embodiments, the 5′UTR truncation is 59 bp, 55 bp, 51 bp, 47 bp, 43 bp, 39 bp, 35 bp, 31 bp, 27 bp, 23 bp, 19 bp, 15 bp, 11 bp, or 7 bp in length.

In some embodiments, the ARHGAP15 5′UTR truncation also comprises the point mutation according to any of the preceding embodiments. In some embodiments, the ARHGAP15 5′UTR truncation also comprises a nucleotide sequence in which an AUG is mutated into a GUG. In some embodiments, the aforementioned 5′UTR derived from the ARHGAP15 gene comprises or is the nucleotide sequences set forth in SEQ ID NOs: 63-65 or nucleotide sequences having at least 80%, 85%, 90%, 95%, 96%, 97%, 98%, 99%, or 100% identity thereto.

In some embodiments, the ARHGAP15 5′UTR truncation does not comprise the point mutation according to any of the preceding embodiments. In some embodiments, the aforementioned 5′UTR derived from the ARHGAP15 gene comprises or is the nucleotide sequences set forth in SEQ ID NOs: 66-75, CUAUAAU, or nucleotide sequences having at least 80%, 85%, 90%, 95%, 96%, 97%, 98%, 99%, or 100% identity thereto.

In some embodiments, the aforementioned 5′UTR derived from the ARHGAP15 gene comprises or is sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto.

In some embodiments, the aforementioned 5′UTR derived from the HSPB1 gene comprises or is the nucleotide sequence set forth in SEQ ID NO: 47 or a nucleotide sequence having at least 80%, 85%, 90%, 95%, 96%, 97%, 98%, 99%, or 100% identity thereto.

In some embodiments, the aforementioned 5′UTR derived from the HBB gene comprises or is the nucleotide sequence set forth in SEQ ID NO: 48 or a nucleotide sequence having at least 80%, 85%, 90%, 95%, 96%, 97%, 98%, 99%, or 100% identity thereto.

In some embodiments, the aforementioned 5′UTR derived from the CCL13 gene comprises or is the nucleotide sequence set forth in SEQ ID NO: 49 or a nucleotide sequence having at least 80%, 85%, 90%, 95%, 96%, 97%, 98%, 99%, or 100% identity thereto.

In some embodiments, the 5′UTR in the RNA molecule described in the present disclosure comprises or is the nucleotide sequence set forth in any one of SEQ ID NOs: 45-49, 63-75, and 77-78 and CUAUAAU or a nucleotide sequence having at least 80%, 85%, 90%, 95%, 96%, 97%, 98%, 99%, or 100% identity thereto. Alternatively, the 5′UTR in the RNA molecule comprises sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto.

In some specific embodiments, the 5′UTR described in the RNA molecule according to any one of the above comprises or is the nucleotide sequence set forth in any one of SEQ ID NOs: 45-49, 63-75, and 77-78 and CUAUAAU. Alternatively, the 5′UTR in the RNA molecule comprises sequences corresponding to the sequences set forth in SEQ ID NOs: 41, 76, and 137-196 (replacing T in the sequences with U).

In some embodiments, the RNA molecule of the present disclosure also further comprises: (c) a 3′ untranslated region element (3′UTR).

In some embodiments, the open reading frame of the present disclosure is derived from a different gene than the 5′UTR and/or the 3′UTR. In some embodiments, the RNA molecule of the present disclosure comprises at least one open reading frame, at least one 5′UTR, or at least one 3′UTR. In some embodiments, the 5′UTR and 3′UTR in the RNA molecule of the present disclosure are from the same source or different sources, e.g., derived from the same gene or different genes. In some embodiments, the 5′UTR and 3′UTR in the RNA molecule of the present disclosure are derived from the same species or different species.

In some embodiments, the 3′UTR in the RNA molecule of the present disclosure is located downstream of the open reading frame. In some embodiments, the 3′UTR in the RNA molecule is located at the 3′ end of the open reading frame. In some embodiments, the 3′UTR in the RNA molecule of the present disclosure is selected from the group consisting of or is a 3′UTR derived from any one of the genes hemoglobin subunit beta (HBB), ARHGAP15, coronin 1A (CORO1A), hemopexin (HPX), and the like, or a derivative sequence thereof.

In some embodiments, the 3′UTR is derived from or is a 3′UTR sequence of the HBB or ARHGAP15 gene or a derivative sequence thereof.

In some embodiments, the aforementioned 3′UTR derived from the HBB gene comprises or is the nucleotide sequence set forth in SEQ ID NO: 50 or a nucleotide sequence having at least 80%, 85%, 90%, 95%, 96%, 97%, 98%, 99%, or 100% identity thereto.

In some embodiments, the aforementioned 3′UTR derived from the ARHGAP15 gene comprises or is the nucleotide sequence set forth in SEQ ID NO: 51 or a nucleotide sequence having at least 80%, 85%, 90%, 95%, 96%, 97%, 98%, 99%, or 100% identity thereto.

In some embodiments, the aforementioned 3′UTR derived from the CORO1A gene comprises or is the nucleotide sequence set forth in SEQ ID NO: 52 or a nucleotide sequence having at least 80%, 85%, 90%, 95%, 96%, 97%, 98%, 99%, or 100% identity thereto.

In some embodiments, the aforementioned 3′UTR derived from the HPX gene comprises or is the nucleotide sequence set forth in SEQ ID NO: 53 or a nucleotide sequence having at least 80%, 85%, 90%, 95%, 96%, 97%, 98%, 99%, or 100% identity thereto.

In some embodiments, the 3′UTR in the RNA molecule of the present disclosure comprises or is the nucleotide sequence set forth in any one of SEQ ID NOs: 50-53 or a nucleotide sequence having at least 80%, 85%, 90%, 95%, 96%, 97%, 98%, 99%, or 100% identity thereto.

In some specific embodiments, the 3′UTR comprises or is the nucleotide sequence set forth in SEQ ID NO: 50 or 51.

In some embodiments, the RNA molecule comprises a 5′UTR and a 3′UTR, wherein:
the 5′UTR is selected from the group consisting of or is a 5′UTR derived from any one of the genes ARHGAP (e.g., ARHGAP15), HSPB1, HBB, CCL13, and the like, or a derivative sequence thereof, and the 3′UTR is selected from the group consisting of or is a 3′UTR derived from any one of the genes HBB, ARHGAP15, CORO1A, HPX, and the like, or a derivative sequence thereof.

In some embodiments, the RNA molecule comprises a 5′UTR and a 3′UTR, the 5′UTR and the 3′UTR being selected from the group consisting of any one of the following:
the 5′UTR is derived from or is a 5′UTR of the ARHGAP15 gene or a derivative sequence thereof, and the 3′UTR is derived from or is a 3′UTR of the HBB gene or a derivative sequence thereof;
the 5′UTR is derived from or is a 5′UTR of the ARHGAP15 gene or a derivative sequence thereof, and the 3′UTR is derived from or is a 3′UTR of the ARHGAP15 gene or a derivative sequence thereof;
the 5′UTR is derived from or is a 5′UTR of the ARHGAP15 gene or a derivative sequence thereof, and the 3′UTR is derived from or is a 3′UTR of the CORO1A gene or a derivative sequence thereof;
the 5′UTR is derived from or is a 5′UTR of the ARHGAP15 gene or a derivative sequence thereof, and the 3′UTR is derived from or is a 3′UTR of the HPX gene or a derivative sequence thereof;
the 5′UTR is derived from or is a 5′UTR of the HBB gene or a derivative sequence thereof, and the 3′UTR is derived from or is a 3′UTR of the HBB gene or a derivative sequence thereof;
the 5′UTR is derived from or is a 5′UTR of the HBB gene or a derivative sequence thereof, and the 3′UTR is derived from or is a 3′UTR of the ARHGAP15 gene or a derivative sequence thereof;
the 5′UTR is derived from or is a 5′UTR of the HBB gene or a derivative sequence thereof, and the 3′UTR is derived from or is a 3′UTR of the CORO1A gene or a derivative sequence thereof;
the 5′UTR is derived from or is a 5′UTR of the HBB gene or a derivative sequence thereof, and the 3′UTR is derived from or is a 3′UTR of the HPX gene or a derivative sequence thereof;
the 5′UTR is derived from or is a 5′UTR of the HSPB1 gene or a derivative sequence thereof, and the 3′UTR is derived from or is a 3′UTR of the HBB gene or a derivative sequence thereof;
the 5′UTR is derived from or is a 5′UTR of the HSPB1 gene or a derivative sequence thereof, and the 3′UTR is derived from or is a 3′UTR of the ARHGAP15 gene or a derivative sequence thereof;
the 5′UTR is derived from or is a 5′UTR of the HSPB1 gene or a derivative sequence thereof, and the 3′UTR is derived from or is a 3′UTR of the CORO1A gene or a derivative sequence thereof;
the 5′UTR is derived from or is a 5′UTR of the HSPB1 gene or a derivative sequence thereof, and the 3′UTR is derived from or is a 3′UTR of the HPX gene or a derivative sequence thereof; or
the 5′UTR is derived from or is a 5′UTR of the CCL13 gene or a derivative sequence thereof, and the 3′UTR is derived from or is a 3′UTR of the HBB gene or a derivative sequence thereof;
the 5′UTR is derived from or is a 5′UTR of the CCL13 gene or a derivative sequence thereof, and the 3′UTR is derived from or is a 3′UTR of the ARHGAP15 gene or a derivative sequence thereof;
the 5′UTR is derived from or is a 5′UTR of the CCL13 gene or a derivative sequence thereof, and the 3′UTR is derived from or is a 3′UTR of the CORO1A gene or a derivative sequence thereof; or
the 5′UTR is derived from or is a 5′UTR of the CCL13 gene or a derivative sequence thereof, and the 3′UTR is derived from or is a 3′UTR of the HPX gene or a derivative sequence thereof.

In some embodiments, the RNA molecule of the present disclosure comprises a 5′UTR and a 3′UTR, the 5′UTR and the 3′UTR being selected from the group consisting of any one of the following:
the 5′UTR comprises or is the nucleotide sequence set forth in any one of SEQ ID NOs: 45-46, 63-75, and 77-78 and CUAUAAU or a nucleotide sequence having at least 80%, 85%, 90%, 95%, 96%, 97%, 98%, 99%, or 100% identity thereto, or the 5′UTR comprises sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto; and the 3′UTR comprises or is the nucleotide sequence set forth in SEQ ID NO: 50 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 45-46, 63-75, and 77-78 and CUAUAAU or a nucleotide sequence having at least 80%, 85%, 90%, 95%, 96%, 97%, 98%, 99%, or 100% identity thereto, or the 5′UTR comprises sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto; and the 3′UTR comprises or is the nucleotide sequence set forth in SEQ ID NO: 51 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 45-46, 63-75, and 77-78 and CUAUAAU or a nucleotide sequence having at least 80%, 85%, 90%, 95%, 96%, 97%, 98%, 99%, or 100% identity thereto, or the 5′UTR comprises sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto; and the 3′UTR comprises or is the nucleotide sequence set forth in SEQ ID NO: 52 or a nucleotide sequence having at least 80%, 85%, 90%, 95%, 96%, 97%, 98%, 99%, or 100% identity thereto;
the 5′UTR comprises or is the nucleotide sequence set forth in any one of SEQ ID NOs: 45-46, 63-75, and 77-78 and CUAUAAU or a nucleotide sequence having at least 80%, 85%, 90%, 95%, 96%, 97%, 98%, 99%, or 100% identity thereto, or the 5′UTR comprises sequences corresponding to the sequences set forth in SEQ ID NOs: 41, 76, and 137-196 (replacing T in the sequences with U) or nucleotide sequences having at least 80%, 85%, 90%, 95%, 96%, 97%, 98%, 99%, or 100% identity thereto; and the 3′UTR comprises or is the nucleotide sequence set forth in SEQ ID NO: 53 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 47 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 50 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 47 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 51 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 47 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 52 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 47 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 53 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 48 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 50 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 48 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 51 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 48 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 52 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 48 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 53 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 49 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 50 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 49 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 51 or a nucleotide sequence having at least 80%, 85%, 90%, 95%, 96%, 97%, 98%, 99%, or 100% identity thereto;
the 5′UTR comprises or is the nucleotide sequence set forth in SEQ ID NO: 49 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 52 or a nucleotide sequence having at least 80%, 85%, 90%, 95%, 96%, 97%, 98%, 99%, or 100% identity thereto; and
the 5′UTR comprises or is the nucleotide sequence set forth in SEQ ID NO: 49 or a nucleotide sequence having at least 80%, 85%, 90%, 95%, 96%, 97%, 98%, 99%, or 100% identity thereto, and the 3′UTR comprises or is the nucleotide sequence set forth in SEQ ID NO: 53 or a nucleotide sequence having at least 80%, 85%, 90%, 95%, 96%, 97%, 98%, 99%, or 100% identity thereto.

In some embodiments, the RNA molecule comprises a 5′UTR and a 3′UTR, wherein:
the 5′UTR comprises or is the nucleotide sequence set forth in any one of SEQ ID NOs: 45-46, 63-75, and 77-78 and CUAUAAU, or the 5′UTR comprises sequences corresponding to the sequences set forth in SEQ ID NOs: 41, 76, and 137-196 (replacing T in the sequences with U); and the 3′UTR is derived from or is a 3′UTR of any one of the genes HBB, ARHGAP15, CORO1A, and HPX gene or a derivative sequence thereof.

In some specific embodiments, the RNA molecule comprises a 5′UTR and a 3′UTR, wherein:
the 5′UTR comprises or is the nucleotide sequence set forth in any one of SEQ ID NOs: 45-46, 63-75, and 77-78 and CUAUAAU, or the 5′UTR comprises sequences corresponding to the sequences set forth in SEQ ID NOs: 41, 76, and 137-196 (replacing T in the sequences with U); and the 3′UTR comprises or is the nucleotide sequence set forth in any one of SEQ ID NOs: 50-53.

In some embodiments, the RNA molecule of the present disclosure also further comprises: (d) a polyadenylic acid (poly-A) tail.

In some embodiments, the poly-A tail in the RNA molecule is located downstream of the 3′UTR. In some embodiments, the poly-A tail in the RNA molecule is located at the 3′ end of the 3′UTR. In some embodiments, the poly-A tail is at the 3′ end of the RNA molecule. In some embodiments, the poly-A tail is at least about 50, 100, 150, 200, 300, 400, or 500 nucleotides in length.

In some embodiments, the poly-A tail includes, but is not limited to, HGH polyA, SV40polyA, BGH polyA, rbGlob polyA, or SV40late polyA.

In some specific embodiments, the poly-A tail comprises or is the nucleotide sequence set forth in SEQ ID NO: 56 or 135.

In some embodiments, the RNA molecule of the present disclosure also further comprises: (e) a 5′ cap structure (5′Cap).

In some embodiments, the 5′ cap structure in the RNA molecule is located upstream of the 5′UTR. In some embodiments, the 5′ cap structure in the RNA molecule is located at the 5′ end of the 5′UTR. In some embodiments, the 5′ cap structure is a cap structure known to those skilled in the art, such as Cap0 (methylation of the first nucleobase, e.g., m7GpppN), Cap1 (additional methylation of the ribose of a nucleotide adjacent to m7GpppN, e.g., m7G(5′)ppp(5′)(2′OMeA)pG), Cap2 (additional methylation of the ribose of the 3rd nucleotide downstream of m7GpppN), Cap3 (additional methylation of the ribose of the 3rd nucleotide downstream of m7GpppN), Cap4 (additional methylation of the ribose of the 4th nucleotide downstream of m7GpppN), ARCA (anti-reverse cap analog), modified ARCA (e.g., phosphorothioate-modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

In some embodiments, the 5′-cap structure (e.g., Cap0 or Cap1) is formed using chemical RNA synthesis or RNA in vitro transcription (co-transcriptional capping).

In some embodiments, the 5′-cap structure (e.g., Cap0 or Cap1) is formed via enzymatic capping using a capping enzyme (e.g., vaccinia virus capping enzyme and/or cap-dependent 2′-0 methyltransferase). In some embodiments, the 5′ cap structure (Cap0 or Cap1) is added using an immobilized capping enzyme. The capping method and means in WO2016/193226 are incorporated herein by reference in their entirety.

In some embodiments, the 5′ cap structure includes, but is not limited to, ARCA, 3′OMe-m7G(5′)ppp(5′)G, m7G(5′)ppp(5′)(2′OMeA)pU, m7Gppp(A2′O-MOE)pG, m7G(5′)ppp(5′)(2′OMeA)pG, m7G(5′)ppp(5′)(2′OMeG)pG, m7(3′OMeG)(5′)ppp(5′) (2′OMeG)pG, or m7(3′OMeG)(5′)ppp(5′)(2′OMeA)pG.

In some specific embodiments, the 5′ cap structure is m7G(5′)ppp(5′)(2′OMeA)pG. Other 5′ cap structures or cap structure analogs may also be used.

In some embodiments, in the 5′-to-3′ direction, the RNA molecule according to any one of the above comprises any one of i)-v):
i) a 5′UTR, and an open reading frame (ORF);
ii) an open reading frame (ORF), and a 3′UTR;
iii) a 5′UTR, an open reading frame (ORF), and a 3′UTR; and
iv) a 5′UTR, an open reading frame (ORF), a 3′UTR, and a poly-A tail;
v) a 5′ cap structure (5′Cap), a 5′UTR, an open reading frame (ORF), a 3′UTR, and a poly-A tail;
wherein the ORF is derived from a different gene than the 5′UTR and/or the 3′UTR.

In some embodiments, the 5′UTR in i) and iii) to v) is selected from the group consisting of a 5′UTR derived from any one of the genes ARHGAP, HSPB1, HBB, CCL13, and the like, or a derivative sequence thereof (e.g., a 5′UTR of ARHGAP15 or a derivative sequence thereof).

In some embodiments, the 3′UTR in ii) to v) is selected from the group consisting of a 3′UTR derived from any one of the genes HBB, ARHGAP15, CORO1A, HPX, and the like, or a derivative sequence thereof (e.g., a 3′UTR of HBB or ARHGAP15 or a derivative sequence thereof).

In some embodiments, the poly-A tail in iv) to v) comprises, for example, the nucleotide sequence set forth in SEQ ID NO: 56.

In some embodiments, the 5′ cap structure in v) includes, but is not limited to, Cap0, Cap1 (e.g., m7G(5′)ppp(5′)(2′OMeA)pG), Cap2, Cap3, Cap4, and ARCA.

In some embodiments, the 5′UTR in i) comprises or is the nucleotide sequence set forth in any one of SEQ ID NOs: 45-49, 63-75, and 77-78 and CUAUAAU; or the 5′UTR comprises sequences corresponding to the sequences set forth in SEQ ID NOs: 41, 76, and 137-196 (replacing T in the sequences with U).

In some embodiments, the 3′UTR in ii) comprises or is the nucleotide sequence set forth in any one of SEQ ID NOs: 50-53.

In some embodiments, the 5′UTR in iii) comprises or is the nucleotide sequence set forth in any one of SEQ ID NOs: 45-49, 63-75, and 77-78 and CUAUAAU, or the 5′UTR comprises sequences corresponding to the sequences set forth in SEQ ID NOs: 41, 76, and 137-196 (replacing T in the sequences with U); and the 3′UTR comprises or is the nucleotide sequence set forth in any one of SEQ ID NOs: 50-53.

In some embodiments, the 5′UTR in iv) comprises or is the nucleotide sequence set forth in any one of SEQ ID NOs: 45-49, 63-75, and 77-78 and CUAUAAU, or the 5′UTR comprises sequences corresponding to the sequences set forth in SEQ ID NOs: 41, 76, and 137-196 (replacing T in the sequences with U); the 3′UTR comprises or is the nucleotide sequence set forth in any one of SEQ ID NOs: 50-53, and the poly-A tail comprises or is the nucleotide sequence set forth in SEQ ID NO: 56.

In some embodiments, the 5′UTR in v) comprises or is the nucleotide sequence set forth in any one of SEQ ID NOs: 45-49, 63-75, and 77-78 and CUAUAAU, or the 5′UTR comprises sequences corresponding to the sequences set forth in SEQ ID NOs: 41, 76, and 137-196 (replacing T in the sequences with U); the 3′UTR comprises or is the nucleotide sequence set forth in any one of SEQ ID NOs: 50-53, the poly-A tail comprises or is the nucleotide sequence set forth in SEQ ID NO: 56, and the 5′ cap structure is m7G(5′)ppp(5′)(2′OMeA)pG.

In the present disclosure, in the RNA molecule according to any one of the above, the ORF comprises a nucleotide sequence encoding at least one polypeptide or protein. In some embodiments, the nucleotide sequence may be a codon-optimized nucleotide sequence.

In some embodiments, the polypeptide or protein encoded by the ORF is a fluorescent protein or a luciferase.

In some embodiments, the polypeptide or protein encoded by the ORF is a viral antigen. Illustratively, the viral antigen includes, but is not limited to, antigens of influenza viruses, coronaviruses, respiratory syncytial virus, human immunodeficiency viruses, herpes simplex virus, rabies virus, or EB virus, among others.

In some embodiments, the viral antigen is a coronavirus antigen. In some embodiments, the coronavirus is a human-infecting coronavirus, such as SARS-CoV-2 (COVID-19), SARS-CoV, HCoV-229E, HCoV-OC43, HCoV-NL63, HCoV-HKU1, or MERS-CoV. In some embodiments, the coronavirus is SARS-COV-2. In some embodiments, the coronavirus antigen is a structural protein. In some embodiments, the structural protein is selected from the group consisting of a spike protein (S protein), an envelope protein (E protein), a membrane protein (M protein), and a nucleocapsid protein (N protein). In some embodiments, the structural protein is a spike protein. In some embodiments, the spike protein is a SARS-COV-2 spike protein. In some embodiments, the SARS-COV-2 spike protein is selected from the group consisting of a spike protein of any one of the viral strains SARS-COV-2 (e.g., wild-type SARS-COV-2), SARS-COV-2 Alpha(B.1.1.7), SARS-COV-2 Beta(B.1.351), SARS-COV-2 Gamma(P.1), SARS-COV-2 Kappa(B.1.617.1), SARS-COV-2 Delta(B.1.617.2), SARS-COV-2 Omicron(B.1.1.529), SARS-COV-2 Omicron(BA.4), and the like. In some embodiments, the SARS-COV-2 spike protein comprises or is the amino acid sequence set forth in any one of SEQ ID NOs: 12, 14, 21, 23, 25, 80, and 136 or a sequence having at least 80%, 85%, 90%, 95%, 96%, 97%, 98%, 99%, or 100% amino acid identity thereto.

In some embodiments, the ORF comprises a codon-optimized nucleotide sequence comprising a sequence having at least 80%, 85%, 90%, 95%, 96%, 97%, 98%, 99%, or 100% identity to a wild-type nucleotide sequence encoding a SARS-COV-2 antigen (e.g., wild-type SARS-COV-2, SEQ ID NO: 82).

In some embodiments, the ORF encoding the polypeptide or protein comprises or is the nucleotide sequence set forth in any one of SEQ ID NOs: 54-55, 59-61, 82, and 130 or a nucleotide sequence having at least 80%, 85%, 90%, 95%, 96%, 97%, 98%, 99%, or 100% identity thereto.

In some embodiments, the ORF encodes an influenza virus antigen. In some embodiments, the influenza virus is selected from the group consisting of influenza A virus and influenza B virus; illustratively, the influenza virus is influenza A virus H1N1, influenza A virus H3N2, influenza A virus H3N8, influenza A virus H2N2, influenza A virus H5N1, influenza A virus H9N2, influenza A virus H7N7, influenza B virus/Victoria (e.g., influenza B/Washington/02/2019), influenza B virus/Yamagata (e.g., influenza B/Phuket/3073/2013), or the like. In some embodiments, the influenza virus antigen is a structural protein of an influenza virus, e.g., hemagglutinin (HA), neuraminidase (NA), M2 ion channel, matrix protein M1, or nucleoprotein (NP). In some specific embodiments, the influenza virus antigen is an HA protein of an influenza virus, e.g., an HA protein of influenza A virus H1N1, influenza A virus H3N2, influenza B virus/Victoria (e.g., influenza B/Washington/02/2019), or influenza B virus/Yamagata (e.g., influenza B/Phuket/3073/2013). In some specific embodiments, the influenza virus antigen is an NA protein of an influenza virus, e.g., an NA protein of influenza A virus H1N1, influenza A virus H3N2, influenza B virus Victoria (e.g., influenza B/Washington/02/2019), or influenza B virus Yamagata (e.g., influenza B/Phuket/3073/2013). In some embodiments, the HA protein comprises or is the amino acid sequence set forth in any one of SEQ ID NOs: 83-86 and 98-101 or a sequence having at least 80%, 85%, 90%, 95%, 96%, 97%, 98%, 99%, or 100% amino acid identity thereto.

In some embodiments, the ORF comprises a codon-optimized nucleotide sequence comprising a sequence having at least 80%, 85%, 90%, 95%, 96%, 97%, 98%, 99%, or 100% identity to a wild-type nucleotide sequence encoding an HA antigen.

In some embodiments, the ORF encoding the polypeptide or protein comprises or is the nucleotide sequence set forth in any one of SEQ ID NOs: 126-129 and 131-134 or a sequence having at least 80%, 85%, 90%, 95%, 96%, 97%, 98%, 99%, or 100% identity thereto.

In some embodiments, the present disclosure provides an RNA molecule comprising, in the 5′-to-3′ direction, a 5′UTR, an ORF, a 3′UTR, and a poly-A tail in sequence. In some embodiments, the 5′UTR comprises or is the nucleotide sequence set forth in any one of SEQ ID NOs: 45-49, 45-49, 63-75, and 77-78 and CUAUAAU, or the 5′UTR comprises sequences corresponding to the sequences set forth in SEQ ID NOs: 41, 76, and 137-196 (replacing T in the sequences with U); the ORF comprises or is the nucleotide sequences set forth in SEQ ID NOs: 54-55, 59-61, 82, and 126-134, the 3′UTR comprises or is the nucleotide sequence set forth in any one of SEQ ID NOs: 50-53, and the poly-A tail comprises or is the nucleotide sequence set forth in SEQ ID NO: 56 or 135. In some specific embodiments, the RNA molecule comprises the nucleotide sequences set forth in SEQ ID NOs: 57-58 and 106-125.

In the present disclosure, including any embodiment thereof, the RNA molecule may be an mRNA.

The present disclosure uses a 5′UTR sequence derived from human Rho GTPase activating protein (ARHGAP) for recombinant expression of a target gene (e.g., SARS-CoV-2 viral protein) for the first time, and screens, designs, and successfully verifies that a 5′UTR derived from ARHGAP15 and an engineered and modified 5′UTR thereof can initiate efficient expression of different target genes. Rho GTPase activating protein 15 (ARHGAP15), a Rac1-specific GTPase activating protein (GAP), belongs to the ARHGAP family, and it is a major negative regulator of Rho family GTPase activity. The present disclosure provides more 5′UTR and 3′UTR options and combinations with excellent expression efficiency. Additionally, The present disclosure provides mRNA vaccines with the potential to efficiently prevent infection with SARS-CoV-2 and influenza viruses, particularly against various variants thereof.

### Polynucleotide

The present disclosure also provides an isolated polynucleotide encoding one or more polypeptides or proteins, wherein the polypeptides or proteins are coronavirus antigens. In some embodiments, the coronavirus is SARS-COV-2. In some embodiments, the coronavirus antigen is a structural protein. In some embodiments, the structural protein is selected from the group consisting of a spike protein (S protein), an envelope protein (E protein), a membrane protein (M protein), and a nucleocapsid protein (N protein). In some embodiments, the structural protein is a spike protein. In some embodiments, the spike protein is a SARS-COV-2 spike protein. In some embodiments, the SARS-COV-2 spike protein is selected from the group consisting of a spike protein of any one of the viral strains SARS-COV-2 (e.g., wild-type SARS-COV-2), SARS-COV-2 Alpha(B.1.1.7), SARS-COV-2 Beta(B.1.351), SARS-COV-2 Gamma(P.1), SARS-COV-2 Kappa(B.1.617.1), SARS-COV-2 Delta(B.1.617.2), SARS-COV-2 Omicron(B.1.1.529), SARS-COV-2 Omicron(BA.4), and the like. In some embodiments, the SARS-COV-2 spike protein comprises or is the amino acid sequence set forth in any one of SEQ ID NOs: 12, 14, 21, 23, 25, 80, and 136 or a sequence having at least 80%, 85%, 90%, 95%, 96%, 97%, 98%, 99%, or 100% amino acid identity thereto.

In some embodiments, the ORF encodes an influenza virus antigen. In some embodiments, the influenza virus is selected from the group consisting of influenza A virus and influenza B virus; illustratively, the influenza virus is influenza A virus H1N1, influenza A virus H3N2, influenza A virus H3N8, influenza A virus H2N2, influenza A virus H5N1, influenza A virus H9N2, influenza A virus H7N7, influenza B virus/Victoria (e.g., influenza B/Washington/02/2019), influenza B virus/Yamagata (e.g., influenza B/Phuket/3073/2013), or the like. In some embodiments, the influenza virus antigen is a structural protein of an influenza virus, e.g., hemagglutinin (HA), neuraminidase (NA), M2 ion channel, matrix protein M1, or nucleoprotein (NP). In some specific embodiments, the influenza virus antigen is an HA protein of an influenza virus, e.g., an HA protein of influenza A virus H1N1, influenza A virus H3N2, influenza B virus/Victoria (e.g., influenza B/Washington/02/2019), or influenza B virus/Yamagata (e.g., influenza B/Phuket/3073/2013). In some specific embodiments, the influenza virus antigen is an NA protein of an influenza virus, e.g., an NA protein of influenza A virus H1N1, influenza A virus H3N2, influenza B virus Victoria (e.g., influenza B/Washington/02/2019), or influenza B virus Yamagata (e.g., influenza B/Phuket/3073/2013). In some embodiments, the influenza virus HA protein comprises or is the amino acid sequence set forth in any one of SEQ ID NOs: 83-86 and 98-101 or a sequence having at least 80%, 85%, 90%, 95%, 96%, 97%, 98%, 99%, or 100% amino acid identity thereto.

In some embodiments, the ORF comprises a codon-optimized nucleotide sequence comprising a sequence having at least 80%, 85%, 90%, 95%, 96%, 97%, 98%, 99%, or 100% identity to a wild-type nucleotide sequence encoding a SARS-COV-2 antigen (e.g., wild-type SARS-COV-2 mRNA, SEQ ID NO: 82).

In some embodiments, the isolated polynucleotide comprises or is the nucleotide sequences set forth in SEQ ID NOs: 54-55, 59-61, 82, and 126-134 or nucleotide sequences having at least 80%, 85%, 90%, 95%, 96%, 97%, 98%, 99%, or 100% identity thereto.

In some embodiments, the polynucleotide is an RNA, e.g., an mRNA. In some embodiments, the polynucleotide is an ORF region of an mRNA.

In some embodiments, the present disclosure provides use of any one of the aforementioned nucleic acid constructs or RNA molecules in any one of the following: (1) preparing a vaccine; (2) encoding a viral antigen in a subject or in vitro; (3) preparing a medicament encoding a viral antigen in a subject or in vitro; and (4) preparing a medicament.

In some embodiments, the nucleic acid construct or RNA molecule in the present disclosure encodes a target protein, wherein the open reading frame (ORF) has enhanced expression of the target protein

### Modification

To further improve the stability of protein expression of the RNA or polynucleotide molecule of the present disclosure, the RNA or polynucleotide molecule may further comprise one or more modifications (including chemical modifications), such as backbone modifications, sugar modifications, base modifications, and/or lipid modifications. In some embodiments, the RNA or polynucleotide molecule is uniformly modified to a particular modification (e.g., complete modification throughout the sequence). For example, the RNA may be uniformly modified with pseudouridines such that each U in the sequence is a pseudouridine.

Backbone modifications in connection with the present disclosure refer to chemical modifications of the phosphate of the backbone of the nucleotides that the RNA or polynucleotide molecule of the present disclosure comprises. In some embodiments, the backbone modifications include, but are not limited to, complete substitution of unmodified phosphate moieties in the backbone with modified phosphates; for example, backbone phosphate groups may be modified by substituting one or more oxygen atoms with a different substituent. In some embodiments, the modified phosphates include, but are not limited to, phosphorothioates, phosphoroselenates, boranophosphates, boranophosphates, hydrogen phosphonates, phosphoramidates, alkyl or aryl phosphonates, and phosphotriesters.

Sugar modifications in connection with the present disclosure refer to chemical modifications of the sugar of the nucleotides that the RNA or polynucleotide molecule of the present disclosure comprises. In some embodiments, the sugar modifications include, but are not limited to, modifications or replacements of the 2′ hydroxy (OH) of the RNA molecule with many different “oxy” or “deoxy” substituents. In some embodiments, the “oxy” modifications include, but are not limited to, substitution modifications with alkoxy, aryloxy, polyethylene glycol (PEG), and the like. In some embodiments, “deoxy” modifications include, but are not limited to, hydrogen and amino (e.g., NH2, alkylamino, dialkylamino, heterocyclyl, arylamino, diarylamino, heteroarylamino, diheteroarylamino, or amino acid) modifications.

Base modifications in connection with the present disclosure refer to chemical modifications of the base moiety of the nucleotides that the RNA or polynucleotide molecule of the present disclosure comprises. In some embodiments, the base modifications include modifications to adenine, guanine, cytosine, and uracil in nucleotides. For example, the nucleosides and nucleotides described herein may be chemically modified on the surface of the major groove. In some embodiments, chemical modifications to the major groove may include amino, thiol, alkyl, or halogen groups. In some embodiments, the base modifications include, but are not limited to, modifications with pseudouridine, 1-methyl-pseudouridine, 5-azacytidine, 5-methylcytosine-5′-triphosphate, or 2-methoxyadenine. In some embodiments, the base modifications are pseudouridine modifications. In some specific embodiments, the RNA may be uniformly modified with pseudouridines such that each U in the sequence is a pseudouridine.

Lipid modifications in connection with the present disclosure mean that the RNA or polynucleotide molecule of the present disclosure comprises lipid modifications. In some embodiments, the lipid modifications include, but are not limited to, covalent attachment of the RNA or polynucleotide molecule of the present disclosure to at least one adapter, and covalent attachment of the corresponding adapter to at least one lipid. In some embodiments, the lipid modifications include, but are not limited to, covalent attachment of the RNA or polynucleotide molecule of the present disclosure to at least one lipid (no adapter).

### UTRs

UTRs (5′UTR and/or 3′UTR) may be provided as a flanking region to the nucleic acid construct, RNA, or polynucleotide molecule of the present disclosure. UTRs may be homologous or heterologous with coding regions in the nucleic acid construct, RNA, or polynucleotide molecule of the present disclosure. The flanking region may comprise one or more 5′UTRs and/or 3′UTRs, and the UTRs may be identical or different sequences. Any portion of the flanking region may be codon-optimized. Before and/or after codon optimization, any portion of the flanking region may independently comprise one or more different structural or chemical modifications.

To alter one or more properties of the nucleic acid construct, RNA, or polynucleotide of the present disclosure, UTRs heterologous with the ORF of the present disclosure are introduced or engineering-synthesized into the nucleic acid construct, RNA, or polynucleotide of the present disclosure. The recombinant nucleic acid construct, RNA, or polynucleotide is then administered to a cell, tissue, or organism, and the results, such as protein levels, localization, and/or half-life, are measured to assess the beneficial effects of the heterologous UTRs on the RNA or polynucleotide of the present disclosure. In some embodiments, the UTRs include a wild UTR or a variant thereof, the UTR variant comprising the addition or removal of one or more nucleotides, including A, T, C, or G, to or from an end. In some embodiments, the UTR variant also comprises codon optimization or modification performed in any way. In some embodiments, the UTR variant also comprises the derivative sequence according to any embodiment of the present disclosure; for example, on the basis of a natural UTR sequence, an ATG is point-mutated into a GTG in order to inhibit the ATG within the UTR from initiating translation.

### Vector

The present disclosure also provides a vector comprising the nucleic acid construct, RNA, or polynucleotide according to any one of the foregoing. The nucleic acid construct, RNA, or polynucleotide may be present in the vector and/or may be part of the vector, the vector being, for example, a plasmid, cosmid, YAC, or viral vector. The vector may be an expression vector, i.e., a vector capable of providing for the expression of a polypeptide encoded by the nucleic acid construct, RNA, or polynucleotide. The expression vector generally comprises at least one of the nucleic acids of the present disclosure, which is operatively linked to one or more suitable expression regulatory elements (e.g., promoters and terminators). The selection of the elements and their sequences for expression in a particular host is within the knowledge of those skilled in the art. Regulatory elements and other elements useful or necessary for expression of the encoded polypeptide of the present disclosure include, for example, promoters, terminators, selection labels, leader sequences, and reporter genes.

The nucleic acid construct of the present disclosure may be prepared or obtained by known means (e.g., by automatic DNA synthesis and/or recombinant DNA technology) based on nucleotide sequence information of the present disclosure, and/or may be isolated from a suitable natural source.

In some embodiments, the vector of the present disclosure also comprises a promoter; for example, the promoter is at the 5′ end of the 5′UTR of the nucleic acid construct, and for example, the promoter is a T7 promoter, a T7 lac promoter, a Tac promoter, a Lac promoter, or a Trp promoter.

In some specific embodiments, the T7 promoter comprises or is the nucleotide sequence set forth in SEQ ID NO: 17.

### Host Cell

The present disclosure also provides a host cell comprising the nucleic acid construct, RNA, or polynucleotide according to any one of the foregoing. In some embodiments, the cell is capable of expressing one or more polypeptides encoded by the nucleic acid construct, RNA, or polynucleotide of the present disclosure. In some embodiments, the host cell is a bacterial cell, a fungal cell, or a mammalian cell.

Bacterial cells include, e.g., cells of gram-negative bacterial strains (e.g., Escherichia coli strains, Proteus strains, and Pseudomonas strains), and gram-positive bacterial strains (e.g., Bacillus strains, Streptomyces strains, Staphylococcus strains, and Lactococcus strains).

Fungal cells include, e.g., cells of the species Trichoderma, Neurospora, and Aspergillus; or cells of the species Saccharomyces (e.g., Saccharomyces cerevisiae), Schizosaccharomyces (e.g., Schizosaccharomyces pombe), Pichia (Pichia pastoris and Pichia methanolica), and HansenuLa.

Mammalian cells include, for example, HEK293 cells, CHO cells, BHK cells, HeLa cells, COS cells, and the like.

However, amphibian cells, insect cells, plant cells, and any other cells used in the art for expressing heterologous proteins may also be used in the present disclosure.

### Production or Preparation Method

The present disclosure provides a method for preparing the nucleic acid construct, RNA, or polynucleotide of the present disclosure, and a method for preparing the encoded polypeptide thereof.

Methods and reagents for preparation and production of the nucleic acid construct, RNA, or polynucleotide and the encoded polypeptide thereof, e.g., specific suitable vectors, transformation or transfection methods, selection labels, methods for inducing protein expression, and culture conditions, are known in the art. Similarly, protein isolation and purification techniques suitable for use in the method for manufacturing the encoded polypeptide of the present disclosure are well known to those skilled in the art.

In some embodiments, the method for preparing the nucleic acid construct, RNA, or polynucleotide comprises culturing the aforementioned host cell and collecting the produced nucleic acid construct, RNA, or polynucleotide from the culture. The nucleic acid construct, RNA, or polynucleotide of the present disclosure and the encoded polypeptide thereof may also be obtained by other production methods known in the art, such as chemical synthesis, including solid-phase, or liquid-phase synthesis.

In some embodiments, a method for preparing an RNA molecule comprises: preparing a nucleic acid construct or a vector, and then using the nucleic acid construct or vector to perform reverse transcription to obtain the RNA molecule. In some specific embodiments, the method also comprises adding a 5′Cap to the 5′ end of the RNA molecule.

### Vaccine

The present disclosure also provides a vaccine comprising the nucleic acid construct according to any one of the foregoing, the RNA according to any one of the foregoing, and/or the polynucleotide according to any one of the foregoing. In some embodiments, the nucleic acid construct, RNA, or polynucleotide encodes one or more antigens of one or more viral strains. The vaccine provided by the present disclosure may be a monovalent vaccine, a multivalent vaccine, or a combination vaccine.

### Monovalent Vaccine

The present disclosure provides a monovalent vaccine comprising encoding an antigen of an organism. In some embodiments, the monovalent vaccine comprises encoding one antigen of one viral strain.

### Multivalent/Combination Vaccine

In some embodiments, the vaccine may comprise a nucleic acid construct, RNA, or polynucleotide molecule or multiple nucleic acid constructs, RNAs, or polynucleotide molecules encoding two or more antigens of the same species or different species. In some embodiments, the vaccine comprises an RNA or multiple RNAs encoding two or more antigens of the same viral strain or different viral strains. In some embodiments, the RNA may encode 1, 2, 3, 4, 5, 6, 7, 8, 9, 10, 11, 12, or more viral antigens.

In some embodiments, in the monovalent vaccine and the multivalent/combination vaccine described above, the antigen is a coronavirus antigen, the coronavirus being, for example, SARS-CoV-2 (COVID-19), SARS-CoV, HCoV-229E, HCoV-OC43, HCoV-NL63, HCoV-HKU1, or MERS-CoV. In some embodiments, the coronavirus antigen is a structural protein, e.g., selected from the group consisting of a spike protein (S protein), an envelope protein (E protein), a membrane protein (M protein), and a nucleocapsid protein (N protein). In some embodiments, the structural protein is a spike protein, e.g., a SARS-COV-2 spike protein. In some embodiments, the SARS-COV-2 spike protein is selected from the group consisting of a spike protein of any one of the viral strains SARS-COV-2 (e.g., wild-type SARS-COV-2 mRNA), SARS-COV-2 Alpha(B.1.1.7), SARS-COV-2 Beta(B.1.351), SARS-COV-2 Gamma(P.1), SARS-COV-2 Kappa(B.1.617.1), SARS-COV-2 Delta(B.1.617.2), SARS-COV-2 Omicron(B.1.1.529), SARS-COV-2 Omicron(BA.4), and the like. In some embodiments, the SARS-COV-2 spike protein comprises or is the amino acid sequence set forth in any one of SEQ ID NOs: 12, 14, 21, 23, 25, 80, and 136 or a sequence having at least 80%, 85%, 90%, 95%, 96%, 97%, 98%, 99%, or 100% amino acid identity thereto. In some embodiments, a DNA sequence encoding the SARS-COV-2 spike protein comprises or is the nucleotide sequence set forth in any one of SEQ ID NOs: 13, 15, 20, 22, 24, 81, and 97 or a nucleotide sequence having at least 80%, 85%, 90%, 95%, 96%, 97%, 98%, 99%, or 100% identity thereto. In some embodiments, an RNA sequence encoding the SARS-COV-2 spike protein comprises or is the nucleotide sequences set forth in SEQ ID NOs: 54-55, 59-61, 82, and 130 or nucleotide sequences having at least 80%, 85%, 90%, 95%, 96%, 97%, 98%, 99%, or 100% identity thereto.

In some embodiments, the ORF encodes an influenza virus antigen. In some embodiments, the influenza virus is selected from the group consisting of influenza A virus and influenza B virus; illustratively, the influenza virus is influenza A virus H1N1, influenza A virus H3N2, influenza A virus H3N8, influenza A virus H2N2, influenza A virus H5N1, influenza A virus H9N2, influenza A virus H7N7, influenza B virus/Victoria (e.g., influenza B/Washington/02/2019), influenza B virus/Yamagata (e.g., influenza B/Phuket/3073/2013), or the like. In some embodiments, the influenza virus antigen is a structural protein of an influenza virus, e.g., hemagglutinin (HA), neuraminidase (NA), M2 ion channel, matrix protein M1, or nucleoprotein (NP). In some specific embodiments, the influenza virus antigen is an HA protein of an influenza virus, e.g., an HA protein of influenza A virus H1N1, influenza A virus H3N2, influenza B virus/Victoria (e.g., influenza B/Washington/02/2019), or influenza B virus/Yamagata (e.g., influenza B/Phuket/3073/2013). In some embodiments, the HA protein comprises or is the amino acid sequence set forth in any one of SEQ ID NOs: 83-86 and 98-101 or a sequence having at least 80%, 85%, 90%, 95%, 96%, 97%, 98%, 99%, or 100% amino acid identity thereto. In some embodiments, a DNA sequence encoding the HA protein comprises or is the nucleotide sequence set forth in any one of SEQ ID NOs: 87-90, 93-97, and 102-105 or a nucleotide sequence having at least 80%, 85%, 90%, 95%, 96%, 97%, 98%, 99%, or 100% identity thereto. In some embodiments, an RNA sequence encoding the HA protein comprises or is the nucleotide sequence set forth in any one of SEQ ID NOs: 126-129 and 131-134 or a nucleotide sequence having at least 80%, 85%, 90%, 95%, 96%, 97%, 98%, 99%, or 100% identity thereto.

In some embodiments, two or more different RNAs (e.g., mRNAs) may be formulated in the same lipid nanoparticle. In other embodiments, two or more different RNAs may be formulated separately in separate lipid nanoparticles, and then the lipid nanoparticles may be combined and administered as a single vaccine composition (e.g., comprising multiple RNAs encoding multiple antigens), or may be administered separately.

The present disclosure also provides a multivalent/combination vaccine comprising an RNA encoding one or more coronaviruses or antigens of one or more different organisms. That is, the vaccine of the present disclosure may be a multivalent/combination vaccine that targets one or more antigens of the same viral strain/species, or one or more antigens of different viral strains/species.

### Delivery System

The present disclosure also provides a delivery system comprising the nucleic acid construct according to any one of the foregoing, or the RNA molecule according to any one of the foregoing, wherein the delivery vehicle is a cationic lipid delivery particle. In some embodiments, the particle is a nanoparticle. In some embodiments, the delivery vehicle is a lipid nanoparticle. The RNA molecule in the present disclosure may be delivered into cells and/or in vivo using any type of lipid nanoparticle in the art; illustratively, lipid nanoparticles include, but are not limited to, lipid particles disclosed in WO2017075531, WO2018081480A1, WO2017049245A2, WO2017099823A1, WO2022245888A1, WO2022150717A1, CN101291653A, CN102119217A, WO2011000107A1, and CN107028886A, which are incorporated herein by reference in their entirety.

### Pharmaceutical Composition

The present disclosure also provides a pharmaceutical composition comprising the nucleic acid construct according to any one of the foregoing, the polynucleotide according to any one of the foregoing, the vaccine according to any one of the foregoing, the vector according to any one of the foregoing, and/or the delivery vehicle according to any one of the foregoing, and a pharmaceutically acceptable carrier, diluent, or excipient; specifically, the pharmaceutical composition is a solid formulation, an injection, a topical formulation, a spray, a liquid formulation, or a composite formulation.

### Product or Kit

The present disclosure provides a product or kit comprising the nucleic acid construct according to any one of the foregoing, the polynucleotide according to any one of the foregoing, the vaccine according to any one of the foregoing, the vector according to any one of the foregoing, the delivery vehicle according to any one of the foregoing, and/or the pharmaceutical composition according to any one of the foregoing. The kit may be used to provide relevant detection or diagnostic use.

### Method for Treating and/or Preventing Disease and Pharmaceutical Use

The present disclosure also provides use of administering to a subject in need thereof a therapeutically and/or prophylactically effective amount of the nucleic acid construct according to any one of the foregoing, the polynucleotide according to any one of the foregoing, the vaccine according to any one of the foregoing, the vector according to any one of the foregoing, the delivery vehicle according to any one of the foregoing, the pharmaceutical composition according to any one of the foregoing, and/or the product or kit according to any one of the foregoing in the preparation of a medicament for treating and/or preventing a disease. In some embodiments, the disease comprises a viral infectious disease or a respiratory disease associated with viral infection. In some embodiments, the virus is a coronavirus. In some embodiments, the coronavirus is a human-infecting coronavirus, such as SARS-CoV-2 (COVID-19), SARS-CoV, HCoV-229E, HCoV-OC43, HCoV-NL63, HCoV-HKU1, or MERS-CoV. In some embodiments, the coronavirus is SARS-CoV-2. In some embodiments, the virus is an influenza virus. In some embodiments, the coronavirus is an influenza virus that infects humans, such as influenza A virus or influenza B virus. Illustratively, the influenza virus is influenza A virus H1N1, influenza A virus H3N2, influenza B virus/Victoria (e.g., influenza B/Washington/02/2019), influenza B virus/Yamagata (e.g., influenza B/Phuket/3073/2013), or the like. In some embodiments, the respiratory disease associated with viral infection (e.g., respiratory disease associated with SARS-CoV-2 infection) includes simple infection, such as fever, cough, and sore throat, headache, rhinitis, and the like, pneumonia, acute respiratory infection, severe acute respiratory infection (SARI), hypoxemic respiratory failure and acute respiratory distress syndrome, sepsis and septic shock, severe acute respiratory syndrome (SARS), and the like.

The present disclosure also provides a method for treating and/or preventing a disease, the method comprising administering to a subject in need thereof a therapeutically and/or prophylactically effective amount of the nucleic acid construct according to any one of the foregoing, the polynucleotide according to any one of the foregoing, the vaccine according to any one of the foregoing, the vector according to any one of the foregoing, the delivery vehicle according to any one of the foregoing, the pharmaceutical composition according to any one of the foregoing, and/or the product or kit according to any one of the foregoing. In some embodiments, the disease comprises a viral infectious disease or a respiratory disease associated with viral infection. In some embodiments, the virus is a coronavirus. In some embodiments, the coronavirus is a human-infecting coronavirus, such as SARS-CoV-2 (COVID-19), SARS-CoV, HCoV-229E, HCoV-OC43, HCoV-NL63, HCoV-HKU1, or MERS-CoV. In some embodiments, the coronavirus is SARS-CoV-2. In some embodiments, the virus is an influenza virus. In some embodiments, the coronavirus is an influenza virus that infects humans, such as influenza A virus or influenza B virus. Illustratively, the influenza virus is influenza A virus H1N1, influenza A virus H3N2, influenza B virus/Victoria (e.g., influenza B/Washington/02/2019), influenza B virus/Yamagata (e.g., influenza B/Phuket/3073/2013), or the like. In some embodiments, the respiratory disease associated with viral infection (e.g., respiratory disease associated with SARS-CoV-2 infection) includes simple infection, such as fever, cough, and sore throat, headache, rhinitis, and the like, pneumonia, acute respiratory infection, severe acute respiratory infection (SARI), hypoxemic respiratory failure and acute respiratory distress syndrome, sepsis and septic shock, severe acute respiratory syndrome (SARS), and the like.

The present disclosure also provides a method comprising administering to a subject in need thereof an effective amount of the nucleic acid construct according to any one of the foregoing, the polynucleotide according to any one of the foregoing, the vaccine according to any one of the foregoing, the vector according to any one of the foregoing, the delivery vehicle according to any one of the foregoing, the pharmaceutical composition according to any one of the foregoing, and/or the product or kit according to any one of the foregoing, which is capable of inducing in the subject a neutralizing antibody reaction and/or a T cell immune response, e.g., a neutralizing antibody reaction against a viral antigen, e.g., a CD4+ and/or CD8+ T cell immune response against a viral antigen. The antigen-antibody titer of the subject increases after vaccination relative to the antigen-antibody titer of a subject vaccinated with a prophylactically effective dose of a conventional vaccine against the antigen. In some embodiments, the virus is a coronavirus. In some embodiments, the coronavirus is a human-infecting coronavirus, such as SARS-CoV-2 (COVID-19), SARS-CoV, HCoV-229E, HCoV-OC43, HCoV-NL63, HCoV-HKU1, or MERS-CoV. In some embodiments, the coronavirus is SARS-CoV-2. In some embodiments, the virus is an influenza virus. In some embodiments, the coronavirus is an influenza virus that infects humans, such as influenza A virus or influenza B virus. Illustratively, the influenza virus is influenza A virus H1N1, influenza A virus H3N2, influenza B virus/Victoria (e.g., influenza B/Washington/02/2019), influenza B virus/Yamagata (e.g., influenza B/Phuket/3073/2013), or the like.

In some embodiments, the subject is immunized. In some embodiments, the subject has a lung disease. In some embodiments, the subject is 5 years old or younger, or 65 years old or older.

In some embodiments, the method comprises administering to the subject at least one, two, three, four, and more doses of the nucleic acid construct according to any one of the foregoing, the polynucleotide according to any one of the foregoing, the vaccine according to any one of the foregoing, the vector according to any one of the foregoing, the delivery vehicle according to any one of the foregoing, the pharmaceutical composition according to any one of the foregoing, or the product or kit according to any one of the foregoing.

In some embodiments, a detectable level of a viral antigen (such as a coronavirus antigen) is produced in the serum of the subject 1-72 hours after administration of the nucleic acid construct according to any one of the foregoing, the polynucleotide according to any one of the foregoing, the vaccine according to any one of the foregoing, the vector according to any one of the foregoing, the delivery vehicle according to any one of the foregoing, the pharmaceutical composition according to any one of the foregoing, or the product or kit according to any one of the foregoing. In some embodiments, a neutralizing antibody titer of at least 100 NU/mL, 200 NU/mL, 300 NU/mL, 400 NU/mL, 500 NU/mL, 600 NU/mL, 700 NU/mL, 800 NU/mL, 900 NU/mL, or 1000 NU/mL is produced in the serum of the subject 1-72 hours after administration.

In some embodiments, the antibody titer produced in the subject is increased by at least 1 log relative to a control. For example, the antibody titer produced in the subject may be increased by at least 2, 3, 4, 5, 6, 7, 8, 9, or 10 log relative to the control.

In some embodiments, the antibody titer produced in the subject is increased at least 2-fold relative to the control. For example, the antibody titer produced in the subject is increased at least 3, 4, 5, 6, 7, 8, 9, or 10-fold relative to the control. In some examples, a geometric mean is the nth power of the product of n numbers and is generally used to describe proportional growth. In some embodiments, the geometric mean is used to characterize the antibody titer produced in the subject. In some embodiments, the control may be a subject who has not been vaccinated, or a subject who has been administered a live attenuated virus vaccine, an inactivated virus vaccine, or a protein subunit vaccine.

### Vaccine Efficacy

In some embodiments, an antigen-specific immune response is characterized by measuring an anti-(corona)virus antigen antibody titer produced in a subject who has undergone the administration of the preceding embodiments. An antibody titer is a measurement of the amount of antibodies within a subject, e.g., antibodies specific to a particular antigen or epitope of an antigen. Antibody titers are typically expressed as the reciprocal of the maximum dilution that provides a positive result. Enzyme-linked immunosorbent assay (ELISA) is a common assay for determining antibody titers.

In some embodiments, the antibody titer is used to assess whether a subject has been infected or to determine whether the immunization is required. In some embodiments, the antibody titer is used to determine the intensity of the autoimmune response, to determine whether the boost immunization is required, to determine whether a previous vaccine dose is effective, and to identify any recent or previous infections. According to the present disclosure, the antibody titer can be used to determine the intensity of an immune response in a subject induced by an immune composition (e.g., an RNA vaccine).

In some embodiments, the antibody titer against a virus (coronavirus) antigen produced in the subject is increased by at least 1 log relative to the control. For example, the antibody titer produced in the subject may be increased by at least 2, 3, 4, 5, 6, 7, 8, 9, or 10 log relative to the control.

In some embodiments, the antibody titer produced in the subject is increased at least 2-fold relative to the control. For example, the antibody titer produced in the subject is increased at least 3, 4, 5, 6, 7, 8, 9, or 10-fold relative to the control.

In some embodiments, the antigen-specific immune response is measured by the ratio of the neutralizing antibody titer in serum to the geometric mean titer (GMT) of the coronavirus, referred to as the geometric mean ratio (GMR). The geometric mean titer (GMT) is the mean antibody titer in a group of subjects calculated by multiplying all values and taking the n-th root, where n is the number of subjects with available data.

In some embodiments, the control may be a subject who has not been vaccinated, or a subject who has been administered a live attenuated virus vaccine, an inactivated virus vaccine, or a protein subunit vaccine.

In some embodiments, the vaccine efficacy can be assessed using standard analytical methods (see, e.g., Weinberg et al., J. Infect. Dis., Jun. 1, 2010; 201 (11):1607-10). For example, the vaccine efficacy can be measured in a double-blind, randomized, controlled clinical trial. The vaccine efficacy may be expressed as a proportional reduction in the attack rates (ARs) of unvaccinated study cohort (ARU) and vaccinated study cohort (ARV) and can be calculated from the relative risk (RR) of a disease in the vaccinated group using the following equation.

$$Efficacy = ( A R U - A RV ) / ARU \times 100 ; and$$

$$Efficacy = ( 1 - RR ) \times 100$$

In some other embodiments, the vaccine effectiveness can be assessed using standard analytical methods (see, e.g., Weinberg et al., J. Infect. Dis., Jun. 1, 2010; 201 (11):1607-10). The vaccine effectiveness is an assessment of how a vaccine (which may have been demonstrated to have high vaccine efficacy) reduces a disease in a population. This measure allows to assess the net balance between advantageous and disadvantageous effects of vaccination programs but not the vaccine in a natural condition rather than in a controlled clinical trial. The vaccine effectiveness is proportional to the vaccine efficacy, but is also affected by the degree of immunity of the target population, as well as other non-vaccine related factors such as hospitalization, outpatient visits, or costs. For example, a retrospective case-control analysis can be conducted, i.e., comparing the vaccination rates in a group of infected subjects with an appropriate control group. The vaccine effectiveness can be expressed as a ratio difference, i.e., the probability of infection after vaccination (odds ratio, OR).

$$Effectiveness = ( 1 - OR ) \times 100.$$

### BRIEF DESCRIPTION OF THE DRAWINGS

FIG. 1 illustrates a schematic representation of a plasmid template containing a T7 promoter, a 5′UTR, a 3′UTR, a polyA tail, and an exogenous gene CDS sequence.

FIG. 2 illustrates the chip electrophoresis photo of the mRNA synthesized by in vitro transcription using a linearized plasmid as the template. L denotes the RNA ladder, and 1 denotes the purified mRNA sample.

FIG. 3 illustrates the difference in expression level of SAS-Cov-2 B.1.351 spike protein caused by different combinations of 5′UTRs and 3′UTRs as measured by ELISA. The 5′UTRs and 3′UTRs are abbreviated as shown in Table 3.

FIG. 4 illustrates the difference in expression level of SAS-Cov-2 B.1.1.7 spike protein caused by different combinations of 5′UTRs and 3′UTRs as measured by ELISA. The 5′UTRs and 3′UTRs are abbreviated as shown in Table 3.

FIG. 5 illustrates a schematic representation of the point mutation from 5′UTR I to 5′UTR I′.

FIG. 6 illustrates the difference in expression level of SAS-Cov-2 B.1.617.2 spike protein caused by the 5′UTR I point mutation as measured by Western Blot. 5′UTR I was used for samples 1 and 3, and 5′UTR I′ was used for samples 2 and 4. The mRNA synthesis templates for samples 1 and 2 were from plasmid minipreparation, and the mRNA synthesis templates for samples 3 and 4 were from plasmid maxipreparation; sample 5 was an untransfected cell supernatant control; m denotes the molecular weight markers. FIG. 6A illustrates a Western Blot assay, and FIG. 6B illustrates a sample ratio graph after quantification of the Western Blot assay results.

FIG. 7 illustrates the effects of 5′UTR I′ on expression levels of different heterologous genes as measured by Western Blot. FIG. 7A illustrates a Western Blot assay, and FIG. 7B illustrates a sample relative value graph after quantification of the Western Blot assay results.

FIG. 8 illustrates the luciferase expression mediated by 5′UTR I′-3′UTR B and 5′UTR I-3′UTR B compared to the BNT162b2 UTR combination.

FIG. 9 illustrates the luciferase expression regulated by 5′UTR I′ truncations and 5′UTR I′ mutants.

FIG. 10 illustrates the difference in HA mRNA expression level regulated by 5′UTR I′ versus a control 5′UTR

### DETAILED DESCRIPTION

### Terminology

In order to facilitate the understanding of the present disclosure, some technical and scientific terms are specifically defined below. Unless otherwise specifically defined herein, all other technical and scientific terms used herein have the meanings generally understood by those of ordinary skill in the art to which the present disclosure pertains.

The “2019-nCoV” is known as severe acute respiratory syndrome coronavirus 2 (SARS-CoV-2).

The “disease caused by 2019-nCoV” is known as COVID-19.

The “nucleic acid” or “nucleotide” includes RNA, DNA, and cDNA molecules. It will be appreciated that due to the degeneracy of the genetic codes, a large number of nucleotide sequences encoding a given protein may be generated. The term nucleic acid can be used interchangeably with the term “polynucleotide”. The “oligonucleotide” refers to a short nucleic acid molecule. The “primer” is an oligonucleotide, whether naturally occurring in a purified restriction enzymatic digest or produced synthetically, that is capable of acting as a start point of synthesis when placed in a condition inducing the synthesis of a primer extension product complementary to a nucleic acid strand (i.e., in the presence of nucleotides and an inducing agent such as DNA polymerase, and at a suitable temperature and pH). To maximize the amplification efficiency, the primer is preferably single-stranded, but may optionally be double-stranded. If double-stranded, the primer is first treated to separate the strands before use in preparing an extension product. Preferably, the primer is a deoxyribonucleotide. The primer must be long enough to initiate the synthesis of the extension product in the presence of an inducer. The exact length of the primer will depend on many factors, including temperature, source of the primer, and the method used.

The “vector” or “expression vector” is meant a replicon, such as a plasmid, bacmid, phage, virus, virion, or cosmid, to which another DNA segment, i.e., an “insert”, may be attached, so as to achieve the replication of the attached segment in a cell. The vector may be a nucleic acid construct designed for the delivery to a host cell or for the transfer among different host cells. As used herein, the vector may be viral or non-viral in its original and/or final form, such as the PUC57 DNA vector used herein. The term “vector” encompasses any genetic element capable of replicating when combined with appropriate control elements and capable of transferring a gene sequence to a cell. In some embodiments, the vector may be an expression vector or a recombinant vector.

The “promoter” refers to any nucleic acid sequence that regulates the expression of another nucleic acid sequence by driving the transcription of the nucleic acid sequence, which may be a heterologous target gene encoding a protein or RNA. The promoter may be constitutive, inducible, repressible, or tissue-specific, or any combination thereof. The promoter is a control region of a nucleic acid sequence where the initiation and rate of the transcription of the remainder of the nucleic acid sequence is controlled.

The “gene” refers to a segment of DNA involved in the production of a polypeptide chain, which may or may not include preceding and succeeding coding regions, e.g., 5′ untranslated region (5′UTR) or “leader” sequence and 3′UTR or “non-transcribed tail” sequence, as well as intervening sequences (introns) between the coding segments (exons).

The “recombinant” refers to that a polynucleotide is a product of various combinations of cloning, restriction, or ligation procedures, as well as other procedures resulting in constructs different and/or distinct from naturally occurring polynucleotides.

The “introduction”, in the context of inserting a nucleic acid sequence into a cell, refers to “transfection”, “transformation”, or “transduction” and includes reference to the incorporation of a nucleic acid sequence into a eukaryotic or prokaryotic cell, where the nucleic acid sequence may be incorporated into the genome of the cell (e.g., chromosome, plasmid, plastidial, or mitochondrial DNA), transformed to autonomous replication, or transiently expressed (e.g., transfected mRNA).

The “nucleic acid construct” refers to a nucleic acid molecule, e.g., a DNA fragment, either single- or double-stranded, that is modified or synthesized to comprise a nucleic acid segment in a manner that does not naturally occur; the nucleic acid molecule comprises one or more control sequences or regulatory elements. In the context of the present disclosure, the nucleic acid construct contains a recombinant nucleotide sequence consisting essentially of optionally one, two, three, or more isolated nucleotide sequences including 5′UTR, open reading frame (ORF), and 3′UTR. In embodiments involving constructs comprising two or more sequences, the sequences are operably linked to each other in the construct.

The “derivative sequence” refers to a nucleotide sequence that is highly homologous (e.g., having at least 80%, 85%, 88%, 90%, 93%, 95%, 96%, 97%, 98%, 99%, or 100% identity) to the UTR sequence of the present disclosure and still retains the same or similar functional activity as the UTR sequence of the present disclosure. In some embodiments, the derivative sequence is a nucleotide sequence having one or more substituted, deleted, or added nucleotides based on a natural UTR sequence. In some embodiments, the derivative sequence is a nucleotide sequence that is truncated on the basis of the natural UTR sequence. In some embodiments, the derivative sequence has a point mutation of an ATG into a GTG, CTG, or TTG on the basis of a natural UTR sequence, for inhibiting the ATG within the UTR from initiating translation.

The “operably linked” is defined herein as a structure where a control sequence, i.e., a promoter sequence and/or a 5′UTR sequence, are suitably positioned relative to the coding DNA sequence such that the control sequence directs the transcription of the coding sequence and the translation of the mRNA into a polypeptide sequence encoded by the coding DNA.

The “open reading frame”, abbreviated as “ORF”, refers to a segment or region encoding an mRNA molecule of a polypeptide. The ORF comprises contiguous, non-overlapping, in-frame codons, starting with a start codon and ending with a stop codon, which are translated by the ribosome.

The “endogenous” refers to any substance derived from or produced in an organism, cell, tissue, or system.

The “exogenous” refers to any substance introduced or produced from outside an organism, cell, tissue, or system.

The “sequence identity” refers to sequence identity between genes or proteins at the nucleotide or amino acid level, respectively. The “sequence identity” is a measure of identity between proteins at the amino acid level and between nucleic acids at the nucleotide level. The protein sequence identity can be determined by comparing the amino acid sequences at given positions in the aligned sequences. Similarly, the nucleic acid sequence identity can be determined by comparing the nucleotide sequences at given positions in the aligned sequences. Methods for aligning sequences for comparison are well known in the art, including GAP, BESTFIT, BLAST, FASTA, and TFASTA. The BLAST algorithm calculates the percent sequence identity and performs a statistical analysis of the similarity between two sequences. Software for performing BLAST analysis is publicly available through the National Center for Biotechnology Information (NCBI) website.

The “homologous” or “homology” is defined as the percentage of nucleotide residues in a target chromosome identical to those in a corresponding sequence after aligning the sequences and introducing gaps, if necessary, to achieve the maximum percentage sequence identity. Alignment to determine percentage nucleotide sequence homology can be accomplished in a variety of ways within the skill in the art, for example, using publicly available computer software such as BLAST, BLAST-2, ALIGN, ClustalW2, or Megalign (DNASTAR). In some specific embodiments, the present disclosure calculates percentage sequence homology on the basis of BLAST. Those skilled in the art can determine suitable parameters for aligning the sequences, including any algorithms necessary to achieve the maximum alignment over the full length of the sequences being compared. In some embodiments, the sequences are considered “homologous” when, for example, a nucleic acid sequence (e.g., a DNA sequence) of a homology arm has at least 70%, at least 75%, at least 80%, at least 85%, at least 90%, at least 91%, at least 92%, at least 93%, at least 94%, at least 95%, at least 96%, at least 97%, at least 98%, at least 99%, or higher identity to a corresponding natural or unedited nucleic acid sequence (e.g., a genomic sequence) of a host cell.

The “substitution” is defined as a change in amino acid or nucleotide sequence as follows: one or more amino acids or nucleotides are substituted with a different amino acid or nucleotide, as compared to the amino acid sequence or nucleotide sequence of a reference polypeptide. If the substitution is conservative, the substituent amino acid has similar structural or chemical properties (e.g., charge, polarity, hydrophobicity, etc.) as the substituted amino acid. In some embodiments, a polypeptide variant may have a “non-conservative” change, where the amino acids before and after the substitution differ in structural and/or chemical properties.

The “deletion” is defined as a change in amino acid or nucleotide sequence as follows: one or more amino acid or nucleotide residues are deleted, as compared to the amino acid sequence or nucleotide sequence of a reference polypeptide. In the case of a polypeptide or polynucleotide sequence, considering the length of the polypeptide or polynucleotide sequence being modified, the deletion may involve deletions of 2, 5, 10, up to 20, up to 30, or up to 50 or more amino acid or nucleotide residues.

The “insertion” or “addition” refers to a change in amino acid or nucleotide sequence as follows: one or more amino acids or nucleotides are added, as compared to the amino acid sequence or nucleotide sequence of a reference polypeptide. The “insertion” generally refers to the addition of one or more amino acid residues within the amino acid sequence of a polypeptide (or nucleotide residues within a polynucleotide), while the “addition” may be an insertion or the addition of amino acid residues at the N- or C-terminus of a polypeptide (or the addition of nucleotide residues at the 5′ or 3′ terminus of a polynucleotide). In the case of a polypeptide or polynucleotide sequence, the addition or insertion may involve up to 10, up to 20, up to 30, or up to 50 or more amino acids (or nucleotide residues).

The “codon optimization” refers to the replacement of a codon in the target sequence that is rarely seen in highly expressed genes of a given species with a codon commonly seen in highly expressed genes of the species, with the codons before and after the replacement encoding the same amino acid. Various species may exhibit specific usage biases for certain codons for a particular amino acid. The codon usage bias (the difference in codon usage between organisms) is often correlated with the translation efficiency of messenger RNA (mRNA), which in turn is believed to particularly depend on the characteristics of the codons translated and the availability of specific transfer RNA (tRNA) molecules. The predominance of a selected tRNA in a cell is typically a reflection of the codons most frequently used in peptide synthesis. Thus, on the basis of codon optimization, genes can be modified for the optimal gene expression in a given organism. Thus, the option of optimal codons depends on the codon usage bias of the host genome.

The “antigen antibody” refers to a serum antibody that specifically binds to an antigen.

The “cell” or “host cell” includes any cell type susceptible to transformation, transfection, transduction, and the like with the nucleic acid construct or vector of the present disclosure. As a non-limiting example, the host cell may be an isolated primary cell, a pluripotent stem cell, a CD34+ cell, an induced pluripotent stem cell, or any one of a number of immortalized cell lines (e.g., a HepG2 cell). Alternatively, the host cell may be an in situ or in vivo cell in a tissue, organ, or organism.

The “treatment” refers to administering a therapeutic agent, such as a composition comprising any one of the nucleic acid constructs of the present disclosure, either internally or externally to a patient with one or more symptoms of a disease on which the therapeutic agent is known to have a therapeutic effect. Typically, the therapeutic agent is administered in an amount effective to alleviate one or more symptoms of the disease in the patient or population being treated to induce regression of such symptoms or to inhibit the development of such symptoms to any clinically measurable degree. The amount of therapeutic agent effective to alleviate any particular symptom of the disease (also known as a “therapeutically effective amount”) may vary depending on a variety of factors, such as the disease state, age, and weight of the patient, and the ability of the drug to produce a desired therapeutic effect in the patient. Whether a disease symptom has been alleviated can be evaluated by any clinical testing methods commonly used by doctors or other health care professionals to evaluate the severity or progression of the symptom.

The “effective amount” or “pharmaceutically effective amount” comprises an amount sufficient to ameliorate or prevent a symptom or condition of a medical disorder. The effective amount also refers to an amount sufficient to allow or facilitate diagnosis. The effective amount for a particular patient or veterinary subject may vary depending on the factors such as the disorder to be treated, the general health of the patient, the method and route and dose of administration, and the severity of adverse effects. The effective amount may be the maximum dose or administration regimen to avoid significant adverse effects or toxic effects. In some embodiments, the “effective amount” is an effective amount of RNA that can produce an antigen-specific immune response.

The “prophylactically effective dose” refers to an effective dose to prevent viral infection at a clinically acceptable level. In some embodiments, the effective dose is a dose listed in the vaccine package insert. The conventional vaccine, as used herein, refers to a vaccine other than the mRNA vaccine of the present disclosure. For example, the conventional vaccine includes, but is not limited to, a live microbial vaccine, an inactivated microbial vaccine, a subunit vaccine, a protein antigen vaccine, a DNA vaccine, a virus-like particle (VLP) vaccine, and the like. In exemplary embodiments, the conventional vaccine is one that has been approved by regulatory authorities and/or listed by a national drug administration, such as the Chinese National Medical Products Administration.

The term “pharmaceutically acceptable” refers to those therapeutic agents, materials, compositions, and/or dosage forms that are, within the scope of sound medical judgment, suitable for use in contact with the tissues of patients without excessive toxicity, irritation, allergic reaction, or other problems or complications, and are commensurate with a proper benefit/risk ratio and effective for the intended use.

The “monovalent vaccine” refers to a vaccine preparation for immunization made from an antigen of one serotype from a pathogenic organism. For example, a vaccine containing only one antigen of one SARS-CoV-2 strain of the present disclosure is a monovalent vaccine.

The “multivalent vaccine” refers to a vaccine preparation for immunization made from antigens of more than one serotype from the same pathogenic organism. For example, a vaccine containing an antigen from one SARS-CoV-2 variant mixed with one or more antigens from other SARS-CoV-2 variants of different subtypes is a multivalent vaccine.

The “combined vaccine” refers to a vaccine preparation for immunization made from antigens of more than one serotype from different pathogenic organisms. For example, a vaccine containing an antigen of the SARS-CoV-2 strain of the present disclosure mixed with several pathogenic microorganisms that can prevent and/or treat other viral infections is a combined vaccine. The combined vaccine is generally prepared from monovalent vaccines prepared by the same preparation method, and then is mixed in the same injection for immunization; alternatively, antigens of several pathogenic microorganisms are mixed and then prepared into a vaccine product. In some specific embodiments, antigens of pathogenic microorganisms are first mixed and then prepared into a vaccine preparation. Combined and multivalent vaccines are the future development trends due to their advantages of: (1) cost-efficiency in vaccination; (2) cost-efficiency in packaging in doses, transportation, and assembly; (3) reduced damage to vaccinees, particularly infants; (4) increased vaccination coverage; (5) efficiency in vaccination; (6) compactness for vaccine storage, etc. The combined or multivalent vaccine can save direct and indirect costs including multiple visits, missed vaccination doses, and the like. For example, combined, multivalent vaccines commonly used at present include vaccines against Streptococcus pneumoniae, Neisseria meningitidis, poliovirus, rotavirus, influenza, HPV, and the like.

The “immune response” of the vaccine of the present disclosure refers to the development of a humoral and/or cellular immune response in a subject to protein(s) of a virus (e.g., coronavirus) present in the vaccine. For the purposes of the present disclosure, the “humoral” immune response refers to an immune response mediated by antibody molecules, including, for example, secretory IgA or IgG molecules, while the “cellular” immune response is an immune response mediated by T lymphocytes (e.g., CD4+ helper and/or CD8+ T cells (e.g., CTLs) and/or other leukocytes). An important aspect of the cellular immunity involves the antigen-specific reactions of cytolytic T lymphocytes (CTLs). The CTLs are specific for peptide antigens that are presented in combination with proteins encoded by the major histocompatibility complex (MHC) and expressed on the cell surface, and help induce and promote the destruction of intracellular microorganisms or lyse cells infected with these microorganisms. Another aspect of the cellular immunity involves antigen-specific responses by helper T cells, which help stimulate function and concentrate the activities of non-specific effector cells on cells with peptidic antigens associated with MHC molecules. The cellular immune responses also result in cytokines, chemokines, and other like molecules produced by activated T cells and/or other leukocytes.

### EXAMPLES

The present disclosure is further described below with reference to examples; however, these examples are not intended to limit the scope of the present disclosure. Experimental procedures without conditions specified in the examples of the present disclosure were generally conducted according to conventional conditions such as cell culture manuals or molecular cloning manuals, or according to conditions recommended by the manufacturers of the raw material or the goods. Reagents without specific origins indicated are commercially available conventional reagents.

The following general experimental procedures 1) to 4) are given by means of examples:

1) mRNA Production

To generate in vitro transcribed mRNA, the plasmid was linearized downstream of the polyA tail using BspQ 1 (Vazyme, DD4302-PC, China) and purified by a PCR purification kit (QIAGEN, 28106, Germany). In vitro transcription was performed using the T7 in vitro transcription kit (Invitrogen, AM1333, USA) with the purified linearized plasmid as template. The mRNA synthesized by in vitro transcription was purified by MEGAclear™ Kit (Invitrogen, AM1908, USA) to give an mRNA with a relatively higher purity for subsequent in vitro cell transfection and other experiments.

### 2) Chip Electrophoresis

To examine the integrity and purity of the mRNA obtained by in vitro transcription, the mRNA was analyzed using an on-chip electrophoresis system (Agilent 2100, USA) and the RNA 6000 Nano kit (Agilent, 5067-1511, USA). The procedures are as follows: the mRNA was denatured (incubated for 2 min at 70° C. and quickly transferred on ice), and the treated samples were sequentially transferred on corresponding wells of the chip, loaded for analysis until the analysis results were generated.

### 3) Cell Culture

Materials: FBS (Gibco), DMEM medium (Gibco), Opti-MEM (Gibco), PBS (Gibco), trypsin-EDTA (Gibco), antibiotics (pen/strep, Gibco), and Lipofectamine™ 2000 (Invitrogen, 52887).

Human embryonic kidney cells (293T, ATCC) were cultured in DMEM medium supplemented with 10% of FBS and 1% of the antibiotics in a humidified environment at 5% CO₂.

### 4) In Vitro Transfection

Cells were seeded in 12-well cell culture plates 8 h before the transfection at 8×10⁵ cells/well. The cells were transfected using a commercially available transfection reagent Lipofectamine™ 2000 in a ratio of 1 μg of mRNA to 2.5 μL of Lipofectamine™ 2000, at 2 μg of mRNA/well. The procedures are as follows: Lipofectamine™ 2000 and mRNA were separately diluted in Opti-MEM medium in a final volume of 100 μL, and the Lipofectamine™ 2000 solution and the mRNA solution were mixed and incubated at room temperature for 10-15 min to give a liposome complex containing mRNA. After the DMEM medium in the cell culture plates was discarded, the liposome complexes containing different mRNAs were added to the corresponding plates. Opti-MEM medium containing 10% of FBS was then added at 400 μL/well, and the cells were cultured at 37° C. (5% CO₂) for 4-6 h. The transfection medium was then discarded. DMEM medium containing 10% of FBS and 1% of antibiotics was added at 1 mL/well, and the cells were cultured at 37° C. (5% CO₂) for 48 h. Meanwhile, part of the cells were transfected with Lipofectamine™ 2000 solution with no mRNA as a negative control in the above manner.

The sequences of the target genes used in the present disclosure are summarized in Table 1.

TABLE 1
Sequence information of target genes of the present disclosure

| | | | | | | Mutation |
|---|---|---|---|---|---|---|
| | | | | | | position |
| | | | | | | compared to |
| | | | | Optimized | Optimized | wildtype |
| Protein encoded | Polypeptide | DNA | mRNA | DNA | mRNA | SARS-CoV-2 |
| by target gene | sequence | sequence | sequence | sequence | sequence | (KV-PP) |
| B.1.1.7 (Alpha) | SEQ ID | / | / | SEQ ID | SEQ ID | 983-984 |
| spike protein | NO: 23 | | | NO: 22 | NO: 60 | |
| B.1.351 (Beta) | SEQ ID | / | / | SEQ ID | SEQ ID | 983-984 |
| spike protein | NO: 21 | | | NO: 20 | NO: 59 | |
| B.1.617.2 (Delta) | SEQ ID | / | / | SEQ ID | SEQ ID | 984-985 |
| spike protein | NO: 12 | | | NO: 13 | NO: 54 | |
| B.1.1.529 | SEQ ID | / | / | SEQ ID | SEQ ID | 983-985 |
| (Omicron) spike | NO: 14 | | | NO: 15 | NO: 55 | |
| protein | | | | | | |
| SARS-CoV-2 | SEQ ID | / | / | SEQ ID | SEQ ID | 986-987 |
| spike protein | NO: 25 | | | NO: 24 | NO: 61 | |
| Wildtype SARS- | SEQ ID | SEQ ID | SEQ ID | / | / | / |
| CoV-2 spike | NO: 80 | NO: 81 | NO: 82 | | | |
| protein | | | | | | |

The mutation from amino acids KV to PP in SARS-CoV-2 spike protein can change the spike protein from an unstable prefusion conformation to a stable postfusion conformation (Structure-based design of prefusion-stabilized SARS-CoV-2 spikes, Science, VOL. 369, NO. 6510), such that the spike protein is in a stable state in conformation, which is favorable for vaccine design and production by taking the spike protein as immunogen.

### Example 1. Screening and Preparation of 5′UTR Sequences

### 1. Screening of UTRs

In order to give a regulatory element capable of highly expressing a target protein in dendritic cells (DCs), this example was conducted with four-stage screening to give a new UTR.

The procedures are as follows: I, a high-protein-expression gene library was established, including genes with a high protein expression in monocytes, genes with a higher protein expression in DCs than in monocytes, genes with a higher protein level, and genes of a UTR with confirmed capability of increasing the protein expression, thus giving 2140 genes. II, a UTR library was established by downloading all sequences in the high-protein-expression gene library from Genbank database (https://www.ncbi.nlm.nih.gov/genbank/), analyzed for the integrity, and then analyzed by methods such as repeat combination, CDS (coding sequence) interception and the like to give UTR data, thus giving 940 UTRs. III, a UTR selection library was established by analyzing the UTR library, screening sequences with a proper sequence length (40 to 70 for 5′UTR sequences and 100 to 150 or 300 to 400 for 3′UTR sequences) and free energy, predicting the free energy of the sequences one by one, selecting sequences with a high 5′UTR free energy, and selecting sequences with a low 3′UTR free energy, thus acquiring 64 UTRs from the selection library. IV, final candidate sequences were determined by a screening strategy with a 5′UTR free energy of −10 or higher and a low 3′UTR free energy, finally giving 20 5′UTRs and 3′UTRs for screening and intracellular verification.

On this basis, we believe that the 5′UTR sequence (designated as 5′UTR I, with the DNA sequence set forth in SEQ ID NO: 1 and the RNA sequence set forth in SEQ ID NO: 45) derived from human Rho GTPase activating protein 15 (abbreviated as ARHGAP15) has the potential of efficiently expressing target proteins in human cells (particularly DCs).

### 2. Preparation of UTR

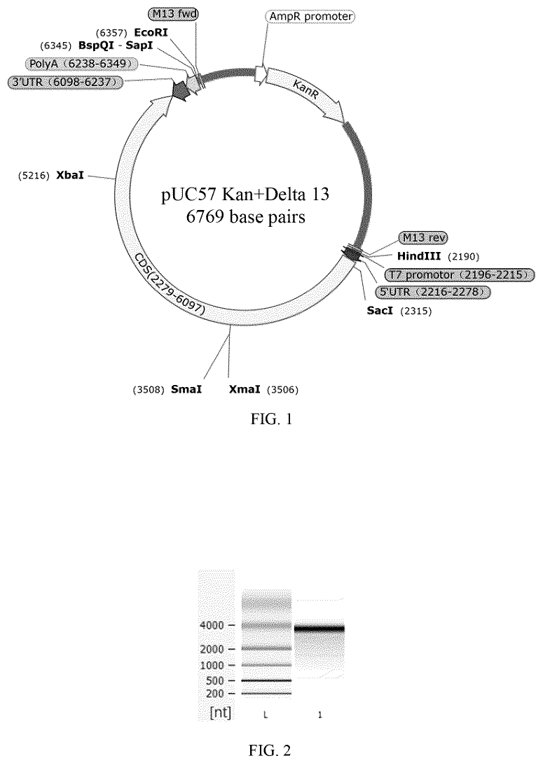

This example illustrates the preparation of 5′UTR I-containing mRNA. First, a sequence containing 5′UTR, 3′UTR, polyA tail, and the CDS of a heterologous target gene was artificially synthesized and inserted into pUC57 vector to give a template vector, as shown in FIG. 1. The sequence of the target gene inserted in the vector was confirmed by sequencing.

As shown in the pUC57 Delta13 vector in FIG. 1, the 5′UTR is set forth in SEQ ID NO: 1; the 3′UTR was a human hemoglobin beta subunit (HBB) 3′UTR sequence (SEQ ID NO: 7, designated as 3′UTR B); the CDS sequence was a base sequence (SEQ ID NO: 13) with optimized codons encoding the B.1.617.2 (Delta) spike protein; the polyA tail was a polyadenylic acid sequence (with the DNA sequence set forth in SEQ ID NO: 16 and the RNA sequence set forth in SEQ ID NO: 56); the T7 promoter is set forth in SEQ ID NO: 17.

To generate in vitro transcribed mRNA, the plasmid was linearized downstream of the poly A tail using BspQ I (Vazyme, DD4302-PC, China) and purified by a PCR purification kit (QIAGEN, 28106, Germany). In vitro transcription was performed to synthesize the mRNA with the purified linearized plasmid as template according to the following procedures. A 100 μL mRNA reaction system, as shown in Table 2, was synthesized. In the reaction system, 2 μg of linearized template was diluted to 100 μL with the non-nucleoside enzyme water. The reaction system was incubated for 4 h at 37° C. and digested for 30 min by DNase I. Detections showed a small amount of byproduct dsRNA in the synthesized mRNA and thus a good yield. The capping process was achieved by chemical capping in the in vitro transcription synthesis, with the cap structure being m7G(5′)ppp(5′)(2′OMeA)pG·NH₄ (Hongene, ON-134). The synthesized mRNA was purified by MEGAclear™ Kit (Invitrogen, AM1908, USA) to give an mRNA with a relatively higher purity for subsequent in vitro cell transfection and other experiments.

TABLE 2
mRNA reaction system

| Component | Cat. No. | Manufacturer | System (μL) |
|---|---|---|---|
| ATP solution (100 mM) | R1331 | Hongene | 4.41 |
| CTP solution (100 mM) | R3331 | Hongene | 5 |
| GTP solution (100 mM) | R2331 | Hongene | 2.79 |
| 10× transcription buffer | DD4101R-02 | Vazyme | 10 |
| T7 RNA polymerase | DD4101-PC-02 | Vazyme | 6.6 |
| Mouse RNase inhibitor | DD4102-PA | Vazyme | 2.75 |
| Pyrophosphatase | DD4103-PC-01 | Vazyme | 0.66 |
| Pseudo-UTP | R5-022 | Hongene | 2.79 |
| Cap-GAG Trimer | m7G(5′)ppp(5′)(2′OMeA)pG | Hongene | 1 |

Through screenings and functional verification, the 5′UTR I with the potential of efficiently expressing the target protein was acquired, with a sequence set forth SEQ ID NO: 1; and the construct containing the 5′UTR I can efficiently transcribe the corresponding mRNA, with a size consistent with the theoretical value as determined by chip electrophoresis (see FIG. 2).

### Example 2. Screening of Combinations of Different 5′UTRs and 3′UTRs for Efficiently Expressing Heterologous Proteins

In this example, different combinations of 5′UTRs and different combinations of 3′UTRs were screened to give UTR combinations capable of efficiently expressing a target gene.

The procedures are as follows: Constructs of plasmids containing the CDS of the same target gene with different UTR combinations were artificially synthesized. The basic plasmid was constructed according to the procedures in Example 1. The target gene CDS sequence was introduced by recombinant construction via artificial synthesis and HindIII/XhoI digestion. The reconstructed plasmid was linearized and purified mRNA was synthesized according to the procedures in Example 1. mRNA was transfected into 293T cells by Lipofectamine™ 2000. 293T cell culture supernatant was collected after 48 h and detected by Western Blot and ELISA to determine the level of secreted spike proteins in the cell supernatant.

The target genes used were: the gene encoding the SARS-CoV-2 B.1.351 spike protein (with a CDS set forth in SEQ ID NO: 20), or the gene encoding the SARS-CoV-2 B.1.1.7 spike protein (with a CDS set forth in SEQ ID NO: 22). The information of different 5′UTRs and 3′UTRs and their combinations are shown in Tables 3 and 4.

Western Blot procedures: To detect the in vitro expression level of the constructed mRNAs, the cell supernatant in the plate was collected after the transfection was completed, and the in vitro expression level of the constructed mRNAs was finally obtained by polyacrylamide gel electrophoresis, membrane transfer, co-incubation with the primary antibody and secondary antibody, and color development. The primary antibody was a SARS-CoV-2 (2019-nCoV) spike RBD antibody (Sino Biology, 40592-T62), and the secondary antibody was an HRP-linked anti-rabbit IgG (Transgene, HS101).

ELISA procedures: after the transfection was completed, the cell supernatant was collected, diluted in a ratio of 1:50 to 1:100, and detected for the expression level of the constructed mRNAs in vitro using an ELISA kit (SARS-CoV-2 (2019-nCoV) spike detection ELISA KIT, KIT40591, Sino Biology). The absorbance at 450 nm was read and the concentration of the relevant antigen was calculated.

TABLE 3
Sources and sequences of 5′UTR and 3′UTR

| | | | | DNA | RNA |
|---|---|---|---|---|---|
| | | | | sequence | sequence |
| | | | | (SEQ | (SEQ |
| UTR | Abbreviation | Source | GenBank ID | ID NO:) | ID NO:) |
| 5′UTR | I | Human ARHGAP15 | XM_003822828.3 | 1 | 45 |
| | G | Human heat shock 27 kDa | BC012292.1 | 3 | 47 |
| | | protein 1 (HSPB1) | | | |
| | A | Human DNA, containing HBB | LC507571.1 | 4 | 48 |
| | | gene | | | |
| | J | Human C-C motif chemokine | NM_005408.3 | 5 | 49 |
| | | ligand 13 (CCL13) | | | |
| 3′UTR | B | Human HBB | MK476504.1 | 7 | 50 |
| | F | Human ARHGAP15 | NM_018460.4 | 8 | 51 |
| | E | Coronin 1A (CORO1A) | NM_007074.4 | 9 | 52 |
| | D | Hemopexin (HPX) | NM_000613.3 | 10 | 53 |

TABLE 4
Different combinations of 5′UTR and 3′UTR

| Name | 5′UTR | 3′UTR |
|---|---|---|
| A-B | A | B |
| A-E | A | E |
| A-D | A | D |
| A-F | A | F |
| G-E | G | E |
| G-B | G | B |
| G-D | G | D |
| I-E | I | E |
| I-B | I | B |
| I-F | I | F |
| I-D | I | D |
| J-E | J | E |

In Table 4, 5′UTRs A, G, I, and J are set forth in SEQ ID NOs: 4. 3, 1, and 5, respectively; 3′UTRs B, E, D, and F are set forth in SEQ ID NO: 7. 9, 10, and 8, respectively.

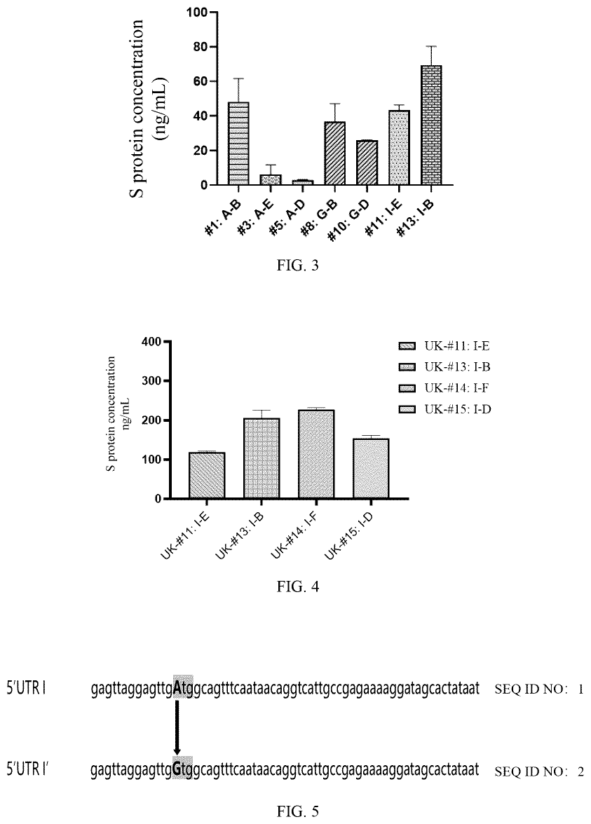

The effects of different combinations of 5′UTR with 3′UTR on the expression of SARS-CoV-2 B.1.351 spike protein and SARS-CoV-2 B.1.1.7 spike protein are shown in FIG. 3 and Table 5 and FIG. 4 and Table 6, respectively.

TABLE 5
Regulatory result of different UTR combinations on B.1.351 spike protein expression level (ELISA)

| Sample No. | #1: A-B | #3: A-E | #5: A-D | #8: G-B | #10: G-D | #11: I-E | #13: I-B |
|---|---|---|---|---|---|---|---|
| Spike protein | 48.2 ± 13.4 | 6.3 ± 5.4 | 2.9 ± 0.28 | 36.7 ± 10.5 | 26 ± 0.14 | 43.4 ± 2.97 | 69.4 ± 10.88 |
| concentration | | | | | | | |
| (mean ± SD, ng/mL) | | | | | | | |

(sample numbering rule is that, for example, #1: A-B denotes sample #1 and a UTR combination of A-B)

TABLE 6
Regulatory result of different UTR combinations
on B.1.1.7 spike protein expression level (ELISA)

| | Sample No. | | | | |
|---|---|---|---|---|---|
| | | | | | NC |
| | UK-#11: | UK-#13: | UK-#14: | UK-#15: | (Lipo |
| | I-E | I-B | I-F | I-D | control) |
| Spike protein | 118.35 ± | 206.1 ± | 227.6 ± | 153.55 ± | −9.65 ± |
| concentration | 3.61 | 19.66 | 4.38 | 7.99 | 0.64 |
| (mean ± SD, | | | | | |
| ng/mL) | | | | | |

(sample numbering rule is that, for example, UK-#11: I-E denotes sample UK-#11 and a UTR combination of I-E)

As can be seen from FIG. 3 and Table 5, the comparison of B.1.351 spike protein expression level mediated by different UTR combinations is as follows: I-B>A-B>G-B, and I-B>I-E. For the 5′UTR, I is superior to A and G; for relevant combinations of I, I-B is superior to I-E. As can be seen from FIG. 4 and Table 6, the comparison of B.1.1.7 spike protein expression level mediated by different UTR combinations is as follows: I-F>I-B>I-D>I-E. By analyzing the results of B.1.351 and B.1.1.7 spike protein expression levels, the I-B UTR combination exhibited more significant advantages in mediating the efficient expression of the target gene, while the I-F combination also exhibited advantages in high-level expression of the target gene.

Furthermore, the mRNAs containing I-B or I-F regulated B.1.617.2 spike protein were prepared, transfected into cells, and tested by ELISA. The spike protein expression level results are shown in Table 7.

TABLE 7
I-B and I-F UTR regulated B.1.617.2 spike
protein expression level (ELISA)

| Sample | I-B | I-F |
|---|---|---|
| Spike protein concentration (ng/mL) | 57.1 | 58.8 |

Unlike the 5′UTR and 3′UTR in the I-B combination that are from different genes, the 5′UTR and 3′UTR in the I-F combination come from the same gene ARHGAP15. The results in Table 7 show that the I-B (SEQ ID NO: 1 and SEQ ID NO: 7) and I-F (SEQ ID NO: 1 and SEQ ID NO: 8) combinations regulate the expression of the target gene at comparable levels, further illustrating the universality of 5′UTR I for combination with different 3′UTRs to mediate efficient expression of the target gene (e.g., the gene encoding the spike protein).

### Example 3. Sequence Engineering and Preliminary Functional Validation of 5′UTR I

### 1) Bioinformatic Analysis and Mutation Design

5′UTR I (SEQ ID NO: 1) is a naturally occurring promoter of the human Rho GTPase activating protein family. The data of Examples 1-2 support that 5′UTR I assists in efficiently expressing the SARS-CoV-2 spike protein. However, 5′UTR I contains an ATG and may express a 27aa short peptide, which may affect normal protein expression of the target gene. In order to avoid the phenomenon that ribosome initiates the translation from the ATG inside the UTR, increase the probability of translation from the ATG in the CDS region, and increase the expression, a point mutation (A→G) was introduced to give a mutant 5′UTR I′ (with a DNA sequence set forth in SEQ ID NO: 2 and an RNA sequence set forth in SEQ ID NO: 46). The mutation positions are shown in FIG. 5.

2) Point Mutation from 5′UTR I to 5′UTR I′ Effectively Increasing the Expression of Target Genes

Plasmids containing 5′UTR I-B and 5′UTR I′-B to regulate the target gene expression were prepared according to the method in Example 1. The target gene was the gene encoding B.1.617.2 spike protein (SEQ ID NO: 13). The plasmids were designated as Delta 13 and Delta 24, respectively, and the mRNAs were synthesized by PCR and transcription and purified (the cap-free I′-Delta24-B mRNA sequence is set forth in SEQ ID NO: 57). The prepared mRNA was transfected into 293T cells, and the B.1.617.2 spike protein expression was detected using the Western Blot method in Example 2. By gray scanning using Image software, the grayscale value was calculated and normalized by the expression level of sample 1 (Delta 13) to give the ratio in Table 8.

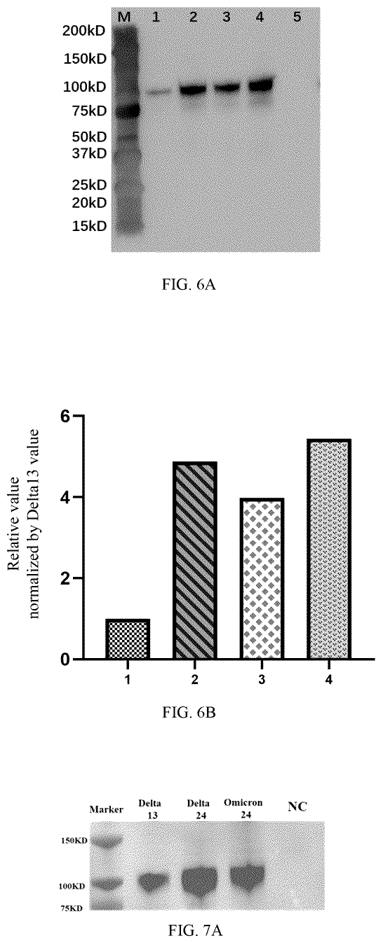

The results are shown in FIGS. 6A and 6B and Table 8. The results show that for mRNAs prepared using either the minipreparation plasmid (with no endotoxin removal) or the maxipreparation plasmid (with endotoxins removed) as the template, the expression level of the spike protein mediated by the construct of 5′UTR I′ after point mutation was greater than that mediated by the construct containing 5′UTR I. Thus, the 5′UTR I modified by the point mutation in Example 3 effectively improves the expression of heterologous proteins.

TABLE 8
Target gene expression levels regulated
by I-B and I′-B UTRs (Western Blot assay)

| | Plasmid mini-pre | | Plasmid maxi-pre | |
|---|---|---|---|---|
| | Sample 1 | Sample 2 | Sample 3 | Sample 4 |
| Sample | (Delta 13) | (Delta 24) | (Delta 13) | (Delta 24) |
| Ratio to expression | 1.00 | 4.88 | 3.98 | 5.44 |
| of Sample 1 | | | | |

### Example 4. Assay of mRNA Expression of Different Target Genes Regulated by 5′UTR I

Plasmids containing the gene encoding B.1.617.2 (Delta) spike protein (SEQ ID NO: 13) and the gene encoding B.1.1.529 (Omicron) spike protein (SEQ ID NO: 15) were constructed by replacing the target gene via digestion, with the 5′UTR I′ and the 3′UTR B retained. The target gene sequences contained in the plasmids, as set forth in SEQ ID NO: 13 and SEQ ID NO: 15, were codon-optimized sequences encoding target protein sequences set forth in SEQ ID NO: 12 and SEQ ID NO: 14. mRNAs were prepared and purified as in Example 1. 2 μg of mRNA was transfected to 293T cells by Lipofectamine™ 2000, and the cell supernatant was harvested after 2 days. As per the Western Blot method in Example 2, the expression of I-B regulated B.1.617.2 spike protein mRNA (designated as Delta 13), I′-B regulated B.1.617.2 spike protein mRNA (designated as Delta 24), and I′-B regulated B.1.1.529 spike protein mRNA (designated as Omicron 24) were determined. The sequences of cap-free Delta 24 mRNA and Omicron 24 mRNA are set forth in SEQ ID NO: 57 and SEQ ID NO: 58. By gray scanning using Image software, the grayscale value was calculated and normalized by the expression level of Delta 13 to give the ratio in Table 9.

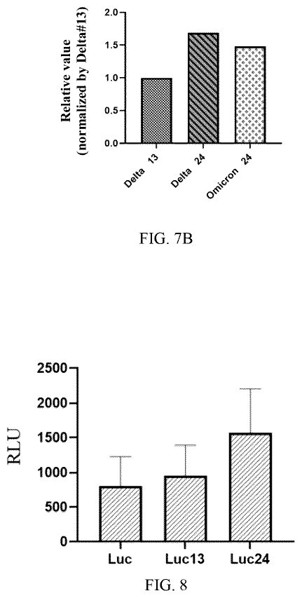

FIGS. 7A and 7B show that under the regulation of I′-B UTR combination, the SARS-CoV-2 B.1.617.2 and B.1.1.529 genes were expressed efficiently. The results suggest that 5′UTR I′ has universality and can initiate the efficient expression of different target genes. Comparing 5′UTRs I and I′, the Delta 24 spike protein exhibited an expression level significantly higher than that of the Delta 13 spike protein, indicating that the point mutation modification of 5′UTR I′ effectively improves the protein expression efficiency, and the result further confirms the conclusion of Example 3.

TABLE 9
Target gene expression levels regulated by I-B
and I′-B UTR combinations (Western Blot assay)

| Sample | Delta 13 | Delta 24 | Omicron 24 |
|---|---|---|---|
| Ratio to expression | 1.00 | 1.69 | 1.48 |
| of sample Delta 13 | | | |

### Example 5. Assay of Luciferase Expression Regulated by I′-B UTR Combination

A vector containing the luciferase CDS sequence (SEQ ID NO: 26) and the same T7 promoter (SEQ ID NO: 17) and polyA tail as in the previous vector containing the SARS-CoV-2 gene was constructed. Three vectors Luc, Luc13, and Luc24 with different UTRs were separately constructed, and the UTRs contained in the vectors are shown in Table 10. Among these, the 5′UTR and 3′UTR combination of BNT162b2 in Luc was used as the control, which is from CN113521269A. Capped mRNA was synthesized by in vitro transcription as in Example 1 and transfected in vitro. The supernatant was collected and luciferase substrate was added for detection. The plate was read, and the results are shown in Table 11 and FIG. 8.

TABLE 10
Vector structure

| | Sample No. | | |
|---|---|---|---|
| Component | Luc | Luc13 | Luc24 |
| 5′UTR | BNT162b2(SEQ ID NO: 6) | I | I′ |
| 3′UTR | BNT162b2(SEQ ID NO: 11) | B | B |

TABLE 11
Luciferase expression levels regulated by I′-B and I-B

| Sample | Luc | Luc13 | Luc24 |
|---|---|---|---|
| Run I | 774.81 | 992.36 | 1619.76 |
| Run II | 841.14 | 923.83 | 1519.50 |

Note:
The parameters are expressed in relative light unit (RLU).

The results show that the luciferase protein expression regulated by 5′UTR I′ after point mutation was higher than that by 5′UTR I. Compared to the 5′UTR and 3′UTR combination of BNT162b2 in Luc control, the I-B combination of the present disclosure results in a higher expression level of the target gene, while the I′-B combination results in a significant increase in the expression level of the target gene. The assay results using luciferase as the target gene also suggest that the 5′UTR I or 5′UTR I′ can regulate the efficient expression of any target protein with universality.

### Example 6. Assay of Luciferase Expression Regulated by F′UTR I′ Truncations and Mutants

6.1 The 5′ end of the 5′UTR I′ was successively truncated, and the efficiency of the 5′UTR I′ truncations of different lengths in regulating protein expression was analyzed by bioinformatic methods (Nat Biotechnol. July, 2019; 37(7):803-809). The relative MRL values of the 5′UTR I′ truncations to the 5′UTR I′ were calculated, and the results are shown in Table 12 below. The results in Table 12 show that successive truncations of the nucleotide sequence at the 5′ end of the 5′UTR I′ did not cause significant reduction in the MRL value, indicating that the 5′UTR I′ truncations can retain the functional activity of regulating the expression of the target protein. The 3′ end of the 5′UTR I′ truncation set forth in SEQ ID NO: 153 was successively truncated, and the relative MRL values of the 3′ end truncations to the nucleotide sequence set forth in SEQ ID NO: 153 were calculated, as shown in Table 13. As can be seen from Table 13, the 3′ end truncations did not exhibit a significant effect on the regulatory efficiency of the 5′UTR I′ truncations.

TABLE 12
Relative MRL values of 5′UTR I′
truncations and 5′UTR I′

| | Relative | |
|---|---|---|
| | MRL | SEQ ID |
| 5′UTR I′ truncation | value | NO: |
| AGTTAGGAGTTGGTGGCAGTTTCAATAAC | 1 | SEQ ID |
| AGGTCATTGCCGAGAAAAGGATAGCACTA | | NO: 137 |
| TAAT | | |
| GTTAGGAGTTGGTGGCAGTTTCAATAACA | 1 | SEQ ID |
| GGTCATTGCCGAGAAAAGGATAGCACTAT | | NO: 138 |
| AAT | | |
| TTAGGAGTTGGTGGCAGTTTCAATAACAG | 1 | SEQ ID |
| GTCATTGCCGAGAAAAGGATAGCACTATA | | NO: 139 |
| AT | | |
| TAGGAGTTGGTGGCAGTTTCAATAACAGG | 1 | SEQ ID |
| TCATTGCCGAGAAAAGGATAGCACTATAA | | NO: 28 |
| T | | |
| AGGAGTTGGTGGCAGTTTCAATAACAGGT | 1 | SEQ ID |
| CATTGCCGAGAAAAGGATAGCACTATAAT | | NO: 140 |
| GGAGTTGGTGGCAGTTTCAATAACAGGTC | 1 | SEQ ID |
| ATTGCCGAGAAAAGGATAGCACTATAAT | | NO: 141 |
| GAGTTGGTGGCAGTTTCAATAACAGGTCA | 1 | SEQ ID |
| TTGCCGAGAAAAGGATAGCACTATAAT | | NO: 142 |
| AGTTGGTGGCAGTTTCAATAACAGGTCAT | 1 | SEQ ID |
| TGCCGAGAAAAGGATAGCACTATAAT | | NO: 29 |
| GTTGGTGGCAGTTTCAATAACAGGTCATT | 1 | SEQ ID |
| GCCGAGAAAAGGATAGCACTATAAT | | NO: 143 |
| TTGGTGGCAGTTTCAATAACAGGTCATTG | 1 | SEQ ID |
| CCGAGAAAAGGATAGCACTATAAT | | NO: 144 |
| TGGTGGCAGTTTCAATAACAGGTCATTGC | 1 | SEQ ID |
| CGAGAAAAGGATAGCACTATAAT | | NO: 145 |
| GGTGGCAGTTTCAATAACAGGTCATTGCC | 1 | SEQ ID |
| GAGAAAAGGATAGCACTATAAT | | NO: 30 |
| GTGGCAGTTTCAATAACAGGTCATTGCCG | 1 | SEQ ID |
| AGAAAAGGATAGCACTATAAT | | NO: 146 |
| TGGCAGTTTCAATAACAGGTCATTGCCGA | 0.8 | SEQ ID |
| GAAAAGGATAGCACTATAAT | | NO: 147 |
| GGCAGTTTCAATAACAGGTCATTGCCGAG | 1 | SEQ ID |
| AAAAGGATAGCACTATAAT | | NO: 148 |
| GCAGTTTCAATAACAGGTCATTGCCGAGA | 1.02 | SEQ ID |
| AAAGGATAGCACTATAAT | | NO: 31 |
| CAGTTTCAATAACAGGTCATTGCCGAGAA | 1.02 | SEQ ID |
| AAGGATAGCACTATAAT | | NO: 149 |
| AGTTTCAATAACAGGTCATTGCCGAGAAA | 1.02 | SEQ ID |
| AGGATAGCACTATAAT | | NO: 150 |
| GTTTCAATAACAGGTCATTGCCGAGAAAA | 1 | SEQ ID |
| GGATAGCACTATAAT | | NO: 151 |
| TTTCAATAACAGGTCATTGCCGAGAAAAG | 1.01 | SEQ ID |
| GATAGCACTATAAT | | NO: 32 |
| TTCAATAACAGGTCATTGCCGAGAAAAGG | 1.01 | SEQ ID |
| ATAGCACTATAAT | | NO: 152 |
| TCAATAACAGGTCATTGCCGAGAAAAGGA | 1.01 | SEQ ID |
| TAGCACTATAAT | | NO: 153 |
| CAATAACAGGTCATTGCCGAGAAAAGGAT | 1.02 | SEQ ID |
| AGCACTATAAT | | NO: 154 |
| AATAACAGGTCATTGCCGAGAAAAGGATA | 1.02 | SEQ ID |
| GCACTATAAT | | NO: 33 |
| ATAACAGGTCATTGCCGAGAAAAGGATAG | 1.02 | SEQ ID |
| CACTATAAT | | NO: 155 |
| TAACAGGTCATTGCCGAGAAAAGGATAGC | 1.01 | SEQ ID |
| ACTATAAT | | NO: 156 |
| AACAGGTCATTGCCGAGAAAAGGATAGCA | 1.01 | SEQ ID |
| CTATAAT | | NO: 157 |
| ACAGGTCATTGCCGAGAAAAGGATAGCAC | 1 | SEQ ID |
| TATAAT | | NO: 34 |
| CAGGTCATTGCCGAGAAAAGGATAGCACT | 1 | SEQ ID |
| ATAAT | | NO: 158 |
| AGGTCATTGCCGAGAAAAGGATAGCACTA | 1 | SEQ ID |
| TAAT | | NO: 159 |
| GGTCATTGCCGAGAAAAGGATAGCACTAT | 0.97 | SEQ ID |
| AAT | | NO: 160 |
| GTCATTGCCGAGAAAAGGATAGCACTATA | 0.98 | SEQ ID |
| AT | | NO: 35 |
| TCATTGCCGAGAAAAGGATAGCACTATAA | 0.99 | SEQ ID |
| T | | NO: 161 |
| CATTGCCGAGAAAAGGATAGCACTATAAT | 1 | SEQ ID |
| | | NO: 162 |
| ATTGCCGAGAAAAGGATAGCACTATAAT | 1 | SEQ ID |
| | | NO: 163 |
| TTGCCGAGAAAAGGATAGCACTATAAT | 0.98 | SEQ ID |
| | | NO: 36 |
| TGCCGAGAAAAGGATAGCACTATAAT | 0.93 | SEQ ID |
| | | NO: 164 |
| GCCGAGAAAAGGATAGCACTATAAT | 0.99 | SEQ ID |
| | | NO: 165 |
| CCGAGAAAAGGATAGCACTATAAT | 1 | SEQ ID |
| | | NO: 166 |
| CGAGAAAAGGATAGCACTATAAT | 1 | SEQ ID |
| | | NO: 37 |
| GAGAAAAGGATAGCACTATAAT | 1 | SEQ ID |
| | | NO: 167 |
| AGAAAAGGATAGCACTATAAT | 1.01 | SEQ ID |
| | | NO: 168 |
| GAAAAGGATAGCACTATAAT | 1 | SEQ ID |
| | | NO: 169 |
| AAAAGGATAGCACTATAAT | 1 | SEQ ID |
| | | NO: 38 |
| AAAGGATAGCACTATAAT | 1 | SEQ ID |
| | | NO: 170 |
| AAGGATAGCACTATAAT | 0.99 | SEQ ID |
| | | NO: 171 |
| AGGATAGCACTATAAT | 0.99 | SEQ ID |
| | | NO: 172 |
| GGATAGCACTATAAT | 0.97 | SEQ ID |
| | | NO: 39 |

TABLE 13
Relative MRL values of 3′ end-truncated
5′UTR I′ truncations to untruncated form

| | Relative | SEQ NO |
|---|---|---|
| 5′UTR I′ truncation | MRL value | ID: |
| TCAATAACAGGTCATTGCCGAGAAAAGGAT | 0.99 | SEQ ID |
| AGCA | | NO: 173 |
| TCAATAACAGGTCATTGCCGAGAAAAGGAT | 0.98 | SEQ ID |
| AGCACTA | | NO: 174 |
| TCAATAACAGGTCATTGCCGAGAAAAGGAT | 1 | SEQ ID |
| AGCACT | | NO: 175 |
| TCAATAACAGGTCATTGCCGAGAAAAGGAT | 0.99 | SEQ ID |
| AGCACTATA | | NO: 176 |
| TCAATAACAGGTCATTGCCGAGAAAAG | 0.97 | SEQ ID |
| | | NO: 177 |
| TCAATAACAGGTCATTGCCGAGAAAAGGAT | 0.97 | SEQ ID |
| A | | NO: 178 |
| TCAATAACAGGTCATTGCCGAGAAAAGGAT | 0.98 | SEQ ID |
| | | NO: 179 |
| TCAATAACAGGTCATTGCCGAGAAAAGGAT | 0.99 | SEQ ID |
| AGC | | NO: 180 |
| TCAATAACAGGTCATTGCCGAGAAAA | 0.95 | SEQ ID |
| | | NO: 181 |
| TCAATAACAGGTCATTGCCGAGAAAAGG | 0.95 | SEQ ID |
| | | NO: 182 |
| TCAATAACAGGTCATTGCCGAGAAA | 0.96 | SEQ ID |
| | | NO: 183 |
| TCAATAACAGGTCATT | 0.93 | SEQ ID |
| | | NO: 184 |
| TCAATAACAGGTCATTGCCGAGAAAAGGAT | 1.01 | SEQ ID |
| AGCACTATAA | | NO: 185 |
| TCAATAACAGGTCATTGCCGAGAAAAGGA | 0.97 | SEQ ID |
| | | NO: 186 |
| TCAATAACAGGTCATTGCCGAG | 0.94 | SEQ ID |
| | | NO: 187 |
| TCAATAACAGGTCATTGCCGAGAAAAGGAT | 0.93 | SEQ ID |
| AGCACTAT | | NO: 188 |
| TCAATAACAGGTCATTGCCGAGAA | 0.96 | SEQ ID |
| | | NO: 189 |
| TCAATAACAGGTCATTG | 0.9 | SEQ ID |
| | | NO: 190 |
| TCAATAACAGGTCATTGCCGAGAAAAGGAT | 0.98 | SEQ ID |
| AG | | NO: 191 |
| TCAATAACAGGTCATTGCCGAGA | 0.94 | SEQ ID |
| | | NO: 192 |
| TCAATAACAGGTCAT | 0.91 | SEQ ID |
| | | NO: 193 |
| TCAATAACAGGTCATTGCC | 0.92 | SEQ ID |
| | | NO: 194 |
| TCAATAACAGGTCATTGCCGAGAAAAGGAT | 0.94 | SEQ ID |
| AGCAC | | NO: 195 |
| TCAATAACAGGTCATTGCCG | 0.93 | SEQ ID |
| | | NO: 196 |
| TCAATAACAGGTCATTGCCGA | 0.92 | SEQ ID |
| | | NO: 41 |
| TCAATAACAGGTCATTGC | 0.85 | SEQ ID |
| | | NO: 76 |

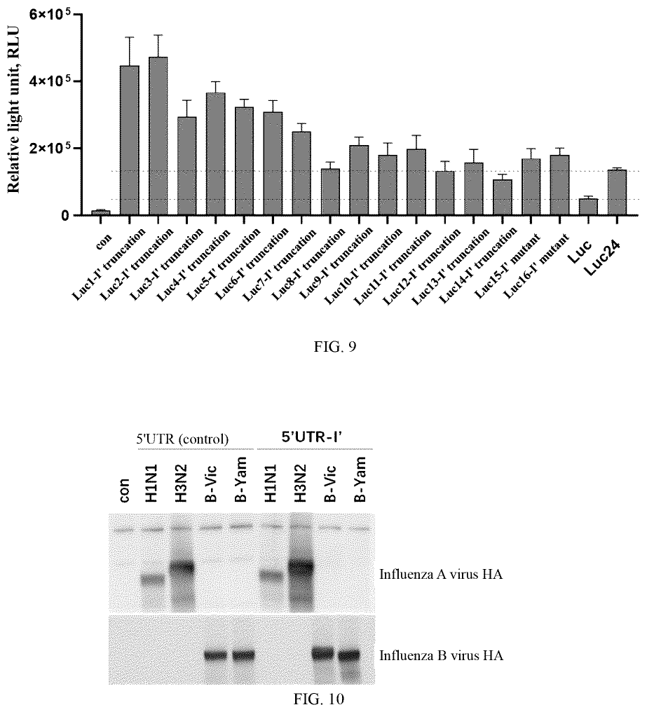

6.2 16 constructs containing 5′UTR I′ truncations with different lengths (SEQ ID NOs: 28-40, CTATAAT) and 2 point mutations A→C and A→T (SEQ ID NOs: 42 and 43) different from the A→G point mutation were synthesized by Genewiz, Suzhou. The 16 synthesized sequences were inserted into Luc 24 in Example 5 by HindIII and ScaI digestion and ligation, thereby given constructs with corresponding luciferase 5′UTR I′ truncations and point mutations, designated as LUC1-16, with corresponding sequences of the 5′UTR I′ truncations (1-I′ to 14-I′) and mutants (15-I′ and 16-I′) set forth in SEQ ID NO: 63-78, respectively. Capped mRNA was synthesized by in vitro transcription as in Example 1 and transfected into 293T cells in vitro for 24 h. The luciferase substrate was added for detection, and the plate was read. The results are shown in FIG. 9. The luciferase expression levels mediated by Luc24 (I′-B combination) and 5′UTR truncations and mutants thereof were significantly higher than that mediated by Luc (5′ and 3′UTRs of BNT162b2). The 5′UTR I′ truncations can retain the activity of 5′UTR I′ in regulating the expression of the target protein. Part of the 5′UTR I′ truncations (1-I′, 2-I′, 3-I′, 4-I′, 5-I′, 6-I′, 7-I′, 9-I′, 10-I′, 11-I′, and 13-I′) exhibited increased activity in regulating the expression of the target protein. Meanwhile, the mutants 15-I′ and 16-I′ of the 5′UTR I′ mediated higher expression levels of the target gene than that of 5′UTR I′, indicating that the functional activity of 5′UTR in regulating the expression of the target gene can be further enhanced by truncating the 5′ end of 5′UTR I′ or point mutation inside.

### Example 7. Differences in Influenza HA mRNA Expression Levels Regulated by Control 5′UTR and 5′UTR I′

Plasmids of the gene encoding influenza A virus H1N1 (A/Wisconsin/588/2019) HA protein (SEQ ID NO: 83), the gene encoding influenza A virus H3N2 (A/Cambodia/e0826360/2020) HA protein (SEQ ID NO: 84), the gene encoding influenza B virus (Washington/02/2019) HA protein (SEQ ID NO: 85), and the gene encoding influenza B virus (Phuket/3073/2013) HA protein (SEQ ID NO: 86) were constructed by replacing the target gene via digestion, with the 5′UTR I′ and the 3′UTR B retained. The target gene sequences contained in the plasmids were SEQ ID NOs: 87-90, which were all codon-optimized sequences. Four HA CDS sequences encoding SEQ ID NOs: 83-86 were synthesized with reference to 5′ and 3′UTR sequences (SEQ ID NOs: 91 and 92) in Patent Nos. WO2022/245888A1 and WO2022/150717A1, as set forth in SEQ ID NOs: 93-96, respectively. mRNAs were prepared and purified as in Example 1. 1 μg of mRNA was transfected to 293T cells by Lipofectamine™ 2000, and the lysate was harvested after 1 day. The expression of the four influenza HA protein mRNAs regulated by I′-B was detected by the Western Blot method in Example 2. The primary antibody was an influenza A virus hemagglutinin/HA antibody (Sino Biology, 86001-RMO1) or an influenza B virus hemagglutinin/HA antibody against (Sino Biology, 11053-R004), and the secondary antibody was an HRP-linked anti-rabbit IgG (Transgene, HS101). The results are shown in FIGS. 10 and 11. The expression levels of the four influenza HA regulated by 5′UTR-I′ were significantly higher as compared to the control 5′UTR.

Part of the sequences of the present disclosure are shown as follows:

>5′UTR I (63 bp)
SEQ ID NO: 1
GAGTTAGGAGTTGATGGCAGTTTCAATAACAGGTCATTGCCGAGAAAAGGATAGC
ACTATAAT
>5′UTR I′ (63 bp)
SEQ ID NO: 2
GAGTTAGGAGTTGGTGGCAGTTTCAATAACAGGTCATTGCCGAGAAAAGGATAGC
ACTATAAT
>5′UTR G
SEQ ID NO: 3
GCACTTTTCTGAGCAGACGTCCAGAGCAGAGTCAGCCGCCACC
>5′UTR A
SEQ ID NO: 4
ACATTTGCTTCTGACACAACTGTGTTCACTAGCAACCTCAAACGCCGCCACC
>5′UTR J
SEQ ID NO: 5
AGAGAGGCAAAGAAACATTGTGAAATCTCCAACTCTTAACCTTCAAGCCGCCACC
>5′UTR BNT162b2
SEQ ID NO: 6
GAATAAACTAGTATTCTTCTGGTCCCCACAGACTCAGAGAGAACCCGCCACC
>3′UTR B
SEQ ID NO: 7
GCTCGCTTTCTTGCTGTCCAATTTCTATTAAAGGTTCCTTTGTTCCCTAAGTCCAAC
TACTAAACTGGGGGATATTATGAAGGGCCTTGAGCATCTGGATTCTGCCTAATAAA
AAACATTTATTTTCATTGCAA
>3′UTR F
SEQ ID NO: 8
CAGACAAGACAAGCTACTGAATACGTTCACATCTGTCTTGATGCCTAATATTTTTAC
ATTTCTGTAAACATATTTCTGAAATATTTTTTGCCTTTCAAGCGACAGATGCCTCATT
TTGTGAAAACTTAATGATGATTTTGTGTTTAAGTTCCAAACATTTGAATAAAATAAT
TGACAATA
>3′UTR E
SEQ ID NO: 9
CCTGAGGCCTCAGAACACAGGTTTCAGATTGATAGGCCTGCAGGTCTCCAGGCAG
CAACCAGCTGAGCGACTAAAGGGCCCAAGGCCAGGGCTCTAGGGATGGGGCTCA
GCAGAGGCTGGGGTAAGGGGAGCCAGGGAGGAGCTGGGCCTAATGCAGCACCGG
GTCCCCAGGATGCCGTGTCTCGGCTGGAGGAGGAGATGCGGAAGCTCCAGGCCA
CGGTGCAGGAGCTCCAGAAGCGCTTGGACAGGCTGGAGGAGACAGTCCAGGCCA
AGTAGAGCCCCGCAGGGCCTCCAGCAGGGTCAGCCATTCACACCCATCCACTCAC
CTCCCATTCCCAGCCACATGGCAGAGAAAAAAATCATAATAAAATGGCTTTATTTT
CTGGTA
>3′UTR D
SEQ ID NO: 10
GGGGCCTTCTGACATGAGTCTGGCCTGGCCCCACCTCCTAGTTCCTCATAATAAAG
ACAGATTGCTTCTTCGCTTCTCACTGAGGGGCCTTCTGACATGAGTCTGGCCTGGC
CCCACCTCCCCAGTTTCTCATAATAAAGACAGATTGCTTCTTCACTTGAAGCTCGC
TTTCTTGCTGTCCAATTTCTATTAAAGGTTCCTTTGTTCCCTAAGTCCAACTACTAA
ACTGGGGGATATTATGAAGGGCCTTGAGCATCTGGATTCTGCCTAATAAAAAACAT
TTATTTTCATTGCAA
>3′UTR BNT162b2
SEQ ID NO: 11
CTCGAGCTGGTACTGCATGCACGCAATGCTAGCTGCCCCTTTCCCGTCCTGGGTAC
CCCGAGTCTCCCCCGACCTCGGGTCCCAGGTATGCTCCCACCTCCACCTGCCCCAC
TCACCACCTCTGCTAGTTCCAGACACCTCCCAAGCACGCAGCAATGCAGCTCAAA
ACGCTTAGCCTAGCCACACCCCCACGGGAAACAGCAGTGATTAACCTTTAGCAAT
AAACGAAAGTTTAACTAAGCTATACTAACCCCAGGGTTGGTCAATTTCGTGCCAGC
CACACCCTGGA
>B.1.617.2 (Delta) spike protein amino acid sequence
SEQ ID NO: 12
MFVFLVLLPLVSSQCVNLRTRTQLPPAYTNSFTRGVYYPDKVFRSSVLHSTQDLFLPFF
SNVTWFHAIHVSGTNGTKRFDNPVLPFNDGVYFASTEKSNIIRGWIFGTTLDSKTQSL
LIVNNATNVVIKVCEFQFCNDPFLDVYYHKNNKSWMESGVYSSANNCTFEYVSQPF
LMDLEGKQGNFKNLREFVFKNIDGYFKIYSKHTPINLVRDLPQGFSALEPLVDLPIGIN
ITRFQTLLALHRSYLTPGDSSSGWTAGAAAYYVGYLQPRTFLLKYNENGTITDAVDC
ALDPLSETKCTLKSFTVEKGIYQTSNFRVQPTESIVRFPNITNLCPFGEVFNATRFASVY
AWNRKRISNCVADYSVLYNSASFSTFKCYGVSPTKLNDLCFTNVYADSFVIRGDEVR
QIAPGQTGKIADYNYKLPDDFTGCVIAWNSNNLDSKVGGNYNYRYRLFRKSNLKPFE
RDISTEIYQAGSKPCNGVEGFNCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSNQVAVLYQGVNCTEVPVAIHADQLTPTWRVYSTG
SNVFQTRAGCLIGAEHVNNSYECDIPIGAGICASYQTQTNSRRRARSVASQSIIAYTMS
LGAENSVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPDPSKPSKRSFI
EDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKI
QDSLSSTASALGKLQNVVNQNAQALNTLVKQLSSNFGAISSVLNDILSRLDPPEAEVQ
IDRLITGRLQSLQTYVTQQLIRAAEIRASANLAATKMSECVLGQSKRVDFCGKGYHL
MSFPQSAPHGVVFLHVTYVPAQEKNFTTAPAICHDGKAHFPREGVFVSNGTHWFVTQ
RNFYEPQIITTDNTFVSGNCDVVIGIVNNTVYDPLQPELDSFKEELDKYFKNHTSPDV
DLGDISGINASVVNIQKEIDRLNEVAKNLNESLIDLQELGKYEQYIKWPWYIWLGFIA
GLIAIVMVTIMLCCMTSCCSCLKGCCSCGSCCKFDEDDSEPVLKGVKLHYT
>B.1.617.2 (Delta) spike protein CDS
SEQ ID NO: 13
ATGTTCGTGTTCCTGGTGCTGCTGCCCCTGGTGAGCTCTCAGTGCGTGAACCTGA
GGACAAGAACACAGCTGCCCCCCGCCTACACCAACAGCTTCACAAGAGGCGTGT
ACTACCCCGACAAGGTGTTCAGAAGCAGCGTGCTGCACTCCACCCAAGACCTGTT
TCTGCCCTTCTTCAGCAACGTGACCTGGTTCCACGCCATCCACGTCAGCGGCACC
AACGGCACCAAGAGATTCGACAACCCCGTGCTGCCCTTCAACGACGGCGTGTACT
TCGCTAGCACCGAAAAGAGCAACATCATCAGAGGCTGGATCTTCGGCACCACCCT
AGACTCCAAGACACAAAGCCTGCTGATCGTTAACAACGCGACCAACGTGGTGATC
AAGGTGTGCGAGTTTCAGTTCTGCAACGACCCCTTCCTGGACGTGTACTACCACA
AGAACAACAAGAGCTGGATGGAGAGCGGGGTCTACAGCAGCGCCAACAACTGCA
CCTTCGAGTACGTGTCTCAGCCCTTCCTGATGGACCTGGAGGGCAAGCAAGGCAA
TTTCAAGAACCTAAGAGAGTTCGTGTTCAAGAACATCGACGGCTACTTCAAGATC
TACAGCAAGCACACCCCCATCAACCTGGTGAGAGACCTGCCCCAAGGCTTCAGCG
CCCTGGAGCCCCTGGTGGACCTGCCCATCGGCATCAACATCACAAGATTTCAGAC
CCTGCTGGCCCTGCACCGTAGCTACCTAACCCCCGGCGATAGTTCCTCTGGCTGGA
CGGCCGGCGCTGCCGCCTACTACGTGGGCTACCTGCAGCCTAGAACCTTCCTGCT
GAAGTACAACGAGAACGGCACTATCACTGATGCCGTGGACTGTGCCCTGGACCCC
CTGAGCGAGACCAAGTGCACCCTCAAGAGCTTCACCGTGGAGAAGGGCATCTATC
AGACAAGCAACTTCAGAGTGCAGCCCACCGAGAGCATCGTGAGATTCCCCAACAT
CACCAACCTGTGCCCCTTCGGCGAGGTGTTCAACGCCACAAGATTCGCTAGCGTG
TACGCGTGGAACAGAAAGAGAATCAGCAACTGCGTGGCCGACTACAGCGTGCTG
TACAACAGCGCTAGCTTCAGCACCTTCAAGTGCTATGGCGTCAGCCCCACCAAGC
TGAACGACCTGTGCTTCACCAACGTGTACGCCGACAGCTTCGTGATCAGAGGCGA
CGAGGTGAGACAGATTGCCCCCGGGCAAACCGGCAAGATCGCCGACTACAACTA
CAAGCTGCCCGACGACTTCACCGGCTGCGTGATCGCGTGGAACAGCAATAACCTG
GATTCCAAGGTGGGCGGCAACTACAACTACCGGTACAGACTGTTCAGAAAGAGC
AACCTGAAGCCCTTCGAGAGAGACATCAGCACCGAGATCTACCAAGCCGGCAGC
AAGCCCTGCAACGGCGTGGAGGGCTTCAACTGCTACTTCCCCCTGCAGAGCTACG
GCTTTCAGCCCACCAACGGCGTGGGCTATCAGCCCTACAGAGTGGTGGTGCTGAG
CTTCGAGCTGCTGCACGCCCCCGCAACCGTGTGCGGCCCCAAAAAGAGCACCAA
CCTGGTGAAGAACAAGTGCGTGAACTTCAACTTTAATGGCCTTACCGGCACGGGC
GTGCTGACCGAGAGCAACAAGAAGTTCCTGCCATTTCAGCAATTCGGCAGAGAC
ATCGCCGACACCACCGACGCGGTCAGAGACCCTCAGACCCTGGAGATCCTGGAC
ATCACCCCCTGTAGCTTCGGCGGCGTGAGCGTGATCACCCCCGGCACTAACACAA
GCAACCAAGTGGCCGTGCTGTACCAAGGCGTGAACTGCACCGAGGTGCCCGTGG
CCATCCACGCCGATCAGCTGACCCCCACCTGGAGAGTATACAGCACCGGAAGCAA
CGTGTTTCAGACAAGAGCCGGCTGCCTGATCGGCGCCGAGCACGTGAACAACAG
CTACGAGTGCGACATCCCCATCGGCGCCGGCATCTGCGCTAGCTATCAGACACAG
ACCAACAGCCGCCGGAGAGCTAGAAGCGTGGCTTCTCAGAGCATCATCGCCTACA
CCATGAGCCTGGGCGCCGAGAACAGCGTGGCCTACAGCAACAACAGCATCGCCAT
CCCCACCAACTTCACCATCAGCGTGACCACCGAGATCCTGCCCGTAAGTATGACC
AAGACAAGCGTGGACTGCACCATGTACATCTGCGGCGACAGCACCGAGTGCAGC
AACCTGCTGCTGCAGTACGGCAGCTTCTGCACACAGCTGAACAGAGCCCTGACCG
GCATCGCCGTGGAGCAAGACAAGAACACCCAAGAGGTGTTCGCCCAAGTGAAGC
AGATCTACAAGACCCCCCCCATCAAGGACTTCGGCGGCTTCAACTTCAGCCAAAT
CCTGCCCGACCCTAGCAAGCCTAGCAAGCGGAGCTTCATCGAGGACCTGCTGTTC
AACAAGGTGACCCTGGCCGACGCCGGCTTCATCAAGCAGTACGGCGACTGCCTAG
GCGACATCGCAGCTAGAGACCTGATCTGCGCTCAGAAGTTCAATGGACTCACCGT
GCTCCCCCCCCTGCTGACCGACGAGATGATCGCTCAGTACACCTCTGCCCTGCTGG
CGGGAACCATAACAAGCGGTTGGACATTTGGCGCCGGGGCCGCACTGCAAATCCC
CTTCGCCATGCAGATGGCCTACAGATTCAACGGCATCGGCGTGACACAGAACGTG
CTGTACGAGAATCAGAAGCTGATCGCCAATCAGTTCAACAGCGCCATCGGCAAGA
TCCAAGACAGCCTGAGCAGCACCGCTAGCGCCCTGGGCAAGCTGCAAAACGTGG
TGAATCAGAACGCCCAAGCCCTGAACACCCTGGTGAAGCAGCTGAGCAGCAACT
TCGGCGCCATCAGCAGCGTATTGAATGACATCCTGTCTAGACTGGACCCCCCCGAG
GCCGAGGTGCAGATCGACAGACTGATCACCGGCAGACTGCAGAGCCTGCAGACC
TACGTGACACAGCAGCTGATCAGAGCCGCCGAGATCAGAGCTAGCGCCAACCTG
GCCGCCACCAAGATGAGCGAGTGCGTGCTGGGGCAGAGCAAGAGAGTGGACTTC
TGCGGCAAGGGCTACCACCTGATGAGCTTCCCTCAGAGCGCCCCCCACGGCGTGG
TGTTCCTGCACGTGACCTACGTGCCCGCCCAAGAGAAGAACTTCACCACCGCTCC
CGCCATCTGCCACGACGGCAAGGCCCACTTCCCTAGAGAGGGCGTGTTCGTGAGC
AACGGCACCCACTGGTTCGTGACACAGAGAAACTTCTACGAGCCTCAGATCATCA
CCACCGACAACACCTTCGTGAGCGGCAACTGCGACGTGGTGATCGGCATAGTGAA
CAACACCGTATACGACCCCCTGCAGCCCGAGCTGGACAGCTTCAAGGAGGAGCT
GGACAAGTACTTCAAAAACCATACTAGCCCCGACGTGGACCTGGGAGACATCTCT
GGCATCAACGCTAGCGTGGTGAACATTCAGAAGGAGATCGACCGGCTGAATGAGG
TGGCCAAGAACCTGAACGAGAGCCTGATCGACCTGCAAGAGCTGGGCAAGTACG
AGCAGTACATCAAGTGGCCCTGGTACATCTGGCTGGGCTTCATCGCCGGCCTGATC
GCCATCGTGATGGTGACCATCATGCTGTGCTGCATGACAAGCTGTTGCAGCTGCCT
GAAAGGTTGCTGCAGCTGTGGCTCGTGCTGCAAGTTCGACGAGGACGACAGCGA
GCCCGTGCTGAAGGGCGTGAAGCTGCACTACACCTGATGA
>B.1.1.529 (Omicron) spike protein amino acid sequence
SEQ ID NO: 14
MFVFLVLLPLVSSQCVNLTTRTQLPPAYTNSFTRGVYYPDKVFRSSVLHSTQDLFLPFF
SNVTWFHVISGTNGTKRFDNPVLPFNDGVYFASIEKSNIIRGWIFGTTLDSKTQSLLIV
NNATNVVIKVCEFQFCNDPFLDHKNNKSWMESEFRVYSSANNCTFEYVSQPFLMDL
EGKQGNFKNLREFVFKNIDGYFKIYSKHTPIIVREPEDLPQGFSALEPLVDLPIGINITRF
QTLLALHRSYLTPGDSSSGWTAGAAAYYVGYLQPRTFLLKYNENGTITDAVDCALDP
LSETKCTLKSFTVEKGIYQTSNFRVQPTESIVRFPNITNLCPFDEVFNATRFASVYAWN
RKRISNCVADYSVLYNLAPFFTFKCYGVSPTKLNDLCFTNVYADSFVIRGDEVRQIAP
GQTGNIADYNYKLPDDFTGCVIAWNSNKLDSKVSGNYNYLYRLFRKSNLKPFERDIS
TEIYQAGNKPCNGVAGFNCYFPLRSYSFRPTYGVGHQPYRVVVLSFELLHAPATVCGP
KKSTNLVKNKCVNFNFNGLKGTGVLTESNKKFLPFQQFGRDIADTTDAVRDPQTLEIL
DITPCSFGGVSVITPGTNTSNQVAVLYQGVNCTEVPVAIHADQLTPTWRVYSTGSNVF
QTRAGCLIGAEYVNNSYECDIPIGAGICASYQTQTKSHRRARSVASQSIIAYTMSLGAE
NSVAYSNNSIAIPTNFTISVTTEILPVSMTKTSVDCTMYICGDSTECSNLLLQYGSFCTQ
LKRALTGIAVEQDKNTQEVFAQVKQIYKTPPIKYFGGFNFSQILPDPSKPSKRSFIEDLL
FNKVTLADAGFIKQYGDCLGDIAARDLICAQKFKGLTVLPPLLTDEMIAQYTSALLAG
TITSGWTFGAGAALQIPFAMQMAYRFNGIGVTQNVLYENQKLIANQFNSAIGKIQDSL
SSTASALGKLQDVVNHNAQALNTLVKQLSSKFGAISSVLNDIFSRLDPPEAEVQIDRLI
TGRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDIS
GINASVVNIQKEIDRLNEVAKNLNESLIDLQELGKYEQYIKWPWYIWLGFIAGLIAIV
MVTIMLCCMTSCCSCLKGCCSCGSCCKFDEDDSEPVLKGVKLHYT
>B.1.1.529 (Omicron) spike protein CDS
SEQ ID NO: 15
ATGTTCGTGTTCCTGGTGCTGCTGCCCCTGGTGAGCTCTCAGTGCGTGAACCTGAC
CACAAGAACACAGCTGCCCCCCGCCTACACCAACAGCTTCACAAGAGGCGTGTA
CTACCCCGACAAGGTGTTCAGAAGCAGCGTGCTGCACTCCACCCAAGACCTGTTT
CTGCCCTTCTTCAGCAACGTGACCTGGTTCCACGTGATCAGCGGCACCAACGGCA
CCAAGAGATTCGACAACCCCGTGCTGCCCTTCAACGACGGCGTGTACTTCGCTAG
CATCGAAAAGAGCAACATCATCAGAGGCTGGATCTTCGGCACCACCCTAGACTCC
AAGACACAAAGCCTGCTGATCGTTAACAACGCGACCAACGTGGTGATCAAGGTGT
GCGAGTTTCAGTTCTGCAACGACCCCTTCCTGGACCACAAGAACAACAAGAGCT
GGATGGAGAGCGAGTTCAGAGTGTACAGCAGCGCCAACAACTGCACCTTCGAGT
ACGTGTCTCAGCCCTTCCTGATGGACCTGGAGGGCAAGCAAGGCAATTTCAAGAA
CCTAAGAGAGTTCGTGTTCAAGAACATCGACGGCTACTTCAAGATCTACAGCAAG
CACACCCCCATCATCGTGAGAGAGCCCGAGGACCTGCCCCAAGGCTTCAGCGCCC
TGGAGCCCCTGGTGGACCTGCCCATCGGCATCAACATCACAAGATTTCAGACCCT
GCTGGCCCTGCACCGTAGCTACCTAACCCCCGGCGATAGTTCCTCTGGCTGGACG
GCCGGCGCTGCCGCCTACTACGTGGGCTACCTGCAGCCTAGAACCTTCCTGCTGA
AGTACAACGAGAACGGCACTATCACTGATGCCGTGGACTGTGCCCTGGACCCCCT
GAGCGAGACCAAGTGCACCCTCAAGAGCTTCACCGTGGAGAAGGGCATCTATCA
GACAAGCAACTTCAGAGTGCAGCCCACCGAGAGCATCGTGAGATTCCCCAACATC
ACCAACCTGTGCCCCTTCGACGAGGTGTTCAACGCCACCAGATTTGCTAGCGTGT
ACGCCTGGAACCGGAAGAGAATCAGCAACTGCGTGGCCGACTACAGCGTGCTGT
ACAACCTGGCCCCCTTCTTCACCTTCAAGTGCTATGGCGTCAGCCCCACCAAGCT
GAACGACCTGTGCTTCACCAACGTGTACGCCGACAGCTTCGTGATCAGAGGCGAC
GAGGTGAGACAGATCGCCCCCGGGCAGACCGGCAACATCGCCGACTACAACTAC
AAGCTGCCCGACGACTTCACCGGCTGCGTGATCGCCTGGAACAGCAACAAGCTG
GATAGCAAGGTGAGCGGCAACTACAATTACCTGTACAGACTGTTCAGAAAGAGCA
ACCTGAAGCCCTTCGAGAGAGACATCAGCACCGAGATCTACCAAGCCGGCAACA
AGCCCTGCAACGGCGTGGCGGGCTTCAACTGCTACTTCCCCCTCAGGAGCTACAG
CTTCAGACCCACCTACGGCGTGGGCCATCAGCCCTACAGAGTGGTCGTGCTGAGC
TTCGAGCTGCTGCACGCCCCTGCCACAGTGTGCGGCCCCAAAAAGAGCACCAAC
CTGGTGAAGAACAAGTGCGTGAACTTCAACTTCAACGGCCTGAAGGGCACCGGC
GTGCTGACCGAGAGCAACAAGAAGTTCCTGCCATTTCAGCAATTCGGCAGAGAC
ATCGCCGACACCACCGACGCGGTCAGAGACCCTCAGACCCTGGAGATCCTGGAC
ATCACCCCCTGTAGCTTCGGCGGCGTGAGCGTGATCACCCCCGGCACTAACACAA
GCAACCAAGTGGCCGTGCTGTACCAAGGCGTGAACTGCACCGAGGTGCCCGTGG
CCATCCACGCCGATCAGCTGACCCCCACCTGGAGAGTGTATAGCACCGGCAGCAA
CGTGTTTCAGACAAGAGCCGGCTGCCTGATCGGCGCCGAGTACGTGAACAACAG
CTACGAGTGCGACATCCCCATCGGCGCCGGCATCTGCGCTAGCTATCAGACACAG
ACCAAGAGCCACCGGAGAGCTAGAAGCGTGGCTAGCCAAAGCATCATCGCCTAC
ACCATGAGCCTGGGCGCCGAGAACAGCGTGGCCTACAGCAACAACAGCATCGCC
ATCCCCACCAACTTCACCATCAGCGTGACCACCGAGATCCTGCCCGTAAGTATGAC
CAAGACAAGCGTGGACTGCACCATGTACATCTGCGGCGACAGCACCGAGTGCAG
CAACCTGCTCCTGCAGTACGGCAGCTTCTGCACACAGCTGAAGAGAGCCCTGACC
GGCATCGCCGTGGAGCAAGACAAGAACACCCAAGAGGTGTTCGCCCAAGTGAAG
CAGATCTACAAGACCCCCCCCATCAAGTACTTCGGCGGCTTCAACTTCAGCCAAAT
CCTGCCCGACCCTAGCAAGCCTAGCAAGCGGAGCTTCATCGAGGACCTGCTGTTC
AACAAGGTGACCCTGGCCGACGCCGGCTTCATCAAGCAGTACGGCGACTGCCTAG
GCGACATCGCAGCTAGAGACCTGATCTGCGCTCAGAAGTTCAAGGGACTCACCGT
GCTCCCCCCCCTGCTGACCGACGAGATGATCGCTCAGTACACCTCTGCCCTGCTGG
CGGGAACCATAACAAGCGGTTGGACATTTGGCGCCGGGGCCGCACTGCAAATCCC
CTTCGCCATGCAGATGGCCTACAGATTCAACGGCATCGGCGTGACACAGAACGTG
CTGTACGAGAATCAGAAGCTGATCGCCAATCAGTTCAACAGCGCCATCGGCAAGA
TCCAAGACAGCCTGAGCAGCACCGCTAGCGCCCTGGGCAAGCTGCAAGACGTGG
TGAACCACAACGCCCAAGCCCTGAACACCCTGGTGAAGCAGCTGAGCAGCAAGT
TCGGCGCCATCAGCAGCGTGCTGAATGACATCTTCTCTAGACTGGACCCCCCCGA
GGCCGAGGTGCAGATCGACAGACTGATCACCGGCAGACTGCAGAGCCTGCAGAC
CTACGTGACACAGCAGCTGATCAGAGCCGCCGAGATCAGAGCTAGCGCCAACCTG
GCCGCCACCAAGATGAGCGAGTGCGTGCTGGGGCAGAGCAAGAGAGTGGACTTC
TGCGGCAAGGGCTACCACCTGATGAGCTTCCCTCAGAGCGCCCCCCACGGCGTGG
TGTTCCTGCACGTGACCTACGTGCCCGCCCAAGAGAAGAACTTCACCACCGCTCC
CGCCATCTGCCACGACGGCAAGGCCCACTTCCCTAGAGAGGGCGTGTTCGTGAGC
AACGGCACCCACTGGTTCGTGACACAGAGAAACTTCTACGAGCCTCAGATCATCA
CCACCGACAACACCTTCGTGAGCGGCAACTGCGACGTGGTGATCGGCATAGTGAA
CAACACCGTATACGACCCCCTGCAGCCCGAGCTGGACAGCTTCAAGGAGGAGCT
GGACAAGTACTTCAAAAACCATACTAGCCCCGACGTGGACCTGGGAGACATCTCT
GGCATCAACGCTAGCGTGGTGAACATTCAGAAGGAGATCGACCGGCTGAATGAGG
TGGCCAAGAACCTGAACGAGAGCCTGATCGACCTGCAAGAGCTGGGCAAGTACG
AGCAGTACATCAAGTGGCCCTGGTACATCTGGCTGGGCTTCATCGCCGGCCTGATC
GCCATCGTGATGGTGACCATCATGCTGTGCTGCATGACAAGCTGTTGCAGCTGCCT
GAAAGGTTGCTGCAGCTGTGGCTCGTGCTGCAAGTTCGACGAGGACGACAGCGA
GCCCGTGCTGAAGGGCGTGAAGCTGCACTACACCTGATGA
>poly A
SEQ ID NO: 16
AAAAAAAAAAAAAAAAAAAAAAAAAAAAAAGCATATGACTAAAAAAAAAAAAA
AAAAAAAAAAAAAAAAAAAAAAAAAAAAAAAAAAAAAAAAAAAAAAAAAAAA
AAAAAAA
>T7 promoter
SEQ ID NO: 17
TAATACGACTCACTATAAGG
>I′-Delta24-B
SEQ ID NO: 18
GAGTTAGGAGTTGGTGGCAGTTTCAATAACAGGTCATTGCCGAGAAAAGGATAGC
ACTATAATATGTTCGTGTTCCTGGTGCTGCTGCCCCTGGTGAGCTCTCAGTGCGTG
AACCTGAGGACAAGAACACAGCTGCCCCCCGCCTACACCAACAGCTTCACAAGA
GGCGTGTACTACCCCGACAAGGTGTTCAGAAGCAGCGTGCTGCACTCCACCCAAG
ACCTGTTTCTGCCCTTCTTCAGCAACGTGACCTGGTTCCACGCCATCCACGTCAGC
GGCACCAACGGCACCAAGAGATTCGACAACCCCGTGCTGCCCTTCAACGACGGC
GTGTACTTCGCTAGCACCGAAAAGAGCAACATCATCAGAGGCTGGATCTTCGGCA
CCACCCTAGACTCCAAGACACAAAGCCTGCTGATCGTTAACAACGCGACCAACGT
GGTGATCAAGGTGTGCGAGTTTCAGTTCTGCAACGACCCCTTCCTGGACGTGTAC
TACCACAAGAACAACAAGAGCTGGATGGAGAGCGGGGTCTACAGCAGCGCCAAC
AACTGCACCTTCGAGTACGTGTCTCAGCCCTTCCTGATGGACCTGGAGGGCAAGC
AAGGCAATTTCAAGAACCTAAGAGAGTTCGTGTTCAAGAACATCGACGGCTACTT
CAAGATCTACAGCAAGCACACCCCCATCAACCTGGTGAGAGACCTGCCCCAAGGC
TTCAGCGCCCTGGAGCCCCTGGTGGACCTGCCCATCGGCATCAACATCACAAGAT
TTCAGACCCTGCTGGCCCTGCACCGTAGCTACCTAACCCCCGGCGATAGTTCCTCT
GGCTGGACGGCCGGCGCTGCCGCCTACTACGTGGGCTACCTGCAGCCTAGAACCT
TCCTGCTGAAGTACAACGAGAACGGCACTATCACTGATGCCGTGGACTGTGCCCT
GGACCCCCTGAGCGAGACCAAGTGCACCCTCAAGAGCTTCACCGTGGAGAAGGG
CATCTATCAGACAAGCAACTTCAGAGTGCAGCCCACCGAGAGCATCGTGAGATTC
CCCAACATCACCAACCTGTGCCCCTTCGGCGAGGTGTTCAACGCCACAAGATTCG
CTAGCGTGTACGCGTGGAACAGAAAGAGAATCAGCAACTGCGTGGCCGACTACA
GCGTGCTGTACAACAGCGCTAGCTTCAGCACCTTCAAGTGCTATGGCGTCAGCCC
CACCAAGCTGAACGACCTGTGCTTCACCAACGTGTACGCCGACAGCTTCGTGATC
AGAGGCGACGAGGTGAGACAGATTGCCCCCGGGCAAACCGGCAAGATCGCCGAC
TACAACTACAAGCTGCCCGACGACTTCACCGGCTGCGTGATCGCGTGGAACAGCA
ATAACCTGGATTCCAAGGTGGGCGGCAACTACAACTACCGGTACAGACTGTTCAG
AAAGAGCAACCTGAAGCCCTTCGAGAGAGACATCAGCACCGAGATCTACCAAGC
CGGCAGCAAGCCCTGCAACGGCGTGGAGGGCTTCAACTGCTACTTCCCCCTGCAG
AGCTACGGCTTTCAGCCCACCAACGGCGTGGGCTATCAGCCCTACAGAGTGGTGG
TGCTGAGCTTCGAGCTGCTGCACGCCCCCGCAACCGTGTGCGGCCCCAAAAAGA
GCACCAACCTGGTGAAGAACAAGTGCGTGAACTTCAACTTTAATGGCCTTACCGG
CACGGGCGTGCTGACCGAGAGCAACAAGAAGTTCCTGCCATTTCAGCAATTCGGC
AGAGACATCGCCGACACCACCGACGCGGTCAGAGACCCTCAGACCCTGGAGATC
CTGGACATCACCCCCTGTAGCTTCGGCGGCGTGAGCGTGATCACCCCCGGCACTA
ACACAAGCAACCAAGTGGCCGTGCTGTACCAAGGCGTGAACTGCACCGAGGTGC
CCGTGGCCATCCACGCCGATCAGCTGACCCCCACCTGGAGAGTATACAGCACCGG
AAGCAACGTGTTTCAGACAAGAGCCGGCTGCCTGATCGGCGCCGAGCACGTGAA
CAACAGCTACGAGTGCGACATCCCCATCGGCGCCGGCATCTGCGCTAGCTATCAG
ACACAGACCAACAGCCGCCGGAGAGCTAGAAGCGTGGCTTCTCAGAGCATCATC
GCCTACACCATGAGCCTGGGCGCCGAGAACAGCGTGGCCTACAGCAACAACAGC
ATCGCCATCCCCACCAACTTCACCATCAGCGTGACCACCGAGATCCTGCCCGTAAG
TATGACCAAGACAAGCGTGGACTGCACCATGTACATCTGCGGCGACAGCACCGAG
TGCAGCAACCTGCTGCTGCAGTACGGCAGCTTCTGCACACAGCTGAACAGAGCCC
TGACCGGCATCGCCGTGGAGCAAGACAAGAACACCCAAGAGGTGTTCGCCCAAG
TGAAGCAGATCTACAAGACCCCCCCCATCAAGGACTTCGGCGGCTTCAACTTCAG
CCAAATCCTGCCCGACCCTAGCAAGCCTAGCAAGCGGAGCTTCATCGAGGACCTG
CTGTTCAACAAGGTGACCCTGGCCGACGCCGGCTTCATCAAGCAGTACGGCGACT
GCCTAGGCGACATCGCAGCTAGAGACCTGATCTGCGCTCAGAAGTTCAATGGACT
CACCGTGCTCCCCCCCCTGCTGACCGACGAGATGATCGCTCAGTACACCTCTGCCC
TGCTGGCGGGAACCATAACAAGCGGTTGGACATTTGGCGCCGGGGCCGCACTGCA
AATCCCCTTCGCCATGCAGATGGCCTACAGATTCAACGGCATCGGCGTGACACAG
AACGTGCTGTACGAGAATCAGAAGCTGATCGCCAATCAGTTCAACAGCGCCATCG
GCAAGATCCAAGACAGCCTGAGCAGCACCGCTAGCGCCCTGGGCAAGCTGCAAA
ACGTGGTGAATCAGAACGCCCAAGCCCTGAACACCCTGGTGAAGCAGCTGAGCA
GCAACTTCGGCGCCATCAGCAGCGTATTGAATGACATCCTGTCTAGACTGGACCCC
CCCGAGGCCGAGGTGCAGATCGACAGACTGATCACCGGCAGACTGCAGAGCCTG
CAGACCTACGTGACACAGCAGCTGATCAGAGCCGCCGAGATCAGAGCTAGCGCC
AACCTGGCCGCCACCAAGATGAGCGAGTGCGTGCTGGGGCAGAGCAAGAGAGTG
GACTTCTGCGGCAAGGGCTACCACCTGATGAGCTTCCCTCAGAGCGCCCCCCACG
GCGTGGTGTTCCTGCACGTGACCTACGTGCCCGCCCAAGAGAAGAACTTCACCAC
CGCTCCCGCCATCTGCCACGACGGCAAGGCCCACTTCCCTAGAGAGGGCGTGTTC
GTGAGCAACGGCACCCACTGGTTCGTGACACAGAGAAACTTCTACGAGCCTCAG
ATCATCACCACCGACAACACCTTCGTGAGCGGCAACTGCGACGTGGTGATCGGCA
TAGTGAACAACACCGTATACGACCCCCTGCAGCCCGAGCTGGACAGCTTCAAGGA
GGAGCTGGACAAGTACTTCAAAAACCATACTAGCCCCGACGTGGACCTGGGAGA
CATCTCTGGCATCAACGCTAGCGTGGTGAACATTCAGAAGGAGATCGACCGGCTG
AATGAGGTGGCCAAGAACCTGAACGAGAGCCTGATCGACCTGCAAGAGCTGGGC
AAGTACGAGCAGTACATCAAGTGGCCCTGGTACATCTGGCTGGGCTTCATCGCCG
GCCTGATCGCCATCGTGATGGTGACCATCATGCTGTGCTGCATGACAAGCTGTTGC
AGCTGCCTGAAAGGTTGCTGCAGCTGTGGCTCGTGCTGCAAGTTCGACGAGGAC
GACAGCGAGCCCGTGCTGAAGGGCGTGAAGCTGCACTACACCTGATGACTCGAG
GCTCGCTTTCTTGCTGTCCAATTTCTATTAAAGGTTCCTTTGTTCCCTAAGTCCAAC
TACTAAACTGGGGGATATTATGAAGGGCCTTGAGCATCTGGATTCTGCCTAATAAA
AAACATTTATTTTCATTGCAAAAAAAAAAAAAAAAAAAAAAAAAAAAAAAAGCA
TATGACTAAAAAAAAAAAAAAAAAAAAAAAAAAAAAAAAAAAAAAAAAAAAA
AAAAAAAAAAAAAAAAAAAAAAAAAAA
>I′-OC24-B
SEQ ID NO: 19
GAGTTAGGAGTTGGTGGCAGTTTCAATAACAGGTCATTGCCGAGAAAAGGATAGC
ACTATAATATGTTCGTGTTCCTGGTGCTGCTGCCCCTGGTGAGCTCTCAGTGCGTG
AACCTGACCACAAGAACACAGCTGCCCCCCGCCTACACCAACAGCTTCACAAGA
GGCGTGTACTACCCCGACAAGGTGTTCAGAAGCAGCGTGCTGCACTCCACCCAAG
ACCTGTTTCTGCCCTTCTTCAGCAACGTGACCTGGTTCCACGTGATCAGCGGCACC
AACGGCACCAAGAGATTCGACAACCCCGTGCTGCCCTTCAACGACGGCGTGTACT
TCGCTAGCATCGAAAAGAGCAACATCATCAGAGGCTGGATCTTCGGCACCACCCT
AGACTCCAAGACACAAAGCCTGCTGATCGTTAACAACGCGACCAACGTGGTGATC
AAGGTGTGCGAGTTTCAGTTCTGCAACGACCCCTTCCTGGACCACAAGAACAAC
AAGAGCTGGATGGAGAGCGAGTTCAGAGTGTACAGCAGCGCCAACAACTGCACC
TTCGAGTACGTGTCTCAGCCCTTCCTGATGGACCTGGAGGGCAAGCAAGGCAATT
TCAAGAACCTAAGAGAGTTCGTGTTCAAGAACATCGACGGCTACTTCAAGATCTA
CAGCAAGCACACCCCCATCATCGTGAGAGAGCCCGAGGACCTGCCCCAAGGCTT
CAGCGCCCTGGAGCCCCTGGTGGACCTGCCCATCGGCATCAACATCACAAGATTT
CAGACCCTGCTGGCCCTGCACCGTAGCTACCTAACCCCCGGCGATAGTTCCTCTGG
CTGGACGGCCGGCGCTGCCGCCTACTACGTGGGCTACCTGCAGCCTAGAACCTTC
CTGCTGAAGTACAACGAGAACGGCACTATCACTGATGCCGTGGACTGTGCCCTGG
ACCCCCTGAGCGAGACCAAGTGCACCCTCAAGAGCTTCACCGTGGAGAAGGGCA
TCTATCAGACAAGCAACTTCAGAGTGCAGCCCACCGAGAGCATCGTGAGATTCCC
CAACATCACCAACCTGTGCCCCTTCGACGAGGTGTTCAACGCCACCAGATTTGCT
AGCGTGTACGCCTGGAACCGGAAGAGAATCAGCAACTGCGTGGCCGACTACAGC
GTGCTGTACAACCTGGCCCCCTTCTTCACCTTCAAGTGCTATGGCGTCAGCCCCAC
CAAGCTGAACGACCTGTGCTTCACCAACGTGTACGCCGACAGCTTCGTGATCAGA
GGCGACGAGGTGAGACAGATCGCCCCCGGGCAGACCGGCAACATCGCCGACTAC
AACTACAAGCTGCCCGACGACTTCACCGGCTGCGTGATCGCCTGGAACAGCAACA
AGCTGGATAGCAAGGTGAGCGGCAACTACAATTACCTGTACAGACTGTTCAGAAA
GAGCAACCTGAAGCCCTTCGAGAGAGACATCAGCACCGAGATCTACCAAGCCGG
CAACAAGCCCTGCAACGGCGTGGCGGGCTTCAACTGCTACTTCCCCCTCAGGAGC
TACAGCTTCAGACCCACCTACGGCGTGGGCCATCAGCCCTACAGAGTGGTCGTGC
TGAGCTTCGAGCTGCTGCACGCCCCTGCCACAGTGTGCGGCCCCAAAAAGAGCA
CCAACCTGGTGAAGAACAAGTGCGTGAACTTCAACTTCAACGGCCTGAAGGGCA
CCGGCGTGCTGACCGAGAGCAACAAGAAGTTCCTGCCATTTCAGCAATTCGGCAG
AGACATCGCCGACACCACCGACGCGGTCAGAGACCCTCAGACCCTGGAGATCCT
GGACATCACCCCCTGTAGCTTCGGCGGCGTGAGCGTGATCACCCCCGGCACTAAC
ACAAGCAACCAAGTGGCCGTGCTGTACCAAGGCGTGAACTGCACCGAGGTGCCC
GTGGCCATCCACGCCGATCAGCTGACCCCCACCTGGAGAGTGTATAGCACCGGCA
GCAACGTGTTTCAGACAAGAGCCGGCTGCCTGATCGGCGCCGAGTACGTGAACA
ACAGCTACGAGTGCGACATCCCCATCGGCGCCGGCATCTGCGCTAGCTATCAGAC
ACAGACCAAGAGCCACCGGAGAGCTAGAAGCGTGGCTAGCCAAAGCATCATCGC
CTACACCATGAGCCTGGGCGCCGAGAACAGCGTGGCCTACAGCAACAACAGCAT
CGCCATCCCCACCAACTTCACCATCAGCGTGACCACCGAGATCCTGCCCGTAAGT
ATGACCAAGACAAGCGTGGACTGCACCATGTACATCTGCGGCGACAGCACCGAGT
GCAGCAACCTGCTCCTGCAGTACGGCAGCTTCTGCACACAGCTGAAGAGAGCCCT
GACCGGCATCGCCGTGGAGCAAGACAAGAACACCCAAGAGGTGTTCGCCCAAGT
GAAGCAGATCTACAAGACCCCCCCCATCAAGTACTTCGGCGGCTTCAACTTCAGC
CAAATCCTGCCCGACCCTAGCAAGCCTAGCAAGCGGAGCTTCATCGAGGACCTGC
TGTTCAACAAGGTGACCCTGGCCGACGCCGGCTTCATCAAGCAGTACGGCGACTG
CCTAGGCGACATCGCAGCTAGAGACCTGATCTGCGCTCAGAAGTTCAAGGGACTC
ACCGTGCTCCCCCCCCTGCTGACCGACGAGATGATCGCTCAGTACACCTCTGCCCT
GCTGGCGGGAACCATAACAAGCGGTTGGACATTTGGCGCCGGGGCCGCACTGCA
AATCCCCTTCGCCATGCAGATGGCCTACAGATTCAACGGCATCGGCGTGACACAG
AACGTGCTGTACGAGAATCAGAAGCTGATCGCCAATCAGTTCAACAGCGCCATCG
GCAAGATCCAAGACAGCCTGAGCAGCACCGCTAGCGCCCTGGGCAAGCTGCAAG
ACGTGGTGAACCACAACGCCCAAGCCCTGAACACCCTGGTGAAGCAGCTGAGCA
GCAAGTTCGGCGCCATCAGCAGCGTGCTGAATGACATCTTCTCTAGACTGGACCC
CCCCGAGGCCGAGGTGCAGATCGACAGACTGATCACCGGCAGACTGCAGAGCCT
GCAGACCTACGTGACACAGCAGCTGATCAGAGCCGCCGAGATCAGAGCTAGCGC
CAACCTGGCCGCCACCAAGATGAGCGAGTGCGTGCTGGGGCAGAGCAAGAGAGT
GGACTTCTGCGGCAAGGGCTACCACCTGATGAGCTTCCCTCAGAGCGCCCCCCAC
GGCGTGGTGTTCCTGCACGTGACCTACGTGCCCGCCCAAGAGAAGAACTTCACCA
CCGCTCCCGCCATCTGCCACGACGGCAAGGCCCACTTCCCTAGAGAGGGCGTGTT
CGTGAGCAACGGCACCCACTGGTTCGTGACACAGAGAAACTTCTACGAGCCTCA
GATCATCACCACCGACAACACCTTCGTGAGCGGCAACTGCGACGTGGTGATCGGC
ATAGTGAACAACACCGTATACGACCCCCTGCAGCCCGAGCTGGACAGCTTCAAGG
AGGAGCTGGACAAGTACTTCAAAAACCATACTAGCCCCGACGTGGACCTGGGAG
ACATCTCTGGCATCAACGCTAGCGTGGTGAACATTCAGAAGGAGATCGACCGGCT
GAATGAGGTGGCCAAGAACCTGAACGAGAGCCTGATCGACCTGCAAGAGCTGGG
CAAGTACGAGCAGTACATCAAGTGGCCCTGGTACATCTGGCTGGGCTTCATCGCC
GGCCTGATCGCCATCGTGATGGTGACCATCATGCTGTGCTGCATGACAAGCTGTTG
CAGCTGCCTGAAAGGTTGCTGCAGCTGTGGCTCGTGCTGCAAGTTCGACGAGGA
CGACAGCGAGCCCGTGCTGAAGGGCGTGAAGCTGCACTACACCTGATGACTCGA
GGCTCGCTTTCTTGCTGTCCAATTTCTATTAAAGGTTCCTTTGTTCCCTAAGTCCAA
CTACTAAACTGGGGGATATTATGAAGGGCCTTGAGCATCTGGATTCTGCCTAATAA
AAAACATTTATTTTCATTGCAAAAAAAAAAAAAAAAAAAAAAAAAAAAAAAAGC
ATATGACTAAAAAAAAAAAAAAAAAAAAAAAAAAAAAAAAAAAAAAAAAAAAA
AAAAAAAAAAAAAAAAAAAAAAAAAAA
>B.1.351 spike protein CDS
SEQ ID NO: 20
ATGTTCGTGTTCCTGGTGCTGCTGCCCCTGGTGAGCTCTCAGTGCGTGAACTTCAC
GACAAGAACACAGCTGCCCCCCGCCTACACCAACAGCTTCACAAGAGGCGTGTA
CTACCCCGACAAGGTGTTCAGAAGCAGCGTGCTGCACTCCACCCAAGACCTGTTT
CTGCCCTTCTTCAGCAACGTGACCTGGTTCCACGCCATCCACGTGAGCGGCACCA
ACGGCACCAAGAGATTCGCCAACCCCGTGCTGCCCTTCAACGACGGCGTGTACTT
CGCTAGCACCGAAAAGAGCAACATCATCAGAGGCTGGATCTTCGGCACCACCCTA
GACTCCAAGACACAAAGCCTGCTGATCGTTAACAACGCGACCAACGTGGTGATCA
AGGTGTGCGAGTTTCAGTTCTGCAACGACCCCTTCCTGGGCGTGTACTACCACAA
GAACAACAAGAGCTGGATGGAGAGCGAGTTCCGTGTCTACAGCAGCGCCAACAA
CTGCACCTTCGAGTACGTGTCTCAGCCCTTCCTGATGGACCTGGAGGGCAAGCAA
GGCAATTTCAAGAACCTAAGAGAGTTCGTGTTCAAGAACATCGACGGCTACTTCA
AGATCTACAGCAAGCACACCCCCATCAACCTGGTGAGAGGCCTGCCCCAAGGCTT
CAGCGCCCTGGAGCCCCTGGTGGACCTGCCCATCGGCATCAACATCACAAGATTT
CAGACCCTGCACCGTAGCTACCTAACCCCCGGCGATAGTTCCTCTGGCTGGACGG
CCGGCGCTGCCGCCTACTACGTGGGCTACCTGCAGCCTAGAACCTTCCTGCTGAA
GTACAACGAGAACGGCACTATCACTGATGCCGTGGACTGTGCCCTGGACCCCCTG
AGCGAGACCAAGTGCACCCTCAAGAGCTTCACCGTGGAGAAGGGCATCTATCAG
ACAAGCAACTTCAGAGTGCAGCCCACCGAGAGCATCGTGAGATTCCCCAACATCA
CCAACCTGTGCCCCTTCGGCGAGGTGTTCAACGCCACAAGATTCGCTAGCGTGTA
CGCGTGGAACAGAAAGAGAATCAGCAACTGCGTGGCCGACTACAGCGTGCTGTA
CAACAGCGCTAGCTTCAGCACCTTCAAGTGCTATGGCGTCAGCCCCACCAAGCTG
AACGACCTGTGCTTCACCAACGTGTACGCCGACAGCTTCGTGATCAGAGGCGACG
AGGTGAGACAGATTGCCCCCGGGCAAACCGGCAACATCGCCGACTACAACTACA
AGCTGCCCGACGACTTCACCGGCTGCGTGATCGCGTGGAACAGCAATAACCTGGA
TTCCAAGGTGGGCGGCAACTACAACTACCTGTACAGACTGTTCAGAAAGAGCAAC
CTGAAGCCCTTCGAGAGAGACATCAGCACCGAGATCTACCAAGCCGGCAGCACC
CCCTGCAACGGCGTGAAGGGCTTCAACTGCTACTTCCCCCTGCAGAGCTACGGCT
TTCAGCCCACCTACGGCGTGGGCTATCAGCCCTACAGAGTGGTGGTGCTGAGCTT
CGAGCTGCTGCACGCCCCCGCAACCGTGTGCGGCCCCAAAAAGAGCACCAACCT
GGTGAAGAACAAGTGCGTGAACTTCAACTTTAATGGCCTTACCGGCACGGGCGTG
CTGACCGAGAGCAACAAGAAGTTCCTGCCATTTCAGCAATTCGGCAGAGACATCG
CCGACACCACCGACGCGGTCAGAGACCCTCAGACCCTGGAGATCCTGGACATCA
CCCCCTGTAGCTTCGGCGGCGTGAGCGTGATCACCCCCGGCACTAACACAAGCAA
CCAAGTGGCCGTGCTGTACCAAGGCGTGAACTGCACCGAGGTGCCCGTGGCCATC
CACGCCGATCAGCTGACCCCCACCTGGAGAGTATACAGCACCGGAAGCAACGTGT
TTCAGACAAGAGCCGGCTGCCTGATCGGCGCCGAGCACGTGAACAACAGCTACG
AGTGCGACATCCCCATCGGCGCCGGCATCTGCGCTAGCTATCAGACACAGACCAA
CAGCCCCCGGAGAGCTAGAAGCGTGGCTTCTCAGAGCATCATCGCCTACACCATG
AGCCTGGGCGTCGAGAACAGCGTGGCCTACAGCAACAACAGCATCGCCATCCCC
ACCAACTTCACCATCAGCGTGACCACCGAGATCCTGCCCGTAAGTATGACCAAGA
CAAGCGTGGACTGCACCATGTACATCTGCGGCGACAGCACCGAGTGCAGCAACCT
GCTGCTGCAGTACGGCAGCTTCTGCACACAGCTGAACAGAGCCCTGACCGGCATC
GCCGTGGAGCAAGACAAGAACACCCAAGAGGTGTTCGCCCAAGTGAAGCAGATC
TACAAGACCCCCCCCATCAAGGACTTCGGCGGCTTCAACTTCAGCCAAATCCTGC
CCGACCCTAGCAAGCCTAGCAAGCGGAGCTTCATCGAGGACCTGCTGTTCAACAA
GGTGACCCTGGCCGACGCCGGCTTCATCAAGCAGTACGGCGACTGCCTAGGCGAC
ATCGCAGCTAGAGACCTGATCTGCGCTCAGAAGTTCAATGGACTCACCGTGCTCC
CCCCCCTGCTGACCGACGAGATGATCGCTCAGTACACCTCTGCCCTGCTGGCGGG
AACCATAACAAGCGGTTGGACATTTGGCGCCGGGGCCGCACTGCAAATCCCCTTC
GCCATGCAGATGGCCTACAGATTCAACGGCATCGGCGTGACACAGAACGTGCTGT
ACGAGAATCAGAAGCTGATCGCCAATCAGTTCAACAGCGCCATCGGCAAGATCCA
AGACAGCCTGAGCAGCACCGCTAGCGCCCTGGGCAAGCTGCAAGACGTGGTGAA
TCAGAACGCCCAAGCCCTGAACACCCTGGTGAAGCAGCTGAGCAGCAACTTCGG
CGCCATCAGCAGCGTATTGAATGACATCCTGTCTAGACTGGACCCCCCCGAGGCC
GAGGTGCAGATCGACAGACTGATCACCGGCAGACTGCAGAGCCTGCAGACCTAC
GTGACACAGCAGCTGATCAGAGCCGCCGAGATCAGAGCTAGCGCCAACCTGGCC
GCCACCAAGATGAGCGAGTGCGTGCTGGGGCAGAGCAAGAGAGTGGACTTCTGC
GGCAAGGGCTACCACCTGATGAGCTTCCCTCAGAGCGCCCCCCACGGCGTGGTGT
TCCTGCACGTGACCTACGTGCCCGCCCAAGAGAAGAACTTCACCACCGCTCCCGC
CATCTGCCACGACGGCAAGGCCCACTTCCCTAGAGAGGGCGTGTTCGTGAGCAAC
GGCACCCACTGGTTCGTGACACAGAGAAACTTCTACGAGCCTCAGATCATCACCA
CCGACAACACCTTCGTGAGCGGCAACTGCGACGTGGTGATCGGCATAGTGAACAA
CACCGTATACGACCCCCTGCAGCCCGAGCTGGACAGCTTCAAGGAGGAGCTGGA
CAAGTACTTCAAAAACCATACTAGCCCCGACGTGGACCTGGGAGACATCTCTGGC
ATCAACGCTAGCGTGGTGAACATTCAGAAGGAGATCGACCGGCTGAATGAGGTGG
CCAAGAACCTGAACGAGAGCCTGATCGACCTGCAAGAGCTGGGCAAGTACGAGC
AGTACATCAAGTGGCCCTGGTACATCTGGCTGGGCTTCATCGCCGGCCTGATCGCC
ATCGTGATGGTGACCATCATGCTGTGCTGCATGACAAGCTGTTGCAGCTGCCTGAA
AGGTTGCTGCAGCTGTGGCTCGTGCTGCAAGTTCGACGAGGACGACAGCGAGCC
CGTGCTGAAGGGCGTGAAGCTGCACTACACCTGATGA
>B.1.351 spike protein amino acid sequence
SEQ ID NO: 21
MFVFLVLLPLVSSQCVNFTTRTQLPPAYTNSFTRGVYYPDKVFRSSVLHSTQDLFLPFF
SNVTWFHAIHVSGTNGTKRFANPVLPFNDGVYFASTEKSNIIRGWIFGTTLDSKTQSL
LIVNNATNVVIKVCEFQFCNDPFLGVYYHKNNKSWMESEFRVYSSANNCTFEYVSQP
FLMDLEGKQGNFKNLREFVFKNIDGYFKIYSKHTPINLVRGLPQGFSALEPLVDLPIGI
NITRFQTLHRSYLTPGDSSSGWTAGAAAYYVGYLQPRTFLLKYNENGTITDAVDCAL
DPLSETKCTLKSFTVEKGIYQTSNFRVQPTESIVRFPNITNLCPFGEVFNATRFASVYAW
NRKRISNCVADYSVLYNSASFSTFKCYGVSPTKLNDLCFTNVYADSFVIRGDEVRQIA
PGQTGNIADYNYKLPDDFTGCVIAWNSNNLDSKVGGNYNYLYRLFRKSNLKPFERDI
STEIYQAGSTPCNGVKGFNCYFPLQSYGFQPTYGVGYQPYRVVVLSFELLHAPATVC
GPKKSTNLVKNKCVNFNFNGLTGTGVLTESNKKFLPFQQFGRDIADTTDAVRDPQTL
EILDITPCSFGGVSVITPGTNTSNQVAVLYQGVNCTEVPVAIHADQLTPTWRVYSTGSN
VFQTRAGCLIGAEHVNNSYECDIPIGAGICASYQTQTNSPRRARSVASQSIIAYTMSLG
VENSVAYSNNSIAIPTNFTISVTTEILPVSMTKTSVDCTMYICGDSTECSNLLLQYGSFC
TQLNRALTGIAVEQDKNTQEVFAQVKQIYKTPPIKDFGGFNFSQILPDPSKPSKRSFIED
LLFNKVTLADAGFIKQYGDCLGDIAARDLICAQKFNGLTVLPPLLTDEMIAQYTSALL
AGTITSGWTFGAGAALQIPFAMQMAYRFNGIGVTQNVLYENQKLIANQFNSAIGKIQ
DSLSSTASALGKLQDVVNQNAQALNTLVKQLSSNFGAISSVLNDILSRLDPPEAEVQI
DRLITGRLQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGTHWFVTQR
NFYEPQIITTDNTFVSGNCDVVIGIVNNTVYDPLQPELDSFKEELDKYFKNHTSPDVD
LGDISGINASVVNIQKEIDRLNEVAKNLNESLIDLQELGKYEQYIKWPWYIWLGFIAG
LIAIVMVTIMLCCMTSCCSCLKGCCSCGSCCKFDEDDSEPVLKGVKLHYT
>B.1.1.7 spike protein CDS
SEQ ID NO: 22
ATGTTCGTGTTCCTGGTGCTGCTGCCCCTGGTGAGCTCTCAGTGCGTGAACCTGAC
CACAAGAACACAGCTGCCCCCCGCCTACACCAACAGCTTCACAAGAGGCGTGTA
CTACCCCGACAAGGTGTTCAGAAGCAGCGTGCTGCACTCCACCCAAGACCTGTTT
CTGCCCTTCTTCAGCAACGTGACCTGGTTCCACGCCATCAGCGGCACCAACGGCA
CCAAGAGATTCGACAACCCCGTGCTGCCCTTCAACGACGGCGTGTACTTCGCTAG
CACCGAAAAGAGCAACATCATCAGAGGCTGGATCTTCGGCACCACCCTAGACTCC
AAGACACAAAGCCTGCTGATCGTTAACAACGCGACCAACGTGGTGATCAAGGTGT
GCGAGTTTCAGTTCTGCAACGACCCCTTCCTGGGCGTGTACCACAAGAACAACAA
GAGCTGGATGGAGAGCGAGTTCCGTGTCTACAGCAGCGCCAACAACTGCACCTTC
GAGTACGTGTCTCAGCCCTTCCTGATGGACCTGGAGGGCAAGCAAGGCAATTTCA
AGAACCTAAGAGAGTTCGTGTTCAAGAACATCGACGGCTACTTCAAGATCTACAG
CAAGCACACCCCCATCAACCTGGTGAGAGACCTGCCCCAAGGCTTCAGCGCCCTG
GAGCCCCTGGTGGACCTGCCCATCGGCATCAACATCACAAGATTTCAGACCCTGC
TGGCCCTGCACCGTAGCTACCTAACCCCCGGCGATAGTTCCTCTGGCTGGACGGCC
GGCGCTGCCGCCTACTACGTGGGCTACCTGCAGCCTAGAACCTTCCTGCTGAAGT
ACAACGAGAACGGCACTATCACTGATGCCGTGGACTGTGCCCTGGACCCCCTGAG
CGAGACCAAGTGCACCCTCAAGAGCTTCACCGTGGAGAAGGGCATCTATCAGAC
AAGCAACTTCAGAGTGCAGCCCACCGAGAGCATCGTGAGATTCCCCAACATCACC
AACCTGTGCCCCTTCGGCGAGGTGTTCAACGCCACAAGATTCGCTAGCGTGTACG
CGTGGAACAGAAAGAGAATCAGCAACTGCGTGGCCGACTACAGCGTGCTGTACA
ACAGCGCTAGCTTCAGCACCTTCAAGTGCTATGGCGTCAGCCCCACCAAGCTGAA
CGACCTGTGCTTCACCAACGTGTACGCCGACAGCTTCGTGATCAGAGGCGACGAG
GTGAGACAGATTGCCCCCGGGCAAACCGGCAAGATCGCCGACTACAACTACAAG
CTGCCCGACGACTTCACCGGCTGCGTGATCGCGTGGAACAGCAATAACCTGGATT
CCAAGGTGGGCGGCAACTACAACTACCTGTACAGACTGTTCAGAAAGAGCAACC
TGAAGCCCTTCGAGAGAGACATCAGCACCGAGATCTACCAAGCCGGCAGCACCC
CCTGCAACGGCGTGGAGGGCTTCAACTGCTACTTCCCCCTGCAGAGCTACGGCTT
TCAGCCCACCTACGGCGTGGGCTATCAGCCCTACAGAGTGGTGGTGCTGAGCTTC
GAGCTGCTGCACGCCCCCGCAACCGTGTGCGGCCCCAAAAAGAGCACCAACCTG
GTGAAGAACAAGTGCGTGAACTTCAACTTTAATGGCCTTACCGGCACGGGCGTGC
TGACCGAGAGCAACAAGAAGTTCCTGCCATTTCAGCAATTCGGCAGAGACATCGA
CGACACCACCGACGCGGTCAGAGACCCTCAGACCCTGGAGATCCTGGACATCAC
CCCCTGTAGCTTCGGCGGCGTGAGCGTGATCACCCCCGGCACTAACACAAGCAAC
CAAGTGGCCGTGCTGTACCAAGGCGTGAACTGCACCGAGGTGCCCGTGGCCATCC
ACGCCGATCAGCTGACCCCCACCTGGAGAGTATACAGCACCGGAAGCAACGTGTT
TCAGACAAGAGCCGGCTGCCTGATCGGCGCCGAGCACGTGAACAACAGCTACGA
GTGCGACATCCCCATCGGCGCCGGCATCTGCGCTAGCTATCAGACACAGACCAAC
AGCCACCGGAGAGCTAGAAGCGTGGCTTCTCAGAGCATCATCGCCTACACCATGA
GCCTGGGCGCCGAGAACAGCGTGGCCTACAGCAACAACAGCATCGCCATCCCCAT
CAACTTCACCATCAGCGTGACCACCGAGATCCTGCCCGTAAGTATGACCAAGACA
AGCGTGGACTGCACCATGTACATCTGCGGCGACAGCACCGAGTGCAGCAACCTGC
TGCTGCAGTACGGCAGCTTCTGCACACAGCTGAACAGAGCCCTGACCGGCATCGC
CGTGGAGCAAGACAAGAACACCCAAGAGGTGTTCGCCCAAGTGAAGCAGATCTA
CAAGACCCCCCCCATCAAGGACTTCGGCGGCTTCAACTTCAGCCAAATCCTGCCC
GACCCTAGCAAGCCTAGCAAGCGGAGCTTCATCGAGGACCTGCTGTTCAACAAGG
TGACCCTGGCCGACGCCGGCTTCATCAAGCAGTACGGCGACTGCCTAGGCGACAT
CGCAGCTAGAGACCTGATCTGCGCTCAGAAGTTCAATGGACTCACCGTGCTCCCC
CCCCTGCTGACCGACGAGATGATCGCTCAGTACACCTCTGCCCTGCTGGCGGGAA
CCATAACAAGCGGTTGGACATTTGGCGCCGGGGCCGCACTGCAAATCCCCTTCGC
CATGCAGATGGCCTACAGATTCAACGGCATCGGCGTGACACAGAACGTGCTGTAC
GAGAATCAGAAGCTGATCGCCAATCAGTTCAACAGCGCCATCGGCAAGATCCAAG
ACAGCCTGAGCAGCACCGCTAGCGCCCTGGGCAAGCTGCAAGACGTGGTGAATC
AGAACGCCCAAGCCCTGAACACCCTGGTGAAGCAGCTGAGCAGCAACTTCGGCG
CCATCAGCAGCGTATTGAATGACATCCTGGCTAGACTGGACCCCCCCGAGGCCGA
GGTGCAGATCGACAGACTGATCACCGGCAGACTGCAGAGCCTGCAGACCTACGT
GACACAGCAGCTGATCAGAGCCGCCGAGATCAGAGCTAGCGCCAACCTGGCCGC
CACCAAGATGAGCGAGTGCGTGCTGGGGCAGAGCAAGAGAGTGGACTTCTGCGG
CAAGGGCTACCACCTGATGAGCTTCCCTCAGAGCGCCCCCCACGGCGTGGTGTTC
CTGCACGTGACCTACGTGCCCGCCCAAGAGAAGAACTTCACCACCGCTCCCGCCA
TCTGCCACGACGGCAAGGCCCACTTCCCTAGAGAGGGCGTGTTCGTGAGCAACG
GCACCCACTGGTTCGTGACACAGAGAAACTTCTACGAGCCTCAGATCATCACCAC
CCACAACACCTTCGTGAGCGGCAACTGCGACGTGGTGATCGGCATAGTGAACAAC
ACCGTATACGACCCCCTGCAGCCCGAGCTGGACAGCTTCAAGGAGGAGCTGGAC
AAGTACTTCAAAAACCATACTAGCCCCGACGTGGACCTGGGAGACATCTCTGGCA
TCAACGCTAGCGTGGTGAACATTCAGAAGGAGATCGACCGGCTGAATGAGGTGGC
CAAGAACCTGAACGAGAGCCTGATCGACCTGCAAGAGCTGGGCAAGTACGAGCA
GTACATCAAGTGGCCCTGGTACATCTGGCTGGGCTTCATCGCCGGCCTGATCGCCA
TCGTGATGGTGACCATCATGCTGTGCTGCATGACAAGCTGTTGCAGCTGCCTGAA
AGGTTGCTGCAGCTGTGGCTCGTGCTGCAAGTTCGACGAGGACGACAGCGAGCC
CGTGCTGAAGGGCGTGAAGCTGCACTACACCTGATGA
>B.1.1.7 spike protein amino acid sequence
SEQ ID NO: 23
MFVFLVLLPLVSSQCVNLTTRTQLPPAYTNSFTRGVYYPDKVFRSSVLHSTQDLFLPFF
SNVTWFHAISGTNGTKRFDNPVLPFNDGVYFASTEKSNIIRGWIFGTTLDSKTQSLLIV
NNATNVVIKVCEFQFCNDPFLGVYHKNNKSWMESEFRVYSSANNCTFEYVSQPFLM
DLEGKQGNFKNLREFVFKNIDGYFKIYSKHTPINLVRDLPQGFSALEPLVDLPIGINITR
FQTLLALHRSYLTPGDSSSGWTAGAAAYYVGYLQPRTFLLKYNENGTITDAVDCALD
PLSETKCTLKSFTVEKGIYQTSNFRVQPTESIVRFPNITNLCPFGEVFNATRFASVYAWN
RKRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERDIS
TEIYQAGSTPCNGVEGFNCYFPLQSYGFQPTYGVGYQPYRVVVLSFELLHAPATVCG
PKKSTNLVKNKCVNFNFNGLTGTGVLTESNKKFLPFQQFGRDIDDTTDAVRDPQTLEI
LDITPCSFGGVSVITPGTNTSNQVAVLYQGVNCTEVPVAIHADQLTPTWRVYSTGSNV
FQTRAGCLIGAEHVNNSYECDIPIGAGICASYQTQTNSHRRARSVASQSIIAYTMSLGA
ENSVAYSNNSIAIPINFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKRSFIEDL
LFNKVTLADAGFIKQYGDCLGDIAARDLICAQKFNGLTVLPPLLTDEMIAQYTSALLA
GTITSGWTFGAGAALQIPFAMQMAYRFNGIGVTQNVLYENQKLIANQFNSAIGKIQDS
LSSTASALGKLQDVVNQNAQALNTLVKQLSSNFGAISSVLNDILARLDPPEAEVQIDR
LITGRLQSLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHWFVTQRNF
YEPQIITTHNTFVSGNCDVVIGIVNNTVYDPLQPELDSFKEELDKYFKNHTSPDVDLG
DISGINASVVNIQKEIDRLNEVAKNLNESLIDLQELGKYEQYIKWPWYIWLGFIAGLIA
IVMVTIMLCCMTSCCSCLKGCCSCGSCCKFDEDDSEPVLKGVKLHYT
>SARS-CoV-2 spike protein CDS
SEQ ID NO: 24
ATGTTCGTGTTCCTGGTGCTGCTGCCCCTGGTGAGCTCTCAGTGCGTGAACCTGAC
CACAAGAACACAGCTGCCCCCCGCCTACACCAACAGCTTCACAAGAGGCGTGTA
CTACCCCGACAAGGTGTTCAGAAGCAGCGTGCTGCACTCCACCCAAGACCTGTTT
CTGCCCTTCTTCAGCAACGTGACCTGGTTCCACGCCATCCACGTGAGCGGCACCA
ACGGCACCAAGAGATTCGACAACCCCGTGCTGCCCTTCAACGACGGCGTGTACTT
CGCTAGCACCGAAAAGAGCAACATCATCAGAGGCTGGATCTTCGGCACCACCCTA
GACTCCAAGACACAAAGCCTGCTGATCGTTAACAACGCGACCAACGTGGTGATCA
AGGTGTGCGAGTTTCAGTTCTGCAACGACCCCTTCCTGGGCGTGTACTACCACAA
GAACAACAAGAGCTGGATGGAGAGCGAGTTCCGTGTCTACAGCAGCGCCAACAA
CTGCACCTTCGAGTACGTGTCTCAGCCCTTCCTGATGGACCTGGAGGGCAAGCAA
GGCAATTTCAAGAACCTAAGAGAGTTCGTGTTCAAGAACATCGACGGCTACTTCA
AGATCTACAGCAAGCACACCCCCATCAACCTGGTGAGAGACCTGCCCCAAGGCTT
CAGCGCCCTGGAGCCCCTGGTGGACCTGCCCATCGGCATCAACATCACAAGATTT
CAGACCCTGCTGGCCCTGCACCGTAGCTACCTAACCCCCGGCGATAGTTCCTCTGG
CTGGACGGCCGGCGCTGCCGCCTACTACGTGGGCTACCTGCAGCCTAGAACCTTC
CTGCTGAAGTACAACGAGAACGGCACTATCACTGATGCCGTGGACTGTGCCCTGG
ACCCCCTGAGCGAGACCAAGTGCACCCTCAAGAGCTTCACCGTGGAGAAGGGCA
TCTATCAGACAAGCAACTTCAGAGTGCAGCCCACCGAGAGCATCGTGAGATTCCC
CAACATCACCAACCTGTGCCCCTTCGGCGAGGTGTTCAACGCCACAAGATTCGCT
AGCGTGTACGCGTGGAACAGAAAGAGAATCAGCAACTGCGTGGCCGACTACAGC
GTGCTGTACAACAGCGCTAGCTTCAGCACCTTCAAGTGCTATGGCGTCAGCCCCA
CCAAGCTGAACGACCTGTGCTTCACCAACGTGTACGCCGACAGCTTCGTGATCAG
AGGCGACGAGGTGAGACAGATTGCCCCCGGGCAAACCGGCAAGATCGCCGACTA
CAACTACAAGCTGCCCGACGACTTCACCGGCTGCGTGATCGCGTGGAACAGCAAT
AACCTGGATTCCAAGGTGGGCGGCAACTACAACTACCTGTACAGACTGTTCAGAA
AGAGCAACCTGAAGCCCTTCGAGAGAGACATCAGCACCGAGATCTACCAAGCCG
GCAGCACCCCCTGCAACGGCGTGGAGGGCTTCAACTGCTACTTCCCCCTGCAGAG
CTACGGCTTTCAGCCCACCAATGGCGTGGGCTATCAGCCCTACAGAGTGGTGGTG
CTGAGCTTCGAGCTGCTGCACGCCCCCGCAACCGTGTGCGGCCCCAAAAAGAGC
ACCAACCTGGTGAAGAACAAGTGCGTGAACTTCAACTTTAATGGCCTTACCGGCA
CGGGCGTGCTGACCGAGAGCAACAAGAAGTTCCTGCCATTTCAGCAATTCGGCAG
AGACATCGCCGACACCACCGACGCGGTCAGAGACCCTCAGACCCTGGAGATCCT
GGACATCACCCCCTGTAGCTTCGGCGGCGTGAGCGTGATCACCCCCGGCACTAAC
ACAAGCAACCAAGTGGCCGTGCTGTACCAAGACGTGAACTGCACCGAGGTGCCC
GTGGCCATCCACGCCGATCAGCTGACCCCCACCTGGAGAGTATACAGCACCGGAA
GCAACGTGTTTCAGACAAGAGCCGGCTGCCTGATCGGCGCCGAGCACGTGAACA
ACAGCTACGAGTGCGACATCCCCATCGGCGCCGGCATCTGCGCTAGCTATCAGAC
ACAGACCAACAGCCCTCGGAGAGCTAGAAGCGTGGCTTCTCAGAGCATCATCGCC
TACACCATGAGCCTGGGCGCCGAGAACAGCGTGGCCTACAGCAACAACAGCATC
GCCATCCCCACCAACTTCACCATCAGCGTGACCACCGAGATCCTGCCCGTAAGTAT
GACCAAGACAAGCGTGGACTGCACCATGTACATCTGCGGCGACAGCACCGAGTG
CAGCAACCTGCTGCTGCAGTACGGCAGCTTCTGCACACAGCTGAACAGAGCCCTG
ACCGGCATCGCCGTGGAGCAAGACAAGAACACCCAAGAGGTGTTCGCCCAAGTG
AAGCAGATCTACAAGACCCCCCCCATCAAGGACTTCGGCGGCTTCAACTTCAGCC
AAATCCTGCCCGACCCTAGCAAGCCTAGCAAGCGGAGCTTCATCGAGGACCTGCT
GTTCAACAAGGTGACCCTGGCCGACGCCGGCTTCATCAAGCAGTACGGCGACTGC
CTAGGCGACATCGCAGCTAGAGACCTGATCTGCGCTCAGAAGTTCAATGGACTCA
CCGTGCTCCCCCCCCTGCTGACCGACGAGATGATCGCTCAGTACACCTCTGCCCTG
CTGGCGGGAACCATAACAAGCGGTTGGACATTTGGCGCCGGGGCCGCACTGCAA
ATCCCCTTCGCCATGCAGATGGCCTACAGATTCAACGGCATCGGCGTGACACAGA
ACGTGCTGTACGAGAATCAGAAGCTGATCGCCAATCAGTTCAACAGCGCCATCGG
CAAGATCCAAGACAGCCTGAGCAGCACCGCTAGCGCCCTGGGCAAGCTGCAAGA
CGTGGTGAATCAGAACGCCCAAGCCCTGAACACCCTGGTGAAGCAGCTGAGCAG
CAACTTCGGCGCCATCAGCAGCGTATTGAATGACATCCTGAGCAGACTGGACCCC
CCCGAGGCCGAGGTGCAGATCGACAGACTGATCACCGGCAGACTGCAGAGCCTG
CAGACCTACGTGACACAGCAGCTGATCAGAGCCGCCGAGATCAGAGCTAGCGCC
AACCTGGCCGCCACCAAGATGAGCGAGTGCGTGCTGGGGCAGAGCAAGAGAGTG
GACTTCTGCGGCAAGGGCTACCACCTGATGAGCTTCCCTCAGAGCGCCCCCCACG
GCGTGGTGTTCCTGCACGTGACCTACGTGCCCGCCCAAGAGAAGAACTTCACCAC
CGCTCCCGCCATCTGCCACGACGGCAAGGCCCACTTCCCTAGAGAGGGCGTGTTC
GTGAGCAACGGCACCCACTGGTTCGTGACACAGAGAAACTTCTACGAGCCTCAG
ATCATCACCACCGACAACACCTTCGTGAGCGGCAACTGCGACGTGGTGATCGGCA
TAGTGAACAACACCGTATACGACCCCCTGCAGCCCGAGCTGGACAGCTTCAAGGA
GGAGCTGGACAAGTACTTCAAAAACCATACTAGCCCCGACGTGGACCTGGGAGA
CATCTCTGGCATCAACGCTAGCGTGGTGAACATTCAGAAGGAGATCGACCGGCTG
AATGAGGTGGCCAAGAACCTGAACGAGAGCCTGATCGACCTGCAAGAGCTGGGC
AAGTACGAGCAGTACATCAAGTGGCCCTGGTACATCTGGCTGGGCTTCATCGCCG
GCCTGATCGCCATCGTGATGGTGACCATCATGCTGTGCTGCATGACAAGCTGTTGC
AGCTGCCTGAAAGGTTGCTGCAGCTGTGGCTCGTGCTGCAAGTTCGACGAGGAC
GACAGCGAGCCCGTGCTGAAGGGCGTGAAGCTGCACTACACCTGATGA
>SARS-CoV-2 spike protein amino acid sequence
SEQ ID NO: 25
MFVFLVLLPLVSSQCVNLTTRTQLPPAYTNSFTRGVYYPDKVFRSSVLHSTQDLFLPFF
SNVTWFHAIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTFEYVSQP
FLMDLEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGEVFNATRFASV
YAWNRKRISNCVADYSVLYNSASFSTFKCYGVSPTKLNDLCFTNVYADSFVIRGDEV
RQIAPGQTGKIADYNYKLPDDFTGCVIAWNSNNLDSKVGGNYNYLYRLFRKSNLKPF
ERDISTEIYQAGSTPCNGVEGFNCYFPLQSYGFQPTNGVGYQPYRVVVLSFELLHAPA
TVCGPKKSTNLVKNKCVNFNFNGLTGTGVLTESNKKFLPFQQFGRDIADTTDAVRDP
QTLEILDITPCSFGGVSVITPGTNTSNQVAVLYQDVNCTEVPVAIHADQLTPTWRVYST
GSNVFQTRAGCLIGAEHVNNSYECDIPIGAGICASYQTQTNSPRRARSVASQSIIAYTM
SLGAENSVAYSNNSIAIPTNFTISVTTEILPVSMTKTSVDCTMYICGDSTECSNLLLQYG
SFCTQLNRALTGIAVEQDKNTQEVFAQVKQIYKTPPIKDFGGFNFSQILPDPSKPSKRS
FIEDLLFNKVTLADAGFIKQYGDCLGDIAARDLICAQKFNGLTVLPPLLTDEMIAQYT
SALLAGTITSGWTFGAGAALQIPFAMQMAYRFNGIGVTQNVLYENQKLIANQFNSAI
GKIQDSLSSTASALGKLQDVVNQNAQALNTLVKQLSSNFGAISSVLNDILSRLDPPEA
EVQIDRLITGRLQSLQTYVTQQLIRAAEIRASANLAATKMSECVLGQSKRVDFCGKG
YHLMSFPQSAPHGVVFLHVTYVPAQEKNFTTAPAICHDGKAHFPREGVFVSNGTHWF
VTQRNFYEPQIITTDNTFVSGNCDVVIGIVNNTVYDPLQPELDSFKEELDKYFKNHTS
PDVDLGDISGINASVVNIQKEIDRLNEVAKNLNESLIDLQELGKYEQYIKWPWYIWLG
FIAGLIAIVMVTIMLCCMTSCCSCLKGCCSCGSCCKFDEDDSEPVLKGVKLHYT
>Luciferase CDS
SEQ ID NO: 26
ATGGAGGACGCCAAGAACATCAAGAAAGGCCCCGCCCCCTTCTACCCCCTGGAG
GACGGCACCGCCGGCGAGCAGCTGCACAAAGCCATGAAAAGATACGCTCTGGTG
CCCGGCACCATCGCCTTCACCGACGCCCACATCGAGGTGAACATCACCTACGCCG
AGTACTTCGAGATGAGCGTGAGACTGGCCGAGGCTATGAAGAGATACGGCTTGAA
CACCAACCACAGAATCGTGGTGTGCAGCGAGAACAGCCTGCAGTTCTTCATGCCC
GTGCTGGGCGCCCTGTTCATCGGCGTGGCCGTTGCCCCCGCCAACGACATCTACA
ACGAGAGAGAGCTGCTGAACAGCATGAACATCTCTCAGCCCACCGTGGTTTTCGT
GTCCAAAAAGGGCCTGCAGAAGATCCTGAACGTGCAGAAGAAGCTGCCCATCAT
TCAGAAGATCATCATCATGGACAGCAAGACCGACTACCAAGGCTTTCAGAGCATG
TACACCTTCGTGACAAGCCACCTGCCCCCCGGCTTCAACGAGTACGACTTCGTGC
CCGAGAGCTTCGACAGAGACAAGACCATCGCCCTGATCATGAACAGCAGCGGCA
GCACCGGCCTGCCCAAGGGCGTGGCCCTGCCCCACAGAACCGCCTGCGTGAGAT
TCAGCCACGCTAGAGACCCCATCTTCGGCAATCAGATCATCCCCGACACCGCCATC
CTGAGCGTAGTGCCTTTCCATCATGGCTTCGGCATGTTCACCACCCTGGGCTACCT
GATCTGCGGCTTCAGAGTGGTGCTGATGTACAGATTCGAGGAGGAGCTGTTCCTG
AGAAGCCTGCAAGACTACAAGATTCAGAGCGCCCTGCTGGTGCCCACCCTGTTCA
GCTTCTTCGCCAAGAGCACCCTGATCGACAAGTATGACCTGAGCAACCTGCACGA
AATTGCGAGCGGCGGAGCGCCGCTCAGCAAAGAGGTGGGCGAGGCCGTGGCCAA
GAGATTCCACCTGCCCGGCATCAGACAAGGCTACGGCCTGACCGAGACCACAAG
CGCCATCCTGATCACCCCCGAGGGCGACGATAAGCCCGGCGCCGTGGGCAAGGTG
GTACCATTCTTCGAGGCCAAGGTGGTGGACCTGGACACCGGCAAGACCCTGGGC
GTGAATCAGAGAGGCGAGCTGTGCGTGAGAGGCCCCATGATCATGAGCGGCTACG
TGAACAACCCCGAGGCCACCAACGCCCTCATCGATAAGGATGGGTGGCTGCACAG
CGGCGACATCGCCTACTGGGACGAGGACGAGCACTTCTTCATCGTGGACAGACTC
AAGAGCCTGATCAAGTACAAGGGCTACCAAGTGGCCCCCGCCGAGTTGGAGAGC
ATCCTGCTGCAGCACCCCAATATCTTCGACGCTGGCGTGGCCGGACTACCCGACG
ACGACGCCGGCGAGTTACCCGCCGCCGTGGTGGTGCTGGAGCACGGCAAGACCA
TGACCGAGAAGGAGATCGTGGACTACGTGGCTAGCCAAGTGACCACCGCCAAGA
AGCTGAGAGGCGGCGTGGTGTTCGTTGACGAAGTGCCCAAGGGCCTGACCGGCA
AGCTGGACGCTAGAAAGATCAGAGAGATCCTGATCAAGGCCAAGAAGGGCGGCA
AGAGCAAGCTGTGA
>Luciferase amino acid sequence
SEQ ID NO: 27
MEDAKNIKKGPAPFYPLEDGTAGEQLHKAMKRYALVPGTIAFTDAHIEVNITYAEYFE
MSVRLAEAMKRYGLNTNHRIVVCSENSLQFFMPVLGALFIGVAVAPANDIYNERELL
NSMNISQPTVVFVSKKGLQKILNVQKKLPIIQKIIIMDSKTDYQGFQSMYTFVTSHLPP
GFNEYDFVPESFDRDKTIALIMNSSGSTGLPKGVALPHRTACVRFSHARDPIFGNQIIP
DTAILSVVPFHHGFGMFTTLGYLICGFRVVLMYRFEEELFLRSLQDYKIQSALLVPTLF
SFFAKSTLIDKYDLSNLHEIASGGAPLSKEVGEAVAKRFHLPGIRQGYGLTETTSAILIT
PEGDDKPGAVGKVVPFFEAKVVDLDTGKTLGVNQRGELCVRGPMIMSGYVNNPEAT
NALIDKDGWLHSGDIAYWDEDEHFFIVDRLKSLIKYKGYQVAPAELESILLQHPNIFD
AGVAGLPDDDAGELPAAVVVLEHGKTMTEKEIVDYVASQVTTAKKLRGGVVFVDEV
PKGLTGKLDARKIREILIKAKKGGKSKL
>LUC1- I′ truncation
SEQ ID NO: 28
TAGGAGTTGGTGGCAGTTTCAATAACAGGTCATTGCCGAGAAAAGGATAGCACTA
TAAT
>LUC2- I′ truncation
SEQ ID NO: 29
AGTTGGTGGCAGTTTCAATAACAGGTCATTGCCGAGAAAAGGATAGCACTATAAT
>LUC3- I′ truncation
SEQ ID NO: 30
GGTGGCAGTTTCAATAACAGGTCATTGCCGAGAAAAGGATAGCACTATAAT
>LUC4- I′ truncation
SEQ ID NO: 31
GCAGTTTCAATAACAGGTCATTGCCGAGAAAAGGATAGCACTATAAT
>LUC5- I′ truncation
SEQ ID NO: 32
TTTCAATAACAGGTCATTGCCGAGAAAAGGATAGCACTATAAT
>LUC6- I′ truncation
SEQ ID NO: 33
AATAACAGGTCATTGCCGAGAAAAGGATAGCACTATAAT
>LUC7- I′ truncation
SEQ ID NO: 34
ACAGGTCATTGCCGAGAAAAGGATAGCACTATAAT
>LUC8- I′ truncation
SEQ ID NO: 35
GTCATTGCCGAGAAAAGGATAGCACTATAAT
>LUC9- I′ truncation
SEQ ID NO: 36
TTGCCGAGAAAAGGATAGCACTATAAT
>LUC10- I′ truncation
SEQ ID NO: 37
CGAGAAAAGGATAGCACTATAAT
>LUC11- I′ truncation
SEQ ID NO: 38
AAAAGGATAGCACTATAAT
>LUC12- I′ truncation
SEQ ID NO: 39
GGATAGCACTATAAT
>LUC13- I′ truncation
SEQ ID NO: 40
AGCACTATAAT
>LUC14- I′ truncation
CTATAAT
>LUC15- I′ mutant
SEQ ID NO: 42
GAGTTAGGAGTTGCTGGCAGTTTCAATAACAGGTCATTGCCGAGAAAAGGATAGC
ACTATAAT
>LUC16- I′ mutant
SEQ ID NO: 43
GAGTTAGGAGTTGTTGGCAGTTTCAATAACAGGTCATTGCCGAGAAAAGGATAGC
ACTATAAT
>5′UTR I general formula
SEQ ID NO: 44
GAGTTAGGAGTTGN₁TGGCAGTTTCAATAACAGGTCATTGCCGAGAAAAGGATAG
CACTATAAT

N₁ is selected from the group consisting of A, G, C, and T

The following SEQ ID NOs: 45-79 are RNA sequences.

>5′UTR I (63 bp)
SEQ ID NO: 45
GAGUUAGGAGUUGAUGGCAGUUUCAAUAACAGGUCAUUGCCGAGAAAAGGAU
AGCACUAUAAU
>5′UTR I′ (63 bp)
SEQ ID NO: 46
GAGUUAGGAGUUGGUGGCAGUUUCAAUAACAGGUCAUUGCCGAGAAAAGGAU
AGCACUAUAAU
>5′UTR G
SEQ ID NO: 47
GCACUUUUCUGAGCAGACGUCCAGAGCAGAGUCAGCCGCCACC
>5′UTR A
SEQ ID NO: 48
ACAUUUGCUUCUGACACAACUGUGUUCACUAGCAACCUCAAACGCCGCCACC
>5′UTR J
SEQ ID NO: 49
AGAGAGGCAAAGAAACAUUGUGAAAUCUCCAACUCUUAACCUUCAAGCCGCCA
CC
>3′UTR B
SEQ ID NO: 50
GCUCGCUUUCUUGCUGUCCAAUUUCUAUUAAAGGUUCCUUUGUUCCCUAAGUC
CAACUACUAAACUGGGGGAUAUUAUGAAGGGCCUUGAGCAUCUGGAUUCUGC
CUAAUAAAAAACAUUUAUUUUCAUUGCAA
>3′UTR F
SEQ ID NO: 51
CAGACAAGACAAGCUACUGAAUACGUUCACAUCUGUCUUGAUGCCUAAUAUU
UUUACAUUUCUGUAAACAUAUUUCUGAAAUAUUUUUUGCCUUUCAAGCGACA
GAUGCCUCAUUUUGUGAAAACUUAAUGAUGAUUUUGUGUUUAAGUUCCAAAC
AUUUGAAUAAAAUAAUUGACAAUA
>3′UTR E
SEQ ID NO: 52
CCUGAGGCCUCAGAACACAGGUUUCAGAUUGAUAGGCCUGCAGGUCUCCAGGC
AGCAACCAGCUGAGCGACUAAAGGGCCCAAGGCCAGGGCUCUAGGGAUGGGGC
UCAGCAGAGGCUGGGGUAAGGGGAGCCAGGGAGGAGCUGGGCCUAAUGCAGC
ACCGGGUCCCCAGGAUGCCGUGUCUCGGCUGGAGGAGGAGAUGCGGAAGCUCC
AGGCCACGGUGCAGGAGCUCCAGAAGCGCUUGGACAGGCUGGAGGAGACAGUC
CAGGCCAAGUAGAGCCCCGCAGGGCCUCCAGCAGGGUCAGCCAUUCACACCCA
UCCACUCACCUCCCAUUCCCAGCCACAUGGCAGAGAAAAAAAUCAUAAUAAAA
UGGCUUUAUUUUCUGGUA
>3′UTR D
SEQ ID NO: 53
GGGGCCUUCUGACAUGAGUCUGGCCUGGCCCCACCUCCUAGUUCCUCAUAAUA
AAGACAGAUUGCUUCUUCGCUUCUCACUGAGGGGCCUUCUGACAUGAGUCUGG
CCUGGCCCCACCUCCCCAGUUUCUCAUAAUAAAGACAGAUUGCUUCUUCACUU
GAAGCUCGCUUUCUUGCUGUCCAAUUUCUAUUAAAGGUUCCUUUGUUCCCUAA
GUCCAACUACUAAACUGGGGGAUAUUAUGAAGGGCCUUGAGCAUCUGGAUUC
UGCCUAAUAAAAAACAUUUAUUUUCAUUGCAA
>B.1.617.2 (Delta) spike protein mRNA
SEQ ID NO: 54
AUGUUCGUGUUCCUGGUGCUGCUGCCCCUGGUGAGCUCUCAGUGCGUGAACCU
GAGGACAAGAACACAGCUGCCCCCCGCCUACACCAACAGCUUCACAAGAGGCG
UGUACUACCCCGACAAGGUGUUCAGAAGCAGCGUGCUGCACUCCACCCAAGAC
CUGUUUCUGCCCUUCUUCAGCAACGUGACCUGGUUCCACGCCAUCCACGUCAG
CGGCACCAACGGCACCAAGAGAUUCGACAACCCCGUGCUGCCCUUCAACGACG
GCGUGUACUUCGCUAGCACCGAAAAGAGCAACAUCAUCAGAGGCUGGAUCUUC
GGCACCACCCUAGACUCCAAGACACAAAGCCUGCUGAUCGUUAACAACGCGAC
CAACGUGGUGAUCAAGGUGUGCGAGUUUCAGUUCUGCAACGACCCCUUCCUGG
ACGUGUACUACCACAAGAACAACAAGAGCUGGAUGGAGAGCGGGGUCUACAG
CAGCGCCAACAACUGCACCUUCGAGUACGUGUCUCAGCCCUUCCUGAUGGACC
UGGAGGGCAAGCAAGGCAAUUUCAAGAACCUAAGAGAGUUCGUGUUCAAGAA
CAUCGACGGCUACUUCAAGAUCUACAGCAAGCACACCCCCAUCAACCUGGUGA
GAGACCUGCCCCAAGGCUUCAGCGCCCUGGAGCCCCUGGUGGACCUGCCCAUC
GGCAUCAACAUCACAAGAUUUCAGACCCUGCUGGCCCUGCACCGUAGCUACCU
AACCCCCGGCGAUAGUUCCUCUGGCUGGACGGCCGGCGCUGCCGCCUACUACG
UGGGCUACCUGCAGCCUAGAACCUUCCUGCUGAAGUACAACGAGAACGGCACU
AUCACUGAUGCCGUGGACUGUGCCCUGGACCCCCUGAGCGAGACCAAGUGCAC
CCUCAAGAGCUUCACCGUGGAGAAGGGCAUCUAUCAGACAAGCAACUUCAGAG
UGCAGCCCACCGAGAGCAUCGUGAGAUUCCCCAACAUCACCAACCUGUGCCCC
UUCGGCGAGGUGUUCAACGCCACAAGAUUCGCUAGCGUGUACGCGUGGAACAG
AAAGAGAAUCAGCAACUGCGUGGCCGACUACAGCGUGCUGUACAACAGCGCUA
GCUUCAGCACCUUCAAGUGCUAUGGCGUCAGCCCCACCAAGCUGAACGACCUG
UGCUUCACCAACGUGUACGCCGACAGCUUCGUGAUCAGAGGCGACGAGGUGAG
ACAGAUUGCCCCCGGGCAAACCGGCAAGAUCGCCGACUACAACUACAAGCUGC
CCGACGACUUCACCGGCUGCGUGAUCGCGUGGAACAGCAAUAACCUGGAUUCC
AAGGUGGGCGGCAACUACAACUACCGGUACAGACUGUUCAGAAAGAGCAACCU
GAAGCCCUUCGAGAGAGACAUCAGCACCGAGAUCUACCAAGCCGGCAGCAAGC
CCUGCAACGGCGUGGAGGGCUUCAACUGCUACUUCCCCCUGCAGAGCUACGGC
UUUCAGCCCACCAACGGCGUGGGCUAUCAGCCCUACAGAGUGGUGGUGCUGAG
CUUCGAGCUGCUGCACGCCCCCGCAACCGUGUGCGGCCCCAAAAAGAGCACCA
ACCUGGUGAAGAACAAGUGCGUGAACUUCAACUUUAAUGGCCUUACCGGCACG
GGCGUGCUGACCGAGAGCAACAAGAAGUUCCUGCCAUUUCAGCAAUUCGGCAG
AGACAUCGCCGACACCACCGACGCGGUCAGAGACCCUCAGACCCUGGAGAUCC
UGGACAUCACCCCCUGUAGCUUCGGCGGCGUGAGCGUGAUCACCCCCGGCACU
AACACAAGCAACCAAGUGGCCGUGCUGUACCAAGGCGUGAACUGCACCGAGGU
GCCCGUGGCCAUCCACGCCGAUCAGCUGACCCCCACCUGGAGAGUAUACAGCA
CCGGAAGCAACGUGUUUCAGACAAGAGCCGGCUGCCUGAUCGGCGCCGAGCAC
GUGAACAACAGCUACGAGUGCGACAUCCCCAUCGGCGCCGGCAUCUGCGCUAG
CUAUCAGACACAGACCAACAGCCGCCGGAGAGCUAGAAGCGUGGCUUCUCAGA
GCAUCAUCGCCUACACCAUGAGCCUGGGCGCCGAGAACAGCGUGGCCUACAGC
AACAACAGCAUCGCCAUCCCCACCAACUUCACCAUCAGCGUGACCACCGAGAU
CCUGCCCGUAAGUAUGACCAAGACAAGCGUGGACUGCACCAUGUACAUCUGCG
GCGACAGCACCGAGUGCAGCAACCUGCUGCUGCAGUACGGCAGCUUCUGCACA
CAGCUGAACAGAGCCCUGACCGGCAUCGCCGUGGAGCAAGACAAGAACACCCA
AGAGGUGUUCGCCCAAGUGAAGCAGAUCUACAAGACCCCCCCCAUCAAGGACU
UCGGCGGCUUCAACUUCAGCCAAAUCCUGCCCGACCCUAGCAAGCCUAGCAAG
CGGAGCUUCAUCGAGGACCUGCUGUUCAACAAGGUGACCCUGGCCGACGCCGG
CUUCAUCAAGCAGUACGGCGACUGCCUAGGCGACAUCGCAGCUAGAGACCUGA
UCUGCGCUCAGAAGUUCAAUGGACUCACCGUGCUCCCCCCCCUGCUGACCGAC
GAGAUGAUCGCUCAGUACACCUCUGCCCUGCUGGCGGGAACCAUAACAAGCGG
UUGGACAUUUGGCGCCGGGGCCGCACUGCAAAUCCCCUUCGCCAUGCAGAUGG
CCUACAGAUUCAACGGCAUCGGCGUGACACAGAACGUGCUGUACGAGAAUCAG
AAGCUGAUCGCCAAUCAGUUCAACAGCGCCAUCGGCAAGAUCCAAGACAGCCU
GAGCAGCACCGCUAGCGCCCUGGGCAAGCUGCAAAACGUGGUGAAUCAGAACG
CCCAAGCCCUGAACACCCUGGUGAAGCAGCUGAGCAGCAACUUCGGCGCCAUC
AGCAGCGUAUUGAAUGACAUCCUGUCUAGACUGGACCCCCCCGAGGCCGAGGU
GCAGAUCGACAGACUGAUCACCGGCAGACUGCAGAGCCUGCAGACCUACGUGA
CACAGCAGCUGAUCAGAGCCGCCGAGAUCAGAGCUAGCGCCAACCUGGCCGCC
ACCAAGAUGAGCGAGUGCGUGCUGGGGCAGAGCAAGAGAGUGGACUUCUGCG
GCAAGGGCUACCACCUGAUGAGCUUCCCUCAGAGCGCCCCCCACGGCGUGGUG
UUCCUGCACGUGACCUACGUGCCCGCCCAAGAGAAGAACUUCACCACCGCUCC
CGCCAUCUGCCACGACGGCAAGGCCCACUUCCCUAGAGAGGGCGUGUUCGUGA
GCAACGGCACCCACUGGUUCGUGACACAGAGAAACUUCUACGAGCCUCAGAUC
AUCACCACCGACAACACCUUCGUGAGCGGCAACUGCGACGUGGUGAUCGGCAU
AGUGAACAACACCGUAUACGACCCCCUGCAGCCCGAGCUGGACAGCUUCAAGG
AGGAGCUGGACAAGUACUUCAAAAACCAUACUAGCCCCGACGUGGACCUGGGA
GACAUCUCUGGCAUCAACGCUAGCGUGGUGAACAUUCAGAAGGAGAUCGACCG
GCUGAAUGAGGUGGCCAAGAACCUGAACGAGAGCCUGAUCGACCUGCAAGAGC
UGGGCAAGUACGAGCAGUACAUCAAGUGGCCCUGGUACAUCUGGCUGGGCUUC
AUCGCCGGCCUGAUCGCCAUCGUGAUGGUGACCAUCAUGCUGUGCUGCAUGAC
AAGCUGUUGCAGCUGCCUGAAAGGUUGCUGCAGCUGUGGCUCGUGCUGCAAG
UUCGACGAGGACGACAGCGAGCCCGUGCUGAAGGGCGUGAAGCUGCACUACAC
CUGAUGA
>B. 1.1.529 (Omicron) spike protein mRNA
SEQ ID NO: 55
AUGUUCGUGUUCCUGGUGCUGCUGCCCCUGGUGAGCUCUCAGUGCGUGAACCU
GACCACAAGAACACAGCUGCCCCCCGCCUACACCAACAGCUUCACAAGAGGCG
UGUACUACCCCGACAAGGUGUUCAGAAGCAGCGUGCUGCACUCCACCCAAGAC
CUGUUUCUGCCCUUCUUCAGCAACGUGACCUGGUUCCACGUGAUCAGCGGCAC
CAACGGCACCAAGAGAUUCGACAACCCCGUGCUGCCCUUCAACGACGGCGUGU
ACUUCGCUAGCAUCGAAAAGAGCAACAUCAUCAGAGGCUGGAUCUUCGGCACC
ACCCUAGACUCCAAGACACAAAGCCUGCUGAUCGUUAACAACGCGACCAACGU
GGUGAUCAAGGUGUGCGAGUUUCAGUUCUGCAACGACCCCUUCCUGGACCACA
AGAACAACAAGAGCUGGAUGGAGAGCGAGUUCAGAGUGUACAGCAGCGCCAA
CAACUGCACCUUCGAGUACGUGUCUCAGCCCUUCCUGAUGGACCUGGAGGGCA
AGCAAGGCAAUUUCAAGAACCUAAGAGAGUUCGUGUUCAAGAACAUCGACGG
CUACUUCAAGAUCUACAGCAAGCACACCCCCAUCAUCGUGAGAGAGCCCGAGG
ACCUGCCCCAAGGCUUCAGCGCCCUGGAGCCCCUGGUGGACCUGCCCAUCGGC
AUCAACAUCACAAGAUUUCAGACCCUGCUGGCCCUGCACCGUAGCUACCUAAC
CCCCGGCGAUAGUUCCUCUGGCUGGACGGCCGGCGCUGCCGCCUACUACGUGG
GCUACCUGCAGCCUAGAACCUUCCUGCUGAAGUACAACGAGAACGGCACUAUC
ACUGAUGCCGUGGACUGUGCCCUGGACCCCCUGAGCGAGACCAAGUGCACCCU
CAAGAGCUUCACCGUGGAGAAGGGCAUCUAUCAGACAAGCAACUUCAGAGUGC
AGCCCACCGAGAGCAUCGUGAGAUUCCCCAACAUCACCAACCUGUGCCCCUUC
GACGAGGUGUUCAACGCCACCAGAUUUGCUAGCGUGUACGCCUGGAACCGGAA
GAGAAUCAGCAACUGCGUGGCCGACUACAGCGUGCUGUACAACCUGGCCCCCU
UCUUCACCUUCAAGUGCUAUGGCGUCAGCCCCACCAAGCUGAACGACCUGUGC
UUCACCAACGUGUACGCCGACAGCUUCGUGAUCAGAGGCGACGAGGUGAGACA
GAUCGCCCCCGGGCAGACCGGCAACAUCGCCGACUACAACUACAAGCUGCCCG
ACGACUUCACCGGCUGCGUGAUCGCCUGGAACAGCAACAAGCUGGAUAGCAAG
GUGAGCGGCAACUACAAUUACCUGUACAGACUGUUCAGAAAGAGCAACCUGA
AGCCCUUCGAGAGAGACAUCAGCACCGAGAUCUACCAAGCCGGCAACAAGCCC
UGCAACGGCGUGGCGGGCUUCAACUGCUACUUCCCCCUCAGGAGCUACAGCUU
CAGACCCACCUACGGCGUGGGCCAUCAGCCCUACAGAGUGGUCGUGCUGAGCU
UCGAGCUGCUGCACGCCCCUGCCACAGUGUGCGGCCCCAAAAAGAGCACCAAC
CUGGUGAAGAACAAGUGCGUGAACUUCAACUUCAACGGCCUGAAGGGCACCGG
CGUGCUGACCGAGAGCAACAAGAAGUUCCUGCCAUUUCAGCAAUUCGGCAGAG
ACAUCGCCGACACCACCGACGCGGUCAGAGACCCUCAGACCCUGGAGAUCCUG
GACAUCACCCCCUGUAGCUUCGGCGGCGUGAGCGUGAUCACCCCCGGCACUAA
CACAAGCAACCAAGUGGCCGUGCUGUACCAAGGCGUGAACUGCACCGAGGUGC
CCGUGGCCAUCCACGCCGAUCAGCUGACCCCCACCUGGAGAGUGUAUAGCACC
GGCAGCAACGUGUUUCAGACAAGAGCCGGCUGCCUGAUCGGCGCCGAGUACGU
GAACAACAGCUACGAGUGCGACAUCCCCAUCGGCGCCGGCAUCUGCGCUAGCU
AUCAGACACAGACCAAGAGCCACCGGAGAGCUAGAAGCGUGGCUAGCCAAAGC
AUCAUCGCCUACACCAUGAGCCUGGGCGCCGAGAACAGCGUGGCCUACAGCAA
CAACAGCAUCGCCAUCCCCACCAACUUCACCAUCAGCGUGACCACCGAGAUCC
UGCCCGUAAGUAUGACCAAGACAAGCGUGGACUGCACCAUGUACAUCUGCGGC
GACAGCACCGAGUGCAGCAACCUGCUCCUGCAGUACGGCAGCUUCUGCACACA
GCUGAAGAGAGCCCUGACCGGCAUCGCCGUGGAGCAAGACAAGAACACCCAAG
AGGUGUUCGCCCAAGUGAAGCAGAUCUACAAGACCCCCCCCAUCAAGUACUUC
GGCGGCUUCAACUUCAGCCAAAUCCUGCCCGACCCUAGCAAGCCUAGCAAGCG
GAGCUUCAUCGAGGACCUGCUGUUCAACAAGGUGACCCUGGCCGACGCCGGCU
UCAUCAAGCAGUACGGCGACUGCCUAGGCGACAUCGCAGCUAGAGACCUGAUC
UGCGCUCAGAAGUUCAAGGGACUCACCGUGCUCCCCCCCCUGCUGACCGACGA
GAUGAUCGCUCAGUACACCUCUGCCCUGCUGGCGGGAACCAUAACAAGCGGUU
GGACAUUUGGCGCCGGGGCCGCACUGCAAAUCCCCUUCGCCAUGCAGAUGGCC
UACAGAUUCAACGGCAUCGGCGUGACACAGAACGUGCUGUACGAGAAUCAGA
AGCUGAUCGCCAAUCAGUUCAACAGCGCCAUCGGCAAGAUCCAAGACAGCCUG
AGCAGCACCGCUAGCGCCCUGGGCAAGCUGCAAGACGUGGUGAACCACAACGC
CCAAGCCCUGAACACCCUGGUGAAGCAGCUGAGCAGCAAGUUCGGCGCCAUCA
GCAGCGUGCUGAAUGACAUCUUCUCUAGACUGGACCCCCCCGAGGCCGAGGUG
CAGAUCGACAGACUGAUCACCGGCAGACUGCAGAGCCUGCAGACCUACGUGAC
ACAGCAGCUGAUCAGAGCCGCCGAGAUCAGAGCUAGCGCCAACCUGGCCGCCA
CCAAGAUGAGCGAGUGCGUGCUGGGGCAGAGCAAGAGAGUGGACUUCUGCGG
CAAGGGCUACCACCUGAUGAGCUUCCCUCAGAGCGCCCCCCACGGCGUGGUGU
UCCUGCACGUGACCUACGUGCCCGCCCAAGAGAAGAACUUCACCACCGCUCCC
GCCAUCUGCCACGACGGCAAGGCCCACUUCCCUAGAGAGGGCGUGUUCGUGAG
CAACGGCACCCACUGGUUCGUGACACAGAGAAACUUCUACGAGCCUCAGAUCA
UCACCACCGACAACACCUUCGUGAGCGGCAACUGCGACGUGGUGAUCGGCAUA
GUGAACAACACCGUAUACGACCCCCUGCAGCCCGAGCUGGACAGCUUCAAGGA
GGAGCUGGACAAGUACUUCAAAAACCAUACUAGCCCCGACGUGGACCUGGGAG
ACAUCUCUGGCAUCAACGCUAGCGUGGUGAACAUUCAGAAGGAGAUCGACCGG
CUGAAUGAGGUGGCCAAGAACCUGAACGAGAGCCUGAUCGACCUGCAAGAGCU
GGGCAAGUACGAGCAGUACAUCAAGUGGCCCUGGUACAUCUGGCUGGGCUUCA
UCGCCGGCCUGAUCGCCAUCGUGAUGGUGACCAUCAUGCUGUGCUGCAUGACA
AGCUGUUGCAGCUGCCUGAAAGGUUGCUGCAGCUGUGGCUCGUGCUGCAAGU
UCGACGAGGACGACAGCGAGCCCGUGCUGAAGGGCGUGAAGCUGCACUACACC
UGAUGA
>poly A
SEQ ID NO: 56
AAAAAAAAAAAAAAAAAAAAAAAAAAAAAAGCAUAUGACUAAAAAAAAAAAA
AAAAAAAAAAAAAAAAAAAAAAAAAAAAAAAAAAAAAAAAAAAAAAAAAAAA
AAAAAAAA
>I′-Delta24-B mRNA
SEQ ID NO: 57
GAGUUAGGAGUUGGUGGCAGUUUCAAUAACAGGUCAUUGCCGAGAAAAGGAU
AGCACUAUAAUAUGUUCGUGUUCCUGGUGCUGCUGCCCCUGGUGAGCUCUCAG
UGCGUGAACCUGAGGACAAGAACACAGCUGCCCCCCGCCUACACCAACAGCUU
CACAAGAGGCGUGUACUACCCCGACAAGGUGUUCAGAAGCAGCGUGCUGCACU
CCACCCAAGACCUGUUUCUGCCCUUCUUCAGCAACGUGACCUGGUUCCACGCC
AUCCACGUCAGCGGCACCAACGGCACCAAGAGAUUCGACAACCCCGUGCUGCC
CUUCAACGACGGCGUGUACUUCGCUAGCACCGAAAAGAGCAACAUCAUCAGAG
GCUGGAUCUUCGGCACCACCCUAGACUCCAAGACACAAAGCCUGCUGAUCGUU
AACAACGCGACCAACGUGGUGAUCAAGGUGUGCGAGUUUCAGUUCUGCAACG
ACCCCUUCCUGGACGUGUACUACCACAAGAACAACAAGAGCUGGAUGGAGAGC
GGGGUCUACAGCAGCGCCAACAACUGCACCUUCGAGUACGUGUCUCAGCCCUU
CCUGAUGGACCUGGAGGGCAAGCAAGGCAAUUUCAAGAACCUAAGAGAGUUC
GUGUUCAAGAACAUCGACGGCUACUUCAAGAUCUACAGCAAGCACACCCCCAU
CAACCUGGUGAGAGACCUGCCCCAAGGCUUCAGCGCCCUGGAGCCCCUGGUGG
ACCUGCCCAUCGGCAUCAACAUCACAAGAUUUCAGACCCUGCUGGCCCUGCAC
CGUAGCUACCUAACCCCCGGCGAUAGUUCCUCUGGCUGGACGGCCGGCGCUGC
CGCCUACUACGUGGGCUACCUGCAGCCUAGAACCUUCCUGCUGAAGUACAACG
AGAACGGCACUAUCACUGAUGCCGUGGACUGUGCCCUGGACCCCCUGAGCGAG
ACCAAGUGCACCCUCAAGAGCUUCACCGUGGAGAAGGGCAUCUAUCAGACAAG
CAACUUCAGAGUGCAGCCCACCGAGAGCAUCGUGAGAUUCCCCAACAUCACCA
ACCUGUGCCCCUUCGGCGAGGUGUUCAACGCCACAAGAUUCGCUAGCGUGUAC
GCGUGGAACAGAAAGAGAAUCAGCAACUGCGUGGCCGACUACAGCGUGCUGU
ACAACAGCGCUAGCUUCAGCACCUUCAAGUGCUAUGGCGUCAGCCCCACCAAG
CUGAACGACCUGUGCUUCACCAACGUGUACGCCGACAGCUUCGUGAUCAGAGG
CGACGAGGUGAGACAGAUUGCCCCCGGGCAAACCGGCAAGAUCGCCGACUACA
ACUACAAGCUGCCCGACGACUUCACCGGCUGCGUGAUCGCGUGGAACAGCAAU
AACCUGGAUUCCAAGGUGGGCGGCAACUACAACUACCGGUACAGACUGUUCAG
AAAGAGCAACCUGAAGCCCUUCGAGAGAGACAUCAGCACCGAGAUCUACCAAG
CCGGCAGCAAGCCCUGCAACGGCGUGGAGGGCUUCAACUGCUACUUCCCCCUG
CAGAGCUACGGCUUUCAGCCCACCAACGGCGUGGGCUAUCAGCCCUACAGAGU
GGUGGUGCUGAGCUUCGAGCUGCUGCACGCCCCCGCAACCGUGUGCGGCCCCA
AAAAGAGCACCAACCUGGUGAAGAACAAGUGCGUGAACUUCAACUUUAAUGG
CCUUACCGGCACGGGCGUGCUGACCGAGAGCAACAAGAAGUUCCUGCCAUUUC
AGCAAUUCGGCAGAGACAUCGCCGACACCACCGACGCGGUCAGAGACCCUCAG
ACCCUGGAGAUCCUGGACAUCACCCCCUGUAGCUUCGGCGGCGUGAGCGUGAU
CACCCCCGGCACUAACACAAGCAACCAAGUGGCCGUGCUGUACCAAGGCGUGA
ACUGCACCGAGGUGCCCGUGGCCAUCCACGCCGAUCAGCUGACCCCCACCUGG
AGAGUAUACAGCACCGGAAGCAACGUGUUUCAGACAAGAGCCGGCUGCCUGAU
CGGCGCCGAGCACGUGAACAACAGCUACGAGUGCGACAUCCCCAUCGGCGCCG
GCAUCUGCGCUAGCUAUCAGACACAGACCAACAGCCGCCGGAGAGCUAGAAGC
GUGGCUUCUCAGAGCAUCAUCGCCUACACCAUGAGCCUGGGCGCCGAGAACAG
CGUGGCCUACAGCAACAACAGCAUCGCCAUCCCCACCAACUUCACCAUCAGCG
UGACCACCGAGAUCCUGCCCGUAAGUAUGACCAAGACAAGCGUGGACUGCACC
AUGUACAUCUGCGGCGACAGCACCGAGUGCAGCAACCUGCUGCUGCAGUACGG
CAGCUUCUGCACACAGCUGAACAGAGCCCUGACCGGCAUCGCCGUGGAGCAAG
ACAAGAACACCCAAGAGGUGUUCGCCCAAGUGAAGCAGAUCUACAAGACCCCC
CCCAUCAAGGACUUCGGCGGCUUCAACUUCAGCCAAAUCCUGCCCGACCCUAG
CAAGCCUAGCAAGCGGAGCUUCAUCGAGGACCUGCUGUUCAACAAGGUGACCC
UGGCCGACGCCGGCUUCAUCAAGCAGUACGGCGACUGCCUAGGCGACAUCGCA
GCUAGAGACCUGAUCUGCGCUCAGAAGUUCAAUGGACUCACCGUGCUCCCCCC
CCUGCUGACCGACGAGAUGAUCGCUCAGUACACCUCUGCCCUGCUGGCGGGAA
CCAUAACAAGCGGUUGGACAUUUGGCGCCGGGGCCGCACUGCAAAUCCCCUUC
GCCAUGCAGAUGGCCUACAGAUUCAACGGCAUCGGCGUGACACAGAACGUGCU
GUACGAGAAUCAGAAGCUGAUCGCCAAUCAGUUCAACAGCGCCAUCGGCAAGA
UCCAAGACAGCCUGAGCAGCACCGCUAGCGCCCUGGGCAAGCUGCAAAACGUG
GUGAAUCAGAACGCCCAAGCCCUGAACACCCUGGUGAAGCAGCUGAGCAGCAA
CUUCGGCGCCAUCAGCAGCGUAUUGAAUGACAUCCUGUCUAGACUGGACCCCC
CCGAGGCCGAGGUGCAGAUCGACAGACUGAUCACCGGCAGACUGCAGAGCCUG
CAGACCUACGUGACACAGCAGCUGAUCAGAGCCGCCGAGAUCAGAGCUAGCGC
CAACCUGGCCGCCACCAAGAUGAGCGAGUGCGUGCUGGGGCAGAGCAAGAGAG
UGGACUUCUGCGGCAAGGGCUACCACCUGAUGAGCUUCCCUCAGAGCGCCCCC
CACGGCGUGGUGUUCCUGCACGUGACCUACGUGCCCGCCCAAGAGAAGAACUU
CACCACCGCUCCCGCCAUCUGCCACGACGGCAAGGCCCACUUCCCUAGAGAGG
GCGUGUUCGUGAGCAACGGCACCCACUGGUUCGUGACACAGAGAAACUUCUAC
GAGCCUCAGAUCAUCACCACCGACAACACCUUCGUGAGCGGCAACUGCGACGU
GGUGAUCGGCAUAGUGAACAACACCGUAUACGACCCCCUGCAGCCCGAGCUGG
ACAGCUUCAAGGAGGAGCUGGACAAGUACUUCAAAAACCAUACUAGCCCCGAC
GUGGACCUGGGAGACAUCUCUGGCAUCAACGCUAGCGUGGUGAACAUUCAGA
AGGAGAUCGACCGGCUGAAUGAGGUGGCCAAGAACCUGAACGAGAGCCUGAU
CGACCUGCAAGAGCUGGGCAAGUACGAGCAGUACAUCAAGUGGCCCUGGUACA
UCUGGCUGGGCUUCAUCGCCGGCCUGAUCGCCAUCGUGAUGGUGACCAUCAUG
CUGUGCUGCAUGACAAGCUGUUGCAGCUGCCUGAAAGGUUGCUGCAGCUGUG
GCUCGUGCUGCAAGUUCGACGAGGACGACAGCGAGCCCGUGCUGAAGGGCGUG
AAGCUGCACUACACCUGAUGACUCGAGGCUCGCUUUCUUGCUGUCCAAUUUCU
AUUAAAGGUUCCUUUGUUCCCUAAGUCCAACUACUAAACUGGGGGAUAUUAU
GAAGGGCCUUGAGCAUCUGGAUUCUGCCUAAUAAAAAACAUUUAUUUUCAUU
GCAAAAAAAAAAAAAAAAAAAAAAAAAAAAAAAAGCAUAUGACUAAAAAAAA
AAAAAAAAAAAAAAAAAAAAAAAAAAAAAAAAAAAAAAAAAAAAAAAAAAAA
AAAAAAAAAAAA
>I′-OC24-B mRNA
SEQ ID NO: 58
GAGUUAGGAGUUGGUGGCAGUUUCAAUAACAGGUCAUUGCCGAGAAAAGGAU
AGCACUAUAAUAUGUUCGUGUUCCUGGUGCUGCUGCCCCUGGUGAGCUCUCAG
UGCGUGAACCUGACCACAAGAACACAGCUGCCCCCCGCCUACACCAACAGCUU
CACAAGAGGCGUGUACUACCCCGACAAGGUGUUCAGAAGCAGCGUGCUGCACU
CCACCCAAGACCUGUUUCUGCCCUUCUUCAGCAACGUGACCUGGUUCCACGUG
AUCAGCGGCACCAACGGCACCAAGAGAUUCGACAACCCCGUGCUGCCCUUCAA
CGACGGCGUGUACUUCGCUAGCAUCGAAAAGAGCAACAUCAUCAGAGGCUGGA
UCUUCGGCACCACCCUAGACUCCAAGACACAAAGCCUGCUGAUCGUUAACAAC
GCGACCAACGUGGUGAUCAAGGUGUGCGAGUUUCAGUUCUGCAACGACCCCUU
CCUGGACCACAAGAACAACAAGAGCUGGAUGGAGAGCGAGUUCAGAGUGUAC
AGCAGCGCCAACAACUGCACCUUCGAGUACGUGUCUCAGCCCUUCCUGAUGGA
CCUGGAGGGCAAGCAAGGCAAUUUCAAGAACCUAAGAGAGUUCGUGUUCAAG
AACAUCGACGGCUACUUCAAGAUCUACAGCAAGCACACCCCCAUCAUCGUGAG
AGAGCCCGAGGACCUGCCCCAAGGCUUCAGCGCCCUGGAGCCCCUGGUGGACC
UGCCCAUCGGCAUCAACAUCACAAGAUUUCAGACCCUGCUGGCCCUGCACCGU
AGCUACCUAACCCCCGGCGAUAGUUCCUCUGGCUGGACGGCCGGCGCUGCCGC
CUACUACGUGGGCUACCUGCAGCCUAGAACCUUCCUGCUGAAGUACAACGAGA
ACGGCACUAUCACUGAUGCCGUGGACUGUGCCCUGGACCCCCUGAGCGAGACC
AAGUGCACCCUCAAGAGCUUCACCGUGGAGAAGGGCAUCUAUCAGACAAGCAA
CUUCAGAGUGCAGCCCACCGAGAGCAUCGUGAGAUUCCCCAACAUCACCAACC
UGUGCCCCUUCGACGAGGUGUUCAACGCCACCAGAUUUGCUAGCGUGUACGCC
UGGAACCGGAAGAGAAUCAGCAACUGCGUGGCCGACUACAGCGUGCUGUACAA
CCUGGCCCCCUUCUUCACCUUCAAGUGCUAUGGCGUCAGCCCCACCAAGCUGA
ACGACCUGUGCUUCACCAACGUGUACGCCGACAGCUUCGUGAUCAGAGGCGAC
GAGGUGAGACAGAUCGCCCCCGGGCAGACCGGCAACAUCGCCGACUACAACUA
CAAGCUGCCCGACGACUUCACCGGCUGCGUGAUCGCCUGGAACAGCAACAAGC
UGGAUAGCAAGGUGAGCGGCAACUACAAUUACCUGUACAGACUGUUCAGAAA
GAGCAACCUGAAGCCCUUCGAGAGAGACAUCAGCACCGAGAUCUACCAAGCCG
GCAACAAGCCCUGCAACGGCGUGGCGGGCUUCAACUGCUACUUCCCCCUCAGG
AGCUACAGCUUCAGACCCACCUACGGCGUGGGCCAUCAGCCCUACAGAGUGGU
CGUGCUGAGCUUCGAGCUGCUGCACGCCCCUGCCACAGUGUGCGGCCCCAAAA
AGAGCACCAACCUGGUGAAGAACAAGUGCGUGAACUUCAACUUCAACGGCCUG
AAGGGCACCGGCGUGCUGACCGAGAGCAACAAGAAGUUCCUGCCAUUUCAGCA
AUUCGGCAGAGACAUCGCCGACACCACCGACGCGGUCAGAGACCCUCAGACCC
UGGAGAUCCUGGACAUCACCCCCUGUAGCUUCGGCGGCGUGAGCGUGAUCACC
CCCGGCACUAACACAAGCAACCAAGUGGCCGUGCUGUACCAAGGCGUGAACUG
CACCGAGGUGCCCGUGGCCAUCCACGCCGAUCAGCUGACCCCCACCUGGAGAG
UGUAUAGCACCGGCAGCAACGUGUUUCAGACAAGAGCCGGCUGCCUGAUCGGC
GCCGAGUACGUGAACAACAGCUACGAGUGCGACAUCCCCAUCGGCGCCGGCAU
CUGCGCUAGCUAUCAGACACAGACCAAGAGCCACCGGAGAGCUAGAAGCGUGG
CUAGCCAAAGCAUCAUCGCCUACACCAUGAGCCUGGGCGCCGAGAACAGCGUG
GCCUACAGCAACAACAGCAUCGCCAUCCCCACCAACUUCACCAUCAGCGUGAC
CACCGAGAUCCUGCCCGUAAGUAUGACCAAGACAAGCGUGGACUGCACCAUGU
ACAUCUGCGGCGACAGCACCGAGUGCAGCAACCUGCUCCUGCAGUACGGCAGC
UUCUGCACACAGCUGAAGAGAGCCCUGACCGGCAUCGCCGUGGAGCAAGACAA
GAACACCCAAGAGGUGUUCGCCCAAGUGAAGCAGAUCUACAAGACCCCCCCCA
UCAAGUACUUCGGCGGCUUCAACUUCAGCCAAAUCCUGCCCGACCCUAGCAAG
CCUAGCAAGCGGAGCUUCAUCGAGGACCUGCUGUUCAACAAGGUGACCCUGGC
CGACGCCGGCUUCAUCAAGCAGUACGGCGACUGCCUAGGCGACAUCGCAGCUA
GAGACCUGAUCUGCGCUCAGAAGUUCAAGGGACUCACCGUGCUCCCCCCCCUG
CUGACCGACGAGAUGAUCGCUCAGUACACCUCUGCCCUGCUGGCGGGAACCAU
AACAAGCGGUUGGACAUUUGGCGCCGGGGCCGCACUGCAAAUCCCCUUCGCCA
UGCAGAUGGCCUACAGAUUCAACGGCAUCGGCGUGACACAGAACGUGCUGUAC
GAGAAUCAGAAGCUGAUCGCCAAUCAGUUCAACAGCGCCAUCGGCAAGAUCCA
AGACAGCCUGAGCAGCACCGCUAGCGCCCUGGGCAAGCUGCAAGACGUGGUGA
ACCACAACGCCCAAGCCCUGAACACCCUGGUGAAGCAGCUGAGCAGCAAGUUC
GGCGCCAUCAGCAGCGUGCUGAAUGACAUCUUCUCUAGACUGGACCCCCCCGA
GGCCGAGGUGCAGAUCGACAGACUGAUCACCGGCAGACUGCAGAGCCUGCAGA
CCUACGUGACACAGCAGCUGAUCAGAGCCGCCGAGAUCAGAGCUAGCGCCAAC
CUGGCCGCCACCAAGAUGAGCGAGUGCGUGCUGGGGCAGAGCAAGAGAGUGG
ACUUCUGCGGCAAGGGCUACCACCUGAUGAGCUUCCCUCAGAGCGCCCCCCAC
GGCGUGGUGUUCCUGCACGUGACCUACGUGCCCGCCCAAGAGAAGAACUUCAC
CACCGCUCCCGCCAUCUGCCACGACGGCAAGGCCCACUUCCCUAGAGAGGGCG
UGUUCGUGAGCAACGGCACCCACUGGUUCGUGACACAGAGAAACUUCUACGAG
CCUCAGAUCAUCACCACCGACAACACCUUCGUGAGCGGCAACUGCGACGUGGU
GAUCGGCAUAGUGAACAACACCGUAUACGACCCCCUGCAGCCCGAGCUGGACA
GCUUCAAGGAGGAGCUGGACAAGUACUUCAAAAACCAUACUAGCCCCGACGUG
GACCUGGGAGACAUCUCUGGCAUCAACGCUAGCGUGGUGAACAUUCAGAAGG
AGAUCGACCGGCUGAAUGAGGUGGCCAAGAACCUGAACGAGAGCCUGAUCGAC
CUGCAAGAGCUGGGCAAGUACGAGCAGUACAUCAAGUGGCCCUGGUACAUCUG
GCUGGGCUUCAUCGCCGGCCUGAUCGCCAUCGUGAUGGUGACCAUCAUGCUGU
GCUGCAUGACAAGCUGUUGCAGCUGCCUGAAAGGUUGCUGCAGCUGUGGCUCG
UGCUGCAAGUUCGACGAGGACGACAGCGAGCCCGUGCUGAAGGGCGUGAAGCU
GCACUACACCUGAUGACUCGAGGCUCGCUUUCUUGCUGUCCAAUUUCUAUUAA
AGGUUCCUUUGUUCCCUAAGUCCAACUACUAAACUGGGGGAUAUUAUGAAGG
GCCUUGAGCAUCUGGAUUCUGCCUAAUAAAAAACAUUUAUUUUCAUUGCAAA
AAAAAAAAAAAAAAAAAAAAAAAAAAAAAGCAUAUGACUAAAAAAAAAAAAA
AAAAAAAAAAAAAAAAAAAAAAAAAAAAAAAAAAAAAAAAAAAAAAAAAAAA
AAAAAAA
>B.1.351 spike protein mRNA
SEQ ID NO: 59
AUGUUCGUGUUCCUGGUGCUGCUGCCCCUGGUGAGCUCUCAGUGCGUGAACUU
CACGACAAGAACACAGCUGCCCCCCGCCUACACCAACAGCUUCACAAGAGGCG
UGUACUACCCCGACAAGGUGUUCAGAAGCAGCGUGCUGCACUCCACCCAAGAC
CUGUUUCUGCCCUUCUUCAGCAACGUGACCUGGUUCCACGCCAUCCACGUGAG
CGGCACCAACGGCACCAAGAGAUUCGCCAACCCCGUGCUGCCCUUCAACGACG
GCGUGUACUUCGCUAGCACCGAAAAGAGCAACAUCAUCAGAGGCUGGAUCUUC
GGCACCACCCUAGACUCCAAGACACAAAGCCUGCUGAUCGUUAACAACGCGAC
CAACGUGGUGAUCAAGGUGUGCGAGUUUCAGUUCUGCAACGACCCCUUCCUGG
GCGUGUACUACCACAAGAACAACAAGAGCUGGAUGGAGAGCGAGUUCCGUGU
CUACAGCAGCGCCAACAACUGCACCUUCGAGUACGUGUCUCAGCCCUUCCUGA
UGGACCUGGAGGGCAAGCAAGGCAAUUUCAAGAACCUAAGAGAGUUCGUGUU
CAAGAACAUCGACGGCUACUUCAAGAUCUACAGCAAGCACACCCCCAUCAACC
UGGUGAGAGGCCUGCCCCAAGGCUUCAGCGCCCUGGAGCCCCUGGUGGACCUG
CCCAUCGGCAUCAACAUCACAAGAUUUCAGACCCUGCACCGUAGCUACCUAAC
CCCCGGCGAUAGUUCCUCUGGCUGGACGGCCGGCGCUGCCGCCUACUACGUGG
GCUACCUGCAGCCUAGAACCUUCCUGCUGAAGUACAACGAGAACGGCACUAUC
ACUGAUGCCGUGGACUGUGCCCUGGACCCCCUGAGCGAGACCAAGUGCACCCU
CAAGAGCUUCACCGUGGAGAAGGGCAUCUAUCAGACAAGCAACUUCAGAGUGC
AGCCCACCGAGAGCAUCGUGAGAUUCCCCAACAUCACCAACCUGUGCCCCUUC
GGCGAGGUGUUCAACGCCACAAGAUUCGCUAGCGUGUACGCGUGGAACAGAA
AGAGAAUCAGCAACUGCGUGGCCGACUACAGCGUGCUGUACAACAGCGCUAGC
UUCAGCACCUUCAAGUGCUAUGGCGUCAGCCCCACCAAGCUGAACGACCUGUG
CUUCACCAACGUGUACGCCGACAGCUUCGUGAUCAGAGGCGACGAGGUGAGAC
AGAUUGCCCCCGGGCAAACCGGCAACAUCGCCGACUACAACUACAAGCUGCCC
GACGACUUCACCGGCUGCGUGAUCGCGUGGAACAGCAAUAACCUGGAUUCCAA
GGUGGGCGGCAACUACAACUACCUGUACAGACUGUUCAGAAAGAGCAACCUGA
AGCCCUUCGAGAGAGACAUCAGCACCGAGAUCUACCAAGCCGGCAGCACCCCC
UGCAACGGCGUGAAGGGCUUCAACUGCUACUUCCCCCUGCAGAGCUACGGCUU
UCAGCCCACCUACGGCGUGGGCUAUCAGCCCUACAGAGUGGUGGUGCUGAGCU
UCGAGCUGCUGCACGCCCCCGCAACCGUGUGCGGCCCCAAAAAGAGCACCAAC
CUGGUGAAGAACAAGUGCGUGAACUUCAACUUUAAUGGCCUUACCGGCACGG
GCGUGCUGACCGAGAGCAACAAGAAGUUCCUGCCAUUUCAGCAAUUCGGCAGA
GACAUCGCCGACACCACCGACGCGGUCAGAGACCCUCAGACCCUGGAGAUCCU
GGACAUCACCCCCUGUAGCUUCGGCGGCGUGAGCGUGAUCACCCCCGGCACUA
ACACAAGCAACCAAGUGGCCGUGCUGUACCAAGGCGUGAACUGCACCGAGGUG
CCCGUGGCCAUCCACGCCGAUCAGCUGACCCCCACCUGGAGAGUAUACAGCAC
CGGAAGCAACGUGUUUCAGACAAGAGCCGGCUGCCUGAUCGGCGCCGAGCACG
UGAACAACAGCUACGAGUGCGACAUCCCCAUCGGCGCCGGCAUCUGCGCUAGC
UAUCAGACACAGACCAACAGCCCCCGGAGAGCUAGAAGCGUGGCUUCUCAGAG
CAUCAUCGCCUACACCAUGAGCCUGGGCGUCGAGAACAGCGUGGCCUACAGCA
ACAACAGCAUCGCCAUCCCCACCAACUUCACCAUCAGCGUGACCACCGAGAUC
CUGCCCGUAAGUAUGACCAAGACAAGCGUGGACUGCACCAUGUACAUCUGCGG
CGACAGCACCGAGUGCAGCAACCUGCUGCUGCAGUACGGCAGCUUCUGCACAC
AGCUGAACAGAGCCCUGACCGGCAUCGCCGUGGAGCAAGACAAGAACACCCAA
GAGGUGUUCGCCCAAGUGAAGCAGAUCUACAAGACCCCCCCCAUCAAGGACUU
CGGCGGCUUCAACUUCAGCCAAAUCCUGCCCGACCCUAGCAAGCCUAGCAAGC
GGAGCUUCAUCGAGGACCUGCUGUUCAACAAGGUGACCCUGGCCGACGCCGGC
UUCAUCAAGCAGUACGGCGACUGCCUAGGCGACAUCGCAGCUAGAGACCUGAU
CUGCGCUCAGAAGUUCAAUGGACUCACCGUGCUCCCCCCCCUGCUGACCGACG
AGAUGAUCGCUCAGUACACCUCUGCCCUGCUGGCGGGAACCAUAACAAGCGGU
UGGACAUUUGGCGCCGGGGCCGCACUGCAAAUCCCCUUCGCCAUGCAGAUGGC
CUACAGAUUCAACGGCAUCGGCGUGACACAGAACGUGCUGUACGAGAAUCAGA
AGCUGAUCGCCAAUCAGUUCAACAGCGCCAUCGGCAAGAUCCAAGACAGCCUG
AGCAGCACCGCUAGCGCCCUGGGCAAGCUGCAAGACGUGGUGAAUCAGAACGC
CCAAGCCCUGAACACCCUGGUGAAGCAGCUGAGCAGCAACUUCGGCGCCAUCA
GCAGCGUAUUGAAUGACAUCCUGUCUAGACUGGACCCCCCCGAGGCCGAGGUG
CAGAUCGACAGACUGAUCACCGGCAGACUGCAGAGCCUGCAGACCUACGUGAC
ACAGCAGCUGAUCAGAGCCGCCGAGAUCAGAGCUAGCGCCAACCUGGCCGCCA
CCAAGAUGAGCGAGUGCGUGCUGGGGCAGAGCAAGAGAGUGGACUUCUGCGG
CAAGGGCUACCACCUGAUGAGCUUCCCUCAGAGCGCCCCCCACGGCGUGGUGU
UCCUGCACGUGACCUACGUGCCCGCCCAAGAGAAGAACUUCACCACCGCUCCC
GCCAUCUGCCACGACGGCAAGGCCCACUUCCCUAGAGAGGGCGUGUUCGUGAG
CAACGGCACCCACUGGUUCGUGACACAGAGAAACUUCUACGAGCCUCAGAUCA
UCACCACCGACAACACCUUCGUGAGCGGCAACUGCGACGUGGUGAUCGGCAUA
GUGAACAACACCGUAUACGACCCCCUGCAGCCCGAGCUGGACAGCUUCAAGGA
GGAGCUGGACAAGUACUUCAAAAACCAUACUAGCCCCGACGUGGACCUGGGAG
ACAUCUCUGGCAUCAACGCUAGCGUGGUGAACAUUCAGAAGGAGAUCGACCGG
CUGAAUGAGGUGGCCAAGAACCUGAACGAGAGCCUGAUCGACCUGCAAGAGCU
GGGCAAGUACGAGCAGUACAUCAAGUGGCCCUGGUACAUCUGGCUGGGCUUCA
UCGCCGGCCUGAUCGCCAUCGUGAUGGUGACCAUCAUGCUGUGCUGCAUGACA
AGCUGUUGCAGCUGCCUGAAAGGUUGCUGCAGCUGUGGCUCGUGCUGCAAGU
UCGACGAGGACGACAGCGAGCCCGUGCUGAAGGGCGUGAAGCUGCACUACACC
UGAUGA
>B.1.1.7 spike protein mRNA
SEQ ID NO: 60
AUGUUCGUGUUCCUGGUGCUGCUGCCCCUGGUGAGCUCUCAGUGCGUGAACCU
GACCACAAGAACACAGCUGCCCCCCGCCUACACCAACAGCUUCACAAGAGGCG
UGUACUACCCCGACAAGGUGUUCAGAAGCAGCGUGCUGCACUCCACCCAAGAC
CUGUUUCUGCCCUUCUUCAGCAACGUGACCUGGUUCCACGCCAUCAGCGGCAC
CAACGGCACCAAGAGAUUCGACAACCCCGUGCUGCCCUUCAACGACGGCGUGU
ACUUCGCUAGCACCGAAAAGAGCAACAUCAUCAGAGGCUGGAUCUUCGGCACC
ACCCUAGACUCCAAGACACAAAGCCUGCUGAUCGUUAACAACGCGACCAACGU
GGUGAUCAAGGUGUGCGAGUUUCAGUUCUGCAACGACCCCUUCCUGGGCGUGU
ACCACAAGAACAACAAGAGCUGGAUGGAGAGCGAGUUCCGUGUCUACAGCAGC
GCCAACAACUGCACCUUCGAGUACGUGUCUCAGCCCUUCCUGAUGGACCUGGA
GGGCAAGCAAGGCAAUUUCAAGAACCUAAGAGAGUUCGUGUUCAAGAACAUC
GACGGCUACUUCAAGAUCUACAGCAAGCACACCCCCAUCAACCUGGUGAGAGA
CCUGCCCCAAGGCUUCAGCGCCCUGGAGCCCCUGGUGGACCUGCCCAUCGGCA
UCAACAUCACAAGAUUUCAGACCCUGCUGGCCCUGCACCGUAGCUACCUAACC
CCCGGCGAUAGUUCCUCUGGCUGGACGGCCGGCGCUGCCGCCUACUACGUGGG
CUACCUGCAGCCUAGAACCUUCCUGCUGAAGUACAACGAGAACGGCACUAUCA
CUGAUGCCGUGGACUGUGCCCUGGACCCCCUGAGCGAGACCAAGUGCACCCUC
AAGAGCUUCACCGUGGAGAAGGGCAUCUAUCAGACAAGCAACUUCAGAGUGC
AGCCCACCGAGAGCAUCGUGAGAUUCCCCAACAUCACCAACCUGUGCCCCUUC
GGCGAGGUGUUCAACGCCACAAGAUUCGCUAGCGUGUACGCGUGGAACAGAA
AGAGAAUCAGCAACUGCGUGGCCGACUACAGCGUGCUGUACAACAGCGCUAGC
UUCAGCACCUUCAAGUGCUAUGGCGUCAGCCCCACCAAGCUGAACGACCUGUG
CUUCACCAACGUGUACGCCGACAGCUUCGUGAUCAGAGGCGACGAGGUGAGAC
AGAUUGCCCCCGGGCAAACCGGCAAGAUCGCCGACUACAACUACAAGCUGCCC
GACGACUUCACCGGCUGCGUGAUCGCGUGGAACAGCAAUAACCUGGAUUCCAA
GGUGGGCGGCAACUACAACUACCUGUACAGACUGUUCAGAAAGAGCAACCUGA
AGCCCUUCGAGAGAGACAUCAGCACCGAGAUCUACCAAGCCGGCAGCACCCCC
UGCAACGGCGUGGAGGGCUUCAACUGCUACUUCCCCCUGCAGAGCUACGGCUU
UCAGCCCACCUACGGCGUGGGCUAUCAGCCCUACAGAGUGGUGGUGCUGAGCU
UCGAGCUGCUGCACGCCCCCGCAACCGUGUGCGGCCCCAAAAAGAGCACCAAC
CUGGUGAAGAACAAGUGCGUGAACUUCAACUUUAAUGGCCUUACCGGCACGG
GCGUGCUGACCGAGAGCAACAAGAAGUUCCUGCCAUUUCAGCAAUUCGGCAGA
GACAUCGACGACACCACCGACGCGGUCAGAGACCCUCAGACCCUGGAGAUCCU
GGACAUCACCCCCUGUAGCUUCGGCGGCGUGAGCGUGAUCACCCCCGGCACUA
ACACAAGCAACCAAGUGGCCGUGCUGUACCAAGGCGUGAACUGCACCGAGGUG
CCCGUGGCCAUCCACGCCGAUCAGCUGACCCCCACCUGGAGAGUAUACAGCAC
CGGAAGCAACGUGUUUCAGACAAGAGCCGGCUGCCUGAUCGGCGCCGAGCACG
UGAACAACAGCUACGAGUGCGACAUCCCCAUCGGCGCCGGCAUCUGCGCUAGC
UAUCAGACACAGACCAACAGCCACCGGAGAGCUAGAAGCGUGGCUUCUCAGAG
CAUCAUCGCCUACACCAUGAGCCUGGGCGCCGAGAACAGCGUGGCCUACAGCA
ACAACAGCAUCGCCAUCCCCAUCAACUUCACCAUCAGCGUGACCACCGAGAUC
CUGCCCGUAAGUAUGACCAAGACAAGCGUGGACUGCACCAUGUACAUCUGCGG
CGACAGCACCGAGUGCAGCAACCUGCUGCUGCAGUACGGCAGCUUCUGCACAC
AGCUGAACAGAGCCCUGACCGGCAUCGCCGUGGAGCAAGACAAGAACACCCAA
GAGGUGUUCGCCCAAGUGAAGCAGAUCUACAAGACCCCCCCCAUCAAGGACUU
CGGCGGCUUCAACUUCAGCCAAAUCCUGCCCGACCCUAGCAAGCCUAGCAAGC
GGAGCUUCAUCGAGGACCUGCUGUUCAACAAGGUGACCCUGGCCGACGCCGGC
UUCAUCAAGCAGUACGGCGACUGCCUAGGCGACAUCGCAGCUAGAGACCUGAU
CUGCGCUCAGAAGUUCAAUGGACUCACCGUGCUCCCCCCCCUGCUGACCGACG
AGAUGAUCGCUCAGUACACCUCUGCCCUGCUGGCGGGAACCAUAACAAGCGGU
UGGACAUUUGGCGCCGGGGCCGCACUGCAAAUCCCCUUCGCCAUGCAGAUGGC
CUACAGAUUCAACGGCAUCGGCGUGACACAGAACGUGCUGUACGAGAAUCAGA
AGCUGAUCGCCAAUCAGUUCAACAGCGCCAUCGGCAAGAUCCAAGACAGCCUG
AGCAGCACCGCUAGCGCCCUGGGCAAGCUGCAAGACGUGGUGAAUCAGAACGC
CCAAGCCCUGAACACCCUGGUGAAGCAGCUGAGCAGCAACUUCGGCGCCAUCA
GCAGCGUAUUGAAUGACAUCCUGGCUAGACUGGACCCCCCCGAGGCCGAGGUG
CAGAUCGACAGACUGAUCACCGGCAGACUGCAGAGCCUGCAGACCUACGUGAC
ACAGCAGCUGAUCAGAGCCGCCGAGAUCAGAGCUAGCGCCAACCUGGCCGCCA
CCAAGAUGAGCGAGUGCGUGCUGGGGCAGAGCAAGAGAGUGGACUUCUGCGG
CAAGGGCUACCACCUGAUGAGCUUCCCUCAGAGCGCCCCCCACGGCGUGGUGU
UCCUGCACGUGACCUACGUGCCCGCCCAAGAGAAGAACUUCACCACCGCUCCC
GCCAUCUGCCACGACGGCAAGGCCCACUUCCCUAGAGAGGGCGUGUUCGUGAG
CAACGGCACCCACUGGUUCGUGACACAGAGAAACUUCUACGAGCCUCAGAUCA
UCACCACCCACAACACCUUCGUGAGCGGCAACUGCGACGUGGUGAUCGGCAUA
GUGAACAACACCGUAUACGACCCCCUGCAGCCCGAGCUGGACAGCUUCAAGGA
GGAGCUGGACAAGUACUUCAAAAACCAUACUAGCCCCGACGUGGACCUGGGAG
ACAUCUCUGGCAUCAACGCUAGCGUGGUGAACAUUCAGAAGGAGAUCGACCGG
CUGAAUGAGGUGGCCAAGAACCUGAACGAGAGCCUGAUCGACCUGCAAGAGCU
GGGCAAGUACGAGCAGUACAUCAAGUGGCCCUGGUACAUCUGGCUGGGCUUCA
UCGCCGGCCUGAUCGCCAUCGUGAUGGUGACCAUCAUGCUGUGCUGCAUGACA
AGCUGUUGCAGCUGCCUGAAAGGUUGCUGCAGCUGUGGCUCGUGCUGCAAGU
UCGACGAGGACGACAGCGAGCCCGUGCUGAAGGGCGUGAAGCUGCACUACACC
UGAUGA
>SARS-COV-2 spike protein mRNA
SEQ ID NO: 61
AUGUUCGUGUUCCUGGUGCUGCUGCCCCUGGUGAGCUCUCAGUGCGUGAACCU
GACCACAAGAACACAGCUGCCCCCCGCCUACACCAACAGCUUCACAAGAGGCG
UGUACUACCCCGACAAGGUGUUCAGAAGCAGCGUGCUGCACUCCACCCAAGAC
CUGUUUCUGCCCUUCUUCAGCAACGUGACCUGGUUCCACGCCAUCCACGUGAG
CGGCACCAACGGCACCAAGAGAUUCGACAACCCCGUGCUGCCCUUCAACGACG
GCGUGUACUUCGCUAGCACCGAAAAGAGCAACAUCAUCAGAGGCUGGAUCUUC
GGCACCACCCUAGACUCCAAGACACAAAGCCUGCUGAUCGUUAACAACGCGAC
CAACGUGGUGAUCAAGGUGUGCGAGUUUCAGUUCUGCAACGACCCCUUCCUGG
GCGUGUACUACCACAAGAACAACAAGAGCUGGAUGGAGAGCGAGUUCCGUGU
CUACAGCAGCGCCAACAACUGCACCUUCGAGUACGUGUCUCAGCCCUUCCUGA
UGGACCUGGAGGGCAAGCAAGGCAAUUUCAAGAACCUAAGAGAGUUCGUGUU
CAAGAACAUCGACGGCUACUUCAAGAUCUACAGCAAGCACACCCCCAUCAACC
UGGUGAGAGACCUGCCCCAAGGCUUCAGCGCCCUGGAGCCCCUGGUGGACCUG
CCCAUCGGCAUCAACAUCACAAGAUUUCAGACCCUGCUGGCCCUGCACCGUAG
CUACCUAACCCCCGGCGAUAGUUCCUCUGGCUGGACGGCCGGCGCUGCCGCCU
ACUACGUGGGCUACCUGCAGCCUAGAACCUUCCUGCUGAAGUACAACGAGAAC
GGCACUAUCACUGAUGCCGUGGACUGUGCCCUGGACCCCCUGAGCGAGACCAA
GUGCACCCUCAAGAGCUUCACCGUGGAGAAGGGCAUCUAUCAGACAAGCAACU
UCAGAGUGCAGCCCACCGAGAGCAUCGUGAGAUUCCCCAACAUCACCAACCUG
UGCCCCUUCGGCGAGGUGUUCAACGCCACAAGAUUCGCUAGCGUGUACGCGUG
GAACAGAAAGAGAAUCAGCAACUGCGUGGCCGACUACAGCGUGCUGUACAACA
GCGCUAGCUUCAGCACCUUCAAGUGCUAUGGCGUCAGCCCCACCAAGCUGAAC
GACCUGUGCUUCACCAACGUGUACGCCGACAGCUUCGUGAUCAGAGGCGACGA
GGUGAGACAGAUUGCCCCCGGGCA5AACCGGCAAGAUCGCCGACUACAACUAC
AAGCUGCCCGACGACUUCACCGGCUGCGUGAUCGCGUGGAACAGCAAUAACCU
GGAUUCCAAGGUGGGGGCAACUACAACUACCUGUACAGACUGUUCAGAAAG
AGCAACCUGAAGCCCUUCGAGAGAGACAUCAGCACCGAGAUCUACCAAGCCGG
CAGCACCCCCUGCAACGGCGUGGAGGGCUUCAACUGCUACUUCCCCCUGCAGA
GCUACGGCUUUCAGCCCACCAAUGGCGUGGGCUAUCAGCCCUACAGAGUGGUG
GUGCUGAGCUUCGAGCUGCUGCACGCCCCCGCAACCGUGUGCGGCCCCAAAAA
GAGCACCAACCUGGUGAAGAACAAGUGCGUGAACUUCAACUUUAAUGGCCUU
ACCGGCACGGGCGUGCUGACCGAGAGCAACAAGAAGUUCCUGCCAUUUCAGCA
AUUCGGCAGAGACAUCGCCGACACCACCGACGCGGUCAGAGACCCUCAGACCC
UGGAGAUCCUGGACAUCACCCCCUGUAGCUUCGGCGGCGUGAGCGUGAUCACC
CCCGGCACUAACACAAGCAACCAAGUGGCCGUGCUGUACCAAGACGUGAACUG
CACCGAGGUGCCCGUGGCCAUCCACGCCGAUCAGCUGACCCCCACCUGGAGAG
UAUACAGCACCGGAAGCAACGUGUUUCAGACAAGAGCCGGCUGCCUGAUCGGC
GCCGAGCACGUGAACAACAGCUACGAGUGCGACAUCCCCAUCGGCGCCGGCAU
CUGCGCUAGCUAUCAGACACAGACCAACAGCCCUCGGAGAGCUAGAAGCGUGG
CUUCUCAGAGCAUCAUCGCCUACACCAUGAGCCUGGGCGCCGAGAACAGCGUG
GCCUACAGCAACAACAGCAUCGCCAUCCCCACCAACUUCACCAUCAGCGUGAC
CACCGAGAUCCUGCCCGUAAGUAUGACCAAGACAAGCGUGGACUGCACCAUGU
ACAUCUGCGGCGACAGCACCGAGUGCAGCAACCUGCUGCUGCAGUACGGCAGC
UUCUGCACACAGCUGAACAGAGCCCUGACCGGCAUCGCCGUGGAGCAAGACAA
GAACACCCAAGAGGUGUUCGCCCAAGUGAAGCAGAUCUACAAGACCCCCCCCA
UCAAGGACUUCGGCGGCUUCAACUUCAGCCAAAUCCUGCCCGACCCUAGCAAG
CCUAGCAAGCGGAGCUUCAUCGAGGACCUGCUGUUCAACAAGGUGACCCUGGC
CGACGCCGGCUUCAUCAAGCAGUACGGCGACUGCCUAGGCGACAUCGCAGCUA
GAGACCUGAUCUGCGCUCAGAAGUUCAAUGGACUCACCGUGCUCCCCCCCCUG
CUGACCGACGAGAUGAUCGCUCAGUACACCUCUGCCCUGCUGGCGGGAACCAU
AACAAGCGGUUGGACAUUUGGCGCCGGGGCCGCACUGCAAAUCCCCUUCGCCA
UGCAGAUGGCCUACAGAUUCAACGGCAUCGGCGUGACACAGAACGUGCUGUAC
GAGAAUCAGAAGCUGAUCGCCAAUCAGUUCAACAGCGCCAUCGGCAAGAUCCA
AGACAGCCUGAGCAGCACCGCUAGCGCCCUGGGCAAGCUGCAAGACGUGGUGA
AUCAGAACGCCCAAGCCCUGAACACCCUGGUGAAGCAGCUGAGCAGCAACUUC
GGCGCCAUCAGCAGCGUAUUGAAUGACAUCCUGAGCAGACUGGACCCCCCCGA
GGCCGAGGUGCAGAUCGACAGACUGAUCACCGGCAGACUGCAGAGCCUGCAGA
CCUACGUGACACAGCAGCUGAUCAGAGCCGCCGAGAUCAGAGCUAGCGCCAAC
CUGGCCGCCACCAAGAUGAGCGAGUGCGUGCUGGGGCAGAGCAAGAGAGUGG
ACUUCUGCGGCAAGGGCUACCACCUGAUGAGCUUCCCUCAGAGCGCCCCCCAC
GGCGUGGUGUUCCUGCACGUGACCUACGUGCCCGCCCAAGAGAAGAACUUCAC
CACCGCUCCCGCCAUCUGCCACGACGGCAAGGCCCACUUCCCUAGAGAGGGCG
UGUUCGUGAGCAACGGCACCCACUGGUUCGUGACACAGAGAAACUUCUACGAG
CCUCAGAUCAUCACCACCGACAACACCUUCGUGAGCGGCAACUGCGACGUGGU
GAUCGGCAUAGUGAACAACACCGUAUACGACCCCCUGCAGCCCGAGCUGGACA
GCUUCAAGGAGGAGCUGGACAAGUACUUCAAAAACCAUACUAGCCCCGACGUG
GACCUGGGAGACAUCUCUGGCAUCAACGCUAGCGUGGUGAACAUUCAGAAGG
AGAUCGACCGGCUGAAUGAGGUGGCCAAGAACCUGAACGAGAGCCUGAUCGAC
CUGCAAGAGCUGGGCAAGUACGAGCAGUACAUCAAGUGGCCCUGGUACAUCUG
GCUGGGCUUCAUCGCCGGCCUGAUCGCCAUCGUGAUGGUGACCAUCAUGCUGU
GCUGCAUGACAAGCUGUUGCAGCUGCCUGAAAGGUUGCUGCAGCUGUGGCUCG
UGCUGCAAGUUCGACGAGGACGACAGCGAGCCCGUGCUGAAGGGCGUGAAGCU
GCACUACACCUGAUGA
>Luciferase mRNA
SEQ ID NO: 62
AUGGAGGACGCCAAGAACAUCAAGAAAGGCCCCGCCCCCUUCUACCCCCUGGA
GGACGGCACCGCCGGCGAGCAGCUGCACAAAGCCAUGAAAAGAUACGCUCUGG
UGCCCGGCACCAUCGCCUUCACCGACGCCCACAUCGAGGUGAACAUCACCUAC
GCCGAGUACUUCGAGAUGAGCGUGAGACUGGCCGAGGCUAUGAAGAGAUACG
GCUUGAACACCAACCACAGAAUCGUGGUGUGCAGCGAGAACAGCCUGCAGUUC
UUCAUGCCCGUGCUGGGCGCCCUGUUCAUCGGCGUGGCCGUUGCCCCCGCCAA
CGACAUCUACAACGAGAGAGAGCUGCUGAACAGCAUGAACAUCUCUCAGCCCA
CCGUGGUUUUCGUGUCCAAAAAGGGCCUGCAGAAGAUCCUGAACGUGCAGAA
GAAGCUGCCCAUCAUUCAGAAGAUCAUCAUCAUGGACAGCAAGACCGACUACC
AAGGCUUUCAGAGCAUGUACACCUUCGUGACAAGCCACCUGCCCCCCGGCUUC
AACGAGUACGACUUCGUGCCCGAGAGCUUCGACAGAGACAAGACCAUCGCCCU
GAUCAUGAACAGCAGCGGCAGCACCGGCCUGCCCAAGGGCGUGGCCCUGCCCC
ACAGAACCGCCUGCGUGAGAUUCAGCCACGCUAGAGACCCCAUCUUCGGCAAU
CAGAUCAUCCCCGACACCGCCAUCCUGAGCGUAGUGCCUUUCCAUCAUGGCUU
CGGCAUGUUCACCACCCUGGGCUACCUGAUCUGCGGCUUCAGAGUGGUGCUGA
UGUACAGAUUCGAGGAGGAGCUGUUCCUGAGAAGCCUGCAAGACUACAAGAU
UCAGAGCGCCCUGCUGGUGCCCACCCUGUUCAGCUUCUUCGCCAAGAGCACCC
UGAUCGACAAGUAUGACCUGAGCAACCUGCACGAAAUUGCGAGCGGCGGAGCG
CCGCUCAGCAAAGAGGUGGGCGAGGCCGUGGCCAAGAGAUUCCACCUGCCCGG
CAUCAGACAAGGCUACGGCCUGACCGAGACCACAAGCGCCAUCCUGAUCACCC
CCGAGGGCGACGAUAAGCCCGGCGCCGUGGGCAAGGUGGUACCAUUCUUCGAG
GCCAAGGUGGUGGACCUGGACACCGGCAAGACCCUGGGCGUGAAUCAGAGAGG
CGAGCUGUGCGUGAGAGGCCCCAUGAUCAUGAGCGGCUACGUGAACAACCCCG
AGGCCACCAACGCCCUCAUCGAUAAGGAUGGGUGGCUGCACAGCGGCGACAUC
GCCUACUGGGACGAGGACGAGCACUUCUUCAUCGUGGACAGACUCAAGAGCCU
GAUCAAGUACAAGGGCUACCAAGUGGCCCCCGCCGAGUUGGAGAGCAUCCUGC
UGCAGCACCCCAAUAUCUUCGACGCUGGCGUGGCCGGACUACCCGACGACGAC
GCCGGCGAGUUACCCGCCGCCGUGGUGGUGCUGGAGCACGGCAAGACCAUGAC
CGAGAAGGAGAUCGUGGACUACGUGGCUAGCCAAGUGACCACCGCCAAGAAGC
UGAGAGGCGGCGUGGUGUUCGUUGACGAAGUGCCCAAGGGCCUGACCGGCAA
GCUGGACGCUAGAAAGAUCAGAGAGAUCCUGAUCAAGGCCAAGAAGGGCGGC
AAGAGCAAGCUGUGA
>LUC1- I′ truncation
SEQ ID NO: 63
UAGGAGUUGGUGGCAGUUUCAAUAACAGGUCAUUGCCGAGAAAAGGAUAGCA
CUAUAAU
>LUC2- I′ truncation
SEQ ID NO: 64
AGUUGGUGGCAGUUUCAAUAACAGGUCAUUGCCGAGAAAAGGAUAGCACUAU
AAU
>LUC3- I′ truncation
SEQ ID NO: 65
GGUGGCAGUUUCAAUAACAGGUCAUUGCCGAGAAAAGGAUAGCACUAUAAU
>LUC4- I′ truncation
SEQ ID NO: 66
GCAGUUUCAAUAACAGGUCAUUGCCGAGAAAAGGAUAGCACUAUAAU
>LUC5- I′ truncation
SEQ ID NO: 67
UUUCAAUAACAGGUCAUUGCCGAGAAAAGGAUAGCACUAUAAU
>LUC6- I′ truncation
SEQ ID NO: 68
AAUAACAGGUCAUUGCCGAGAAAAGGAUAGCACUAUAAU
>LUC7- I′ truncation
SEQ ID NO: 69
ACAGGUCAUUGCCGAGAAAAGGAUAGCACUAUAAU
>LUC8- I′ truncation
SEQ ID NO: 70
GUCAUUGCCGAGAAAAGGAUAGCACUAUAAU
>LUC9- I′ truncation
SEQ ID NO: 71
UUGCCGAGAAAAGGAUAGCACUAUAAU
>LUC10- I′ truncation
SEQ ID NO: 72
CGAGAAAAGGAUAGCACUAUAAU
>LUC11- I′ truncation
SEQ ID NO: 73
AAAAGGAUAGCACUAUAAU
>LUC12- I′ truncation
SEQ ID NO: 74
GGAUAGCACUAUAAU
>LUC13- I′ truncation
SEQ ID NO: 75
AGCACUAUAAU
>LUC14-I′truncation
CUAUAAU
>LUC15- I′ mutant
SEQ ID NO: 77
GAGUUAGGAGUUGCUGGCAGUUUCAAUAACAGGUCAUUGCCGAGAAAAGGAU
AGCACUAUAAU
>LUC16- I′ mutant
SEQ ID NO: 78
GAGUUAGGAGUUGUUGGCAGUUUCAAUAACAGGUCAUUGCCGAGAAAAGGAU
AGCACUAUAAU
>5′UTR I general formula
SEQ ID NO: 79
GAGUUAGGAGUUGN₂UGGCAGUUUCAAUAACAGGUCAUUGCCGAGAAAAGGAU
AGCACUAUAAU

N₂ is selected from the group consisting of A, G, C, and U

>Wildtype SARS-CoV-2 spike protein amino acid sequence
SEQ ID NO: 80
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMDLE
GKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIAD
YNYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKCVN
FNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSY
ECDIPIGAGICASYQTQTNSPRRARSVASQSIIAYTMSLGAENSVAYSNNSIAIPTNFTI
SVTTEILPVSMTKTSVDCTMYICGDSTECSNLLLQYGSFCTQLNRALTGIAVEQDKNTQE
VFAQVKQIYKTPPIKDFGGFNFSQILPDPSKPSKRSFIEDLLFNKVTLADAGFIKQYGDC
LGDIAARDLICAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQDVVNQNAQALN
TLVKQLSSNFGAISSVLNDILSRLDKVEAEVQIDRLITGRLQSLQTYVTQQLIRAAEIRA
SANLAATKMSECVLGQSKRVDFCGKGYHLMSFPQSAPHGVVFLHVTYVPAQEKNFTTAPA
ICHDGKAHFPREGVFVSNGTHWFVTQRNFYEPQIITTDNTFVSGNCDVVIGIVNNTVYDP
LQPELDSFKEELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSCGSCCKFDEDD
SEPVLKGVKLHYT
>Wildtype SARS-CoV-2 spike protein CDS (NC_045512.2)
SEQ ID NO: 81
ATGTTTGTTTTTCTTGTTTTATTGCCACTAGTCTCTAGTCAGTGTGTTAATCTTACAACC
AGAACTCAATTACCCCCTGCATACACTAATTCTTTCACACGTGGTGTTTATTACCCTGAC
AAAGTTTTCAGATCCTCAGTTTTACATTCAACTCAGGACTTGTTCTTACCTTTCTTTTCC
AATGTTACTTGGTTCCATGCTATACATGTCTCTGGGACCAATGGTACTAAGAGGTTTGAT
AACCCTGTCCTACCATTTAATGATGGTGTTTATTTTGCTTCCACTGAGAAGTCTAACATA
ATAAGAGGCTGGATTTTTGGTACTACTTTAGATTCGAAGACCCAGTCCCTACTTATTGTT
AATAACGCTACTAATGTTGTTATTAAAGTCTGTGAATTTCAATTTTGTAATGATCCATTT
TTGGGTGTTTATTACCACAAAAACAACAAAAGTTGGATGGAAAGTGAGTTCAGAGTTTAT
TCTAGTGCGAATAATTGCACTTTTGAATATGTCTCTCAGCCTTTTCTTATGGACCTTGAA
GGAAAACAGGGTAATTTCAAAAATCTTAGGGAATTTGTGTTTAAGAATATTGATGGTTAT
TTTAAAATATATTCTAAGCACACGCCTATTAATTTAGTGCGTGATCTCCCTCAGGGTTTT
TCGGCTTTAGAACCATTGGTAGATTTGCCAATAGGTATTAACATCACTAGGTTTCAAACT
TTACTTGCTTTACATAGAAGTTATTTGACTCCTGGTGATTCTTCTTCAGGTTGGACAGCT
GGTGCTGCAGCTTATTATGTGGGTTATCTTCAACCTAGGACTTTTCTATTAAAATATAAT
GAAAATGGAACCATTACAGATGCTGTAGACTGTGCACTTGACCCTCTCTCAGAAACAAAG
TGTACGTTGAAATCCTTCACTGTAGAAAAAGGAATCTATCAAACTTCTAACTTTAGAGTC
CAACCAACAGAATCTATTGTTAGATTTCCTAATATTACAAACTTGTGCCCTTTTGGTGAA
GTTTTTAACGCCACCAGATTTGCATCTGTTTATGCTTGGAACAGGAAGAGAATCAGCAAC
TGTGTTGCTGATTATTCTGTCCTATATAATTCCGCATCATTTTCCACTTTTAAGTGTTAT
GGAGTGTCTCCTACTAAATTAAATGATCTCTGCTTTACTAATGTCTATGCAGATTCATTT
GTAATTAGAGGTGATGAAGTCAGACAAATCGCTCCAGGGCAAACTGGAAAGATTGCTGAT
TATAATTATAAATTACCAGATGATTTTACAGGCTGCGTTATAGCTTGGAATTCTAACAAT
CTTGATTCTAAGGTTGGTGGTAATTATAATTACCTGTATAGATTGTTTAGGAAGTCTAAT
CTCAAACCTTTTGAGAGAGATATTTCAACTGAAATCTATCAGGCCGGTAGCACACCTTGT
AATGGTGTTGAAGGTTTTAATTGTTACTTTCCTTTACAATCATATGGTTTCCAACCCACT
AATGGTGTTGGTTACCAACCATACAGAGTAGTAGTACTTTCTTTTGAACTTCTACATGCA
CCAGCAACTGTTTGTGGACCTAAAAAGTCTACTAATTTGGTTAAAAACAAATGTGTCAAT
TTCAACTTCAATGGTTTAACAGGCACAGGTGTTCTTACTGAGTCTAACAAAAAGTTTCTG
CCTTTCCAACAATTTGGCAGAGACATTGCTGACACTACTGATGCTGTCCGTGATCCACAG
ACACTTGAGATTCTTGACATTACACCATGTTCTTTTGGTGGTGTCAGTGTTATAACACCA
GGAACAAATACTTCTAACCAGGTTGCTGTTCTTTATCAGGATGTTAACTGCACAGAAGTC
CCTGTTGCTATTCATGCAGATCAACTTACTCCTACTTGGCGTGTTTATTCTACAGGTTCT
AATGTTTTTCAAACACGTGCAGGCTGTTTAATAGGGGCTGAACATGTCAACAACTCATAT
GAGTGTGACATACCCATTGGTGCAGGTATATGCGCTAGTTATCAGACTCAGACTAATTCT
CCTCGGCGGGCACGTAGTGTAGCTAGTCAATCCATCATTGCCTACACTATGTCACTTGGT
GCAGAAAATTCAGTTGCTTACTCTAATAACTCTATTGCCATACCCACAAATTTTACTATT
AGTGTTACCACAGAAATTCTACCAGTGTCTATGACCAAGACATCAGTAGATTGTACAATG
TACATTTGTGGTGATTCAACTGAATGCAGCAATCTTTTGTTGCAATATGGCAGTTTTTGT
ACACAATTAAACCGTGCTTTAACTGGAATAGCTGTTGAACAAGACAAAAACACCCAAGAA
GTTTTTGCACAAGTCAAACAAATTTACAAAACACCACCAATTAAAGATTTTGGTGGTTTT
AATTTTTCACAAATATTACCAGATCCATCAAAACCAAGCAAGAGGTCATTTATTGAAGAT
CTACTTTTCAACAAAGTGACACTTGCAGATGCTGGCTTCATCAAACAATATGGTGATTGC
CTTGGTGATATTGCTGCTAGAGACCTCATTTGTGCACAAAAGTTTAACGGCCTTACTGTT
TTGCCACCTTTGCTCACAGATGAAATGATTGCTCAATACACTTCTGCACTGTTAGCGGGT
ACAATCACTTCTGGTTGGACCTTTGGTGCAGGTGCTGCATTACAAATACCATTTGCTATG
CAAATGGCTTATAGGTTTAATGGTATTGGAGTTACACAGAATGTTCTCTATGAGAACCAA
AAATTGATTGCCAACCAATTTAATAGTGCTATTGGCAAAATTCAAGACTCACTTTCTTCC
ACAGCAAGTGCACTTGGAAAACTTCAAGATGTGGTCAACCAAAATGCACAAGCTTTAAAC
ACGCTTGTTAAACAACTTAGCTCCAATTTTGGTGCAATTTCAAGTGTTTTAAATGATATC
CTTTCACGTCTTGACAAAGTTGAGGCTGAAGTGCAAATTGATAGGTTGATCACAGGCAGA
CTTCAAAGTTTGCAGACATATGTGACTCAACAATTAATTAGAGCTGCAGAAATCAGAGCT
TCTGCTAATCTTGCTGCTACTAAAATGTCAGAGTGTGTACTTGGACAATCAAAAAGAGTT
GATTTTTGTGGAAAGGGCTATCATCTTATGTCCTTCCCTCAGTCAGCACCTCATGGTGTA
GTCTTCTTGCATGTGACTTATGTCCCTGCACAAGAAAAGAACTTCACAACTGCTCCTGCC
ATTTGTCATGATGGAAAAGCACACTTTCCTCGTGAAGGTGTCTTTGTTTCAAATGGCACA
CACTGGTTTGTAACACAAAGGAATTTTTATGAACCACAAATCATTACTACAGACAACACA
TTTGTGTCTGGTAACTGTGATGTTGTAATAGGAATTGTCAACAACACAGTTTATGATCCT
TTGCAACCTGAATTAGACTCATTCAAGGAGGAGTTAGATAAATATTTTAAGAATCATACA
TCACCAGATGTTGATTTAGGTGACATCTCTGGCATTAATGCTTCAGTTGTAAACATTCAA
AAAGAAATTGACCGCCTCAATGAGGTTGCCAAGAATTTAAATGAATCTCTCATCGATCTC
CAAGAACTTGGAAAGTATGAGCAGTATATAAAATGGCCATGGTACATTTGGCTAGGTTTT
ATAGCTGGCTTGATTGCCATAGTAATGGTGACAATTATGCTTTGCTGTATGACCAGTTGC
TGTAGTTGTCTCAAGGGCTGTTGTTCTTGTGGATCCTGCTGCAAATTTGATGAAGACGAC
TCTGAGCCAGTGCTCAAAGGAGTCAAATTACATTACACATAA
>Wildtype SARS-CoV-2 spike protein mRNA
SEQ ID NO: 82
AUGUUUGUUUUUCUUGUUUUAUUGCCACUAGUCUCUAGUCAGUGUGUUAAUCUUACAACC
AGAACUCAAUUACCCCCUGCAUACACUAAUUCUUUCACACGUGGUGUUUAUUACCCUGAC
AAAGUUUUCAGAUCCUCAGUUUUACAUUCAACUCAGGACUUGUUCUUACCUUUCUUUUCC
AAUGUUACUUGGUUCCAUGCUAUACAUGUCUCUGGGACCAAUGGUACUAAGAGGUUUGAU
AACCCUGUCCUACCAUUUAAUGAUGGUGUUUAUUUUGCUUCCACUGAGAAGUCUAACAUA
AUAAGAGGCUGGAUUUUUGGUACUACUUUAGAUUCGAAGACCCAGUCCCUACUUAUUGUU
AAUAACGCUACUAAUGUUGUUAUUAAAGUCUGUGAAUUUCAAUUUUGUAAUGAUCCAUUU
UUGGGUGUUUAUUACCACAAAAACAACAAAAGUUGGAUGGAAAGUGAGUUCAGAGUUUAU
UCUAGUGCGAAUAAUUGCACUUUUGAAUAUGUCUCUCAGCCUUUUCUUAUGGACCUUGAA
GGAAAACAGGGUAAUUUCAAAAAUCUUAGGGAAUUUGUGUUUAAGAAUAUUGAUGGUUAU
UUUAAAAUAUAUUCUAAGCACACGCCUAUUAAUUUAGUGCGUGAUCUCCCUCAGGGUUUU
UCGGCUUUAGAACCAUUGGUAGAUUUGCCAAUAGGUAUUAACAUCACUAGGUUUCAAACU
UUACUUGCUUUACAUAGAAGUUAUUUGACUCCUGGUGAUUCUUCUUCAGGUUGGACAGCU
GGUGCUGCAGCUUAUUAUGUGGGUUAUCUUCAACCUAGGACUUUUCUAUUAAAAUAUAAU
GAAAAUGGAACCAUUACAGAUGCUGUAGACUGUGCACUUGACCCUCUCUCAGAAACAAAG
UGUACGUUGAAAUCCUUCACUGUAGAAAAAGGAAUCUAUCAAACUUCUAACUUUAGAGUC
CAACCAACAGAAUCUAUUGUUAGAUUUCCUAAUAUUACAAACUUGUGCCCUUUUGGUGAA
GUUUUUAACGCCACCAGAUUUGCAUCUGUUUAUGCUUGGAACAGGAAGAGAAUCAGCAAC
UGUGUUGCUGAUUAUUCUGUCCUAUAUAAUUCCGCAUCAUUUUCCACUUUUAAGUGUUAU
GGAGUGUCUCCUACUAAAUUAAAUGAUCUCUGCUUUACUAAUGUCUAUGCAGAUUCAUUU
GUAAUUAGAGGUGAUGAAGUCAGACAAAUCGCUCCAGGGCAAACUGGAAAGAUUGCUGAU
UAUAAUUAUAAAUUACCAGAUGAUUUUACAGGCUGCGUUAUAGCUUGGAAUUCUAACAAU
CUUGAUUCUAAGGUUGGUGGUAAUUAUAAUUACCUGUAUAGAUUGUUUAGGAAGUCUAAU
CUCAAACCUUUUGAGAGAGAUAUUUCAACUGAAAUCUAUCAGGCCGGUAGCACACCUUGU
AAUGGUGUUGAAGGUUUUAAUUGUUACUUUCCUUUACAAUCAUAUGGUUUCCAACCCACU
AAUGGUGUUGGUUACCAACCAUACAGAGUAGUAGUACUUUCUUUUGAACUUCUACAUGCA
CCAGCAACUGUUUGUGGACCUAAAAAGUCUACUAAUUUGGUUAAAAACAAAUGUGUCAAU
UUCAACUUCAAUGGUUUAACAGGCACAGGUGUUCUUACUGAGUCUAACAAAAAGUUUCUG
CCUUUCCAACAAUUUGGCAGAGACAUUGCUGACACUACUGAUGCUGUCCGUGAUCCACAG
ACACUUGAGAUUCUUGACAUUACACCAUGUUCUUUUGGUGGUGUCAGUGUUAUAACACCA
GGAACAAAUACUUCUAACCAGGUUGCUGUUCUUUAUCAGGAUGUUAACUGCACAGAAGUC
CCUGUUGCUAUUCAUGCAGAUCAACUUACUCCUACUUGGCGUGUUUAUUCUACAGGUUCU
AAUGUUUUUCAAACACGUGCAGGCUGUUUAAUAGGGGCUGAACAUGUCAACAACUCAUAU
GAGUGUGACAUACCCAUUGGUGCAGGUAUAUGCGCUAGUUAUCAGACUCAGACUAAUUCU
CCUCGGCGGGCACGUAGUGUAGCUAGUCAAUCCAUCAUUGCCUACACUAUGUCACUUGGU
GCAGAAAAUUCAGUUGCUUACUCUAAUAACUCUAUUGCCAUACCCACAAAUUUUACUAUU
AGUGUUACCACAGAAAUUCUACCAGUGUCUAUGACCAAGACAUCAGUAGAUUGUACAAUG
UACAUUUGUGGUGAUUCAACUGAAUGCAGCAAUCUUUUGUUGCAAUAUGGCAGUUUUUGU
ACACAAUUAAACCGUGCUUUAACUGGAAUAGCUGUUGAACAAGACAAAAACACCCAAGAA
GUUUUUGCACAAGUCAAACAAAUUUACAAAACACCACCAAUUAAAGAUUUUGGUGGUUUU
AAUUUUUCACAAAUAUUACCAGAUCCAUCAAAACCAAGCAAGAGGUCAUUUAUUGAAGAU
CUACUUUUCAACAAAGUGACACUUGCAGAUGCUGGCUUCAUCAAACAAUAUGGUGAUUGC
CUUGGUGAUAUUGCUGCUAGAGACCUCAUUUGUGCACAAAAGUUUAACGGCCUUACUGUU
UUGCCACCUUUGCUCACAGAUGAAAUGAUUGCUCAAUACACUUCUGCACUGUUAGCGGGU
ACAAUCACUUCUGGUUGGACCUUUGGUGCAGGUGCUGCAUUACAAAUACCAUUUGCUAUG
CAAAUGGCUUAUAGGUUUAAUGGUAUUGGAGUUACACAGAAUGUUCUCUAUGAGAACCAA
AAAUUGAUUGCCAACCAAUUUAAUAGUGCUAUUGGCAAAAUUCAAGACUCACUUUCUUCC
ACAGCAAGUGCACUUGGAAAACUUCAAGAUGUGGUCAACCAAAAUGCACAAGCUUUAAAC
ACGCUUGUUAAACAACUUAGCUCCAAUUUUGGUGCAAUUUCAAGUGUUUUAAAUGAUAUC
CUUUCACGUCUUGACAAAGUUGAGGCUGAAGUGCAAAUUGAUAGGUUGAUCACAGGCAGA
CUUCAAAGUUUGCAGACAUAUGUGACUCAACAAUUAAUUAGAGCUGCAGAAAUCAGAGCU
UCUGCUAAUCUUGCUGCUACUAAAAUGUCAGAGUGUGUACUUGGACAAUCAAAAAGAGUU
GAUUUUUGUGGAAAGGGCUAUCAUCUUAUGUCCUUCCCUCAGUCAGCACCUCAUGGUGUA
GUCUUCUUGCAUGUGACUUAUGUCCCUGCACAAGAAAAGAACUUCACAACUGCUCCUGCC
AUUUGUCAUGAUGGAAAAGCACACUUUCCUCGUGAAGGUGUCUUUGUUUCAAAUGGCACA
CACUGGUUUGUAACACAAAGGAAUUUUUAUGAACCACAAAUCAUUACUACAGACAACACA
UUUGUGUCUGGUAACUGUGAUGUUGUAAUAGGAAUUGUCAACAACACAGUUUAUGAUCCU
UUGCAACCUGAAUUAGACUCAUUCAAGGAGGAGUUAGAUAAAUAUUUUAAGAAUCAUACA
UCACCAGAUGUUGAUUUAGGUGACAUCUCUGGCAUUAAUGCUUCAGUUGUAAACAUUCAA
AAAGAAAUUGACCGCCUCAAUGAGGUUGCCAAGAAUUUAAAUGAAUCUCUCAUCGAUCUC
CAAGAACUUGGAAAGUAUGAGCAGUAUAUAAAAUGGCCAUGGUACAUUUGGCUAGGUUUU
AUAGCUGGCUUGAUUGCCAUAGUAAUGGUGACAAUUAUGCUUUGCUGUAUGACCAGUUGC
UGUAGUUGUCUCAAGGGCUGUUGUUCUUGUGGAUCCUGCUGCAAAUUUGAUGAAGACGAC
UCUGAGCCAGUGCUCAAAGGAGUCAAAUUACAUUACACAUAA
>Influenza A virus H1N1 (A/Wisconsin/588/2019)
HA amino acid sequence
SEQ ID NO: 83
MKAILVVMLYTFTTANADTLCIGYHANNSTDTVDTVLEKNVTVTHSVNLLEDKHNGKLCK
LRGVAPLHLGKCNIAGWILGNPECESLSTARSWSYIVETSNSDNGTCYPGDFINYEELRE
QLSSVSSFERFEIFPKTSSWPNHDSDNGVTAACPHAGAKSFYKNLIWLVKKGKSYPKINQ
TYINDKGKEVLVLWGIHHPPTIADQQSLYQNADAYVFVGTSRYSKKFKPEIATRPKVRDQ
EGRMNYYWTLVEPGDKITFEATGNLVAPRYAFTMERDAGSGIIISDTPVHDCNTTCQTPE
GAINTSLPFQNVHPITIGKCPKYVKSTKLRLATGLRNVPSIQSRGLFGAIAGFIEGGWTG
MVDGWYGYHHQNEQGSGYAADLKSTQNAIDKITNKVNSVIEKMNTQFTAVGKEFNHLEKR
IENLNKKVDDGFLDIWTYNAELLVLLENERTLDYHDSNVKNLYEKVRNQLKNNAKEIGNG
CFEFYHKCDNTCMESVKNGTYDYPKYSEEAKLNREKIDGVKLDSTRIYQILAIYSTVASS
LVLVVSLGAISFWMCSNGSLQCRICI
>Influenza A virus H3N2 (A/Cambodia/e0826360/2020)
HA amino acid sequence
SEQ ID NO: 84
MKTIIALSYILCLVFAQKIPGNDNSTATLCLGHHAVPNGTIVKTITNDRIEVTNATELVQ
NSSIGEICDSPHQILDGGNCTLIDALLGDPQCDGFQNKEWDLFVERSRANSNCYPYDVPD
YASLRSLVASSGTLEFKNESFNWTGVKQNGTSSACIRGSSSSFFSRLNWLTHLNYTYPAL
NVTMPNNEQFDKLYIWGVHHPSTDKDQISLFAQPSGRITVSTKRSQQAVIPNIGSRPRIR
DIPSRISIYWTIVKPGDILLINSTGNLIAPRGYFKIRSGKSSIMRSDAPIGKCKSECITP
NGSIPNDKPFQNVNRITYGACPRYVKQSTLKLATGMRNVPEKQTRGIFGAIAGFIENGWE
GMVDGWYGFRHQNSEGRGQAADLKSTQAAIDQINGKLNRLIGKTNEKFHQIEKEFSEVEG
RVQDLEKYVEDTKIDLWSYNAELLVALENQHTIDLTDSEMNKLFEKTKKQLRENAEDMGN
GCFKIYHKCDNACIGSIRNETYDHNVYRDEALNNRFQIKGVELKSGYKDWILWISFAMSC
FLLCIALLGFIMWACQKGNIRCNICI
>Influenza B virus Victoria lineage (B/Washington/02/2019)
HA amino acid sequence
SEQ ID NO: 85
MKAIIVLLMVVTSNADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIR
QLPNLLRGYEHVRLSTHNVINAEDAPGRPYEIGTSGSCPNITNGNGFFATMAWAVPKNKT
ATNPLTIEVPYICTEGEDQITVWGFHSDNETQMAKLYGDSKPQKFTSSANGVTTHYVSQI
GGFPNQTEDGGLPQSGRIVVDYMVQKSGKTGTITYQRGILLPQKVWCASGRSKVIKGSLP
LIGEADCLHEKYGGLNKSKPYYTGEHAKAIGNCPIWVKTPLKLANGTKYRPPAKLLKERG
FFGAIAGFLEGGWEGMIAGWHGYTSHGAHGVAVAADLKSTQEAINKITKNLNSLSELEVK
NLQRLSGAMDELHNEILELDEKVDDLRADTISSQIELAVLLSNEGIINSEDEHLLALERK
LKKMLGPSAVEIGNGCFETKHKCNQTCLDRIAAGTFDAGEFSLPTFDSLNITAASLNDDG
LDNHTILLYYSTAASSLAVTLMIAIFVVYMVSRDNVSCSICL
>Influenza B virus Yamagata lineage (B/Phuket/3073/2013)
HA amino acid sequence
SEQ ID NO: 86
MKAIIVLLMVVTSNADRICTGITSSNSPHVVKTATQGEVNVTGVIPLTTTPTKSYFANLK
GTRTRGKLCPDCLNCTDLDVALGRPMCVGTTPSAKASILHEVRPVTSGCFPIMHDRTKIR
QLPNLLRGYEKIRLSTQNVIDAEKAPGGPYRLGTSGSCPNATSKIGFFATMAWAVPKDNY
KNATNPLTVEVPYICTEGEDQITVWGFHSDNKTQMKSLYGDSNPQKFTSSANGVTTHYVS
QIGDFPDQTEDGGLPQSGRIVVDYMMQKPGKTGTIVYQRGVLLPQKVWCASGRSKVIKGS
LPLIGEADCLHEEYGGLNKSKPYYTGKHAKAIGNCPIWVKTPLKLANGTKYRPPAKLLKE
RGFFGAIAGFLEGGWEGMIAGWHGYTSHGAHGVAVAADLKSTQEAINKITKNLNSLSELE
VKNLQRLSGAMDELHNEILELDEKVDDLRADTISSQIELAVLLSNEGIINSEDEHLLALE
RKLKKMLGPSAVDIGNGCFETKHKCNQTCLDRIAAGTFNAGEFSLPTFDSLNITAASLND
DGLDNHTILLYYSTAASSLAVTLMLAIFIVYMVSRDNVSCSICL
>Influenza A virus H1N1 (A/Wisconsin/588/2019)
HA CDS
SEQ ID NO: 87
ATGAAGGCCATCCTGGTGGTGATGCTGTACACCTTCACCACCGCCAACGCCGACACCCTG
TGCATCGGCTACCACGCCAACAACAGCACCGACACCGTGGACACCGTGCTGGAGAAGAAC
GTGACCGTGACCCACAGCGTGAACCTGCTGGAGGACAAGCACAACGGCAAGCTGTGCAAG
CTGCGGGGCGTGGCCCCCCTGCACCTGGGCAAGTGCAACATCGCCGGCTGGATCCTGGGC
AACCCCGAGTGCGAGTCCCTGAGCACCGCCCGCTCCTGGAGCTACATCGTGGAGACCTCC
AACAGCGACAACGGCACCTGCTACCCCGGGGACTTCATCAACTACGAGGAGCTGCGGGAG
CAGCTGTCCAGCGTGTCCAGCTTCGAGCGCTTCGAGATCTTCCCCAAGACCTCCAGCTGG
CCCAACCACGACTCCGACAACGGCGTGACCGCCGCCTGCCCCCACGCCGGGGCCAAGAGC
TTCTACAAGAACCTGATCTGGCTGGTGAAGAAGGGCAAGTCCTACCCCAAGATCAACCAG
ACCTACATCAACGACAAGGGGAAGGAGGTCCTCGTGCTGTGGGGCATCCACCACCCCCCC
ACCATCGCCGACCAGCAGAGCCTCTACCAGAACGCCGACGCCTACGTCTTCGTGGGGACC
TCCCGGTACAGCAAGAAGTTCAAGCCCGAGATCGCCACCCGCCCCAAGGTCCGGGACCAG
GAGGGCCGCATGAACTACTACTGGACCCTGGTGGAGCCGGGGGACAAGATCACCTTCGAG
GCCACCGGCAACCTCGTCGCCCCGCGGTACGCCTTCACGATGGAGCGCGACGCCGGGTCC
GGCATCATCATCAGCGACACCCCCGTGCACGACTGCAACACGACCTGCCAGACGCCCGAG
GGGGCGATCAACACCTCCCTGCCGTTCCAGAACGTCCACCCGATCACGATCGGCAAGTGC
CCCAAGTACGTGAAGTCCACCAAGCTCCGGCTGGCCACGGGGCTCCGCAACGTCCCCTCC
ATCCAGAGCCGGGGCCTGTTCGGGGCGATCGCCGGCTTCATCGAGGGGGGCTGGACCGGG
ATGGTGGACGGCTGGTACGGGTACCACCACCAGAACGAGCAGGGCTCCGGGTACGCGGCC
GACCTCAAGTCGACGCAGAACGCGATCGACAAGATCACCAACAAGGTCAACAGCGTGATC
GAGAAGATGAACACGCAGTTCACCGCCGTCGGCAAGGAGTTCAACCACCTGGAGAAGCGC
ATCGAGAACCTCAACAAGAAGGTGGACGACGGGTTCCTGGACATCTGGACGTACAACGCG
GAGCTCCTGGTCCTCCTGGAGAACGAGCGGACCCTCGACTACCACGACTCCAACGTGAAG
AACCTGTACGAGAAGGTCCGCAACCAGCTCAAGAACAACGCCAAGGAGATCGGCAACGGG
TGCTTCGAGTTCTACCACAAGTGCGACAACACGTGCATGGAGTCGGTGAAGAACGGCACC
TACGACTACCCGAAGTACAGCGAGGAGGCGAAGCTGAACCGGGAGAAGATCGACGGGGTC
AAGCTCGACTCCACGCGCATCTACCAGATCCTGGCCATCTACTCGACCGTGGCGAGCTCC
CTCGTCCTGGTGGTCTCGCTCGGCGCCATCAGCTTCTGGATGTGCTCCAACGGGTCGCTG
CAGTGCCGGATCTGCATCTGA
>Influenza A virus H3N2 (A/Cambodia/e0826360/2020)
HA CDS
SEQ ID NO: 88
ATGAAGACCATCATCGCCCTGAGCTACATCCTGTGCCTGGTGTTCGCCCAGAAGATCCCC
GGCAACGACAACAGCACCGCCACCCTGTGCCTGGGCCACCACGCCGTGCCCAACGGCACC
ATCGTGAAGACCATCACCAACGACCGGATCGAGGTGACCAACGCCACCGAGCTGGTGCAG
AACTCCAGCATCGGCGAGATCTGCGACTCCCCCCACCAGATCCTGGACGGCGGCAACTGC
ACCCTGATCGACGCCCTGCTGGGCGACCCCCAGTGCGACGGGTTCCAGAACAAGGAGTGG
GACCTGTTCGTGGAGCGGAGCCGCGCCAACTCCAACTGCTACCCCTACGACGTGCCCGAC
TACGCCAGCCTGCGGTCCCTGGTGGCCAGCTCCGGCACCCTGGAGTTCAAGAACGAGAGC
TTCAACTGGACCGGGGTCAAGCAGAACGGCACCTCCAGCGCCTGCATCCGCGGGTCCAGC
TCCAGCTTCTTCTCCCGGCTGAACTGGCTCACCCACCTGAACTACACCTACCCCGCCCTC
AACGTGACCATGCCCAACAACGAGCAGTTCGACAAGCTGTACATCTGGGGCGTCCACCAC
CCCAGCACCGACAAGGACCAGATCTCCCTCTTCGCCCAGCCCAGCGGGCGCATCACCGTG
TCCACGAAGCGGAGCCAGCAGGCCGTCATCCCCAACATCGGCTCCCGCCCGCGGATCCGC
GACATCCCCAGCCGGATCTCCATCTACTGGACGATCGTGAAGCCGGGGGACATCCTGCTC
ATCAACAGCACCGGCAACCTGATCGCCCCCCGCGGGTACTTCAAGATCCGGTCCGGCAAG
AGCTCGATCATGCGCTCCGACGCCCCGATCGGGAAGTGCAAGAGCGAGTGCATCACCCCC
AACGGCTCGATCCCGAACGACAAGCCCTTCCAGAACGTCAACCGGATCACGTACGGGGCC
TGCCCGCGCTACGTGAAGCAGTCCACGCTCAAGCTGGCCACCGGCATGCGGAACGTCCCC
GAGAAGCAGACCCGCGGGATCTTCGGCGCCATCGCGGGGTTCATCGAGAACGGCTGGGAG
GGGATGGTGGACGGCTGGTACGGGTTCCGGCACCAGAACAGCGAGGGCCGCGGGCAGGCC
GCGGACCTCAAGTCGACGCAGGCCGCGATCGACCAGATCAACGGCAAGCTGAACCGGCTC
ATCGGGAAGACGAACGAGAAGTTCCACCAGATCGAGAAGGAGTTCTCCGAGGTCGAGGGC
CGCGTGCAGGACCTGGAGAAGTACGTCGAGGACACCAAGATCGACCTCTGGAGCTACAAC
GCCGAGCTGCTCGTGGCGCTGGAGAACCAGCACACCATCGACCTCACGGACTCGGAGATG
AACAAGCTGTTCGAGAAGACGAAGAAGCAGCTCCGGGAGAACGCCGAGGACATGGGGAAC
GGCTGCTTCAAGATCTACCACAAGTGCGACAACGCGTGCATCGGGTCCATCCGCAACGAG
ACCTACGACCACAACGTCTACCGGGACGAGGCCCTGAACAACCGCTTCCAGATCAAGGGC
GTGGAGCTCAAGAGCGGGTACAAGGACTGGATCCTGTGGATCTCGTTCGCGATGTCCTGC
TTCCTCCTGTGCATCGCCCTCCTGGGCTTCATCATGTGGGCGTGCCAGAAGGGGAACATC
CGGTGCAACATCTGCATCTGA
>Influenza B virus Victoria lineage (B/Washington/02/2019)
HA CDS
SEQ ID NO: 89
ATGAAGGCCATCATCGTGCTGCTGATGGTGGTGACCAGCAACGCCGACCGGATCTGCACC
GGCATCACCAGCTCCAACAGCCCCCACGTGGTGAAGACCGCCACCCAGGGCGAGGTGAAC
GTGACCGGCGTGATCCCCCTGACCACCACCCCCACCAAGTCCCACTTCGCCAACCTGAAG
GGCACCGAGACCCGGGGCAAGCTGTGCCCCAAGTGCCTGAACTGCACCGACCTGGACGTG
GCCCTGGGCCGCCCCAAGTGCACCGGCAAGATCCCCAGCGCCCGGGTGTCCATCCTGCAC
GAGGTGCGCCCCGTGACCAGCGGCTGCTTCCCCATCATGCACGACCGCACCAAGATCCGG
CAGCTGCCCAACCTGCTGCGGGGGTACGAGCACGTCCGCCTGTCCACCCACAACGTGATC
AACGCCGAGGACGCCCCCGGCCGGCCCTACGAGATCGGGACCAGCGGCTCCTGCCCCAAC
ATCACCAACGGGAACGGCTTCTTCGCCACCATGGCCTGGGCCGTCCCCAAGAACAAGACC
GCCACCAACCCCCTGACCATCGAGGTGCCGTACATCTGCACCGAGGGGGAGGACCAGATC
ACCGTCTGGGGCTTCCACAGCGACAACGAGACGCAGATGGCCAAGCTGTACGGGGACTCC
AAGCCCCAGAAGTTCACGAGCTCCGCCAACGGCGTGACCACCCACTACGTCAGCCAGATC
GGGGGCTTCCCGAACCAGACGGAGGACGGGGGCCTGCCCCAGTCCGGGCGCATCGTGGTC
GACTACATGGTGCAGAAGTCCGGCAAGACGGGGACCATCACGTACCAGCGGGGCATCCTG
CTGCCGCAGAAGGTCTGGTGCGCCTCCGGGCGCAGCAAGGTGATCAAGGGCTCCCTGCCC
CTCATCGGGGAGGCCGACTGCCTGCACGAGAAGTACGGCGGGCTCAACAAGAGCAAGCCG
TACTACACCGGCGAGCACGCCAAGGCCATCGGGAACTGCCCCATCTGGGTCAAGACCCCG
CTGAAGCTCGCCAACGGCACGAAGTACCGGCCCCCGGCCAAGCTGCTCAAGGAGCGCGGG
TTCTTCGGCGCCATCGCCGGGTTCCTGGAGGGCGGGTGGGAGGGCATGATCGCCGGGTGG
CACGGCTACACGTCCCACGGGGCCCACGGCGTGGCGGTCGCCGCGGACCTCAAGAGCACC
CAGGAGGCCATCAACAAGATCACGAAGAACCTGAACTCCCTCAGCGAGCTGGAGGTGAAG
AACCTCCAGCGCCTGTCCGGGGCGATGGACGAGCTCCACAACGAGATCCTGGAGCTCGAC
GAGAAGGTCGACGACCTGCGGGCCGACACCATCAGCTCGCAGATCGAGCTCGCGGTGCTG
CTCTCCAACGAGGGCATCATCAACAGCGAGGACGAGCACCTGCTCGCCCTGGAGCGGAAG
CTCAAGAAGATGCTGGGGCCCTCGGCGGTCGAGATCGGCAACGGGTGCTTCGAGACCAAG
CACAAGTGCAACCAGACGTGCCTCGACCGCATCGCCGCGGGCACGTTCGACGCCGGGGAG
TTCTCCCTGCCGACCTTCGACAGCCTCAACATCACGGCGGCCTCGCTGAACGACGACGGC
CTCGACAACCACACCATCCTGCTCTACTACTCCACCGCGGCCAGCTCGCTGGCGGTGACG
CTCATGATCGCCATCTTCGTCGTGTACATGGTCTCCCGCGACAACGTGAGCTGCTCGATC
TGCCTGTGA
>Influenza B virus/Yamagata lineage (B/Phuket/3073/2013)
HA CDS
SEQ ID NO: 90
ATGAAGGCCATCATCGTGCTGCTGATGGTGGTGACCAGCAACGCCGACCGGATCTGCACC
GGCATCACCAGCTCCAACAGCCCCCACGTGGTGAAGACCGCCACCCAGGGCGAGGTGAAC
GTGACCGGCGTGATCCCCCTGACCACCACCCCCACCAAGTCCTACTTCGCCAACCTGAAG
GGCACCCGGACCCGCGGCAAGCTGTGCCCCGACTGCCTGAACTGCACCGACCTGGACGTG
GCCCTGGGCCGGCCCATGTGCGTGGGCACCACCCCCAGCGCCAAGGCCTCCATCCTGCAC
GAGGTGCGCCCCGTGACCAGCGGCTGCTTCCCCATCATGCACGACCGCACCAAGATCCGG
CAGCTGCCCAACCTGCTGCGGGGGTACGAGAAGATCCGCCTGTCCACCCAGAACGTCATC
GACGCCGAGAAGGCCCCCGGCGGGCCCTACCGGCTGGGCACCAGCGGGTCCTGCCCCAAC
GCCACCAGCAAGATCGGCTTCTTCGCCACCATGGCCTGGGCCGTGCCCAAGGACAACTAC
AAGAACGCCACCAACCCCCTGACCGTCGAGGTGCCCTACATCTGCACCGAGGGGGAGGAC
CAGATCACGGTCTGGGGCTTCCACTCCGACAACAAGACCCAGATGAAGTCCCTGTACGGG
GACTCCAACCCGCAGAAGTTCACGAGCTCCGCCAACGGCGTGACCACGCACTACGTCAGC
CAGATCGGGGACTTCCCGGACCAGACCGAGGACGGCGGGCTGCCCCAGTCCGGCCGCATC
GTGGTCGACTACATGATGCAGAAGCCCGGGAAGACGGGCACCATCGTGTACCAGCGGGGG
GTCCTGCTGCCGCAGAAGGTGTGGTGCGCCAGCGGCCGCTCCAAGGTCATCAAGGGGAGC
CTGCCCCTCATCGGCGAGGCCGACTGCCTGCACGAGGAGTACGGGGGCCTCAACAAGTCC
AAGCCGTACTACACGGGGAAGCACGCCAAGGCCATCGGCAACTGCCCGATCTGGGTGAAG
ACCCCCCTGAAGCTCGCCAACGGGACGAAGTACCGGCCGCCCGCCAAGCTGCTCAAGGAG
CGCGGCTTCTTCGGGGCCATCGCCGGCTTCCTGGAGGGGGGCTGGGAGGGGATGATCGCC
GGCTGGCACGGGTACACCAGCCACGGCGCCCACGGGGTCGCGGTGGCCGCGGACCTCAAG
TCCACGCAGGAGGCCATCAACAAGATCACCAAGAACCTGAACAGCCTCTCCGAGCTGGAG
GTCAAGAACCTCCAGCGCCTGAGCGGCGCGATGGACGAGCTCCACAACGAGATCCTGGAG
CTCGACGAGAAGGTGGACGACCTGCGGGCCGACACGATCTCGTCCCAGATCGAGCTCGCG
GTCCTGCTCAGCAACGAGGGGATCATCAACTCGGAGGACGAGCACCTGCTCGCCCTGGAG
CGGAAGCTCAAGAAGATGCTGGGCCCGTCCGCGGTGGACATCGGGAACGGCTGCTTCGAG
ACCAAGCACAAGTGCAACCAGACGTGCCTCGACCGCATCGCCGCGGGGACCTTCAACGCC
GGCGAGTTCAGCCTGCCCACGTTCGACTCGCTCAACATCACCGCGGCCTCCCTGAACGAC
GACGGGCTCGACAACCACACGATCCTGCTCTACTACAGCACCGCGGCCTCGTCCCTGGCG
GTCACGCTCATGCTGGCCATCTTCATCGTGTACATGGTCAGCCGCGACAACGTGTCGTGC
TCCATCTGCCTCTGA
>5'UTR-Moderna
SEQ ID NO: 91
GGGAAATAAGAGAGAAAAGAAGAGTAAGAAGAAATATAAGACCCCGGCGCCGCCACC
>3'UTR-Moderna
SEQ ID NO: 92
GCTGGAGCCTCGGTGGCCTAGCTTCTTGCCCCTTGGGCCTCCCCCCAGCCCCTCCTCCCC
TTCCTGCACCCGTACCCCCGTGGTCTTTGAATAAAGTCTGAGTGGGCGGC
>Influenza A virus H1N1 (A/Wisconsin/588/2019)
HA Moderna
SEQ ID NO: 93
ATGAAGGCCATCCTGGTCGTGATGCTGTACACCTTCACCACCGCCAACGCCGACACCCTG
TGCATCGGCTACCACGCCAACAACAGCACCGACACCGTGGACACCGTGCTGGAGAAGAAC
GTGACCGTGACCCACAGCGTGAACCTGCTGGAGGACAAGCACAACGGCAAGCTGTGCAAG
CTGAGGGGAGTGGCACCCCTGCACCTGGGCAAGTGCAACATCGCCGGCTGGATCCTGGGC
AACCCCGAGTGCGAGAGCCTGAGCACAGCCCGGAGCTGGAGCTACATCGTGGAGACCAGC
AACAGCGACAACGGCACCTGTTACCCCGGCGACTTCATCAACTACGAGGAGCTGCGGGAG
CAGCTGAGCAGCGTGAGCAGCTTCGAGCGGTTCGAGATCTTCCCCAAGACCAGCAGCTGG
CCCAACCACGACAGCGACAACGGCGTGACAGCAGCCTGTCCACACGCCGGAGCCAAGAGC
TTCTACAAGAACCTGATCTGGCTGGTGAAGAAGGGCAAGAGCTACCCCAAGATCAACCAG
ACCTACATCAACGACAAGGGCAAGGAGGTGCTGGTGCTGTGGGGCATCCACCACCCACCT
ACCATCGCCGACCAGCAGAGCCTGTACCAGAACGCCGACGCCTACGTGTTCGTGGGCACC
AGCCGGTACAGCAAGAAGTTCAAGCCAGAGATCGCCACCCGGCCCAAGGTGAGAGACCAG
GAGGGCCGGATGAACTACTACTGGACCCTGGTGGAGCCCGGAGACAAGATTACCTTCGAG
GCCACCGGCAACCTGGTGGCCCCTCGGTACGCCTTCACCATGGAACGGGACGCTGGCAGC
GGCATCATCATCAGCGACACTCCCGTGCACGACTGCAACACCACCTGCCAGACTCCCGAG
GGCGCTATCAACACCAGCCTGCCCTTCCAGAACGTGCACCCCATCACCATCGGCAAGTGC
CCCAAGTACGTAAAGAGCACCAAATTGCGGCTGGCCACCGGACTCAGGAACGTGCCCAGC
ATCCAAAGCCGGGGCCTGTTTGGCGCAATCGCCGGCTTCATCGAGGGCGGCTGGACTGGC
ATGGTGGACGGCTGGTACGGCTACCACCACCAGAACGAACAGGGGAGCGGCTACGCAGCT
GACCTGAAGAGCACCCAGAACGCCATCGACAAGATCACCAACAAGGTGAACAGCGTGATC
GAGAAGATGAACACCCAGTTCACCGCCGTGGGCAAGGAGTTCAACCACCTGGAGAAGCGG
ATCGAGAACCTGAACAAGAAGGTGGACGACGGCTTCCTGGACATCTGGACCTACAACGCC
GAGCTGCTGGTTCTGCTGGAGAACGAGCGGACCCTGGACTATCACGACAGCAACGTGAAG
AACCTGTACGAGAAGGTGCGGAACCAGCTGAAGAACAACGCCAAGGAGATCGGCAACGGC
TGCTTCGAGTTCTACCACAAGTGCGACAACACCTGCATGGAGAGCGTGAAGAACGGCACC
TACGACTACCCCAAGTACAGCGAGGAGGCCAAGCTGAACCGGGAGAAGATCGACGGCGTG
AAGCTGGACAGCACCCGGATCTACCAGATCCTGGCCATCTACAGCACCGTGGCCAGCAGC
CTGGTGCTGGTGGTGAGCCTGGGCGCCATCAGCTTCTGGATGTGCAGCAACGGCAGCCTG
CAGTGCCGGATCTGCATC
>Influenza A virus H3N2 (A/Cambodia/e0826360/2020)
HA Moderna
SEQ ID NO: 94
ATGAAGACCATCATCGCCCTGAGCTACATCCTGTGCCTGGTGTTCGCCCAGAAGATCCCC
GGCAACGATAACAGCACCGCCACCCTGTGTCTGGGACACCACGCCGTGCCCAACGGCACC
ATCGTGAAGACTATCACCAACGACCGGATCGAGGTGACCAACGCCACCGAGCTGGTGCAG
AACAGCAGCATCGGCGAGATCTGCGACAGCCCTCACCAGATCCTGGACGGCGGCAACTGC
ACCCTGATCGACGCACTGCTGGGCGACCCTCAGTGCGACGGCTTTCAGAACAAGGAGTGG
GACCTGTTCGTGGAGAGATCGCGGGCCAACAGCAACTGCTACCCCTACGACGTCCCCGAC
TACGCAAGCCTGAGAAGCCTCGTGGCCTCAAGCGGCACCCTGGAGTTCAAGAACGAGAGC
TTCAACTGGACCGGCGTGAAGCAGAACGGCACCTCAAGCGCCTGCATCCGGGGCTCCAGC
AGCAGCTTCTTCTCACGGCTGAACTGGCTGACCCACCTGAACTACACCTACCCCGCCCTG
AACGTGACCATGCCCAACAACGAGCAGTTCGACAAGCTGTACATCTGGGGAGTGCACCAT
CCCAGCACCGACAAGGACCAGATTAGCCTGTTCGCCCAGCCCAGCGGCCGGATCACCGTG
AGCACCAAGCGGAGCCAGCAGGCCGTGATCCCCAACATCGGCTCTCGGCCCAGAATCCGG
GACATCCCCAGCCGGATCAGCATCTACTGGACCATTGTGAAGCCCGGCGACATCCTGCTG
ATCAACTCCACCGGCAACCTGATCGCCCCTCGGGGCTATTTCAAGATCCGGAGCGGCAAG
AGCAGCATCATGCGGAGCGACGCCCCTATCGGCAAGTGCAAGAGCGAGTGCATCACACCC
AACGGAAGCATCCCCAACGACAAGCCCTTCCAGAACGTGAACCGGATAACCTACGGCGCC
TGCCCTAGATACGTGAAGCAGAGCACCCTGAAGCTGGCCACCGGCATGCGGAACGTGCCC
GAGAAGCAGACTCGGGGCATCTTCGGCGCCATCGCCGGCTTCATCGAGAACGGCTGGGAG
GGCATGGTGGACGGCTGGTACGGCTTCCGGCACCAGAACTCTGAGGGCAGAGGACAGGCC
GCAGACCTGAAGAGCACCCAGGCCGCCATCGACCAGATCAACGGCAAGCTGAACCGGCTG
ATCGGCAAGACCAACGAGAAGTTCCACCAGATCGAGAAGGAGTTCAGCGAGGTGGAGGGC
AGGGTACAGGACCTGGAGAAGTACGTGGAGGACACCAAGATCGACCTGTGGAGCTACAAC
GCCGAGCTGCTGGTAGCCCTGGAGAACCAGCACACCATCGACCTGACCGACAGCGAGATG
AACAAGCTGTTCGAGAAGACCAAGAAGCAGCTGCGGGAGAACGCCGAGGACATGGGCAAC
GGCTGCTTCAAGATCTACCACAAGTGCGACAACGCCTGCATCGGCAGCATCCGGAACGAG
ACCTACGACCACAACGTGTACCGGGACGAGGCCCTGAACAACCGGTTCCAGATCAAGGGC
GTGGAGCTGAAGAGCGGCTACAAGGACTGGATCCTGTGGATCAGCTTCGCCATGTCCTGC
TTCCTGCTGTGCATCGCCCTGCTGGGTTTCATCATGTGGGCCTGCCAGAAGGGCAACATC
CGGTGCAACATCTGCATC
>Influenza B virus Victoria lineage (B/Washington/02/2019)
HA CDS Moderna
SEQ ID NO: 95
ATGAAGGCCATCATCGTGCTGTTAATGGTGGTGACCAGCAACGCCGACCGGATCTGCACC
GGCATCACCTCTAGCAACAGCCCTCACGTGGTGAAGACCGCCACACAGGGCGAGGTGAAC
GTGACCGGCGTGATTCCCCTGACCACCACCCCTACCAAGAGCCACTTCGCCAACCTGAAG
GGAACCGAGACCCGGGGCAAGCTGTGTCCCAAGTGCCTGAACTGCACCGACCTGGACGTG
GCCCTGGGCAGACCCAAGTGCACCGGCAAGATCCCCAGCGCCCGGGTGTCTATCCTGCAC
GAAGTGCGGCCCGTGACTAGCGGCTGCTTCCCCATCATGCACGACCGGACCAAGATCCGG
CAGCTGCCCAACCTGCTGCGGGGCTACGAGCACGTGCGGCTGAGCACCCACAACGTGATC
AACGCCGAAGACGCACCCGGGAGACCATACGAGATCGGCACCAGCGGCTCTTGCCCCAAC
ATCACCAACGGCAACGGCTTCTTCGCTACCATGGCCTGGGCCGTGCCAAAGAACAAGACT
GCCACCAACCCTCTGACCATCGAGGTGCCCTACATCTGCACCGAGGGCGAGGACCAGATC
ACCGTGTGGGGCTTCCACAGCGACAGCGAGACCCAGATGGCCAAGCTGTACGGCGACAGC
AAGCCCCAGAAGTTCACCAGCAGCGCCAACGGCGTGACCACCCACTACGTGAGCCAGATC
GGCGGCTTCCCCAACCAGACCGAGGACGGCGGCTTACCCCAGAGCGGCCGGATCGTGGTG
GACTACATGGTGCAGAAGAGCGGCAAGACCGGCACCATCACCTACCAGCGGGGCATCCTG
CTGCCACAGAAGGTGTGGTGCGCCTCAGGGCGGTCAAAGGTGATCAAGGGCAGCCTGCCA
CTGATTGGCGAGGCCGACTGCCTGCACGAGAAGTACGGCGGCCTGAACAAGAGCAAGCCC
TACTACACCGGCGAGCACGCCAAGGCAATCGGCAACTGCCCCATCTGGGTGAAGACACCC
CTGAAGCTGGCCAACGGCACCAAGTACCGGCCACCCGCCAAACTGCTGAAGGAGCGGGGC
TTCTTCGGCGCCATTGCCGGCTTCCTCGAAGGCGGTTGGGAGGGCATGATCGCCGGCTGG
CACGGCTACACTAGCCACGGCGCACACGGAGTAGCAGTGGCCGCCGACCTGAAGAGCACC
CAGGAGGCCATCAACAAGATCACCAAGAACCTGAACAGCCTGAGCGAGCTGGAGGTGAAG
AATCTGCAGCGGCTGTCTGGCGCTATGGACGAGCTGCACAACGAGATCCTGGAGCTGGAC
GAGAAGGTGGACGACTTACGGGCCGACACCATCAGCAGCCAGATCGAGCTGGCCGTGCTG
CTCTCCAACGAGGGCATCATCAACAGCGAGGACGAGCACCTGCTCGCCCTGGAGCGGAAG
CTGAAGAAGATGCTGGGCCCTTCTGCCGTGGAGATCGGTAACGGCTGCTTCGAGACCAAG
CACAAGTGCAACCAGACCTGCCTGGATCGGATCGCAGCCGGCACCTTTGACGCCGGGGAG
TTCAGCCTGCCCACCTTCGACAGCCTGAACATCACCGCCGCCAGCCTGAACGACGACGGC
CTGGACAACCACACCATCCTGCTGTACTACTCTACAGCCGCTAGCAGCCTGGCCGTGACC
CTGATGATCGCCATCTTCGTGGTGTACATGGTGAGCCGGGACAACGTGAGCTGCAGCATC
TGCCTG
>Influenza B virus Yamagata lineage (B/Phuket/3073/2013)
HA CDS Moderna
SEQ ID NO: 96
ATGAAGGCCATCATCGTGCTACTGATGGTGGTGACCAGCAACGCCGACCGGATCTGCACC
GGCATCACCAGCAGCAACAGCCCGCACGTGGTGAAGACCGCCACCCAAGGCGAGGTGAAC
GTGACCGGCGTGATCCCACTGACCACCACTCCCACCAAGAGCTACTTCGCCAACCTGAAG
GGCACACGGACTCGGGGCAAGCTGTGCCCCGACTGCCTGAACTGCACCGACCTGGACGTG
GCCCTGGGCAGACCCATGTGCGTGGGCACCACCCCTTCTGCCAAGGCCAGCATCCTGCAC
GAGGTGAGACCCGTGACCAGCGGGTGCTTCCCCATCATGCACGACCGGACCAAGATCCGG
CAGCTGCCCAACCTGCTGCGGGGCTACGAGAAGATCCGGCTGAGCACCCAGAACGTGATC
GACGCCGAGAAGGCCCCTGGAGGTCCCTACCGGCTGGGCACCAGCGGAAGCTGCCCCAAC
GCCACCAGCAAGATCGGCTTCTTCGCCACCATGGCCTGGGCTGTGCCCAAGGACAACTAC
AAGAACGCCACCAATCCCCTGACCGTGGAGGTGCCCTACATCTGCACCGAGGGCGAGGAC
CAGATCACCGTGTGGGGCTTCCACAGCGACAACAAGACCCAGATGAAGAGCCTGTACGGC
GACAGCAATCCCCAGAAGTTCACAAGCAGCGCCAACGGCGTGACCACCCACTACGTGAGC
CAGATCGGCGACTTCCCCGACCAGACCGAGGACGGAGGGCTGCCTCAGAGTGGCCGGATC
GTGGTGGACTACATGATGCAGAAGCCCGGCAAGACCGGCACCATCGTGTACCAGCGGGGC
GTGCTGTTGCCTCAGAAAGTTTGGTGTGCCAGCGGCAGGAGCAAGGTGATCAAGGGCAGC
CTGCCCCTGATCGGCGAGGCAGACTGCCTCCACGAGGAGTACGGCGGCCTGAACAAGAGC
AAGCCCTACTACACCGGCAAGCACGCCAAGGCCATCGGCAACTGCCCCATCTGGGTGAAG
ACCCCTCTGAAGCTGGCCAACGGCACCAAGTACCGGCCACCAGCCAAGCTGCTGAAGGAG
CGGGGCTTCTTTGGCGCCATTGCCGGCTTCCTCGAGGGAGGCTGGGAGGGCATGATCGCC
GGCTGGCACGGCTACACAAGCCACGGCGCACACGGAGTGGCTGTGGCTGCCGACCTGAAG
AGCACCCAGGAGGCCATCAACAAGATCACCAAGAACCTGAACAGCCTGAGCGAGCTGGAG
GTGAAGAACCTGCAGCGGCTGTCAGGCGCCATGGACGAGCTGCACAACGAGATCCTGGAG
CTGGACGAGAAGGTGGACGACCTGCGTGCCGACACCATCAGCAGCCAGATCGAGCTGGCC
GTGCTGCTGAGCAACGAGGGCATCATCAACAGCGAGGACGAGCACCTGCTGGCCCTGGAG
CGGAAACTGAAGAAGATGCTGGGACCCTCTGCCGTGGACATCGGCAACGGCTGCTTCGAG
ACCAAGCACAAGTGCAACCAGACCTGCCTGGATCGGATCGCCGCCGGAACCTTCAACGCC
GGCGAGTTCAGCCTGCCCACCTTCGACAGCCTGAACATCACCGCCGCCAGCCTGAACGAC
GACGGCCTGGACAACCACACCATCCTGCTGTACTACAGCACTGCCGCCTCAAGCCTGGCC
GTGACCCTGATGCTGGCCATCTTCATCGTGTACATGGTGAGCCGGGACAACGTGAGCTGC
AGCATCTGCCTG
> SARS-CoV-2 BA.4 spike protein CDS
SEQ ID NO: 97
ATGTTCGTGTTCCTGGTGCTGCTGCCCCTGGTGAGCTCTCAGTGCGTGAACCTGATCACA
AGAACACAGTCGTACACCAACAGCTTCACAAGAGGCGTGTACTACCCCGACAAGGTGTTC
AGAAGCAGCGTGCTGCACTCCACCCAAGACCTGTTTCTGCCCTTCTTCAGCAACGTGACC
TGGTTCCACGCCATCAGCGGCACCAACGGCACCAAGAGATTCGACAACCCCGTGCTGCCC
TTCAACGACGGCGTGTACTTCGCTAGCACCGAAAAGAGCAACATCATCAGAGGCTGGATC
TTCGGCACCACCCTAGACTCCAAGACACAAAGCCTGCTGATCGTTAACAACGCGACCAAC
GTGGTGATCAAGGTGTGCGAGTTTCAGTTCTGCAACGACCCCTTCCTGGACGTGTACTAC
CACAAGAACAACAAGAGCTGGATGGAGAGCGAGTTCAGAGTCTACAGCAGCGCCAACAAC
TGCACCTTCGAGTACGTGTCTCAGCCCTTCCTGATGGACCTGGAGGGCAAGCAAGGCAAT
TTCAAGAACCTAAGAGAGTTCGTGTTCAAGAACATCGACGGCTACTTCAAGATCTACAGC
AAGCACACCCCCATCAACCTGGGGAGAGACCTGCCCCAAGGCTTCAGCGCCCTGGAGCCC
CTGGTGGACCTGCCCATCGGCATCAACATCACAAGATTTCAGACCCTGCTGGCCCTGCAC
CGTAGCTACCTAACCCCCGGCGATAGTTCCTCTGGCTGGACGGCCGGCGCTGCCGCCTAC
TACGTGGGCTACCTGCAGCCTAGAACCTTCCTGCTGAAGTACAACGAGAACGGCACTATC
ACTGATGCCGTGGACTGTGCCCTGGACCCCCTGAGCGAGACCAAGTGCACCCTCAAGAGC
TTCACCGTGGAGAAGGGCATCTATCAGACAAGCAACTTCAGAGTGCAGCCCACCGAGAGC
ATCGTGAGATTCCCCAACATCACCAACCTGTGCCCCTTCGACGAGGTGTTCAACGCCACA
AGATTCGCTAGCGTGTACGCGTGGAACAGAAAGAGAATCAGCAACTGCGTGGCCGACTAC
AGCGTGCTGTACAACTTCGCTCCCTTCTTCGCCTTCAAGTGCTATGGCGTCAGCCCCACC
AAGCTGAACGACCTGTGCTTCACCAACGTGTACGCCGACAGCTTCGTGATCAGAGGCAAC
GAGGTGAGCCAGATTGCCCCCGGGCAAACCGGCAACATCGCCGACTACAACTACAAGCTG
CCCGACGACTTCACCGGCTGCGTGATCGCGTGGAACAGCAATAAGCTGGATTCCAAGGTG
GGCGGCAACTACAACTACCGGTACAGACTGTTCAGAAAGAGCAACCTGAAGCCCTTCGAG
AGAGACATCAGCACCGAGATCTACCAAGCCGGCAACAAGCCCTGCAACGGCGTGGCGGGC
GTCAACTGCTACTTCCCCCTGCAGAGCTACGGCTTTAGGCCCACCTACGGCGTGGGCCAT
CAGCCCTACAGAGTGGTGGTGCTGAGCTTCGAGCTGCTGCACGCCCCCGCAACCGTGTGC
GGCCCCAAAAAGAGCACCAACCTGGTGAAGAACAAGTGCGTGAACTTCAACTTTAATGGC
CTTAAGGGCACGGGCGTGCTGACCGAGAGCAACAAGAAGTTCCTGCCATTTCAGCAATTC
GGCAGAGACATCGCCGACACCACCGACGCGGTCAGAGACCCTCAGACCCTGGAGATCCTG
GACATCACCCCCTGTAGCTTCGGCGGCGTGAGCGTGATCACCCCCGGCACTAACACAAGC
AACCAAGTGGCCGTGCTGTACCAAGGCGTGAACTGCACCGAGGTGCCCGTGGCCATCCAC
GCCGATCAGCTGACCCCCACCTGGAGAGTATACAGCACCGGAAGCAACGTGTTTCAGACA
AGAGCCGGCTGCCTGATCGGCGCCGAGTACGTGAACAACAGCTACGAGTGCGACATCCCC
ATCGGCGCCGGCATCTGCGCTAGCTATCAGACACAGACCAAGAGCCACCGGAGAGCTAGA
AGCGTGGCTTCTCAGAGCATCATCGCCTACACCATGAGCCTGGGCGCCGAGAACAGCGTG
GCCTACAGCAACAACAGCATCGCCATCCCCACCAACTTCACCATCAGCGTGACCACCGAG
ATCCTGCCCGTAAGTATGACCAAGACAAGCGTGGACTGCACCATGTACATCTGCGGCGAC
AGCACCGAGTGCAGCAACCTGCTGCTGCAGTACGGCAGCTTCTGCACACAGCTGAAGAGA
GCCCTGACCGGCATCGCCGTGGAGCAAGACAAGAACACCCAAGAGGTGTTCGCCCAAGTG
AAGCAGATCTACAAGACCCCCCCCATCAAGTACTTCGGCGGCTTCAACTTCAGCCAAATC
CTGCCCGACCCTAGCAAGCCTAGCAAGCGGAGCTTCATCGAGGACCTGCTGTTCAACAAG
GTGACCCTGGCCGACGCCGGCTTCATCAAGCAGTACGGCGACTGCCTAGGCGACATCGCA
GCTAGAGACCTGATCTGCGCTCAGAAGTTCAATGGACTCACCGTGCTCCCCCCCCTGCTG
ACCGACGAGATGATCGCTCAGTACACCTCTGCCCTGCTGGCGGGAACCATAACAAGCGGT
TGGACATTTGGCGCCGGGGCCGCACTGCAAATCCCCTTCGCCATGCAGATGGCCTACAGA
TTCAACGGCATCGGCGTGACACAGAACGTGCTGTACGAGAATCAGAAGCTGATCGCCAAT
CAGTTCAACAGCGCCATCGGCAAGATCCAAGACAGCCTGAGCAGCACCGCTAGCGCCCTG
GGCAAGCTGCAAGACGTGGTGAATCACAACGCCCAAGCCCTGAACACCCTGGTGAAGCAG
CTGAGCAGCAAGTTCGGCGCCATCAGCAGCGTATTGAATGACATCCTGTCTAGACTGGAC
CCCCCCGAGGCCGAGGTGCAGATCGACAGACTGATCACCGGCAGACTGCAGAGCCTGCAG
ACCTACGTGACACAGCAGCTGATCAGAGCCGCCGAGATCAGAGCTAGCGCCAACCTGGCC
GCCACCAAGATGAGCGAGTGCGTGCTGGGGCAGAGCAAGAGAGTGGACTTCTGCGGCAAG
GGCTACCACCTGATGAGCTTCCCTCAGAGCGCCCCCCACGGCGTGGTGTTCCTGCACGTG
ACCTACGTGCCCGCCCAAGAGAAGAACTTCACCACCGCTCCCGCCATCTGCCACGACGGC
AAGGCCCACTTCCCTAGAGAGGGCGTGTTCGTGAGCAACGGCACCCACTGGTTCGTGACA
CAGAGAAACTTCTACGAGCCTCAGATCATCACCACCGACAACACCTTCGTGAGCGGCAAC
TGCGACGTGGTGATCGGCATAGTGAACAACACCGTATACGACCCCCTGCAGCCCGAGCTG
GACAGCTTCAAGGAGGAGCTGGACAAGTACTTCAAAAACCATACTAGCCCCGACGTGGAC
CTGGGAGACATCTCTGGCATCAACGCTAGCGTGGTGAACATTCAGAAGGAGATCGACCGG
CTGAATGAGGTGGCCAAGAACCTGAACGAGAGCCTGATCGACCTGCAAGAGCTGGGCAAG
TACGAGCAGTACATCAAGTGGCCCTGGTACATCTGGCTGGGCTTCATCGCCGGCCTGATC
GCCATCGTGATGGTGACCATCATGCTGTGCTGCATGACAAGCTGTTGCAGCTGCCTGAAA
GGTTGCTGCAGCTGTGGCTCGTGCTGCAAGTTCGACGAGGACGACAGCGAGCCCGTGCTG
AAGGGCGTGAAGCTGCACTACACCTGATGA
>Influenza A virus H1N1 HAO mutant amino acid sequence
SEQ ID NO: 98
MKAILVVMLYTFTTANADTLCIGYHANNSTDTVDTVLEKNVTVTHSVNLLEDKHNGKLCK
LRGVAPLHLGKCNIAGWILGNPECESLSTARSWSYIVETSNSDNGTCYPGDFINYEELRE
QLSSVSSFERFEIFPKTSSWPNHDSDNGVTAACPHAGAKSFYKNLIWLVKKGKSYPKINQ
TYINDKGKEVLVLWGIHHPPTIADQQSLYQNADAYVFVGTSRYSKKFKPEIATRPKVRDQ
EGRMNYYWTLVEPGDKITFEATGNLVAPRYAFTMERDAGSGIIISDTPVHDCNTTCQTPE
GAINTSLPFQNVHPITIGKCPKYVKSTKLRLATGLRNVPSIQSQGLFGAIAGFIEGGWTG
MVDGWYGYHHQNEQGSGYAADLKSTQNAIDKITNKVNSVIEKMNTQFTAVGKEFNHLEKR
IENLNKKVDDGFLDIWTYNAELLVLLENERTLDYHDSNVKNLYEKVRNQLKNNAKEIGNG
CFEFYHKCDNTCMESVKNGTYDYPKYSEEAKLNREKIDGVKLDSTRIYQILAIYSTVASS
LVLVVSLGAISFWMCSNGSLQCRICI
>Influenza A virus H3N2 HAO mutant amino acid sequence
SEQ ID NO: 99
MKTIIALSYILCLVFAQKIPGNDNSTATLCLGHHAVPNGTIVKTITNDRIEVTNATELVQ
NSSIGEICDSPHQILDGGNCTLIDALLGDPQCDGFQNKEWDLFVERSRANSNCYPYDVPD
YASLRSLVASSGTLEFKNESFNWTGVKQNGTSSACIRGSSSSFFSRLNWLTHLNYTYPAL
NVTMPNNEQFDKLYIWGVHHPSTDKDQISLFAQPSGRITVSTKRSQQAVIPNIGSRPRIR
DIPSRISIYWTIVKPGDILLINSTGNLIAPRGYFKIRSGKSSIMRSDAPIGKCKSECITP
NGSIPNDKPFQNVNRITYGACPRYVKQSTLKLATGMRNVPEKQTQGIFGAIAGFIENGWE
GMVDGWYGFRHQNSEGRGQAADLKSTQAAIDQINGKLNRLIGKTNEKFHQIEKEFSEVEG
RVQDLEKYVEDTKIDLWSYNAELLVALENQHTIDLTDSEMNKLFEKTKKQLRENAEDMGN
GCFKIYHKCDNACIGSIRNETYDHNVYRDEALNNRFQIKGVELKSGYKDWILWISFAMSC
FLLCIALLGFIMWACQKGNIRCNICI
>Influenza B virus/Victoria HAO mutant amino acid sequence
SEQ ID NO: 100
MKAIIVLLMVVTSNADRICTGITSSNSPHVVKTATQGEVNVTGVIPLTTTPTKSHFANLK
GTETRGKLCPKCLNCTDLDVALGRPKCTGKIPSARVSILHEVRPVTSGCFPIMHDRTKIR
QLPNLLRGYEHVRLSTHNVINAEDAPGRPYEIGTSGSCPNITNGNGFFATMAWAVPKNKT
ATNPLTIEVPYICTEGEDQITVWGFHSDNETQMAKLYGDSKPQKFTSSANGVTTHYVSQI
GGFPNQTEDGGLPQSGRIVVDYMVQKSGKTGTITYQRGILLPQKVWCASGRSKVIKGSLP
LIGEADCLHEKYGGLNKSKPYYTGEHAKAIGNCPIWVKTPLKLANGTKYRPPAKLLKEQG
FFGAIAGFLEGGWEGMIAGWHGYTSHGAHGVAVAADLKSTQEAINKITKNLNSLSELEVK
NLQRLSGAMDELHNEILELDEKVDDLRADTISSQIELAVLLSNEGIINSEDEHLLALERK
LKKMLGPSAVEIGNGCFETKHKCNQTCLDRIAAGTFDAGEFSLPTFDSLNITAASLNDDG
LDNHTILLYYSTAASSLAVTLMIAIFVVYMVSRDNVSCSICL
>Influenza B virus/Yamagata HAO mutant amino acid sequence
SEQ ID NO: 101
MKAIIVLLMVVTSNADRICTGITSSNSPHVVKTATQGEVNVTGVIPLTTTPTKSYFANLK
GTRTRGKLCPDCLNCTDLDVALGRPMCVGTTPSAKASILHEVRPVTSGCFPIMHDRTKIR
QLPNLLRGYEKIRLSTQNVIDAEKAPGGPYRLGTSGSCPNATSKIGFFATMAWAVPKDNY
KNATNPLTVEVPYICTEGEDQITVWGFHSDNKTQMKSLYGDSNPQKFTSSANGVTTHYVS
QIGDFPDQTEDGGLPQSGRIVVDYMMQKPGKTGTIVYQRGVLLPQKVWCASGRSKVIKGS
LPLIGEADCLHEEYGGLNKSKPYYTGKHAKAIGNCPIWVKTPLKLANGTKYRPPAKLLKE
QGFFGAIAGFLEGGWEGMIAGWHGYTSHGAHGVAVAADLKSTQEAINKITKNLNSLSELE
VKNLQRLSGAMDELHNEILELDEKVDDLRADTISSQIELAVLLSNEGIINSEDEHLLALE
RKLKKMLGPSAVDIGNGCFETKHKCNQTCLDRIAAGTFNAGEFSLPTFDSLNITAASLND
DGLDNHTILLYYSTAASSLAVTLMLAIFIVYMVSRDNVSCSICL
>Influenza A virus H1N1 HAO mutant CDS
SEQ ID NO: 102
ATGAAGGCCATCCTGGTGGTGATGCTGTACACCTTCACCACCGCCAACGCCGACACCCTG
TGCATCGGCTACCACGCCAACAACAGCACCGACACCGTGGACACCGTGCTGGAGAAGAAC
GTGACCGTGACCCACAGCGTGAACCTGCTGGAGGACAAGCACAACGGCAAGCTGTGCAAG
CTGCGGGGCGTGGCCCCCCTGCACCTGGGCAAGTGCAACATCGCCGGCTGGATCCTGGGC
AACCCCGAGTGCGAGTCCCTGAGCACCGCCCGCTCCTGGAGCTACATCGTGGAGACCTCC
AACAGCGACAACGGCACCTGCTACCCCGGGGACTTCATCAACTACGAGGAGCTGCGGGAG
CAGCTGTCCAGCGTGTCCAGCTTCGAGCGCTTCGAGATCTTCCCCAAGACCTCCAGCTGG
CCCAACCACGACTCCGACAACGGCGTGACCGCCGCCTGCCCCCACGCCGGGGCCAAGAGC
TTCTACAAGAACCTGATCTGGCTGGTGAAGAAGGGCAAGTCCTACCCCAAGATCAACCAG
ACCTACATCAACGACAAGGGGAAGGAGGTCCTCGTGCTGTGGGGCATCCACCACCCCCCC
ACCATCGCCGACCAGCAGAGCCTCTACCAGAACGCCGACGCCTACGTCTTCGTGGGGACC
TCCCGGTACAGCAAGAAGTTCAAGCCCGAGATCGCCACCCGCCCCAAGGTCCGGGACCAG
GAGGGCCGCATGAACTACTACTGGACCCTGGTGGAGCCGGGGGACAAGATCACCTTCGAG
GCCACCGGCAACCTCGTCGCCCCGCGGTACGCCTTCACGATGGAGCGCGACGCCGGGTCC
GGCATCATCATCAGCGACACCCCCGTGCACGACTGCAACACGACCTGCCAGACGCCCGAG
GGGGCGATCAACACCTCCCTGCCGTTCCAGAACGTCCACCCGATCACGATCGGCAAGTGC
CCCAAGTACGTGAAGTCCACCAAGCTCCGGCTGGCCACGGGGCTCCGCAACGTCCCCTCC
ATCCAGAGCCAGGGCCTGTTCGGGGCGATCGCCGGCTTCATCGAGGGGGGCTGGACCGGG
ATGGTGGACGGCTGGTACGGGTACCACCACCAGAACGAGCAGGGCTCCGGGTACGCGGCC
GACCTCAAGTCGACGCAGAACGCGATCGACAAGATCACCAACAAGGTCAACAGCGTGATC
GAGAAGATGAACACGCAGTTCACCGCCGTCGGCAAGGAGTTCAACCACCTGGAGAAGCGC
ATCGAGAACCTCAACAAGAAGGTGGACGACGGGTTCCTGGACATCTGGACGTACAACGCG
GAGCTCCTGGTCCTCCTGGAGAACGAGCGGACCCTCGACTACCACGACTCCAACGTGAAG
AACCTGTACGAGAAGGTCCGCAACCAGCTCAAGAACAACGCCAAGGAGATCGGCAACGGG
TGCTTCGAGTTCTACCACAAGTGCGACAACACGTGCATGGAGTCGGTGAAGAACGGCACC
TACGACTACCCGAAGTACAGCGAGGAGGCGAAGCTGAACCGGGAGAAGATCGACGGGGTC
AAGCTCGACTCCACGCGCATCTACCAGATCCTGGCCATCTACTCGACCGTGGCGAGCTCC
CTCGTCCTGGTGGTCTCGCTCGGCGCCATCAGCTTCTGGATGTGCTCCAACGGGTCGCTG
CAGTGCCGGATCTGCATCTGA
>Influenza A virus H3N2 HAO mutant CDS
SEQ ID NO: 103
ATGAAGACCATCATCGCCCTGAGCTACATCCTGTGCCTGGTGTTCGCCCAGAAGATCCCC
GGCAACGACAACAGCACCGCCACCCTGTGCCTGGGCCACCACGCCGTGCCCAACGGCACC
ATCGTGAAGACCATCACCAACGACCGGATCGAGGTGACCAACGCCACCGAGCTGGTGCAG
AACTCCAGCATCGGCGAGATCTGCGACTCCCCCCACCAGATCCTGGACGGCGGCAACTGC
ACCCTGATCGACGCCCTGCTGGGCGACCCCCAGTGCGACGGGTTCCAGAACAAGGAGTGG
GACCTGTTCGTGGAGCGGAGCCGCGCCAACTCCAACTGCTACCCCTACGACGTGCCCGAC
TACGCCAGCCTGCGGTCCCTGGTGGCCAGCTCCGGCACCCTGGAGTTCAAGAACGAGAGC
TTCAACTGGACCGGGGTCAAGCAGAACGGCACCTCCAGCGCCTGCATCCGCGGGTCCAGC
TCCAGCTTCTTCTCCCGGCTGAACTGGCTCACCCACCTGAACTACACCTACCCCGCCCTC
AACGTGACCATGCCCAACAACGAGCAGTTCGACAAGCTGTACATCTGGGGCGTCCACCAC
CCCAGCACCGACAAGGACCAGATCTCCCTCTTCGCCCAGCCCAGCGGGCGCATCACCGTG
TCCACGAAGCGGAGCCAGCAGGCCGTCATCCCCAACATCGGCTCCCGCCCGCGGATCCGC
GACATCCCCAGCCGGATCTCCATCTACTGGACGATCGTGAAGCCGGGGGACATCCTGCTC
ATCAACAGCACCGGCAACCTGATCGCCCCCCGCGGGTACTTCAAGATCCGGTCCGGCAAG
AGCTCGATCATGCGCTCCGACGCCCCGATCGGGAAGTGCAAGAGCGAGTGCATCACCCCC
AACGGCTCGATCCCGAACGACAAGCCCTTCCAGAACGTCAACCGGATCACGTACGGGGCC
TGCCCGCGCTACGTGAAGCAGTCCACGCTCAAGCTGGCCACCGGCATGCGGAACGTCCCC
GAGAAGCAGACCCAGGGGATCTTCGGCGCCATCGCGGGGTTCATCGAGAACGGCTGGGAG
GGGATGGTGGACGGCTGGTACGGGTTCCGGCACCAGAACAGCGAGGGCCGCGGGCAGGCC
GCGGACCTCAAGTCGACGCAGGCCGCGATCGACCAGATCAACGGCAAGCTGAACCGGCTC
ATCGGGAAGACGAACGAGAAGTTCCACCAGATCGAGAAGGAGTTCTCCGAGGTCGAGGGC
CGCGTGCAGGACCTGGAGAAGTACGTCGAGGACACCAAGATCGACCTCTGGAGCTACAAC
GCCGAGCTGCTCGTGGCGCTGGAGAACCAGCACACCATCGACCTCACGGACTCGGAGATG
AACAAGCTGTTCGAGAAGACGAAGAAGCAGCTCCGGGAGAACGCCGAGGACATGGGGAAC
GGCTGCTTCAAGATCTACCACAAGTGCGACAACGCGTGCATCGGGTCCATCCGCAACGAG
ACCTACGACCACAACGTCTACCGGGACGAGGCCCTGAACAACCGCTTCCAGATCAAGGGC
GTGGAGCTCAAGAGCGGGTACAAGGACTGGATCCTGTGGATCTCGTTCGCGATGTCCTGC
TTCCTCCTGTGCATCGCCCTCCTGGGCTTCATCATGTGGGCGTGCCAGAAGGGGAACATC
CGGTGCAACATCTGCATCTGA
>Influenza B virus/Victoria HAO mutant CDS
SEQ ID NO: 104
ATGAAGGCCATCATCGTGCTGCTGATGGTGGTGACCAGCAACGCCGACCGGATCTGCACC
GGCATCACCAGCTCCAACAGCCCCCACGTGGTGAAGACCGCCACCCAGGGCGAGGTGAAC
GTGACCGGCGTGATCCCCCTGACCACCACCCCCACCAAGTCCCACTTCGCCAACCTGAAG
GGCACCGAGACCCGGGGCAAGCTGTGCCCCAAGTGCCTGAACTGCACCGACCTGGACGTG
GCCCTGGGCCGCCCCAAGTGCACCGGCAAGATCCCCAGCGCCCGGGTGTCCATCCTGCAC
GAGGTGCGCCCCGTGACCAGCGGCTGCTTCCCCATCATGCACGACCGCACCAAGATCCGG
CAGCTGCCCAACCTGCTGCGGGGGTACGAGCACGTCCGCCTGTCCACCCACAACGTGATC
AACGCCGAGGACGCCCCCGGCCGGCCCTACGAGATCGGGACCAGCGGCTCCTGCCCCAAC
ATCACCAACGGGAACGGCTTCTTCGCCACCATGGCCTGGGCCGTCCCCAAGAACAAGACC
GCCACCAACCCCCTGACCATCGAGGTGCCGTACATCTGCACCGAGGGGGAGGACCAGATC
ACCGTCTGGGGCTTCCACAGCGACAACGAGACGCAGATGGCCAAGCTGTACGGGGACTCC
AAGCCCCAGAAGTTCACGAGCTCCGCCAACGGCGTGACCACCCACTACGTCAGCCAGATC
GGGGGCTTCCCGAACCAGACGGAGGACGGGGGCCTGCCCCAGTCCGGGCGCATCGTGGTC
GACTACATGGTGCAGAAGTCCGGCAAGACGGGGACCATCACGTACCAGCGGGGCATCCTG
CTGCCGCAGAAGGTCTGGTGCGCCTCCGGGCGCAGCAAGGTGATCAAGGGCTCCCTGCCC
CTCATCGGGGAGGCCGACTGCCTGCACGAGAAGTACGGCGGGCTCAACAAGAGCAAGCCG
TACTACACCGGCGAGCACGCCAAGGCCATCGGGAACTGCCCCATCTGGGTCAAGACCCCG
CTGAAGCTCGCCAACGGCACGAAGTACCGGCCCCCGGCCAAGCTGCTCAAGGAGCAGGGG
TTCTTCGGCGCCATCGCCGGGTTCCTGGAGGGCGGGTGGGAGGGCATGATCGCCGGGTGG
CACGGCTACACGTCCCACGGGGCCCACGGCGTGGCGGTCGCCGCGGACCTCAAGAGCACC
CAGGAGGCCATCAACAAGATCACGAAGAACCTGAACTCCCTCAGCGAGCTGGAGGTGAAG
AACCTCCAGCGCCTGTCCGGGGCGATGGACGAGCTCCACAACGAGATCCTGGAGCTCGAC
GAGAAGGTCGACGACCTGCGGGCCGACACCATCAGCTCGCAGATCGAGCTCGCGGTGCTG
CTCTCCAACGAGGGCATCATCAACAGCGAGGACGAGCACCTGCTCGCCCTGGAGCGGAAG
CTCAAGAAGATGCTGGGGCCCTCGGCGGTCGAGATCGGCAACGGGTGCTTCGAGACCAAG
CACAAGTGCAACCAGACGTGCCTCGACCGCATCGCCGCGGGCACGTTCGACGCCGGGGAG
TTCTCCCTGCCGACCTTCGACAGCCTCAACATCACGGCGGCCTCGCTGAACGACGACGGC
CTCGACAACCACACCATCCTGCTCTACTACTCCACCGCGGCCAGCTCGCTGGCGGTGACG
CTCATGATCGCCATCTTCGTCGTGTACATGGTCTCCCGCGACAACGTGAGCTGCTCGATC
TGCCTGTGA
>Influenza B virus/Yamagata HAO mutant CDS
SEQ ID NO: 105
ATGAAGGCCATCATCGTGCTGCTGATGGTGGTGACCAGCAACGCCGACCGGATCTGCACC
GGCATCACCAGCTCCAACAGCCCCCACGTGGTGAAGACCGCCACCCAGGGCGAGGTGAAC
GTGACCGGCGTGATCCCCCTGACCACCACCCCCACCAAGTCCTACTTCGCCAACCTGAAG
GGCACCCGGACCCGCGGCAAGCTGTGCCCCGACTGCCTGAACTGCACCGACCTGGACGTG
GCCCTGGGCCGGCCCATGTGCGTGGGCACCACCCCCAGCGCCAAGGCCTCCATCCTGCAC
GAGGTGCGCCCCGTGACCAGCGGCTGCTTCCCCATCATGCACGACCGCACCAAGATCCGG
CAGCTGCCCAACCTGCTGCGGGGGTACGAGAAGATCCGCCTGTCCACCCAGAACGTCATC
GACGCCGAGAAGGCCCCCGGCGGGCCCTACCGGCTGGGCACCAGCGGGTCCTGCCCCAAC
GCCACCAGCAAGATCGGCTTCTTCGCCACCATGGCCTGGGCCGTGCCCAAGGACAACTAC
AAGAACGCCACCAACCCCCTGACCGTCGAGGTGCCCTACATCTGCACCGAGGGGGAGGAC
CAGATCACGGTCTGGGGCTTCCACTCCGACAACAAGACCCAGATGAAGTCCCTGTACGGG
GACTCCAACCCGCAGAAGTTCACGAGCTCCGCCAACGGCGTGACCACGCACTACGTCAGC
CAGATCGGGGACTTCCCGGACCAGACCGAGGACGGCGGGCTGCCCCAGTCCGGCCGCATC
GTGGTCGACTACATGATGCAGAAGCCCGGGAAGACGGGCACCATCGTGTACCAGCGGGGG
GTCCTGCTGCCGCAGAAGGTGTGGTGCGCCAGCGGCCGCTCCAAGGTCATCAAGGGGAGC
CTGCCCCTCATCGGCGAGGCCGACTGCCTGCACGAGGAGTACGGGGGCCTCAACAAGTCC
AAGCCGTACTACACGGGGAAGCACGCCAAGGCCATCGGCAACTGCCCGATCTGGGTGAAG
ACCCCCCTGAAGCTCGCCAACGGGACGAAGTACCGGCCGCCCGCCAAGCTGCTCAAGGAG
CAGGGCTTCTTCGGGGCCATCGCCGGCTTCCTGGAGGGGGGCTGGGAGGGGATGATCGCC
GGCTGGCACGGGTACACCAGCCACGGCGCCCACGGGGTCGCGGTGGCCGCGGACCTCAAG
TCCACGCAGGAGGCCATCAACAAGATCACCAAGAACCTGAACAGCCTCTCCGAGCTGGAG
GTCAAGAACCTCCAGCGCCTGAGCGGCGCGATGGACGAGCTCCACAACGAGATCCTGGAG
CTCGACGAGAAGGTGGACGACCTGCGGGCCGACACGATCTCGTCCCAGATCGAGCTCGCG
GTCCTGCTCAGCAACGAGGGGATCATCAACTCGGAGGACGAGCACCTGCTCGCCCTGGAG
CGGAAGCTCAAGAAGATGCTGGGCCCGTCCGCGGTGGACATCGGGAACGGCTGCTTCGAG
ACCAAGCACAAGTGCAACCAGACGTGCCTCGACCGCATCGCCGCGGGGACCTTCAACGCC
GGCGAGTTCAGCCTGCCCACGTTCGACTCGCTCAACATCACCGCGGCCTCCCTGAACGAC
GACGGGCTCGACAACCACACGATCCTGCTCTACTACAGCACCGCGGCCTCGTCCCTGGCG
GTCACGCTCATGCTGGCCATCTTCATCGTGTACATGGTCAGCCGCGACAACGTGTCGTGC
TCCATCTGCCTCTGA
>'-Delta24-B-120A mRNA
SEQ ID NO: 106
GAGUUAGGAGUUGGUGGCAGUUUCAAUAACAGGUCAUUGCCGAGAAAAGGAUAGCACUAU
AAUAUGUUCGUGUUCCUGGUGCUGCUGCCCCUGGUGAGCUCUCAGUGCGUGAACCUGAGG
ACAAGAACACAGCUGCCCCCCGCCUACACCAACAGCUUCACAAGAGGCGUGUACUACCCC
GACAAGGUGUUCAGAAGCAGCGUGCUGCACUCCACCCAAGACCUGUUUCUGCCCUUCUUC
AGCAACGUGACCUGGUUCCACGCCAUCCACGUCAGCGGCACCAACGGCACCAAGAGAUUC
GACAACCCCGUGCUGCCCUUCAACGACGGCGUGUACUUCGCUAGCACCGAAAAGAGCAAC
AUCAUCAGAGGCUGGAUCUUCGGCACCACCCUAGACUCCAAGACACAAAGCCUGCUGAUC
GUUAACAACGCGACCAACGUGGUGAUCAAGGUGUGCGAGUUUCAGUUCUGCAACGACCCC
UUCCUGGACGUGUACUACCACAAGAACAACAAGAGCUGGAUGGAGAGCGGGGUCUACAGC
AGCGCCAACAACUGCACCUUCGAGUACGUGUCUCAGCCCUUCCUGAUGGACCUGGAGGGC
AAGCAAGGCAAUUUCAAGAACCUAAGAGAGUUCGUGUUCAAGAACAUCGACGGCUACUUC
AAGAUCUACAGCAAGCACACCCCCAUCAACCUGGUGAGAGACCUGCCCCAAGGCUUCAGC
GCCCUGGAGCCCCUGGUGGACCUGCCCAUCGGCAUCAACAUCACAAGAUUUCAGACCCUG
CUGGCCCUGCACCGUAGCUACCUAACCCCCGGCGAUAGUUCCUCUGGCUGGACGGCCGGC
GCUGCCGCCUACUACGUGGGCUACCUGCAGCCUAGAACCUUCCUGCUGAAGUACAACGAG
AACGGCACUAUCACUGAUGCCGUGGACUGUGCCCUGGACCCCCUGAGCGAGACCAAGUGC
ACCCUCAAGAGCUUCACCGUGGAGAAGGGCAUCUAUCAGACAAGCAACUUCAGAGUGCAG
CCCACCGAGAGCAUCGUGAGAUUCCCCAACAUCACCAACCUGUGCCCCUUCGGCGAGGUG
UUCAACGCCACAAGAUUCGCUAGCGUGUACGCGUGGAACAGAAAGAGAAUCAGCAACUGC
GUGGCCGACUACAGCGUGCUGUACAACAGCGCUAGCUUCAGCACCUUCAAGUGCUAUGGC
GUCAGCCCCACCAAGCUGAACGACCUGUGCUUCACCAACGUGUACGCCGACAGCUUCGUG
AUCAGAGGCGACGAGGUGAGACAGAUUGCCCCCGGGCAAACCGGCAAGAUCGCCGACUAC
AACUACAAGCUGCCCGACGACUUCACCGGCUGCGUGAUCGCGUGGAACAGCAAUAACCUG
GAUUCCAAGGUGGGCGGCAACUACAACUACCGGUACAGACUGUUCAGAAAGAGCAACCUG
AAGCCCUUCGAGAGAGACAUCAGCACCGAGAUCUACCAAGCCGGCAGCAAGCCCUGCAAC
GGCGUGGAGGGCUUCAACUGCUACUUCCCCCUGCAGAGCUACGGCUUUCAGCCCACCAAC
GGCGUGGGCUAUCAGCCCUACAGAGUGGUGGUGCUGAGCUUCGAGCUGCUGCACGCCCCC
GCAACCGUGUGCGGCCCCAAAAAGAGCACCAACCUGGUGAAGAACAAGUGCGUGAACUUC
AACUUUAAUGGCCUUACCGGCACGGGCGUGCUGACCGAGAGCAACAAGAAGUUCCUGCCA
UUUCAGCAAUUCGGCAGAGACAUCGCCGACACCACCGACGCGGUCAGAGACCCUCAGACC
CUGGAGAUCCUGGACAUCACCCCCUGUAGCUUCGGCGGCGUGAGCGUGAUCACCCCCGGC
ACUAACACAAGCAACCAAGUGGCCGUGCUGUACCAAGGCGUGAACUGCACCGAGGUGCCC
GUGGCCAUCCACGCCGAUCAGCUGACCCCCACCUGGAGAGUAUACAGCACCGGAAGCAAC
GUGUUUCAGACAAGAGCCGGCUGCCUGAUCGGCGCCGAGCACGUGAACAACAGCUACGAG
UGCGACAUCCCCAUCGGCGCCGGCAUCUGCGCUAGCUAUCAGACACAGACCAACAGCCGC
CGGAGAGCUAGAAGCGUGGCUUCUCAGAGCAUCAUCGCCUACACCAUGAGCCUGGGCGCC
GAGAACAGCGUGGCCUACAGCAACAACAGCAUCGCCAUCCCCACCAACUUCACCAUCAGC
GUGACCACCGAGAUCCUGCCCGUAAGUAUGACCAAGACAAGCGUGGACUGCACCAUGUAC
AUCUGCGGCGACAGCACCGAGUGCAGCAACCUGCUGCUGCAGUACGGCAGCUUCUGCACA
CAGCUGAACAGAGCCCUGACCGGCAUCGCCGUGGAGCAAGACAAGAACACCCAAGAGGUG
UUCGCCCAAGUGAAGCAGAUCUACAAGACCCCCCCCAUCAAGGACUUCGGCGGCUUCAAC
UUCAGCCAAAUCCUGCCCGACCCUAGCAAGCCUAGCAAGCGGAGCUUCAUCGAGGACCUG
CUGUUCAACAAGGUGACCCUGGCCGACGCCGGCUUCAUCAAGCAGUACGGCGACUGCCUA
GGCGACAUCGCAGCUAGAGACCUGAUCUGCGCUCAGAAGUUCAAUGGACUCACCGUGCUC
CCCCCCCUGCUGACCGACGAGAUGAUCGCUCAGUACACCUCUGCCCUGCUGGCGGGAACC
AUAACAAGCGGUUGGACAUUUGGCGCCGGGGCCGCACUGCAAAUCCCCUUCGCCAUGCAG
AUGGCCUACAGAUUCAACGGCAUCGGCGUGACACAGAACGUGCUGUACGAGAAUCAGAAG
CUGAUCGCCAAUCAGUUCAACAGCGCCAUCGGCAAGAUCCAAGACAGCCUGAGCAGCACC
GCUAGCGCCCUGGGCAAGCUGCAAAACGUGGUGAAUCAGAACGCCCAAGCCCUGAACACC
CUGGUGAAGCAGCUGAGCAGCAACUUCGGCGCCAUCAGCAGCGUAUUGAAUGACAUCCUG
UCUAGACUGGACCCCCCCGAGGCCGAGGUGCAGAUCGACAGACUGAUCACCGGCAGACUG
CAGAGCCUGCAGACCUACGUGACACAGCAGCUGAUCAGAGCCGCCGAGAUCAGAGCUAGC
GCCAACCUGGCCGCCACCAAGAUGAGCGAGUGCGUGCUGGGGCAGAGCAAGAGAGUGGAC
UUCUGCGGCAAGGGCUACCACCUGAUGAGCUUCCCUCAGAGCGCCCCCCACGGCGUGGUG
UUCCUGCACGUGACCUACGUGCCCGCCCAAGAGAAGAACUUCACCACCGCUCCCGCCAUC
UGCCACGACGGCAAGGCCCACUUCCCUAGAGAGGGCGUGUUCGUGAGCAACGGCACCCAC
UGGUUCGUGACACAGAGAAACUUCUACGAGCCUCAGAUCAUCACCACCGACAACACCUUC
GUGAGCGGCAACUGCGACGUGGUGAUCGGCAUAGUGAACAACACCGUAUACGACCCCCUG
CAGCCCGAGCUGGACAGCUUCAAGGAGGAGCUGGACAAGUACUUCAAAAACCAUACUAGC
CCCGACGUGGACCUGGGAGACAUCUCUGGCAUCAACGCUAGCGUGGUGAACAUUCAGAAG
GAGAUCGACCGGCUGAAUGAGGUGGCCAAGAACCUGAACGAGAGCCUGAUCGACCUGCAA
GAGCUGGGCAAGUACGAGCAGUACAUCAAGUGGCCCUGGUACAUCUGGCUGGGCUUCAUC
GCCGGCCUGAUCGCCAUCGUGAUGGUGACCAUCAUGCUGUGCUGCAUGACAAGCUGUUGC
AGCUGCCUGAAAGGUUGCUGCAGCUGUGGCUCGUGCUGCAAGUUCGACGAGGACGACAGC
GAGCCCGUGCUGAAGGGCGUGAAGCUGCACUACACCUGAUGACUCGAGGCUCGCUUUCUU
GCUGUCCAAUUUCUAUUAAAGGUUCCUUUGUUCCCUAAGUCCAACUACUAAACUGGGGGA
UAUUAUGAAGGGCCUUGAGCAUCUGGAUUCUGCCUAAUAAAAAACAUUUAUUUUCAUUGC
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAA
AA
>I'-OC24-B-120A mRNA
SEQ ID NO: 107
GAGUUAGGAGUUGGUGGCAGUUUCAAUAACAGGUCAUUGCCGAGAAAAGGAUAGCACUAU
AAUAUGUUCGUGUUCCUGGUGCUGCUGCCCCUGGUGAGCUCUCAGUGCGUGAACCUGACC
ACAAGAACACAGCUGCCCCCCGCCUACACCAACAGCUUCACAAGAGGCGUGUACUACCCC
GACAAGGUGUUCAGAAGCAGCGUGCUGCACUCCACCCAAGACCUGUUUCUGCCCUUCUUC
AGCAACGUGACCUGGUUCCACGUGAUCAGCGGCACCAACGGCACCAAGAGAUUCGACAAC
CCCGUGCUGCCCUUCAACGACGGCGUGUACUUCGCUAGCAUCGAAAAGAGCAACAUCAUC
AGAGGCUGGAUCUUCGGCACCACCCUAGACUCCAAGACACAAAGCCUGCUGAUCGUUAAC
AACGCGACCAACGUGGUGAUCAAGGUGUGCGAGUUUCAGUUCUGCAACGACCCCUUCCUG
GACCACAAGAACAACAAGAGCUGGAUGGAGAGCGAGUUCAGAGUGUACAGCAGCGCCAAC
AACUGCACCUUCGAGUACGUGUCUCAGCCCUUCCUGAUGGACCUGGAGGGCAAGCAAGGC
AAUUUCAAGAACCUAAGAGAGUUCGUGUUCAAGAACAUCGACGGCUACUUCAAGAUCUAC
AGCAAGCACACCCCCAUCAUCGUGAGAGAGCCCGAGGACCUGCCCCAAGGCUUCAGCGCC
CUGGAGCCCCUGGUGGACCUGCCCAUCGGCAUCAACAUCACAAGAUUUCAGACCCUGCUG
GCCCUGCACCGUAGCUACCUAACCCCCGGCGAUAGUUCCUCUGGCUGGACGGCCGGCGCU
GCCGCCUACUACGUGGGCUACCUGCAGCCUAGAACCUUCCUGCUGAAGUACAACGAGAAC
GGCACUAUCACUGAUGCCGUGGACUGUGCCCUGGACCCCCUGAGCGAGACCAAGUGCACC
CUCAAGAGCUUCACCGUGGAGAAGGGCAUCUAUCAGACAAGCAACUUCAGAGUGCAGCCC
ACCGAGAGCAUCGUGAGAUUCCCCAACAUCACCAACCUGUGCCCCUUCGACGAGGUGUUC
AACGCCACCAGAUUUGCUAGCGUGUACGCCUGGAACCGGAAGAGAAUCAGCAACUGCGUG
GCCGACUACAGCGUGCUGUACAACCUGGCCCCCUUCUUCACCUUCAAGUGCUAUGGCGUC
AGCCCCACCAAGCUGAACGACCUGUGCUUCACCAACGUGUACGCCGACAGCUUCGUGAUC
AGAGGCGACGAGGUGAGACAGAUCGCCCCCGGGCAGACCGGCAACAUCGCCGACUACAAC
UACAAGCUGCCCGACGACUUCACCGGCUGCGUGAUCGCCUGGAACAGCAACAAGCUGGAU
AGCAAGGUGAGCGGCAACUACAAUUACCUGUACAGACUGUUCAGAAAGAGCAACCUGAAG
CCCUUCGAGAGAGACAUCAGCACCGAGAUCUACCAAGCCGGCAACAAGCCCUGCAACGGC
GUGGCGGGCUUCAACUGCUACUUCCCCCUCAGGAGCUACAGCUUCAGACCCACCUACGGC
GUGGGCCAUCAGCCCUACAGAGUGGUCGUGCUGAGCUUCGAGCUGCUGCACGCCCCUGCC
ACAGUGUGCGGCCCCAAAAAGAGCACCAACCUGGUGAAGAACAAGUGCGUGAACUUCAAC
UUCAACGGCCUGAAGGGCACCGGCGUGCUGACCGAGAGCAACAAGAAGUUCCUGCCAUUU
CAGCAAUUCGGCAGAGACAUCGCCGACACCACCGACGCGGUCAGAGACCCUCAGACCCUG
GAGAUCCUGGACAUCACCCCCUGUAGCUUCGGCGGCGUGAGCGUGAUCACCCCCGGCACU
AACACAAGCAACCAAGUGGCCGUGCUGUACCAAGGCGUGAACUGCACCGAGGUGCCCGUG
GCCAUCCACGCCGAUCAGCUGACCCCCACCUGGAGAGUGUAUAGCACCGGCAGCAACGUG
UUUCAGACAAGAGCCGGCUGCCUGAUCGGCGCCGAGUACGUGAACAACAGCUACGAGUGC
GACAUCCCCAUCGGCGCCGGCAUCUGCGCUAGCUAUCAGACACAGACCAAGAGCCACCGG
AGAGCUAGAAGCGUGGCUAGCCAAAGCAUCAUCGCCUACACCAUGAGCCUGGGCGCCGAG
AACAGCGUGGCCUACAGCAACAACAGCAUCGCCAUCCCCACCAACUUCACCAUCAGCGUG
ACCACCGAGAUCCUGCCCGUAAGUAUGACCAAGACAAGCGUGGACUGCACCAUGUACAUC
UGCGGCGACAGCACCGAGUGCAGCAACCUGCUCCUGCAGUACGGCAGCUUCUGCACACAG
CUGAAGAGAGCCCUGACCGGCAUCGCCGUGGAGCAAGACAAGAACACCCAAGAGGUGUUC
GCCCAAGUGAAGCAGAUCUACAAGACCCCCCCCAUCAAGUACUUCGGCGGCUUCAACUUC
AGCCAAAUCCUGCCCGACCCUAGCAAGCCUAGCAAGCGGAGCUUCAUCGAGGACCUGCUG
UUCAACAAGGUGACCCUGGCCGACGCCGGCUUCAUCAAGCAGUACGGCGACUGCCUAGGC
GACAUCGCAGCUAGAGACCUGAUCUGCGCUCAGAAGUUCAAGGGACUCACCGUGCUCCCC
CCCCUGCUGACCGACGAGAUGAUCGCUCAGUACACCUCUGCCCUGCUGGCGGGAACCAUA
ACAAGCGGUUGGACAUUUGGCGCCGGGGCCGCACUGCAAAUCCCCUUCGCCAUGCAGAUG
GCCUACAGAUUCAACGGCAUCGGCGUGACACAGAACGUGCUGUACGAGAAUCAGAAGCUG
AUCGCCAAUCAGUUCAACAGCGCCAUCGGCAAGAUCCAAGACAGCCUGAGCAGCACCGCU
AGCGCCCUGGGCAAGCUGCAAGACGUGGUGAACCACAACGCCCAAGCCCUGAACACCCUG
GUGAAGCAGCUGAGCAGCAAGUUCGGCGCCAUCAGCAGCGUGCUGAAUGACAUCUUCUCU
AGACUGGACCCCCCCGAGGCCGAGGUGCAGAUCGACAGACUGAUCACCGGCAGACUGCAG
AGCCUGCAGACCUACGUGACACAGCAGCUGAUCAGAGCCGCCGAGAUCAGAGCUAGCGCC
AACCUGGCCGCCACCAAGAUGAGCGAGUGCGUGCUGGGGCAGAGCAAGAGAGUGGACUUC
UGCGGCAAGGGCUACCACCUGAUGAGCUUCCCUCAGAGCGCCCCCCACGGCGUGGUGUUC
CUGCACGUGACCUACGUGCCCGCCCAAGAGAAGAACUUCACCACCGCUCCCGCCAUCUGC
CACGACGGCAAGGCCCACUUCCCUAGAGAGGGCGUGUUCGUGAGCAACGGCACCCACUGG
UUCGUGACACAGAGAAACUUCUACGAGCCUCAGAUCAUCACCACCGACAACACCUUCGUG
AGCGGCAACUGCGACGUGGUGAUCGGCAUAGUGAACAACACCGUAUACGACCCCCUGCAG
CCCGAGCUGGACAGCUUCAAGGAGGAGCUGGACAAGUACUUCAAAAACCAUACUAGCCCC
GACGUGGACCUGGGAGACAUCUCUGGCAUCAACGCUAGCGUGGUGAACAUUCAGAAGGAG
AUCGACCGGCUGAAUGAGGUGGCCAAGAACCUGAACGAGAGCCUGAUCGACCUGCAAGAG
CUGGGCAAGUACGAGCAGUACAUCAAGUGGCCCUGGUACAUCUGGCUGGGCUUCAUCGCC
GGCCUGAUCGCCAUCGUGAUGGUGACCAUCAUGCUGUGCUGCAUGACAAGCUGUUGCAGC
UGCCUGAAAGGUUGCUGCAGCUGUGGCUCGUGCUGCAAGUUCGACGAGGACGACAGCGAG
CCCGUGCUGAAGGGCGUGAAGCUGCACUACACCUGAUGACUCGAGGCUCGCUUUCUUGCU
GUCCAAUUUCUAUUAAAGGUUCCUUUGUUCCCUAAGUCCAACUACUAAACUGGGGGAUAU
UAUGAAGGGCCUUGAGCAUCUGGAUUCUGCCUAAUAAAAAACAUUUAUUUUCAUUGCAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAA
>I'-BA4-B-120A mRNA
SEQ ID NO: 108
GAGUUAGGAGUUGGUGGCAGUUUCAAUAACAGGUCAUUGCCGAGAAAAGGAUAGCACUAU
AAUAUGUUCGUGUUCCUGGUGCUGCUGCCCCUGGUGAGCUCUCAGUGCGUGAACCUGAUC
ACAAGAACACAGUCGUACACCAACAGCUUCACAAGAGGCGUGUACUACCCCGACAAGGUG
UUCAGAAGCAGCGUGCUGCACUCCACCCAAGACCUGUUUCUGCCCUUCUUCAGCAACGUG
ACCUGGUUCCACGCCAUCAGCGGCACCAACGGCACCAAGAGAUUCGACAACCCCGUGCUG
CCCUUCAACGACGGCGUGUACUUCGCUAGCACCGAAAAGAGCAACAUCAUCAGAGGCUGG
AUCUUCGGCACCACCCUAGACUCCAAGACACAAAGCCUGCUGAUCGUUAACAACGCGACC
AACGUGGUGAUCAAGGUGUGCGAGUUUCAGUUCUGCAACGACCCCUUCCUGGACGUGUAC
UACCACAAGAACAACAAGAGCUGGAUGGAGAGCGAGUUCAGAGUCUACAGCAGCGCCAAC
AACUGCACCUUCGAGUACGUGUCUCAGCCCUUCCUGAUGGACCUGGAGGGCAAGCAAGGC
AAUUUCAAGAACCUAAGAGAGUUCGUGUUCAAGAACAUCGACGGCUACUUCAAGAUCUAC
AGCAAGCACACCCCCAUCAACCUGGGGAGAGACCUGCCCCAAGGCUUCAGCGCCCUGGAG
CCCCUGGUGGACCUGCCCAUCGGCAUCAACAUCACAAGAUUUCAGACCCUGCUGGCCCUG
CACCGUAGCUACCUAACCCCCGGCGAUAGUUCCUCUGGCUGGACGGCCGGCGCUGCCGCC
UACUACGUGGGCUACCUGCAGCCUAGAACCUUCCUGCUGAAGUACAACGAGAACGGCACU
AUCACUGAUGCCGUGGACUGUGCCCUGGACCCCCUGAGCGAGACCAAGUGCACCCUCAAG
AGCUUCACCGUGGAGAAGGGCAUCUAUCAGACAAGCAACUUCAGAGUGCAGCCCACCGAG
AGCAUCGUGAGAUUCCCCAACAUCACCAACCUGUGCCCCUUCGACGAGGUGUUCAACGCC
ACAAGAUUCGCUAGCGUGUACGCGUGGAACAGAAAGAGAAUCAGCAACUGCGUGGCCGAC
UACAGCGUGCUGUACAACUUCGCUCCCUUCUUCGCCUUCAAGUGCUAUGGCGUCAGCCCC
ACCAAGCUGAACGACCUGUGCUUCACCAACGUGUACGCCGACAGCUUCGUGAUCAGAGGC
AACGAGGUGAGCCAGAUUGCCCCCGGGCAAACCGGCAACAUCGCCGACUACAACUACAAG
CUGCCCGACGACUUCACCGGCUGCGUGAUCGCGUGGAACAGCAAUAAGCUGGAUUCCAAG
GUGGGCGGCAACUACAACUACCGGUACAGACUGUUCAGAAAGAGCAACCUGAAGCCCUUC
GAGAGAGACAUCAGCACCGAGAUCUACCAAGCCGGCAACAAGCCCUGCAACGGCGUGGCG
GGCGUCAACUGCUACUUCCCCCUGCAGAGCUACGGCUUUAGGCCCACCUACGGCGUGGGC
CAUCAGCCCUACAGAGUGGUGGUGCUGAGCUUCGAGCUGCUGCACGCCCCCGCAACCGUG
UGCGGCCCCAAAAAGAGCACCAACCUGGUGAAGAACAAGUGCGUGAACUUCAACUUUAAU
GGCCUUAAGGGCACGGGCGUGCUGACCGAGAGCAACAAGAAGUUCCUGCCAUUUCAGCAA
UUCGGCAGAGACAUCGCCGACACCACCGACGCGGUCAGAGACCCUCAGACCCUGGAGAUC
CUGGACAUCACCCCCUGUAGCUUCGGCGGCGUGAGCGUGAUCACCCCCGGCACUAACACA
AGCAACCAAGUGGCCGUGCUGUACCAAGGCGUGAACUGCACCGAGGUGCCCGUGGCCAUC
CACGCCGAUCAGCUGACCCCCACCUGGAGAGUAUACAGCACCGGAAGCAACGUGUUUCAG
ACAAGAGCCGGCUGCCUGAUCGGCGCCGAGUACGUGAACAACAGCUACGAGUGCGACAUC
CCCAUCGGCGCCGGCAUCUGCGCUAGCUAUCAGACACAGACCAAGAGCCACCGGAGAGCU
AGAAGCGUGGCUUCUCAGAGCAUCAUCGCCUACACCAUGAGCCUGGGCGCCGAGAACAGC
GUGGCCUACAGCAACAACAGCAUCGCCAUCCCCACCAACUUCACCAUCAGCGUGACCACC
GAGAUCCUGCCCGUAAGUAUGACCAAGACAAGCGUGGACUGCACCAUGUACAUCUGCGGC
GACAGCACCGAGUGCAGCAACCUGCUGCUGCAGUACGGCAGCUUCUGCACACAGCUGAAG
AGAGCCCUGACCGGCAUCGCCGUGGAGCAAGACAAGAACACCCAAGAGGUGUUCGCCCAA
GUGAAGCAGAUCUACAAGACCCCCCCCAUCAAGUACUUCGGCGGCUUCAACUUCAGCCAA
AUCCUGCCCGACCCUAGCAAGCCUAGCAAGCGGAGCUUCAUCGAGGACCUGCUGUUCAAC
AAGGUGACCCUGGCCGACGCCGGCUUCAUCAAGCAGUACGGCGACUGCCUAGGCGACAUC
GCAGCUAGAGACCUGAUCUGCGCUCAGAAGUUCAAUGGACUCACCGUGCUCCCCCCCCUG
CUGACCGACGAGAUGAUCGCUCAGUACACCUCUGCCCUGCUGGCGGGAACCAUAACAAGC
GGUUGGACAUUUGGCGCCGGGGCCGCACUGCAAAUCCCCUUCGCCAUGCAGAUGGCCUAC
AGAUUCAACGGCAUCGGCGUGACACAGAACGUGCUGUACGAGAAUCAGAAGCUGAUCGCC
AAUCAGUUCAACAGCGCCAUCGGCAAGAUCCAAGACAGCCUGAGCAGCACCGCUAGCGCC
CUGGGCAAGCUGCAAGACGUGGUGAAUCACAACGCCCAAGCCCUGAACACCCUGGUGAAG
CAGCUGAGCAGCAAGUUCGGCGCCAUCAGCAGCGUAUUGAAUGACAUCCUGUCUAGACUG
GACCCCCCCGAGGCCGAGGUGCAGAUCGACAGACUGAUCACCGGCAGACUGCAGAGCCUG
CAGACCUACGUGACACAGCAGCUGAUCAGAGCCGCCGAGAUCAGAGCUAGCGCCAACCUG
GCCGCCACCAAGAUGAGCGAGUGCGUGCUGGGGCAGAGCAAGAGAGUGGACUUCUGCGGC
AAGGGCUACCACCUGAUGAGCUUCCCUCAGAGCGCCCCCCACGGCGUGGUGUUCCUGCAC
GUGACCUACGUGCCCGCCCAAGAGAAGAACUUCACCACCGCUCCCGCCAUCUGCCACGAC
GGCAAGGCCCACUUCCCUAGAGAGGGCGUGUUCGUGAGCAACGGCACCCACUGGUUCGUG
ACACAGAGAAACUUCUACGAGCCUCAGAUCAUCACCACCGACAACACCUUCGUGAGCGGC
AACUGCGACGUGGUGAUCGGCAUAGUGAACAACACCGUAUACGACCCCCUGCAGCCCGAG
CUGGACAGCUUCAAGGAGGAGCUGGACAAGUACUUCAAAAACCAUACUAGCCCCGACGUG
GACCUGGGAGACAUCUCUGGCAUCAACGCUAGCGUGGUGAACAUUCAGAAGGAGAUCGAC
CGGCUGAAUGAGGUGGCCAAGAACCUGAACGAGAGCCUGAUCGACCUGCAAGAGCUGGGC
AAGUACGAGCAGUACAUCAAGUGGCCCUGGUACAUCUGGCUGGGCUUCAUCGCCGGCCUG
AUCGCCAUCGUGAUGGUGACCAUCAUGCUGUGCUGCAUGACAAGCUGUUGCAGCUGCCUG
AAAGGUUGCUGCAGCUGUGGCUCGUGCUGCAAGUUCGACGAGGACGACAGCGAGCCCGUG
CUGAAGGGCGUGAAGCUGCACUACACCUGAUGACUCGAGGCUCGCUUUCUUGCUGUCCAA
UUUCUAUUAAAGGUUCCUUUGUUCCCUAAGUCCAACUACUAAACUGGGGGAUAUUAUGAA
GGGCCUUGAGCAUCUGGAUUCUGCCUAAUAAAAAACAUUUAUUUUCAUUGCAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAA
>I'-BA4-B mRNA
SEQ ID NO: 109
GAGUUAGGAGUUGGUGGCAGUUUCAAUAACAGGUCAUUGCCGAGAAAAGGAUAGCACUAU
AAUAUGUUCGUGUUCCUGGUGCUGCUGCCCCUGGUGAGCUCUCAGUGCGUGAACCUGAUC
ACAAGAACACAGUCGUACACCAACAGCUUCACAAGAGGCGUGUACUACCCCGACAAGGUG
UUCAGAAGCAGCGUGCUGCACUCCACCCAAGACCUGUUUCUGCCCUUCUUCAGCAACGUG
ACCUGGUUCCACGCCAUCAGCGGCACCAACGGCACCAAGAGAUUCGACAACCCCGUGCUG
CCCUUCAACGACGGCGUGUACUUCGCUAGCACCGAAAAGAGCAACAUCAUCAGAGGCUGG
AUCUUCGGCACCACCCUAGACUCCAAGACACAAAGCCUGCUGAUCGUUAACAACGCGACC
AACGUGGUGAUCAAGGUGUGCGAGUUUCAGUUCUGCAACGACCCCUUCCUGGACGUGUAC
UACCACAAGAACAACAAGAGCUGGAUGGAGAGCGAGUUCAGAGUCUACAGCAGCGCCAAC
AACUGCACCUUCGAGUACGUGUCUCAGCCCUUCCUGAUGGACCUGGAGGGCAAGCAAGGC
AAUUUCAAGAACCUAAGAGAGUUCGUGUUCAAGAACAUCGACGGCUACUUCAAGAUCUAC
AGCAAGCACACCCCCAUCAACCUGGGGAGAGACCUGCCCCAAGGCUUCAGCGCCCUGGAG
CCCCUGGUGGACCUGCCCAUCGGCAUCAACAUCACAAGAUUUCAGACCCUGCUGGCCCUG
CACCGUAGCUACCUAACCCCCGGCGAUAGUUCCUCUGGCUGGACGGCCGGCGCUGCCGCC
UACUACGUGGGCUACCUGCAGCCUAGAACCUUCCUGCUGAAGUACAACGAGAACGGCACU
AUCACUGAUGCCGUGGACUGUGCCCUGGACCCCCUGAGCGAGACCAAGUGCACCCUCAAG
AGCUUCACCGUGGAGAAGGGCAUCUAUCAGACAAGCAACUUCAGAGUGCAGCCCACCGAG
AGCAUCGUGAGAUUCCCCAACAUCACCAACCUGUGCCCCUUCGACGAGGUGUUCAACGCC
ACAAGAUUCGCUAGCGUGUACGCGUGGAACAGAAAGAGAAUCAGCAACUGCGUGGCCGAC
UACAGCGUGCUGUACAACUUCGCUCCCUUCUUCGCCUUCAAGUGCUAUGGCGUCAGCCCC
ACCAAGCUGAACGACCUGUGCUUCACCAACGUGUACGCCGACAGCUUCGUGAUCAGAGGC
AACGAGGUGAGCCAGAUUGCCCCCGGGCAAACCGGCAACAUCGCCGACUACAACUACAAG
CUGCCCGACGACUUCACCGGCUGCGUGAUCGCGUGGAACAGCAAUAAGCUGGAUUCCAAG
GUGGGCGGCAACUACAACUACCGGUACAGACUGUUCAGAAAGAGCAACCUGAAGCCCUUC
GAGAGAGACAUCAGCACCGAGAUCUACCAAGCCGGCAACAAGCCCUGCAACGGCGUGGCG
GGCGUCAACUGCUACUUCCCCCUGCAGAGCUACGGCUUUAGGCCCACCUACGGCGUGGGC
CAUCAGCCCUACAGAGUGGUGGUGCUGAGCUUCGAGCUGCUGCACGCCCCCGCAACCGUG
UGCGGCCCCAAAAAGAGCACCAACCUGGUGAAGAACAAGUGCGUGAACUUCAACUUUAAU
GGCCUUAAGGGCACGGGCGUGCUGACCGAGAGCAACAAGAAGUUCCUGCCAUUUCAGCAA
UUCGGCAGAGACAUCGCCGACACCACCGACGCGGUCAGAGACCCUCAGACCCUGGAGAUC
CUGGACAUCACCCCCUGUAGCUUCGGCGGCGUGAGCGUGAUCACCCCCGGCACUAACACA
AGCAACCAAGUGGCCGUGCUGUACCAAGGCGUGAACUGCACCGAGGUGCCCGUGGCCAUC
CACGCCGAUCAGCUGACCCCCACCUGGAGAGUAUACAGCACCGGAAGCAACGUGUUUCAG
ACAAGAGCCGGCUGCCUGAUCGGCGCCGAGUACGUGAACAACAGCUACGAGUGCGACAUC
CCCAUCGGCGCCGGCAUCUGCGCUAGCUAUCAGACACAGACCAAGAGCCACCGGAGAGCU
AGAAGCGUGGCUUCUCAGAGCAUCAUCGCCUACACCAUGAGCCUGGGCGCCGAGAACAGC
GUGGCCUACAGCAACAACAGCAUCGCCAUCCCCACCAACUUCACCAUCAGCGUGACCACC
GAGAUCCUGCCCGUAAGUAUGACCAAGACAAGCGUGGACUGCACCAUGUACAUCUGCGGC
GACAGCACCGAGUGCAGCAACCUGCUGCUGCAGUACGGCAGCUUCUGCACACAGCUGAAG
AGAGCCCUGACCGGCAUCGCCGUGGAGCAAGACAAGAACACCCAAGAGGUGUUCGCCCAA
GUGAAGCAGAUCUACAAGACCCCCCCCAUCAAGUACUUCGGCGGCUUCAACUUCAGCCAA
AUCCUGCCCGACCCUAGCAAGCCUAGCAAGCGGAGCUUCAUCGAGGACCUGCUGUUCAAC
AAGGUGACCCUGGCCGACGCCGGCUUCAUCAAGCAGUACGGCGACUGCCUAGGCGACAUC
GCAGCUAGAGACCUGAUCUGCGCUCAGAAGUUCAAUGGACUCACCGUGCUCCCCCCCCUG
CUGACCGACGAGAUGAUCGCUCAGUACACCUCUGCCCUGCUGGCGGGAACCAUAACAAGC
GGUUGGACAUUUGGCGCCGGGGCCGCACUGCAAAUCCCCUUCGCCAUGCAGAUGGCCUAC
AGAUUCAACGGCAUCGGCGUGACACAGAACGUGCUGUACGAGAAUCAGAAGCUGAUCGCC
AAUCAGUUCAACAGCGCCAUCGGCAAGAUCCAAGACAGCCUGAGCAGCACCGCUAGCGCC
CUGGGCAAGCUGCAAGACGUGGUGAAUCACAACGCCCAAGCCCUGAACACCCUGGUGAAG
CAGCUGAGCAGCAAGUUCGGCGCCAUCAGCAGCGUAUUGAAUGACAUCCUGUCUAGACUG
GACCCCCCCGAGGCCGAGGUGCAGAUCGACAGACUGAUCACCGGCAGACUGCAGAGCCUG
CAGACCUACGUGACACAGCAGCUGAUCAGAGCCGCCGAGAUCAGAGCUAGCGCCAACCUG
GCCGCCACCAAGAUGAGCGAGUGCGUGCUGGGGCAGAGCAAGAGAGUGGACUUCUGCGGC
AAGGGCUACCACCUGAUGAGCUUCCCUCAGAGCGCCCCCCACGGCGUGGUGUUCCUGCAC
GUGACCUACGUGCCCGCCCAAGAGAAGAACUUCACCACCGCUCCCGCCAUCUGCCACGAC
GGCAAGGCCCACUUCCCUAGAGAGGGCGUGUUCGUGAGCAACGGCACCCACUGGUUCGUG
ACACAGAGAAACUUCUACGAGCCUCAGAUCAUCACCACCGACAACACCUUCGUGAGCGGC
AACUGCGACGUGGUGAUCGGCAUAGUGAACAACACCGUAUACGACCCCCUGCAGCCCGAG
CUGGACAGCUUCAAGGAGGAGCUGGACAAGUACUUCAAAAACCAUACUAGCCCCGACGUG
GACCUGGGAGACAUCUCUGGCAUCAACGCUAGCGUGGUGAACAUUCAGAAGGAGAUCGAC
CGGCUGAAUGAGGUGGCCAAGAACCUGAACGAGAGCCUGAUCGACCUGCAAGAGCUGGGC
AAGUACGAGCAGUACAUCAAGUGGCCCUGGUACAUCUGGCUGGGCUUCAUCGCCGGCCUG
AUCGCCAUCGUGAUGGUGACCAUCAUGCUGUGCUGCAUGACAAGCUGUUGCAGCUGCCUG
AAAGGUUGCUGCAGCUGUGGCUCGUGCUGCAAGUUCGACGAGGACGACAGCGAGCCCGUG
CUGAAGGGCGUGAAGCUGCACUACACCUGAUGACUCGAGGCUCGCUUUCUUGCUGUCCAA
UUUCUAUUAAAGGUUCCUUUGUUCCCUAAGUCCAACUACUAAACUGGGGGAUAUUAUGAA
GGGCCUUGAGCAUCUGGAUUCUGCCUAAUAAAAAACAUUUAUUUUCAUUGCAAAAAAAAA
AAAAAAAAAAAAAAAAAAAAAAAGCAUAUGACUAAAAAAAAAAAAAAAAAAAAAAAAAAA
AAAAAAAAAAAAAAAAAAAAAAAAAAAAAAAAAAAAAAAAAAAAA
>I'-H1N1-B mRNA
SEQ ID NO: 110
GAGUUAGGAGUUGGUGGCAGUUUCAAUAACAGGUCAUUGCCGAGAAAAGGAUAGCACUAU
AAUAUGAAGGCCAUCCUGGUGGUGAUGCUGUACACCUUCACCACCGCCAACGCCGACACC
CUGUGCAUCGGCUACCACGCCAACAACAGCACCGACACCGUGGACACCGUGCUGGAGAAG
AACGUGACCGUGACCCACAGCGUGAACCUGCUGGAGGACAAGCACAACGGCAAGCUGUGC
AAGCUGCGGGGCGUGGCCCCCCUGCACCUGGGCAAGUGCAACAUCGCCGGCUGGAUCCUG
GGCAACCCCGAGUGCGAGUCCCUGAGCACCGCCCGCUCCUGGAGCUACAUCGUGGAGACC
UCCAACAGCGACAACGGCACCUGCUACCCCGGGGACUUCAUCAACUACGAGGAGCUGCGG
GAGCAGCUGUCCAGCGUGUCCAGCUUCGAGCGCUUCGAGAUCUUCCCCAAGACCUCCAGC
UGGCCCAACCACGACUCCGACAACGGCGUGACCGCCGCCUGCCCCCACGCCGGGGCCAAG
AGCUUCUACAAGAACCUGAUCUGGCUGGUGAAGAAGGGCAAGUCCUACCCCAAGAUCAAC
CAGACCUACAUCAACGACAAGGGGAAGGAGGUCCUCGUGCUGUGGGGCAUCCACCACCCC
CCCACCAUCGCCGACCAGCAGAGCCUCUACCAGAACGCCGACGCCUACGUCUUCGUGGGG
ACCUCCCGGUACAGCAAGAAGUUCAAGCCCGAGAUCGCCACCCGCCCCAAGGUCCGGGAC
CAGGAGGGCCGCAUGAACUACUACUGGACCCUGGUGGAGCCGGGGGACAAGAUCACCUUC
GAGGCCACCGGCAACCUCGUCGCCCCGCGGUACGCCUUCACGAUGGAGCGCGACGCCGGG
UCCGGCAUCAUCAUCAGCGACACCCCCGUGCACGACUGCAACACGACCUGCCAGACGCCC
GAGGGGGCGAUCAACACCUCCCUGCCGUUCCAGAACGUCCACCCGAUCACGAUCGGCAAG
UGCCCCAAGUACGUGAAGUCCACCAAGCUCCGGCUGGCCACGGGGCUCCGCAACGUCCCC
UCCAUCCAGAGCCGGGGCCUGUUCGGGGCGAUCGCCGGCUUCAUCGAGGGGGGCUGGACC
GGGAUGGUGGACGGCUGGUACGGGUACCACCACCAGAACGAGCAGGGCUCCGGGUACGCG
GCCGACCUCAAGUCGACGCAGAACGCGAUCGACAAGAUCACCAACAAGGUCAACAGCGUG
AUCGAGAAGAUGAACACGCAGUUCACCGCCGUCGGCAAGGAGUUCAACCACCUGGAGAAG
CGCAUCGAGAACCUCAACAAGAAGGUGGACGACGGGUUCCUGGACAUCUGGACGUACAAC
GCGGAGCUCCUGGUCCUCCUGGAGAACGAGCGGACCCUCGACUACCACGACUCCAACGUG
AAGAACCUGUACGAGAAGGUCCGCAACCAGCUCAAGAACAACGCCAAGGAGAUCGGCAAC
GGGUGCUUCGAGUUCUACCACAAGUGCGACAACACGUGCAUGGAGUCGGUGAAGAACGGC
ACCUACGACUACCCGAAGUACAGCGAGGAGGCGAAGCUGAACCGGGAGAAGAUCGACGGG
GUCAAGCUCGACUCCACGCGCAUCUACCAGAUCCUGGCCAUCUACUCGACCGUGGCGAGC
UCCCUCGUCCUGGUGGUCUCGCUCGGCGCCAUCAGCUUCUGGAUGUGCUCCAACGGGUCG
CUGCAGUGCCGGAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAGCAUAUGACUAAAAAAAAAAAAAAAAAAAAAAAAAAAAAAAAA
AAAAAAAAAAAAAAAAAAAAAAAAAAAAAAAAAAAAAAA
>I'-H3N2-B mRNA
SEQ ID NO: 111
GAGUUAGGAGUUGGUGGCAGUUUCAAUAACAGGUCAUUGCCGAGAAAAGGAUAGCACUAU
AAUAUGAAGACCAUCAUCGCCCUGAGCUACAUCCUGUGCCUGGUGUUCGCCCAGAAGAUC
CCCGGCAACGACAACAGCACCGCCACCCUGUGCCUGGGCCACCACGCCGUGCCCAACGGC
ACCAUCGUGAAGACCAUCACCAACGACCGGAUCGAGGUGACCAACGCCACCGAGCUGGUG
CAGAACUCCAGCAUCGGCGAGAUCUGCGACUCCCCCCACCAGAUCCUGGACGGCGGCAAC
UGCACCCUGAUCGACGCCCUGCUGGGCGACCCCCAGUGCGACGGGUUCCAGAACAAGGAG
UGGGACCUGUUCGUGGAGCGGAGCCGCGCCAACUCCAACUGCUACCCCUACGACGUGCCC
GACUACGCCAGCCUGCGGUCCCUGGUGGCCAGCUCCGGCACCCUGGAGUUCAAGAACGAG
AGCUUCAACUGGACCGGGGUCAAGCAGAACGGCACCUCCAGCGCCUGCAUCCGCGGGUCC
AGCUCCAGCUUCUUCUCCCGGCUGAACUGGCUCACCCACCUGAACUACACCUACCCCGCC
CUCAACGUGACCAUGCCCAACAACGAGCAGUUCGACAAGCUGUACAUCUGGGGCGUCCAC
CACCCCAGCACCGACAAGGACCAGAUCUCCCUCUUCGCCCAGCCCAGCGGGCGCAUCACC
GUGUCCACGAAGCGGAGCCAGCAGGCCGUCAUCCCCAACAUCGGCUCCCGCCCGCGGAUC
CGCGACAUCCCCAGCCGGAUCUCCAUCUACUGGACGAUCGUGAAGCCGGGGGACAUCCUG
CUCAUCAACAGCACCGGCAACCUGAUCGCCCCCCGCGGGUACUUCAAGAUCCGGUCCGGC
AAGAGCUCGAUCAUGCGCUCCGACGCCCCGAUCGGGAAGUGCAAGAGCGAGUGCAUCACC
CCCAACGGCUCGAUCCCGAACGACAAGCCCUUCCAGAACGUCAACCGGAUCACGUACGGG
GCCUGCCCGCGCUACGUGAAGCAGUCCACGCUCAAGCUGGCCACCGGCAUGCGGAACGUC
CCCGAGAAGCAGACCCGCGGGAUCUUCGGCGCCAUCGCGGGGUUCAUCGAGAACGGCUGG
GAGGGGAUGGUGGACGGCUGGUACGGGUUCCGGCACCAGAACAGCGAGGGCCGCGGGCAG
GCCGCGGACCUCAAGUCGACGCAGGCCGCGAUCGACCAGAUCAACGGCAAGCUGAACCGG
CUCAUCGGGAAGACGAACGAGAAGUUCCACCAGAUCGAGAAGGAGUUCUCCGAGGUCGAG
GGCCGCGUGCAGGACCUGGAGAAGUACGUCGAGGACACCAAGAUCGACCUCUGGAGCUAC
AACGCCGAGCUGCUCGUGGCGCUGGAGAACCAGCACACCAUCGACCUCACGGACUCGGAG
AUGAACAAGCUGUUCGAGAAGACGAAGAAGCAGCUCCGGGAGAACGCCGAGGACAUGGGG
AACGGCUGCUUCAAGAUCUACCACAAGUGCGACAACGCGUGCAUCGGGUCCAUCCGCAAC
GAGACCUACGACCACAACGUCUACCGGGACGAGGCCCUGAACAACCGCUUCCAGAUCAAG
GGCGUGGAGCUCAAGAGCGGGUACAAGGACUGGAUCCUGUGGAUCUCGUUCGCGAUGUCC
UGCUUCCUCCUGUGCAUCGCCCUCCUGGGCUUCAUCAUGUGGGCGUGCCAGAAGGGGAAC
AUCCGGUGCAACAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAGCAUAUGACUAAAAAAAAAAAAAAAAAAAAAAAAAAAAAAAAA
AAAAAAAAAAAAAAAAAAAAAAAAAAAAAAAAAAAAAAA
>I'-B-Victoria-B mRNA
SEQ ID NO: 112
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCCACUUCGCCAACCUG
AAGGGCACCGAGACCCGGGGCAAGCUGUGCCCCAAGUGCCUGAACUGCACCGACCUGGAC
GUGGCCCUGGGCCGCCCCAAGUGCACCGGCAAGAUCCCCAGCGCCCGGGUGUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGCACGUCCGCCUGUCCACCCACAACGUG
AUCAACGCCGAGGACGCCCCCGGCCGGCCCUACGAGAUCGGGACCAGCGGCUCCUGCCCC
AACAUCACCAACGGGAACGGCUUCUUCGCCACCAUGGCCUGGGCCGUCCCCAAGAACAAG
ACCGCCACCAACCCCCUGACCAUCGAGGUGCCGUACAUCUGCACCGAGGGGGAGGACCAG
AUCACCGUCUGGGGCUUCCACAGCGACAACGAGACGCAGAUGGCCAAGCUGUACGGGGAC
UCCAAGCCCCAGAAGUUCACGAGCUCCGCCAACGGCGUGACCACCCACUACGUCAGCCAG
AUCGGGGGCUUCCCGAACCAGACGGAGGACGGGGGCCUGCCCCAGUCCGGGCGCAUCGUG
GUCGACUACAUGGUGCAGAAGUCCGGCAAGACGGGGACCAUCACGUACCAGCGGGGCAUC
CUGCUGCCGCAGAAGGUCUGGUGCGCCUCCGGGCGCAGCAAGGUGAUCAAGGGCUCCCUG
CCCCUCAUCGGGGAGGCCGACUGCCUGCACGAGAAGUACGGCGGGCUCAACAAGAGCAAG
CCGUACUACACCGGCGAGCACGCCAAGGCCAUCGGGAACUGCCCCAUCUGGGUCAAGACC
CCGCUGAAGCUCGCCAACGGCACGAAGUACCGGCCCCCGGCCAAGCUGCUCAAGGAGCGC
GGGUUCUUCGGCGCCAUCGCCGGGUUCCUGGAGGGCGGGUGGGAGGGCAUGAUCGCCGGG
UGGCACGGCUACACGUCCCACGGGGCCCACGGCGUGGCGGUCGCCGCGGACCUCAAGAGC
ACCCAGGAGGCCAUCAACAAGAUCACGAAGAACCUGAACUCCCUCAGCGAGCUGGAGGUG
AAGAACCUCCAGCGCCUGUCCGGGGCGAUGGACGAGCUCCACAACGAGAUCCUGGAGCUC
GACGAGAAGGUCGACGACCUGCGGGCCGACACCAUCAGCUCGCAGAUCGAGCUCGCGGUG
CUGCUCUCCAACGAGGGCAUCAUCAACAGCGAGGACGAGCACCUGCUCGCCCUGGAGCGG
AAGCUCAAGAAGAUGCUGGGGCCCUCGGCGGUCGAGAUCGGCAACGGGUGCUUCGAGACC
AAGCACAAGUGCAACCAGACGUGCCUCGACCGCAUCGCCGCGGGCACGUUCGACGCCGGG
GAGUUCUCCCUGCCGACCUUCGACAGCCUCAACAUCACGGCGGCCUCGCUGAACGACGAC
GGCCUCGACAACCACACCAUCCUGCUCUACUACUCCACCGCGGCCAGCUCGCUGGCGGUG
ACGCUCAUGAUCGCCAUCUUCGUCGUGUACAUGGUCUCCCGCGACAACGUGAGCUGCUCG
AUCUGCCUGUGAUGACUCGAGGCUCGCUUUCUUGCUGUCCAAUUUCUAUUAAAGGUUCCU
UUGUUCCCUAAGUCCAACUACUAAACUGGGGGAUAUUAUGAAGGGCCUUGAGCAUCUGGA
UUCUGCCUAAUAAAAAACAUUUAUUUUCAUUGCAAAAAAAAAAAAAAAAAAAAAAAAAAA
AAAAAGCAUAUGACUAAAAAAAAAAAAAAAAAAAAAAAAAAAAAAAAAAAAAAAAAAAAA
AAAAAAAAAAAAAAAAAAAAAAAAAAA
>I'-B-Yamagata-B mRNA
SEQ ID NO: 113
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCUACUUCGCCAACCUG
AAGGGCACCCGGACCCGCGGCAAGCUGUGCCCCGACUGCCUGAACUGCACCGACCUGGAC
GUGGCCCUGGGCCGGCCCAUGUGCGUGGGCACCACCCCCAGCGCCAAGGCCUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGAAGAUCCGCCUGUCCACCCAGAACGUC
AUCGACGCCGAGAAGGCCCCCGGCGGGCCCUACCGGCUGGGCACCAGCGGGUCCUGCCCC
AACGCCACCAGCAAGAUCGGCUUCUUCGCCACCAUGGCCUGGGCCGUGCCCAAGGACAAC
UACAAGAACGCCACCAACCCCCUGACCGUCGAGGUGCCCUACAUCUGCACCGAGGGGGAG
GACCAGAUCACGGUCUGGGGCUUCCACUCCGACAACAAGACCCAGAUGAAGUCCCUGUAC
GGGGACUCCAACCCGCAGAAGUUCACGAGCUCCGCCAACGGCGUGACCACGCACUACGUC
AGCCAGAUCGGGGACUUCCCGGACCAGACCGAGGACGGCGGGCUGCCCCAGUCCGGCCGC
AUCGUGGUCGACUACAUGAUGCAGAAGCCCGGGAAGACGGGCACCAUCGUGUACCAGCGG
GGGGUCCUGCUGCCGCAGAAGGUGUGGUGCGCCAGCGGCCGCUCCAAGGUCAUCAAGGGG
AGCCUGCCCCUCAUCGGCGAGGCCGACUGCCUGCACGAGGAGUACGGGGGCCUCAACAAG
UCCAAGCCGUACUACACGGGGAAGCACGCCAAGGCCAUCGGCAACUGCCCGAUCUGGGUG
AAGACCCCCCUGAAGCUCGCCAACGGGACGAAGUACCGGCCGCCCGCCAAGCUGCUCAAG
GAGCGCGGCUUCUUCGGGGCCAUCGCCGGCUUCCUGGAGGGGGGCUGGGAGGGGAUGAUC
GCCGGCUGGCACGGGUACACCAGCCACGGCGCCCACGGGGUCGCGGUGGCCGCGGACCUC
AAGUCCACGCAGGAGGCCAUCAACAAGAUCACCAAGAACCUGAACAGCCUCUCCGAGCUG
GAGGUCAAGAACCUCCAGCGCCUGAGCGGCGCGAUGGACGAGCUCCACAACGAGAUCCUG
GAGCUCGACGAGAAGGUGGACGACCUGCGGGCCGACACGAUCUCGUCCCAGAUCGAGCUC
GCGGUCCUGCUCAGCAACGAGGGGAUCAUCAACUCGGAGGACGAGCACCUGCUCGCCCUG
GAGCGGAAGCUCAAGAAGAUGCUGGGCCCGUCCGCGGUGGACAUCGGGAACGGCUGCUUC
GAGACCAAGCACAAGUGCAACCAGACGUGCCUCGACCGCAUCGCCGCGGGGACCUUCAAC
GCCGGCGAGUUCAGCCUGCCCACGUUCGACUCGCUCAACAUCACCGCGGCCUCCCUGAAC
GACGACGGGCUCGACAACCACACGAUCCUGCUCUACUACAGCACCGCGGCCUCGUCCCUG
GCGGUCACGCUCAUGCUGGCCAUCUUCAUCGUGUACAUGGUCAGCCGCGACAACGUGUCG
UGCUCCAUCUGCCUCUGAUGACUCGAGGCUCGCUUUCUUGCUGUCCAAUUUCUAUUAAAG
GUUCCUUUGUUCCCUAAGUCCAACUACUAAACUGGGGGAUAUUAUGAAGGGCCUUGAGCA
UCUGGAUUCUGCCUAAUAAAAAACAUUUAUUUUCAUUGCAAAAAAAAAAAAAAAAAAAAA
AAAAAAAAAAAGCAUAUGACUAAAAAAAAAAAAAAAAAAAAAAAAAAAAAAAAAAAAAAA
AAAAAAAAAAAAAAAAAAAAAAAAAAAAAAAAA
>I'-H1N1-HAO-mut-B mRNA
SEQ ID NO: 114
GAGUUAGGAGUUGGUGGCAGUUUCAAUAACAGGUCAUUGCCGAGAAAAGGAUAGCACUAU
AAUAUGAAGGCCAUCCUGGUGGUGAUGCUGUACACCUUCACCACCGCCAACGCCGACACC
CUGUGCAUCGGCUACCACGCCAACAACAGCACCGACACCGUGGACACCGUGCUGGAGAAG
AACGUGACCGUGACCCACAGCGUGAACCUGCUGGAGGACAAGCACAACGGCAAGCUGUGC
AAGCUGCGGGGCGUGGCCCCCCUGCACCUGGGCAAGUGCAACAUCGCCGGCUGGAUCCUG
GGCAACCCCGAGUGCGAGUCCCUGAGCACCGCCCGCUCCUGGAGCUACAUCGUGGAGACC
UCCAACAGCGACAACGGCACCUGCUACCCCGGGGACUUCAUCAACUACGAGGAGCUGCGG
GAGCAGCUGUCCAGCGUGUCCAGCUUCGAGCGCUUCGAGAUCUUCCCCAAGACCUCCAGC
UGGCCCAACCACGACUCCGACAACGGCGUGACCGCCGCCUGCCCCCACGCCGGGGCCAAG
AGCUUCUACAAGAACCUGAUCUGGCUGGUGAAGAAGGGCAAGUCCUACCCCAAGAUCAAC
CAGACCUACAUCAACGACAAGGGGAAGGAGGUCCUCGUGCUGUGGGGCAUCCACCACCCC
CCCACCAUCGCCGACCAGCAGAGCCUCUACCAGAACGCCGACGCCUACGUCUUCGUGGGG
ACCUCCCGGUACAGCAAGAAGUUCAAGCCCGAGAUCGCCACCCGCCCCAAGGUCCGGGAC
CAGGAGGGCCGCAUGAACUACUACUGGACCCUGGUGGAGCCGGGGGACAAGAUCACCUUC
GAGGCCACCGGCAACCUCGUCGCCCCGCGGUACGCCUUCACGAUGGAGCGCGACGCCGGG
UCCGGCAUCAUCAUCAGCGACACCCCCGUGCACGACUGCAACACGACCUGCCAGACGCCC
GAGGGGGCGAUCAACACCUCCCUGCCGUUCCAGAACGUCCACCCGAUCACGAUCGGCAAG
UGCCCCAAGUACGUGAAGUCCACCAAGCUCCGGCUGGCCACGGGGCUCCGCAACGUCCCC
UCCAUCCAGAGCCAGGGCCUGUUCGGGGCGAUCGCCGGCUUCAUCGAGGGGGGCUGGACC
GGGAUGGUGGACGGCUGGUACGGGUACCACCACCAGAACGAGCAGGGCUCCGGGUACGCG
GCCGACCUCAAGUCGACGCAGAACGCGAUCGACAAGAUCACCAACAAGGUCAACAGCGUG
AUCGAGAAGAUGAACACGCAGUUCACCGCCGUCGGCAAGGAGUUCAACCACCUGGAGAAG
CGCAUCGAGAACCUCAACAAGAAGGUGGACGACGGGUUCCUGGACAUCUGGACGUACAAC
GCGGAGCUCCUGGUCCUCCUGGAGAACGAGCGGACCCUCGACUACCACGACUCCAACGUG
AAGAACCUGUACGAGAAGGUCCGCAACCAGCUCAAGAACAACGCCAAGGAGAUCGGCAAC
GGGUGCUUCGAGUUCUACCACAAGUGCGACAACACGUGCAUGGAGUCGGUGAAGAACGGC
ACCUACGACUACCCGAAGUACAGCGAGGAGGCGAAGCUGAACCGGGAGAAGAUCGACGGG
GUCAAGCUCGACUCCACGCGCAUCUACCAGAUCCUGGCCAUCUACUCGACCGUGGCGAGC
UCCCUCGUCCUGGUGGUCUCGCUCGGCGCCAUCAGCUUCUGGAUGUGCUCCAACGGGUCG
CUGCAGUGCCGGAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAGCAUAUGACUAAAAAAAAAAAAAAAAAAAAAAAAAAAAAAAAA
AAAAAAAAAAAAAAAAAAAAAAAAAAAAAAAAAAAAAAA
>I'-H3N2-HA0-mut-B mRNA
SEQ ID NO: 115
GAGUUAGGAGUUGGUGGCAGUUUCAAUAACAGGUCAUUGCCGAGAAAAGGAUAGCACUAU
AAUAUGAAGACCAUCAUCGCCCUGAGCUACAUCCUGUGCCUGGUGUUCGCCCAGAAGAUC
CCCGGCAACGACAACAGCACCGCCACCCUGUGCCUGGGCCACCACGCCGUGCCCAACGGC
ACCAUCGUGAAGACCAUCACCAACGACCGGAUCGAGGUGACCAACGCCACCGAGCUGGUG
CAGAACUCCAGCAUCGGCGAGAUCUGCGACUCCCCCCACCAGAUCCUGGACGGCGGCAAC
UGCACCCUGAUCGACGCCCUGCUGGGCGACCCCCAGUGCGACGGGUUCCAGAACAAGGAG
UGGGACCUGUUCGUGGAGCGGAGCCGCGCCAACUCCAACUGCUACCCCUACGACGUGCCC
GACUACGCCAGCCUGCGGUCCCUGGUGGCCAGCUCCGGCACCCUGGAGUUCAAGAACGAG
AGCUUCAACUGGACCGGGGUCAAGCAGAACGGCACCUCCAGCGCCUGCAUCCGCGGGUCC
AGCUCCAGCUUCUUCUCCCGGCUGAACUGGCUCACCCACCUGAACUACACCUACCCCGCC
CUCAACGUGACCAUGCCCAACAACGAGCAGUUCGACAAGCUGUACAUCUGGGGCGUCCAC
CACCCCAGCACCGACAAGGACCAGAUCUCCCUCUUCGCCCAGCCCAGCGGGCGCAUCACC
GUGUCCACGAAGCGGAGCCAGCAGGCCGUCAUCCCCAACAUCGGCUCCCGCCCGCGGAUC
CGCGACAUCCCCAGCCGGAUCUCCAUCUACUGGACGAUCGUGAAGCCGGGGGACAUCCUG
CUCAUCAACAGCACCGGCAACCUGAUCGCCCCCCGCGGGUACUUCAAGAUCCGGUCCGGC
AAGAGCUCGAUCAUGCGCUCCGACGCCCCGAUCGGGAAGUGCAAGAGCGAGUGCAUCACC
CCCAACGGCUCGAUCCCGAACGACAAGCCCUUCCAGAACGUCAACCGGAUCACGUACGGG
GCCUGCCCGCGCUACGUGAAGCAGUCCACGCUCAAGCUGGCCACCGGCAUGCGGAACGUC
CCCGAGAAGCAGACCCAGGGGAUCUUCGGCGCCAUCGCGGGGUUCAUCGAGAACGGCUGG
GAGGGGAUGGUGGACGGCUGGUACGGGUUCCGGCACCAGAACAGCGAGGGCCGCGGGCAG
GCCGCGGACCUCAAGUCGACGCAGGCCGCGAUCGACCAGAUCAACGGCAAGCUGAACCGG
CUCAUCGGGAAGACGAACGAGAAGUUCCACCAGAUCGAGAAGGAGUUCUCCGAGGUCGAG
GGCCGCGUGCAGGACCUGGAGAAGUACGUCGAGGACACCAAGAUCGACCUCUGGAGCUAC
AACGCCGAGCUGCUCGUGGCGCUGGAGAACCAGCACACCAUCGACCUCACGGACUCGGAG
AUGAACAAGCUGUUCGAGAAGACGAAGAAGCAGCUCCGGGAGAACGCCGAGGACAUGGGG
AACGGCUGCUUCAAGAUCUACCACAAGUGCGACAACGCGUGCAUCGGGUCCAUCCGCAAC
GAGACCUACGACCACAACGUCUACCGGGACGAGGCCCUGAACAACCGCUUCCAGAUCAAG
GGCGUGGAGCUCAAGAGCGGGUACAAGGACUGGAUCCUGUGGAUCUCGUUCGCGAUGUCC
UGCUUCCUCCUGUGCAUCGCCCUCCUGGGCUUCAUCAUGUGGGCGUGCCAGAAGGGGAAC
AUCCGGUGCAACAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAGCAUAUGACUAAAAAAAAAAAAAAAAAAAAAAAAAAAAAAAAA
AAAAAAAAAAAAAAAAAAAAAAAAAAAAAAAAAAAAAAA
>I'-B-Victoria-HA0-mut-B mRNA
SEQ ID NO: 116
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCCACUUCGCCAACCUG
AAGGGCACCGAGACCCGGGGCAAGCUGUGCCCCAAGUGCCUGAACUGCACCGACCUGGAC
GUGGCCCUGGGCCGCCCCAAGUGCACCGGCAAGAUCCCCAGCGCCCGGGUGUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGCACGUCCGCCUGUCCACCCACAACGUG
AUCAACGCCGAGGACGCCCCCGGCCGGCCCUACGAGAUCGGGACCAGCGGCUCCUGCCCC
AACAUCACCAACGGGAACGGCUUCUUCGCCACCAUGGCCUGGGCCGUCCCCAAGAACAAG
ACCGCCACCAACCCCCUGACCAUCGAGGUGCCGUACAUCUGCACCGAGGGGGAGGACCAG
AUCACCGUCUGGGGCUUCCACAGCGACAACGAGACGCAGAUGGCCAAGCUGUACGGGGAC
UCCAAGCCCCAGAAGUUCACGAGCUCCGCCAACGGCGUGACCACCCACUACGUCAGCCAG
AUCGGGGGCUUCCCGAACCAGACGGAGGACGGGGGCCUGCCCCAGUCCGGGCGCAUCGUG
GUCGACUACAUGGUGCAGAAGUCCGGCAAGACGGGGACCAUCACGUACCAGCGGGGCAUC
CUGCUGCCGCAGAAGGUCUGGUGCGCCUCCGGGCGCAGCAAGGUGAUCAAGGGCUCCCUG
CCCCUCAUCGGGGAGGCCGACUGCCUGCACGAGAAGUACGGCGGGCUCAACAAGAGCAAG
CCGUACUACACCGGCGAGCACGCCAAGGCCAUCGGGAACUGCCCCAUCUGGGUCAAGACC
CCGCUGAAGCUCGCCAACGGCACGAAGUACCGGCCCCCGGCCAAGCUGCUCAAGGAGCAG
GGGUUCUUCGGCGCCAUCGCCGGGUUCCUGGAGGGCGGGUGGGAGGGCAUGAUCGCCGGG
UGGCACGGCUACACGUCCCACGGGGCCCACGGCGUGGCGGUCGCCGCGGACCUCAAGAGC
ACCCAGGAGGCCAUCAACAAGAUCACGAAGAACCUGAACUCCCUCAGCGAGCUGGAGGUG
AAGAACCUCCAGCGCCUGUCCGGGGCGAUGGACGAGCUCCACAACGAGAUCCUGGAGCUC
GACGAGAAGGUCGACGACCUGCGGGCCGACACCAUCAGCUCGCAGAUCGAGCUCGCGGUG
CUGCUCUCCAACGAGGGCAUCAUCAACAGCGAGGACGAGCACCUGCUCGCCCUGGAGCGG
AAGCUCAAGAAGAUGCUGGGGCCCUCGGCGGUCGAGAUCGGCAACGGGUGCUUCGAGACC
AAGCACAAGUGCAACCAGACGUGCCUCGACCGCAUCGCCGCGGGCACGUUCGACGCCGGG
GAGUUCUCCCUGCCGACCUUCGACAGCCUCAACAUCACGGCGGCCUCGCUGAACGACGAC
GGCCUCGACAACCACACCAUCCUGCUCUACUACUCCACCGCGGCCAGCUCGCUGGCGGUG
ACGCUCAUGAUCGCCAUCUUCGUCGUGUACAUGGUCUCCCGCGACAACGUGAGCUGCUCG
AUCUGCCUGUGAUGACUCGAGGCUCGCUUUCUUGCUGUCCAAUUUCUAUUAAAGGUUCCU
UUGUUCCCUAAGUCCAACUACUAAACUGGGGGAUAUUAUGAAGGGCCUUGAGCAUCUGGA
UUCUGCCUAAUAAAAAACAUUUAUUUUCAUUGCAAAAAAAAAAAAAAAAAAAAAAAAAAA
AAAAAGCAUAUGACUAAAAAAAAAAAAAAAAAAAAAAAAAAAAAAAAAAAAAAAAAAAAA
AAAAAAAAAAAAAAAAAAAAAAAAAAA
>I'-B-Yamagata-HA0-mut-B mRNA
SEQ ID NO: 117
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCUACUUCGCCAACCUG
AAGGGCACCCGGACCCGCGGCAAGCUGUGCCCCGACUGCCUGAACUGCACCGACCUGGAC
GUGGCCCUGGGCCGGCCCAUGUGCGUGGGCACCACCCCCAGCGCCAAGGCCUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGAAGAUCCGCCUGUCCACCCAGAACGUC
AUCGACGCCGAGAAGGCCCCCGGCGGGCCCUACCGGCUGGGCACCAGCGGGUCCUGCCCC
AACGCCACCAGCAAGAUCGGCUUCUUCGCCACCAUGGCCUGGGCCGUGCCCAAGGACAAC
UACAAGAACGCCACCAACCCCCUGACCGUCGAGGUGCCCUACAUCUGCACCGAGGGGGAG
GACCAGAUCACGGUCUGGGGCUUCCACUCCGACAACAAGACCCAGAUGAAGUCCCUGUAC
GGGGACUCCAACCCGCAGAAGUUCACGAGCUCCGCCAACGGCGUGACCACGCACUACGUC
AGCCAGAUCGGGGACUUCCCGGACCAGACCGAGGACGGCGGGCUGCCCCAGUCCGGCCGC
AUCGUGGUCGACUACAUGAUGCAGAAGCCCGGGAAGACGGGCACCAUCGUGUACCAGCGG
GGGGUCCUGCUGCCGCAGAAGGUGUGGUGCGCCAGCGGCCGCUCCAAGGUCAUCAAGGGG
AGCCUGCCCCUCAUCGGCGAGGCCGACUGCCUGCACGAGGAGUACGGGGGCCUCAACAAG
UCCAAGCCGUACUACACGGGGAAGCACGCCAAGGCCAUCGGCAACUGCCCGAUCUGGGUG
AAGACCCCCCUGAAGCUCGCCAACGGGACGAAGUACCGGCCGCCCGCCAAGCUGCUCAAG
GAGCAGGGCUUCUUCGGGGCCAUCGCCGGCUUCCUGGAGGGGGGCUGGGAGGGGAUGAUC
GCCGGCUGGCACGGGUACACCAGCCACGGCGCCCACGGGGUCGCGGUGGCCGCGGACCUC
AAGUCCACGCAGGAGGCCAUCAACAAGAUCACCAAGAACCUGAACAGCCUCUCCGAGCUG
GAGGUCAAGAACCUCCAGCGCCUGAGCGGCGCGAUGGACGAGCUCCACAACGAGAUCCUG
GAGCUCGACGAGAAGGUGGACGACCUGCGGGCCGACACGAUCUCGUCCCAGAUCGAGCUC
GCGGUCCUGCUCAGCAACGAGGGGAUCAUCAACUCGGAGGACGAGCACCUGCUCGCCCUG
GAGCGGAAGCUCAAGAAGAUGCUGGGCCCGUCCGCGGUGGACAUCGGGAACGGCUGCUUC
GAGACCAAGCACAAGUGCAACCAGACGUGCCUCGACCGCAUCGCCGCGGGGACCUUCAAC
GCCGGCGAGUUCAGCCUGCCCACGUUCGACUCGCUCAACAUCACCGCGGCCUCCCUGAAC
GACGACGGGCUCGACAACCACACGAUCCUGCUCUACUACAGCACCGCGGCCUCGUCCCUG
GCGGUCACGCUCAUGCUGGCCAUCUUCAUCGUGUACAUGGUCAGCCGCGACAACGUGUCG
UGCUCCAUCUGCCUCUGAUGACUCGAGGCUCGCUUUCUUGCUGUCCAAUUUCUAUUAAAG
GUUCCUUUGUUCCCUAAGUCCAACUACUAAACUGGGGGAUAUUAUGAAGGGCCUUGAGCA
UCUGGAUUCUGCCUAAUAAAAAACAUUUAUUUUCAUUGCAAAAAAAAAAAAAAAAAAAAA
AAAAAAAAAAAGCAUAUGACUAAAAAAAAAAAAAAAAAAAAAAAAAAAAAAAAAAAAAAA
AAAAAAAAAAAAAAAAAAAAAAAAAAAAAAAAA
>I'-H1N1-B-120A mRNA
SEQ ID NO: 118
GAGUUAGGAGUUGGUGGCAGUUUCAAUAACAGGUCAUUGCCGAGAAAAGGAUAGCACUAU
AAUAUGAAGGCCAUCCUGGUGGUGAUGCUGUACACCUUCACCACCGCCAACGCCGACACC
CUGUGCAUCGGCUACCACGCCAACAACAGCACCGACACCGUGGACACCGUGCUGGAGAAG
AACGUGACCGUGACCCACAGCGUGAACCUGCUGGAGGACAAGCACAACGGCAAGCUGUGC
AAGCUGCGGGGCGUGGCCCCCCUGCACCUGGGCAAGUGCAACAUCGCCGGCUGGAUCCUG
GGCAACCCCGAGUGCGAGUCCCUGAGCACCGCCCGCUCCUGGAGCUACAUCGUGGAGACC
UCCAACAGCGACAACGGCACCUGCUACCCCGGGGACUUCAUCAACUACGAGGAGCUGCGG
GAGCAGCUGUCCAGCGUGUCCAGCUUCGAGCGCUUCGAGAUCUUCCCCAAGACCUCCAGC
UGGCCCAACCACGACUCCGACAACGGCGUGACCGCCGCCUGCCCCCACGCCGGGGCCAAG
AGCUUCUACAAGAACCUGAUCUGGCUGGUGAAGAAGGGCAAGUCCUACCCCAAGAUCAAC
CAGACCUACAUCAACGACAAGGGGAAGGAGGUCCUCGUGCUGUGGGGCAUCCACCACCCC
CCCACCAUCGCCGACCAGCAGAGCCUCUACCAGAACGCCGACGCCUACGUCUUCGUGGGG
ACCUCCCGGUACAGCAAGAAGUUCAAGCCCGAGAUCGCCACCCGCCCCAAGGUCCGGGAC
CAGGAGGGCCGCAUGAACUACUACUGGACCCUGGUGGAGCCGGGGGACAAGAUCACCUUC
GAGGCCACCGGCAACCUCGUCGCCCCGCGGUACGCCUUCACGAUGGAGCGCGACGCCGGG
UCCGGCAUCAUCAUCAGCGACACCCCCGUGCACGACUGCAACACGACCUGCCAGACGCCC
GAGGGGGCGAUCAACACCUCCCUGCCGUUCCAGAACGUCCACCCGAUCACGAUCGGCAAG
UGCCCCAAGUACGUGAAGUCCACCAAGCUCCGGCUGGCCACGGGGCUCCGCAACGUCCCC
UCCAUCCAGAGCCGGGGCCUGUUCGGGGCGAUCGCCGGCUUCAUCGAGGGGGGCUGGACC
GGGAUGGUGGACGGCUGGUACGGGUACCACCACCAGAACGAGCAGGGCUCCGGGUACGCG
GCCGACCUCAAGUCGACGCAGAACGCGAUCGACAAGAUCACCAACAAGGUCAACAGCGUG
AUCGAGAAGAUGAACACGCAGUUCACCGCCGUCGGCAAGGAGUUCAACCACCUGGAGAAG
CGCAUCGAGAACCUCAACAAGAAGGUGGACGACGGGUUCCUGGACAUCUGGACGUACAAC
GCGGAGCUCCUGGUCCUCCUGGAGAACGAGCGGACCCUCGACUACCACGACUCCAACGUG
AAGAACCUGUACGAGAAGGUCCGCAACCAGCUCAAGAACAACGCCAAGGAGAUCGGCAAC
GGGUGCUUCGAGUUCUACCACAAGUGCGACAACACGUGCAUGGAGUCGGUGAAGAACGGC
ACCUACGACUACCCGAAGUACAGCGAGGAGGCGAAGCUGAACCGGGAGAAGAUCGACGGG
GUCAAGCUCGACUCCACGCGCAUCUACCAGAUCCUGGCCAUCUACUCGACCGUGGCGAGC
UCCCUCGUCCUGGUGGUCUCGCUCGGCGCCAUCAGCUUCUGGAUGUGCUCCAACGGGUCG
CUGCAGUGCCGGAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAA
>I'-H3N2-B-120A mRNA
SEQ ID NO: 119
GAGUUAGGAGUUGGUGGCAGUUUCAAUAACAGGUCAUUGCCGAGAAAAGGAUAGCACUAU
AAUAUGAAGACCAUCAUCGCCCUGAGCUACAUCCUGUGCCUGGUGUUCGCCCAGAAGAUC
CCCGGCAACGACAACAGCACCGCCACCCUGUGCCUGGGCCACCACGCCGUGCCCAACGGC
ACCAUCGUGAAGACCAUCACCAACGACCGGAUCGAGGUGACCAACGCCACCGAGCUGGUG
CAGAACUCCAGCAUCGGCGAGAUCUGCGACUCCCCCCACCAGAUCCUGGACGGCGGCAAC
UGCACCCUGAUCGACGCCCUGCUGGGCGACCCCCAGUGCGACGGGUUCCAGAACAAGGAG
UGGGACCUGUUCGUGGAGCGGAGCCGCGCCAACUCCAACUGCUACCCCUACGACGUGCCC
GACUACGCCAGCCUGCGGUCCCUGGUGGCCAGCUCCGGCACCCUGGAGUUCAAGAACGAG
AGCUUCAACUGGACCGGGGUCAAGCAGAACGGCACCUCCAGCGCCUGCAUCCGCGGGUCC
AGCUCCAGCUUCUUCUCCCGGCUGAACUGGCUCACCCACCUGAACUACACCUACCCCGCC
CUCAACGUGACCAUGCCCAACAACGAGCAGUUCGACAAGCUGUACAUCUGGGGCGUCCAC
CACCCCAGCACCGACAAGGACCAGAUCUCCCUCUUCGCCCAGCCCAGCGGGCGCAUCACC
GUGUCCACGAAGCGGAGCCAGCAGGCCGUCAUCCCCAACAUCGGCUCCCGCCCGCGGAUC
CGCGACAUCCCCAGCCGGAUCUCCAUCUACUGGACGAUCGUGAAGCCGGGGGACAUCCUG
CUCAUCAACAGCACCGGCAACCUGAUCGCCCCCCGCGGGUACUUCAAGAUCCGGUCCGGC
AAGAGCUCGAUCAUGCGCUCCGACGCCCCGAUCGGGAAGUGCAAGAGCGAGUGCAUCACC
CCCAACGGCUCGAUCCCGAACGACAAGCCCUUCCAGAACGUCAACCGGAUCACGUACGGG
GCCUGCCCGCGCUACGUGAAGCAGUCCACGCUCAAGCUGGCCACCGGCAUGCGGAACGUC
CCCGAGAAGCAGACCCGCGGGAUCUUCGGCGCCAUCGCGGGGUUCAUCGAGAACGGCUGG
GAGGGGAUGGUGGACGGCUGGUACGGGUUCCGGCACCAGAACAGCGAGGGCCGCGGGCAG
GCCGCGGACCUCAAGUCGACGCAGGCCGCGAUCGACCAGAUCAACGGCAAGCUGAACCGG
CUCAUCGGGAAGACGAACGAGAAGUUCCACCAGAUCGAGAAGGAGUUCUCCGAGGUCGAG
GGCCGCGUGCAGGACCUGGAGAAGUACGUCGAGGACACCAAGAUCGACCUCUGGAGCUAC
AACGCCGAGCUGCUCGUGGCGCUGGAGAACCAGCACACCAUCGACCUCACGGACUCGGAG
AUGAACAAGCUGUUCGAGAAGACGAAGAAGCAGCUCCGGGAGAACGCCGAGGACAUGGGG
AACGGCUGCUUCAAGAUCUACCACAAGUGCGACAACGCGUGCAUCGGGUCCAUCCGCAAC
GAGACCUACGACCACAACGUCUACCGGGACGAGGCCCUGAACAACCGCUUCCAGAUCAAG
GGCGUGGAGCUCAAGAGCGGGUACAAGGACUGGAUCCUGUGGAUCUCGUUCGCGAUGUCC
UGCUUCCUCCUGUGCAUCGCCCUCCUGGGCUUCAUCAUGUGGGCGUGCCAGAAGGGGAAC
AUCCGGUGCAACAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAA
>I'-B-Victoria-B-120A mRNA
SEQ ID NO: 120
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCCACUUCGCCAACCUG
AAGGGCACCGAGACCCGGGGCAAGCUGUGCCCCAAGUGCCUGAACUGCACCGACCUGGAC
GUGGCCCUGGGCCGCCCCAAGUGCACCGGCAAGAUCCCCAGCGCCCGGGUGUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGCACGUCCGCCUGUCCACCCACAACGUG
AUCAACGCCGAGGACGCCCCCGGCCGGCCCUACGAGAUCGGGACCAGCGGCUCCUGCCCC
AACAUCACCAACGGGAACGGCUUCUUCGCCACCAUGGCCUGGGCCGUCCCCAAGAACAAG
ACCGCCACCAACCCCCUGACCAUCGAGGUGCCGUACAUCUGCACCGAGGGGGAGGACCAG
AUCACCGUCUGGGGCUUCCACAGCGACAACGAGACGCAGAUGGCCAAGCUGUACGGGGAC
UCCAAGCCCCAGAAGUUCACGAGCUCCGCCAACGGCGUGACCACCCACUACGUCAGCCAG
AUCGGGGGCUUCCCGAACCAGACGGAGGACGGGGGCCUGCCCCAGUCCGGGCGCAUCGUG
GUCGACUACAUGGUGCAGAAGUCCGGCAAGACGGGGACCAUCACGUACCAGCGGGGCAUC
CUGCUGCCGCAGAAGGUCUGGUGCGCCUCCGGGCGCAGCAAGGUGAUCAAGGGCUCCCUG
CCCCUCAUCGGGGAGGCCGACUGCCUGCACGAGAAGUACGGCGGGCUCAACAAGAGCAAG
CCGUACUACACCGGCGAGCACGCCAAGGCCAUCGGGAACUGCCCCAUCUGGGUCAAGACC
CCGCUGAAGCUCGCCAACGGCACGAAGUACCGGCCCCCGGCCAAGCUGCUCAAGGAGCGC
GGGUUCUUCGGCGCCAUCGCCGGGUUCCUGGAGGGCGGGUGGGAGGGCAUGAUCGCCGGG
UGGCACGGCUACACGUCCCACGGGGCCCACGGCGUGGCGGUCGCCGCGGACCUCAAGAGC
ACCCAGGAGGCCAUCAACAAGAUCACGAAGAACCUGAACUCCCUCAGCGAGCUGGAGGUG
AAGAACCUCCAGCGCCUGUCCGGGGCGAUGGACGAGCUCCACAACGAGAUCCUGGAGCUC
GACGAGAAGGUCGACGACCUGCGGGCCGACACCAUCAGCUCGCAGAUCGAGCUCGCGGUG
CUGCUCUCCAACGAGGGCAUCAUCAACAGCGAGGACGAGCACCUGCUCGCCCUGGAGCGG
AAGCUCAAGAAGAUGCUGGGGCCCUCGGCGGUCGAGAUCGGCAACGGGUGCUUCGAGACC
AAGCACAAGUGCAACCAGACGUGCCUCGACCGCAUCGCCGCGGGCACGUUCGACGCCGGG
GAGUUCUCCCUGCCGACCUUCGACAGCCUCAACAUCACGGCGGCCUCGCUGAACGACGAC
GGCCUCGACAACCACACCAUCCUGCUCUACUACUCCACCGCGGCCAGCUCGCUGGCGGUG
ACGCUCAUGAUCGCCAUCUUCGUCGUGUACAUGGUCUCCCGCGACAACGUGAGCUGCUCG
AUCUGCCUGUGAUGACUCGAGGCUCGCUUUCUUGCUGUCCAAUUUCUAUUAAAGGUUCCU
UUGUUCCCUAAGUCCAACUACUAAACUGGGGGAUAUUAUGAAGGGCCUUGAGCAUCUGGA
UUCUGCCUAAUAAAAAACAUUUAUUUUCAUUGCAAAAAAAAAAAAAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAA
>I'-B-Yamagata-B-120A mRNA
SEQ ID NO: 121
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCUACUUCGCCAACCUG
AAGGGCACCCGGACCCGCGGCAAGCUGUGCCCCGACUGCCUGAACUGCACCGACCUGGAC
GUGGCCCUGGGCCGGCCCAUGUGCGUGGGCACCACCCCCAGCGCCAAGGCCUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGAAGAUCCGCCUGUCCACCCAGAACGUC
AUCGACGCCGAGAAGGCCCCCGGCGGGCCCUACCGGCUGGGCACCAGCGGGUCCUGCCCC
AACGCCACCAGCAAGAUCGGCUUCUUCGCCACCAUGGCCUGGGCCGUGCCCAAGGACAAC
UACAAGAACGCCACCAACCCCCUGACCGUCGAGGUGCCCUACAUCUGCACCGAGGGGGAG
GACCAGAUCACGGUCUGGGGCUUCCACUCCGACAACAAGACCCAGAUGAAGUCCCUGUAC
GGGGACUCCAACCCGCAGAAGUUCACGAGCUCCGCCAACGGCGUGACCACGCACUACGUC
AGCCAGAUCGGGGACUUCCCGGACCAGACCGAGGACGGCGGGCUGCCCCAGUCCGGCCGC
AUCGUGGUCGACUACAUGAUGCAGAAGCCCGGGAAGACGGGCACCAUCGUGUACCAGCGG
GGGGUCCUGCUGCCGCAGAAGGUGUGGUGCGCCAGCGGCCGCUCCAAGGUCAUCAAGGGG
AGCCUGCCCCUCAUCGGCGAGGCCGACUGCCUGCACGAGGAGUACGGGGGCCUCAACAAG
UCCAAGCCGUACUACACGGGGAAGCACGCCAAGGCCAUCGGCAACUGCCCGAUCUGGGUG
AAGACCCCCCUGAAGCUCGCCAACGGGACGAAGUACCGGCCGCCCGCCAAGCUGCUCAAG
GAGCGCGGCUUCUUCGGGGCCAUCGCCGGCUUCCUGGAGGGGGGCUGGGAGGGGAUGAUC
GCCGGCUGGCACGGGUACACCAGCCACGGCGCCCACGGGGUCGCGGUGGCCGCGGACCUC
AAGUCCACGCAGGAGGCCAUCAACAAGAUCACCAAGAACCUGAACAGCCUCUCCGAGCUG
GAGGUCAAGAACCUCCAGCGCCUGAGCGGCGCGAUGGACGAGCUCCACAACGAGAUCCUG
GAGCUCGACGAGAAGGUGGACGACCUGCGGGCCGACACGAUCUCGUCCCAGAUCGAGCUC
GCGGUCCUGCUCAGCAACGAGGGGAUCAUCAACUCGGAGGACGAGCACCUGCUCGCCCUG
GAGCGGAAGCUCAAGAAGAUGCUGGGCCCGUCCGCGGUGGACAUCGGGAACGGCUGCUUC
GAGACCAAGCACAAGUGCAACCAGACGUGCCUCGACCGCAUCGCCGCGGGGACCUUCAAC
GCCGGCGAGUUCAGCCUGCCCACGUUCGACUCGCUCAACAUCACCGCGGCCUCCCUGAAC
GACGACGGGCUCGACAACCACACGAUCCUGCUCUACUACAGCACCGCGGCCUCGUCCCUG
GCGGUCACGCUCAUGCUGGCCAUCUUCAUCGUGUACAUGGUCAGCCGCGACAACGUGUCG
UGCUCCAUCUGCCUCUGAUGACUCGAGGCUCGCUUUCUUGCUGUCCAAUUUCUAUUAAAG
GUUCCUUUGUUCCCUAAGUCCAACUACUAAACUGGGGGAUAUUAUGAAGGGCCUUGAGCA
UCUGGAUUCUGCCUAAUAAAAAACAUUUAUUUUCAUUGCAAAAAAAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAA
>I'-H1N1-HA0-mut-B-120A mRNA
SEQ ID NO: 122
GAGUUAGGAGUUGGUGGCAGUUUCAAUAACAGGUCAUUGCCGAGAAAAGGAUAGCACUAU
AAUAUGAAGGCCAUCCUGGUGGUGAUGCUGUACACCUUCACCACCGCCAACGCCGACACC
CUGUGCAUCGGCUACCACGCCAACAACAGCACCGACACCGUGGACACCGUGCUGGAGAAG
AACGUGACCGUGACCCACAGCGUGAACCUGCUGGAGGACAAGCACAACGGCAAGCUGUGC
AAGCUGCGGGGCGUGGCCCCCCUGCACCUGGGCAAGUGCAACAUCGCCGGCUGGAUCCUG
GGCAACCCCGAGUGCGAGUCCCUGAGCACCGCCCGCUCCUGGAGCUACAUCGUGGAGACC
UCCAACAGCGACAACGGCACCUGCUACCCCGGGGACUUCAUCAACUACGAGGAGCUGCGG
GAGCAGCUGUCCAGCGUGUCCAGCUUCGAGCGCUUCGAGAUCUUCCCCAAGACCUCCAGC
UGGCCCAACCACGACUCCGACAACGGCGUGACCGCCGCCUGCCCCCACGCCGGGGCCAAG
AGCUUCUACAAGAACCUGAUCUGGCUGGUGAAGAAGGGCAAGUCCUACCCCAAGAUCAAC
CAGACCUACAUCAACGACAAGGGGAAGGAGGUCCUCGUGCUGUGGGGCAUCCACCACCCC
CCCACCAUCGCCGACCAGCAGAGCCUCUACCAGAACGCCGACGCCUACGUCUUCGUGGGG
ACCUCCCGGUACAGCAAGAAGUUCAAGCCCGAGAUCGCCACCCGCCCCAAGGUCCGGGAC
CAGGAGGGCCGCAUGAACUACUACUGGACCCUGGUGGAGCCGGGGGACAAGAUCACCUUC
GAGGCCACCGGCAACCUCGUCGCCCCGCGGUACGCCUUCACGAUGGAGCGCGACGCCGGG
UCCGGCAUCAUCAUCAGCGACACCCCCGUGCACGACUGCAACACGACCUGCCAGACGCCC
GAGGGGGCGAUCAACACCUCCCUGCCGUUCCAGAACGUCCACCCGAUCACGAUCGGCAAG
UGCCCCAAGUACGUGAAGUCCACCAAGCUCCGGCUGGCCACGGGGCUCCGCAACGUCCCC
UCCAUCCAGAGCCAGGGCCUGUUCGGGGCGAUCGCCGGCUUCAUCGAGGGGGGCUGGACC
GGGAUGGUGGACGGCUGGUACGGGUACCACCACCAGAACGAGCAGGGCUCCGGGUACGCG
GCCGACCUCAAGUCGACGCAGAACGCGAUCGACAAGAUCACCAACAAGGUCAACAGCGUG
AUCGAGAAGAUGAACACGCAGUUCACCGCCGUCGGCAAGGAGUUCAACCACCUGGAGAAG
CGCAUCGAGAACCUCAACAAGAAGGUGGACGACGGGUUCCUGGACAUCUGGACGUACAAC
GCGGAGCUCCUGGUCCUCCUGGAGAACGAGCGGACCCUCGACUACCACGACUCCAACGUG
AAGAACCUGUACGAGAAGGUCCGCAACCAGCUCAAGAACAACGCCAAGGAGAUCGGCAAC
GGGUGCUUCGAGUUCUACCACAAGUGCGACAACACGUGCAUGGAGUCGGUGAAGAACGGC
ACCUACGACUACCCGAAGUACAGCGAGGAGGCGAAGCUGAACCGGGAGAAGAUCGACGGG
GUCAAGCUCGACUCCACGCGCAUCUACCAGAUCCUGGCCAUCUACUCGACCGUGGCGAGC
UCCCUCGUCCUGGUGGUCUCGCUCGGCGCCAUCAGCUUCUGGAUGUGCUCCAACGGGUCG
CUGCAGUGCCGGAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAA
>I'-H3N2-HA0-mut-B-120A mRNA
SEQ ID NO: 123
GAGUUAGGAGUUGGUGGCAGUUUCAAUAACAGGUCAUUGCCGAGAAAAGGAUAGCACUAU
AAUAUGAAGACCAUCAUCGCCCUGAGCUACAUCCUGUGCCUGGUGUUCGCCCAGAAGAUC
CCCGGCAACGACAACAGCACCGCCACCCUGUGCCUGGGCCACCACGCCGUGCCCAACGGC
ACCAUCGUGAAGACCAUCACCAACGACCGGAUCGAGGUGACCAACGCCACCGAGCUGGUG
CAGAACUCCAGCAUCGGCGAGAUCUGCGACUCCCCCCACCAGAUCCUGGACGGCGGCAAC
UGCACCCUGAUCGACGCCCUGCUGGGCGACCCCCAGUGCGACGGGUUCCAGAACAAGGAG
UGGGACCUGUUCGUGGAGCGGAGCCGCGCCAACUCCAACUGCUACCCCUACGACGUGCCC
GACUACGCCAGCCUGCGGUCCCUGGUGGCCAGCUCCGGCACCCUGGAGUUCAAGAACGAG
AGCUUCAACUGGACCGGGGUCAAGCAGAACGGCACCUCCAGCGCCUGCAUCCGCGGGUCC
AGCUCCAGCUUCUUCUCCCGGCUGAACUGGCUCACCCACCUGAACUACACCUACCCCGCC
CUCAACGUGACCAUGCCCAACAACGAGCAGUUCGACAAGCUGUACAUCUGGGGCGUCCAC
CACCCCAGCACCGACAAGGACCAGAUCUCCCUCUUCGCCCAGCCCAGCGGGCGCAUCACC
GUGUCCACGAAGCGGAGCCAGCAGGCCGUCAUCCCCAACAUCGGCUCCCGCCCGCGGAUC
CGCGACAUCCCCAGCCGGAUCUCCAUCUACUGGACGAUCGUGAAGCCGGGGGACAUCCUG
CUCAUCAACAGCACCGGCAACCUGAUCGCCCCCCGCGGGUACUUCAAGAUCCGGUCCGGC
AAGAGCUCGAUCAUGCGCUCCGACGCCCCGAUCGGGAAGUGCAAGAGCGAGUGCAUCACC
CCCAACGGCUCGAUCCCGAACGACAAGCCCUUCCAGAACGUCAACCGGAUCACGUACGGG
GCCUGCCCGCGCUACGUGAAGCAGUCCACGCUCAAGCUGGCCACCGGCAUGCGGAACGUC
CCCGAGAAGCAGACCCAGGGGAUCUUCGGCGCCAUCGCGGGGUUCAUCGAGAACGGCUGG
GAGGGGAUGGUGGACGGCUGGUACGGGUUCCGGCACCAGAACAGCGAGGGCCGCGGGCAG
GCCGCGGACCUCAAGUCGACGCAGGCCGCGAUCGACCAGAUCAACGGCAAGCUGAACCGG
CUCAUCGGGAAGACGAACGAGAAGUUCCACCAGAUCGAGAAGGAGUUCUCCGAGGUCGAG
GGCCGCGUGCAGGACCUGGAGAAGUACGUCGAGGACACCAAGAUCGACCUCUGGAGCUAC
AACGCCGAGCUGCUCGUGGCGCUGGAGAACCAGCACACCAUCGACCUCACGGACUCGGAG
AUGAACAAGCUGUUCGAGAAGACGAAGAAGCAGCUCCGGGAGAACGCCGAGGACAUGGGG
AACGGCUGCUUCAAGAUCUACCACAAGUGCGACAACGCGUGCAUCGGGUCCAUCCGCAAC
GAGACCUACGACCACAACGUCUACCGGGACGAGGCCCUGAACAACCGCUUCCAGAUCAAG
GGCGUGGAGCUCAAGAGCGGGUACAAGGACUGGAUCCUGUGGAUCUCGUUCGCGAUGUCC
UGCUUCCUCCUGUGCAUCGCCCUCCUGGGCUUCAUCAUGUGGGCGUGCCAGAAGGGGAAC
AUCCGGUGCAACAUCUGCAUCUGAUGACUCGAGGCUCGCUUUCUUGCUGUCCAAUUUCUA
UUAAAGGUUCCUUUGUUCCCUAAGUCCAACUACUAAACUGGGGGAUAUUAUGAAGGGCCU
UGAGCAUCUGGAUUCUGCCUAAUAAAAAACAUUUAUUUUCAUUGCAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAA
>I'-B-Victoria-HA0-mut-B-120A mRNA
SEQ ID NO: 124
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCCACUUCGCCAACCUG
AAGGGCACCGAGACCCGGGGCAAGCUGUGCCCCAAGUGCCUGAACUGCACCGACCUGGAC
GUGGCCCUGGGCCGCCCCAAGUGCACCGGCAAGAUCCCCAGCGCCCGGGUGUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGCACGUCCGCCUGUCCACCCACAACGUG
AUCAACGCCGAGGACGCCCCCGGCCGGCCCUACGAGAUCGGGACCAGCGGCUCCUGCCCC
AACAUCACCAACGGGAACGGCUUCUUCGCCACCAUGGCCUGGGCCGUCCCCAAGAACAAG
ACCGCCACCAACCCCCUGACCAUCGAGGUGCCGUACAUCUGCACCGAGGGGGAGGACCAG
AUCACCGUCUGGGGCUUCCACAGCGACAACGAGACGCAGAUGGCCAAGCUGUACGGGGAC
UCCAAGCCCCAGAAGUUCACGAGCUCCGCCAACGGCGUGACCACCCACUACGUCAGCCAG
AUCGGGGGCUUCCCGAACCAGACGGAGGACGGGGGCCUGCCCCAGUCCGGGCGCAUCGUG
GUCGACUACAUGGUGCAGAAGUCCGGCAAGACGGGGACCAUCACGUACCAGCGGGGCAUC
CUGCUGCCGCAGAAGGUCUGGUGCGCCUCCGGGCGCAGCAAGGUGAUCAAGGGCUCCCUG
CCCCUCAUCGGGGAGGCCGACUGCCUGCACGAGAAGUACGGCGGGCUCAACAAGAGCAAG
CCGUACUACACCGGCGAGCACGCCAAGGCCAUCGGGAACUGCCCCAUCUGGGUCAAGACC
CCGCUGAAGCUCGCCAACGGCACGAAGUACCGGCCCCCGGCCAAGCUGCUCAAGGAGCAG
GGGUUCUUCGGCGCCAUCGCCGGGUUCCUGGAGGGCGGGUGGGAGGGCAUGAUCGCCGGG
UGGCACGGCUACACGUCCCACGGGGCCCACGGCGUGGCGGUCGCCGCGGACCUCAAGAGC
ACCCAGGAGGCCAUCAACAAGAUCACGAAGAACCUGAACUCCCUCAGCGAGCUGGAGGUG
AAGAACCUCCAGCGCCUGUCCGGGGCGAUGGACGAGCUCCACAACGAGAUCCUGGAGCUC
GACGAGAAGGUCGACGACCUGCGGGCCGACACCAUCAGCUCGCAGAUCGAGCUCGCGGUG
CUGCUCUCCAACGAGGGCAUCAUCAACAGCGAGGACGAGCACCUGCUCGCCCUGGAGCGG
AAGCUCAAGAAGAUGCUGGGGCCCUCGGCGGUCGAGAUCGGCAACGGGUGCUUCGAGACC
AAGCACAAGUGCAACCAGACGUGCCUCGACCGCAUCGCCGCGGGCACGUUCGACGCCGGG
GAGUUCUCCCUGCCGACCUUCGACAGCCUCAACAUCACGGCGGCCUCGCUGAACGACGAC
GGCCUCGACAACCACACCAUCCUGCUCUACUACUCCACCGCGGCCAGCUCGCUGGCGGUG
ACGCUCAUGAUCGCCAUCUUCGUCGUGUACAUGGUCUCCCGCGACAACGUGAGCUGCUCG
AUCUGCCUGUGAUGACUCGAGGCUCGCUUUCUUGCUGUCCAAUUUCUAUUAAAGGUUCCU
UUGUUCCCUAAGUCCAACUACUAAACUGGGGGAUAUUAUGAAGGGCCUUGAGCAUCUGGA
UUCUGCCUAAUAAAAAACAUUUAUUUUCAUUGCAAAAAAAAAAAAAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAA
>I'-B-Yamagata-HA0-mut-B-120A mRNA
SEQ ID NO: 125
GAGUUAGGAGUUGGUGGCAGUUUCAAUAACAGGUCAUUGCCGAGAAAAGGAUAGCACUAU
AAUAUGAAGGCCAUCAUCGUGCUGCUGAUGGUGGUGACCAGCAACGCCGACCGGAUCUGC
ACCGGCAUCACCAGCUCCAACAGCCCCCACGUGGUGAAGACCGCCACCCAGGGCGAGGUG
AACGUGACCGGCGUGAUCCCCCUGACCACCACCCCCACCAAGUCCUACUUCGCCAACCUG
AAGGGCACCCGGACCCGCGGCAAGCUGUGCCCCGACUGCCUGAACUGCACCGACCUGGAC
GUGGCCCUGGGCCGGCCCAUGUGCGUGGGCACCACCCCCAGCGCCAAGGCCUCCAUCCUG
CACGAGGUGCGCCCCGUGACCAGCGGCUGCUUCCCCAUCAUGCACGACCGCACCAAGAUC
CGGCAGCUGCCCAACCUGCUGCGGGGGUACGAGAAGAUCCGCCUGUCCACCCAGAACGUC
AUCGACGCCGAGAAGGCCCCCGGCGGGCCCUACCGGCUGGGCACCAGCGGGUCCUGCCCC
AACGCCACCAGCAAGAUCGGCUUCUUCGCCACCAUGGCCUGGGCCGUGCCCAAGGACAAC
UACAAGAACGCCACCAACCCCCUGACCGUCGAGGUGCCCUACAUCUGCACCGAGGGGGAG
GACCAGAUCACGGUCUGGGGCUUCCACUCCGACAACAAGACCCAGAUGAAGUCCCUGUAC
GGGGACUCCAACCCGCAGAAGUUCACGAGCUCCGCCAACGGCGUGACCACGCACUACGUC
AGCCAGAUCGGGGACUUCCCGGACCAGACCGAGGACGGCGGGCUGCCCCAGUCCGGCCGC
AUCGUGGUCGACUACAUGAUGCAGAAGCCCGGGAAGACGGGCACCAUCGUGUACCAGCGG
GGGGUCCUGCUGCCGCAGAAGGUGUGGUGCGCCAGCGGCCGCUCCAAGGUCAUCAAGGGG
AGCCUGCCCCUCAUCGGCGAGGCCGACUGCCUGCACGAGGAGUACGGGGGCCUCAACAAG
UCCAAGCCGUACUACACGGGGAAGCACGCCAAGGCCAUCGGCAACUGCCCGAUCUGGGUG
AAGACCCCCCUGAAGCUCGCCAACGGGACGAAGUACCGGCCGCCCGCCAAGCUGCUCAAG
GAGCAGGGCUUCUUCGGGGCCAUCGCCGGCUUCCUGGAGGGGGGCUGGGAGGGGAUGAUC
GCCGGCUGGCACGGGUACACCAGCCACGGCGCCCACGGGGUCGCGGUGGCCGCGGACCUC
AAGUCCACGCAGGAGGCCAUCAACAAGAUCACCAAGAACCUGAACAGCCUCUCCGAGCUG
GAGGUCAAGAACCUCCAGCGCCUGAGCGGCGCGAUGGACGAGCUCCACAACGAGAUCCUG
GAGCUCGACGAGAAGGUGGACGACCUGCGGGCCGACACGAUCUCGUCCCAGAUCGAGCUC
GCGGUCCUGCUCAGCAACGAGGGGAUCAUCAACUCGGAGGACGAGCACCUGCUCGCCCUG
GAGCGGAAGCUCAAGAAGAUGCUGGGCCCGUCCGCGGUGGACAUCGGGAACGGCUGCUUC
GAGACCAAGCACAAGUGCAACCAGACGUGCCUCGACCGCAUCGCCGCGGGGACCUUCAAC
GCCGGCGAGUUCAGCCUGCCCACGUUCGACUCGCUCAACAUCACCGCGGCCUCCCUGAAC
GACGACGGGCUCGACAACCACACGAUCCUGCUCUACUACAGCACCGCGGCCUCGUCCCUG
GCGGUCACGCUCAUGCUGGCCAUCUUCAUCGUGUACAUGGUCAGCCGCGACAACGUGUCG
UGCUCCAUCUGCCUCUGAUGACUCGAGGCUCGCUUUCUUGCUGUCCAAUUUCUAUUAAAG
GUUCCUUUGUUCCCUAAGUCCAACUACUAAACUGGGGGAUAUUAUGAAGGGCCUUGAGCA
UCUGGAUUCUGCCUAAUAAAAAACAUUUAUUUUCAUUGCAAAAAAAAAAAAAAAAAAAAA
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAA
>Influenza A virus H1N1 (A/Wisconsin/588/2019)
HA mRNA
SEQ ID NO: 126
AUGAAGGCCAUCCUGGUGGUGAUGCUGUACACCUUCACCACCGCCAACGCCGACACCCUG
UGCAUCGGCUACCACGCCAACAACAGCACCGACACCGUGGACACCGUGCUGGAGAAGAAC
GUGACCGUGACCCACAGCGUGAACCUGCUGGAGGACAAGCACAACGGCAAGCUGUGCAAG
CUGCGGGGCGUGGCCCCCCUGCACCUGGGCAAGUGCAACAUCGCCGGCUGGAUCCUGGGC
AACCCCGAGUGCGAGUCCCUGAGCACCGCCCGCUCCUGGAGCUACAUCGUGGAGACCUCC
AACAGCGACAACGGCACCUGCUACCCCGGGGACUUCAUCAACUACGAGGAGCUGCGGGAG
CAGCUGUCCAGCGUGUCCAGCUUCGAGCGCUUCGAGAUCUUCCCCAAGACCUCCAGCUGG
CCCAACCACGACUCCGACAACGGCGUGACCGCCGCCUGCCCCCACGCCGGGGCCAAGAGC
UUCUACAAGAACCUGAUCUGGCUGGUGAAGAAGGGCAAGUCCUACCCCAAGAUCAACCAG
ACCUACAUCAACGACAAGGGGAAGGAGGUCCUCGUGCUGUGGGGCAUCCACCACCCCCCC
ACCAUCGCCGACCAGCAGAGCCUCUACCAGAACGCCGACGCCUACGUCUUCGUGGGGACC
UCCCGGUACAGCAAGAAGUUCAAGCCCGAGAUCGCCACCCGCCCCAAGGUCCGGGACCAG
GAGGGCCGCAUGAACUACUACUGGACCCUGGUGGAGCCGGGGGACAAGAUCACCUUCGAG
GCCACCGGCAACCUCGUCGCCCCGCGGUACGCCUUCACGAUGGAGCGCGACGCCGGGUCC
GGCAUCAUCAUCAGCGACACCCCCGUGCACGACUGCAACACGACCUGCCAGACGCCCGAG
GGGGCGAUCAACACCUCCCUGCCGUUCCAGAACGUCCACCCGAUCACGAUCGGCAAGUGC
CCCAAGUACGUGAAGUCCACCAAGCUCCGGCUGGCCACGGGGCUCCGCAACGUCCCCUCC
AUCCAGAGCCGGGGCCUGUUCGGGGCGAUCGCCGGCUUCAUCGAGGGGGGCUGGACCGGG
AUGGUGGACGGCUGGUACGGGUACCACCACCAGAACGAGCAGGGCUCCGGGUACGCGGCC
GACCUCAAGUCGACGCAGAACGCGAUCGACAAGAUCACCAACAAGGUCAACAGCGUGAUC
GAGAAGAUGAACACGCAGUUCACCGCCGUCGGCAAGGAGUUCAACCACCUGGAGAAGCGC
AUCGAGAACCUCAACAAGAAGGUGGACGACGGGUUCCUGGACAUCUGGACGUACAACGCG
GAGCUCCUGGUCCUCCUGGAGAACGAGCGGACCCUCGACUACCACGACUCCAACGUGAAG
AACCUGUACGAGAAGGUCCGCAACCAGCUCAAGAACAACGCCAAGGAGAUCGGCAACGGG
UGCUUCGAGUUCUACCACAAGUGCGACAACACGUGCAUGGAGUCGGUGAAGAACGGCACC
UACGACUACCCGAAGUACAGCGAGGAGGCGAAGCUGAACCGGGAGAAGAUCGACGGGGUC
AAGCUCGACUCCACGCGCAUCUACCAGAUCCUGGCCAUCUACUCGACCGUGGCGAGCUCC
CUCGUCCUGGUGGUCUCGCUCGGCGCCAUCAGCUUCUGGAUGUGCUCCAACGGGUCGCUG
CAGUGCCGGAUCUGCAUCUGA
>Influenza A virus H3N2 (A/Cambodia/e0826360/2020)
HA mRNA
SEQ ID NO: 127
AUGAAGACCAUCAUCGCCCUGAGCUACAUCCUGUGCCUGGUGUUCGCCCAGAAGAUCCCC
GGCAACGACAACAGCACCGCCACCCUGUGCCUGGGCCACCACGCCGUGCCCAACGGCACC
AUCGUGAAGACCAUCACCAACGACCGGAUCGAGGUGACCAACGCCACCGAGCUGGUGCAG
AACUCCAGCAUCGGCGAGAUCUGCGACUCCCCCCACCAGAUCCUGGACGGCGGCAACUGC
ACCCUGAUCGACGCCCUGCUGGGCGACCCCCAGUGCGACGGGUUCCAGAACAAGGAGUGG
GACCUGUUCGUGGAGCGGAGCCGCGCCAACUCCAACUGCUACCCCUACGACGUGCCCGAC
UACGCCAGCCUGCGGUCCCUGGUGGCCAGCUCCGGCACCCUGGAGUUCAAGAACGAGAGC
UUCAACUGGACCGGGGUCAAGCAGAACGGCACCUCCAGCGCCUGCAUCCGCGGGUCCAGC
UCCAGCUUCUUCUCCCGGCUGAACUGGCUCACCCACCUGAACUACACCUACCCCGCCCUC
AACGUGACCAUGCCCAACAACGAGCAGUUCGACAAGCUGUACAUCUGGGGCGUCCACCAC
CCCAGCACCGACAAGGACCAGAUCUCCCUCUUCGCCCAGCCCAGCGGGCGCAUCACCGUG
UCCACGAAGCGGAGCCAGCAGGCCGUCAUCCCCAACAUCGGCUCCCGCCCGCGGAUCCGC
GACAUCCCCAGCCGGAUCUCCAUCUACUGGACGAUCGUGAAGCCGGGGGACAUCCUGCUC
AUCAACAGCACCGGCAACCUGAUCGCCCCCCGCGGGUACUUCAAGAUCCGGUCCGGCAAG
AGCUCGAUCAUGCGCUCCGACGCCCCGAUCGGGAAGUGCAAGAGCGAGUGCAUCACCCCC
AACGGCUCGAUCCCGAACGACAAGCCCUUCCAGAACGUCAACCGGAUCACGUACGGGGCC
UGCCCGCGCUACGUGAAGCAGUCCACGCUCAAGCUGGCCACCGGCAUGCGGAACGUCCCC
GAGAAGCAGACCCGCGGGAUCUUCGGCGCCAUCGCGGGGUUCAUCGAGAACGGCUGGGAG
GGGAUGGUGGACGGCUGGUACGGGUUCCGGCACCAGAACAGCGAGGGCCGCGGGCAGGCC
GCGGACCUCAAGUCGACGCAGGCCGCGAUCGACCAGAUCAACGGCAAGCUGAACCGGCUC
AUCGGGAAGACGAACGAGAAGUUCCACCAGAUCGAGAAGGAGUUCUCCGAGGUCGAGGGC
CGCGUGCAGGACCUGGAGAAGUACGUCGAGGACACCAAGAUCGACCUCUGGAGCUACAAC
GCCGAGCUGCUCGUGGCGCUGGAGAACCAGCACACCAUCGACCUCACGGACUCGGAGAUG
AACAAGCUGUUCGAGAAGACGAAGAAGCAGCUCCGGGAGAACGCCGAGGACAUGGGGAAC
GGCUGCUUCAAGAUCUACCACAAGUGCGACAACGCGUGCAUCGGGUCCAUCCGCAACGAG
ACCUACGACCACAACGUCUACCGGGACGAGGCCCUGAACAACCGCUUCCAGAUCAAGGGC
GUGGAGCUCAAGAGCGGGUACAAGGACUGGAUCCUGUGGAUCUCGUUCGCGAUGUCCUGC
UUCCUCCUGUGCAUCGCCCUCCUGGGCUUCAUCAUGUGGGCGUGCCAGAAGGGGAACAUC
CGGUGCAACAUCUGCAUCUGA
>Influenza B virus Victoria lineage (B/Washington/02/2019)
HA mRNA
SEQ ID NO: 128
AUGAAGGCCAUCAUCGUGCUGCUGAUGGUGGUGACCAGCAACGCCGACCGGAUCUGCACC
GGCAUCACCAGCUCCAACAGCCCCCACGUGGUGAAGACCGCCACCCAGGGCGAGGUGAAC
GUGACCGGCGUGAUCCCCCUGACCACCACCCCCACCAAGUCCCACUUCGCCAACCUGAAG
GGCACCGAGACCCGGGGCAAGCUGUGCCCCAAGUGCCUGAACUGCACCGACCUGGACGUG
GCCCUGGGCCGCCCCAAGUGCACCGGCAAGAUCCCCAGCGCCCGGGUGUCCAUCCUGCAC
GAGGUGCGCCCCGUGACCAGCGGCUGCUUCCCCAUCAUGCACGACCGCACCAAGAUCCGG
CAGCUGCCCAACCUGCUGCGGGGGUACGAGCACGUCCGCCUGUCCACCCACAACGUGAUC
AACGCCGAGGACGCCCCCGGCCGGCCCUACGAGAUCGGGACCAGCGGCUCCUGCCCCAAC
AUCACCAACGGGAACGGCUUCUUCGCCACCAUGGCCUGGGCCGUCCCCAAGAACAAGACC
GCCACCAACCCCCUGACCAUCGAGGUGCCGUACAUCUGCACCGAGGGGGAGGACCAGAUC
ACCGUCUGGGGCUUCCACAGCGACAACGAGACGCAGAUGGCCAAGCUGUACGGGGACUCC
AAGCCCCAGAAGUUCACGAGCUCCGCCAACGGCGUGACCACCCACUACGUCAGCCAGAUC
GGGGGCUUCCCGAACCAGACGGAGGACGGGGGCCUGCCCCAGUCCGGGCGCAUCGUGGUC
GACUACAUGGUGCAGAAGUCCGGCAAGACGGGGACCAUCACGUACCAGCGGGGCAUCCUG
CUGCCGCAGAAGGUCUGGUGCGCCUCCGGGCGCAGCAAGGUGAUCAAGGGCUCCCUGCCC
CUCAUCGGGGAGGCCGACUGCCUGCACGAGAAGUACGGCGGGCUCAACAAGAGCAAGCCG
UACUACACCGGCGAGCACGCCAAGGCCAUCGGGAACUGCCCCAUCUGGGUCAAGACCCCG
CUGAAGCUCGCCAACGGCACGAAGUACCGGCCCCCGGCCAAGCUGCUCAAGGAGCGCGGG
UUCUUCGGCGCCAUCGCCGGGUUCCUGGAGGGCGGGUGGGAGGGCAUGAUCGCCGGGUGG
CACGGCUACACGUCCCACGGGGCCCACGGCGUGGCGGUCGCCGCGGACCUCAAGAGCACC
CAGGAGGCCAUCAACAAGAUCACGAAGAACCUGAACUCCCUCAGCGAGCUGGAGGUGAAG
AACCUCCAGCGCCUGUCCGGGGCGAUGGACGAGCUCCACAACGAGAUCCUGGAGCUCGAC
GAGAAGGUCGACGACCUGCGGGCCGACACCAUCAGCUCGCAGAUCGAGCUCGCGGUGCUG
CUCUCCAACGAGGGCAUCAUCAACAGCGAGGACGAGCACCUGCUCGCCCUGGAGCGGAAG
CUCAAGAAGAUGCUGGGGCCCUCGGCGGUCGAGAUCGGCAACGGGUGCUUCGAGACCAAG
CACAAGUGCAACCAGACGUGCCUCGACCGCAUCGCCGCGGGCACGUUCGACGCCGGGGAG
UUCUCCCUGCCGACCUUCGACAGCCUCAACAUCACGGCGGCCUCGCUGAACGACGACGGC
CUCGACAACCACACCAUCCUGCUCUACUACUCCACCGCGGCCAGCUCGCUGGCGGUGACG
CUCAUGAUCGCCAUCUUCGUCGUGUACAUGGUCUCCCGCGACAACGUGAGCUGCUCGAUC
UGCCUGUGA
>Influenza B virus /Yamagata lineage (B/Phuket/3073/2013)
HA mRNA
SEQ ID NO: 129
AUGAAGGCCAUCAUCGUGCUGCUGAUGGUGGUGACCAGCAACGCCGACCGGAUCUGCACC
GGCAUCACCAGCUCCAACAGCCCCCACGUGGUGAAGACCGCCACCCAGGGCGAGGUGAAC
GUGACCGGCGUGAUCCCCCUGACCACCACCCCCACCAAGUCCUACUUCGCCAACCUGAAG
GGCACCCGGACCCGCGGCAAGCUGUGCCCCGACUGCCUGAACUGCACCGACCUGGACGUG
GCCCUGGGCCGGCCCAUGUGCGUGGGCACCACCCCCAGCGCCAAGGCCUCCAUCCUGCAC
GAGGUGCGCCCCGUGACCAGCGGCUGCUUCCCCAUCAUGCACGACCGCACCAAGAUCCGG
CAGCUGCCCAACCUGCUGCGGGGGUACGAGAAGAUCCGCCUGUCCACCCAGAACGUCAUC
GACGCCGAGAAGGCCCCCGGCGGGCCCUACCGGCUGGGCACCAGCGGGUCCUGCCCCAAC
GCCACCAGCAAGAUCGGCUUCUUCGCCACCAUGGCCUGGGCCGUGCCCAAGGACAACUAC
AAGAACGCCACCAACCCCCUGACCGUCGAGGUGCCCUACAUCUGCACCGAGGGGGAGGAC
CAGAUCACGGUCUGGGGCUUCCACUCCGACAACAAGACCCAGAUGAAGUCCCUGUACGGG
GACUCCAACCCGCAGAAGUUCACGAGCUCCGCCAACGGCGUGACCACGCACUACGUCAGC
CAGAUCGGGGACUUCCCGGACCAGACCGAGGACGGCGGGCUGCCCCAGUCCGGCCGCAUC
GUGGUCGACUACAUGAUGCAGAAGCCCGGGAAGACGGGCACCAUCGUGUACCAGCGGGGG
GUCCUGCUGCCGCAGAAGGUGUGGUGCGCCAGCGGCCGCUCCAAGGUCAUCAAGGGGAGC
CUGCCCCUCAUCGGCGAGGCCGACUGCCUGCACGAGGAGUACGGGGGCCUCAACAAGUCC
AAGCCGUACUACACGGGGAAGCACGCCAAGGCCAUCGGCAACUGCCCGAUCUGGGUGAAG
ACCCCCCUGAAGCUCGCCAACGGGACGAAGUACCGGCCGCCCGCCAAGCUGCUCAAGGAG
CGCGGCUUCUUCGGGGCCAUCGCCGGCUUCCUGGAGGGGGGCUGGGAGGGGAUGAUCGCC
GGCUGGCACGGGUACACCAGCCACGGCGCCCACGGGGUCGCGGUGGCCGCGGACCUCAAG
UCCACGCAGGAGGCCAUCAACAAGAUCACCAAGAACCUGAACAGCCUCUCCGAGCUGGAG
GUCAAGAACCUCCAGCGCCUGAGCGGCGCGAUGGACGAGCUCCACAACGAGAUCCUGGAG
CUCGACGAGAAGGUGGACGACCUGCGGGCCGACACGAUCUCGUCCCAGAUCGAGCUCGCG
GUCCUGCUCAGCAACGAGGGGAUCAUCAACUCGGAGGACGAGCACCUGCUCGCCCUGGAG
CGGAAGCUCAAGAAGAUGCUGGGCCCGUCCGCGGUGGACAUCGGGAACGGCUGCUUCGAG
ACCAAGCACAAGUGCAACCAGACGUGCCUCGACCGCAUCGCCGCGGGGACCUUCAACGCC
GGCGAGUUCAGCCUGCCCACGUUCGACUCGCUCAACAUCACCGCGGCCUCCCUGAACGAC
GACGGGCUCGACAACCACACGAUCCUGCUCUACUACAGCACCGCGGCCUCGUCCCUGGCG
GUCACGCUCAUGCUGGCCAUCUUCAUCGUGUACAUGGUCAGCCGCGACAACGUGUCGUGC
UCCAUCUGCCUCUGA
>SARS-CoV-2 BA.4 spike protein mRNA
SEQ ID NO: 130
AUGUUCGUGUUCCUGGUGCUGCUGCCCCUGGUGAGCUCUCAGUGCGUGAACCUGAUCACA
AGAACACAGUCGUACACCAACAGCUUCACAAGAGGCGUGUACUACCCCGACAAGGUGUUC
AGAAGCAGCGUGCUGCACUCCACCCAAGACCUGUUUCUGCCCUUCUUCAGCAACGUGACC
UGGUUCCACGCCAUCAGCGGCACCAACGGCACCAAGAGAUUCGACAACCCCGUGCUGCCC
UUCAACGACGGCGUGUACUUCGCUAGCACCGAAAAGAGCAACAUCAUCAGAGGCUGGAUC
UUCGGCACCACCCUAGACUCCAAGACACAAAGCCUGCUGAUCGUUAACAACGCGACCAAC
GUGGUGAUCAAGGUGUGCGAGUUUCAGUUCUGCAACGACCCCUUCCUGGACGUGUACUAC
CACAAGAACAACAAGAGCUGGAUGGAGAGCGAGUUCAGAGUCUACAGCAGCGCCAACAAC
UGCACCUUCGAGUACGUGUCUCAGCCCUUCCUGAUGGACCUGGAGGGCAAGCAAGGCAAU
UUCAAGAACCUAAGAGAGUUCGUGUUCAAGAACAUCGACGGCUACUUCAAGAUCUACAGC
AAGCACACCCCCAUCAACCUGGGGAGAGACCUGCCCCAAGGCUUCAGCGCCCUGGAGCCC
CUGGUGGACCUGCCCAUCGGCAUCAACAUCACAAGAUUUCAGACCCUGCUGGCCCUGCAC
CGUAGCUACCUAACCCCCGGCGAUAGUUCCUCUGGCUGGACGGCCGGCGCUGCCGCCUAC
UACGUGGGCUACCUGCAGCCUAGAACCUUCCUGCUGAAGUACAACGAGAACGGCACUAUC
ACUGAUGCCGUGGACUGUGCCCUGGACCCCCUGAGCGAGACCAAGUGCACCCUCAAGAGC
UUCACCGUGGAGAAGGGCAUCUAUCAGACAAGCAACUUCAGAGUGCAGCCCACCGAGAGC
AUCGUGAGAUUCCCCAACAUCACCAACCUGUGCCCCUUCGACGAGGUGUUCAACGCCACA
AGAUUCGCUAGCGUGUACGCGUGGAACAGAAAGAGAAUCAGCAACUGCGUGGCCGACUAC
AGCGUGCUGUACAACUUCGCUCCCUUCUUCGCCUUCAAGUGCUAUGGCGUCAGCCCCACC
AAGCUGAACGACCUGUGCUUCACCAACGUGUACGCCGACAGCUUCGUGAUCAGAGGCAAC
GAGGUGAGCCAGAUUGCCCCCGGGCAAACCGGCAACAUCGCCGACUACAACUACAAGCUG
CCCGACGACUUCACCGGCUGCGUGAUCGCGUGGAACAGCAAUAAGCUGGAUUCCAAGGUG
GGCGGCAACUACAACUACCGGUACAGACUGUUCAGAAAGAGCAACCUGAAGCCCUUCGAG
AGAGACAUCAGCACCGAGAUCUACCAAGCCGGCAACAAGCCCUGCAACGGCGUGGCGGGC
GUCAACUGCUACUUCCCCCUGCAGAGCUACGGCUUUAGGCCCACCUACGGCGUGGGCCAU
CAGCCCUACAGAGUGGUGGUGCUGAGCUUCGAGCUGCUGCACGCCCCCGCAACCGUGUGC
GGCCCCAAAAAGAGCACCAACCUGGUGAAGAACAAGUGCGUGAACUUCAACUUUAAUGGC
CUUAAGGGCACGGGCGUGCUGACCGAGAGCAACAAGAAGUUCCUGCCAUUUCAGCAAUUC
GGCAGAGACAUCGCCGACACCACCGACGCGGUCAGAGACCCUCAGACCCUGGAGAUCCUG
GACAUCACCCCCUGUAGCUUCGGCGGCGUGAGCGUGAUCACCCCCGGCACUAACACAAGC
AACCAAGUGGCCGUGCUGUACCAAGGCGUGAACUGCACCGAGGUGCCCGUGGCCAUCCAC
GCCGAUCAGCUGACCCCCACCUGGAGAGUAUACAGCACCGGAAGCAACGUGUUUCAGACA
AGAGCCGGCUGCCUGAUCGGCGCCGAGUACGUGAACAACAGCUACGAGUGCGACAUCCCC
AUCGGCGCCGGCAUCUGCGCUAGCUAUCAGACACAGACCAAGAGCCACCGGAGAGCUAGA
AGCGUGGCUUCUCAGAGCAUCAUCGCCUACACCAUGAGCCUGGGCGCCGAGAACAGCGUG
GCCUACAGCAACAACAGCAUCGCCAUCCCCACCAACUUCACCAUCAGCGUGACCACCGAG
AUCCUGCCCGUAAGUAUGACCAAGACAAGCGUGGACUGCACCAUGUACAUCUGCGGCGAC
AGCACCGAGUGCAGCAACCUGCUGCUGCAGUACGGCAGCUUCUGCACACAGCUGAAGAGA
GCCCUGACCGGCAUCGCCGUGGAGCAAGACAAGAACACCCAAGAGGUGUUCGCCCAAGUG
AAGCAGAUCUACAAGACCCCCCCCAUCAAGUACUUCGGCGGCUUCAACUUCAGCCAAAUC
CUGCCCGACCCUAGCAAGCCUAGCAAGCGGAGCUUCAUCGAGGACCUGCUGUUCAACAAG
GUGACCCUGGCCGACGCCGGCUUCAUCAAGCAGUACGGCGACUGCCUAGGCGACAUCGCA
GCUAGAGACCUGAUCUGCGCUCAGAAGUUCAAUGGACUCACCGUGCUCCCCCCCCUGCUG
ACCGACGAGAUGAUCGCUCAGUACACCUCUGCCCUGCUGGCGGGAACCAUAACAAGCGGU
UGGACAUUUGGCGCCGGGGCCGCACUGCAAAUCCCCUUCGCCAUGCAGAUGGCCUACAGA
UUCAACGGCAUCGGCGUGACACAGAACGUGCUGUACGAGAAUCAGAAGCUGAUCGCCAAU
CAGUUCAACAGCGCCAUCGGCAAGAUCCAAGACAGCCUGAGCAGCACCGCUAGCGCCCUG
GGCAAGCUGCAAGACGUGGUGAAUCACAACGCCCAAGCCCUGAACACCCUGGUGAAGCAG
CUGAGCAGCAAGUUCGGCGCCAUCAGCAGCGUAUUGAAUGACAUCCUGUCUAGACUGGAC
CCCCCCGAGGCCGAGGUGCAGAUCGACAGACUGAUCACCGGCAGACUGCAGAGCCUGCAG
ACCUACGUGACACAGCAGCUGAUCAGAGCCGCCGAGAUCAGAGCUAGCGCCAACCUGGCC
GCCACCAAGAUGAGCGAGUGCGUGCUGGGGCAGAGCAAGAGAGUGGACUUCUGCGGCAAG
GGCUACCACCUGAUGAGCUUCCCUCAGAGCGCCCCCCACGGCGUGGUGUUCCUGCACGUG
ACCUACGUGCCCGCCCAAGAGAAGAACUUCACCACCGCUCCCGCCAUCUGCCACGACGGC
AAGGCCCACUUCCCUAGAGAGGGCGUGUUCGUGAGCAACGGCACCCACUGGUUCGUGACA
CAGAGAAACUUCUACGAGCCUCAGAUCAUCACCACCGACAACACCUUCGUGAGCGGCAAC
UGCGACGUGGUGAUCGGCAUAGUGAACAACACCGUAUACGACCCCCUGCAGCCCGAGCUG
GACAGCUUCAAGGAGGAGCUGGACAAGUACUUCAAAAACCAUACUAGCCCCGACGUGGAC
CUGGGAGACAUCUCUGGCAUCAACGCUAGCGUGGUGAACAUUCAGAAGGAGAUCGACCGG
CUGAAUGAGGUGGCCAAGAACCUGAACGAGAGCCUGAUCGACCUGCAAGAGCUGGGCAAG
UACGAGCAGUACAUCAAGUGGCCCUGGUACAUCUGGCUGGGCUUCAUCGCCGGCCUGAUC
GCCAUCGUGAUGGUGACCAUCAUGCUGUGCUGCAUGACAAGCUGUUGCAGCUGCCUGAAA
GGUUGCUGCAGCUGUGGCUCGUGCUGCAAGUUCGACGAGGACGACAGCGAGCCCGUGCUG
AAGGGCGUGAAGCUGCACUACACCUGAUGA
>Influenza A virus H1N1 HAO mutant mRNA
SEQ ID NO: 131
AUGAAGGCCAUCCUGGUGGUGAUGCUGUACACCUUCACCACCGCCAACGCCGACACCCUG
UGCAUCGGCUACCACGCCAACAACAGCACCGACACCGUGGACACCGUGCUGGAGAAGAAC
GUGACCGUGACCCACAGCGUGAACCUGCUGGAGGACAAGCACAACGGCAAGCUGUGCAAG
CUGCGGGGCGUGGCCCCCCUGCACCUGGGCAAGUGCAACAUCGCCGGCUGGAUCCUGGGC
AACCCCGAGUGCGAGUCCCUGAGCACCGCCCGCUCCUGGAGCUACAUCGUGGAGACCUCC
AACAGCGACAACGGCACCUGCUACCCCGGGGACUUCAUCAACUACGAGGAGCUGCGGGAG
CAGCUGUCCAGCGUGUCCAGCUUCGAGCGCUUCGAGAUCUUCCCCAAGACCUCCAGCUGG
CCCAACCACGACUCCGACAACGGCGUGACCGCCGCCUGCCCCCACGCCGGGGCCAAGAGC
UUCUACAAGAACCUGAUCUGGCUGGUGAAGAAGGGCAAGUCCUACCCCAAGAUCAACCAG
ACCUACAUCAACGACAAGGGGAAGGAGGUCCUCGUGCUGUGGGGCAUCCACCACCCCCCC
ACCAUCGCCGACCAGCAGAGCCUCUACCAGAACGCCGACGCCUACGUCUUCGUGGGGACC
UCCCGGUACAGCAAGAAGUUCAAGCCCGAGAUCGCCACCCGCCCCAAGGUCCGGGACCAG
GAGGGCCGCAUGAACUACUACUGGACCCUGGUGGAGCCGGGGGACAAGAUCACCUUCGAG
GCCACCGGCAACCUCGUCGCCCCGCGGUACGCCUUCACGAUGGAGCGCGACGCCGGGUCC
GGCAUCAUCAUCAGCGACACCCCCGUGCACGACUGCAACACGACCUGCCAGACGCCCGAG
GGGGCGAUCAACACCUCCCUGCCGUUCCAGAACGUCCACCCGAUCACGAUCGGCAAGUGC
CCCAAGUACGUGAAGUCCACCAAGCUCCGGCUGGCCACGGGGCUCCGCAACGUCCCCUCC
AUCCAGAGCCAGGGCCUGUUCGGGGCGAUCGCCGGCUUCAUCGAGGGGGGCUGGACCGGG
AUGGUGGACGGCUGGUACGGGUACCACCACCAGAACGAGCAGGGCUCCGGGUACGCGGCC
GACCUCAAGUCGACGCAGAACGCGAUCGACAAGAUCACCAACAAGGUCAACAGCGUGAUC
GAGAAGAUGAACACGCAGUUCACCGCCGUCGGCAAGGAGUUCAACCACCUGGAGAAGCGC
AUCGAGAACCUCAACAAGAAGGUGGACGACGGGUUCCUGGACAUCUGGACGUACAACGCG
GAGCUCCUGGUCCUCCUGGAGAACGAGCGGACCCUCGACUACCACGACUCCAACGUGAAG
AACCUGUACGAGAAGGUCCGCAACCAGCUCAAGAACAACGCCAAGGAGAUCGGCAACGGG
UGCUUCGAGUUCUACCACAAGUGCGACAACACGUGCAUGGAGUCGGUGAAGAACGGCACC
UACGACUACCCGAAGUACAGCGAGGAGGCGAAGCUGAACCGGGAGAAGAUCGACGGGGUC
AAGCUCGACUCCACGCGCAUCUACCAGAUCCUGGCCAUCUACUCGACCGUGGCGAGCUCC
CUCGUCCUGGUGGUCUCGCUCGGCGCCAUCAGCUUCUGGAUGUGCUCCAACGGGUCGCUG
CAGUGCCGGAUCUGCAUCUGA
>Influenza A virus H3N2 HAO mutant mRNA
SEQ ID NO: 132
AUGAAGACCAUCAUCGCCCUGAGCUACAUCCUGUGCCUGGUGUUCGCCCAGAAGAUCCCC
GGCAACGACAACAGCACCGCCACCCUGUGCCUGGGCCACCACGCCGUGCCCAACGGCACC
AUCGUGAAGACCAUCACCAACGACCGGAUCGAGGUGACCAACGCCACCGAGCUGGUGCAG
AACUCCAGCAUCGGCGAGAUCUGCGACUCCCCCCACCAGAUCCUGGACGGCGGCAACUGC
ACCCUGAUCGACGCCCUGCUGGGCGACCCCCAGUGCGACGGGUUCCAGAACAAGGAGUGG
GACCUGUUCGUGGAGCGGAGCCGCGCCAACUCCAACUGCUACCCCUACGACGUGCCCGAC
UACGCCAGCCUGCGGUCCCUGGUGGCCAGCUCCGGCACCCUGGAGUUCAAGAACGAGAGC
UUCAACUGGACCGGGGUCAAGCAGAACGGCACCUCCAGCGCCUGCAUCCGCGGGUCCAGC
UCCAGCUUCUUCUCCCGGCUGAACUGGCUCACCCACCUGAACUACACCUACCCCGCCCUC
AACGUGACCAUGCCCAACAACGAGCAGUUCGACAAGCUGUACAUCUGGGGCGUCCACCAC
CCCAGCACCGACAAGGACCAGAUCUCCCUCUUCGCCCAGCCCAGCGGGCGCAUCACCGUG
UCCACGAAGCGGAGCCAGCAGGCCGUCAUCCCCAACAUCGGCUCCCGCCCGCGGAUCCGC
GACAUCCCCAGCCGGAUCUCCAUCUACUGGACGAUCGUGAAGCCGGGGGACAUCCUGCUC
AUCAACAGCACCGGCAACCUGAUCGCCCCCCGCGGGUACUUCAAGAUCCGGUCCGGCAAG
AGCUCGAUCAUGCGCUCCGACGCCCCGAUCGGGAAGUGCAAGAGCGAGUGCAUCACCCCC
AACGGCUCGAUCCCGAACGACAAGCCCUUCCAGAACGUCAACCGGAUCACGUACGGGGCC
UGCCCGCGCUACGUGAAGCAGUCCACGCUCAAGCUGGCCACCGGCAUGCGGAACGUCCCC
GAGAAGCAGACCCAGGGGAUCUUCGGCGCCAUCGCGGGGUUCAUCGAGAACGGCUGGGAG
GGGAUGGUGGACGGCUGGUACGGGUUCCGGCACCAGAACAGCGAGGGCCGCGGGCAGGCC
GCGGACCUCAAGUCGACGCAGGCCGCGAUCGACCAGAUCAACGGCAAGCUGAACCGGCUC
AUCGGGAAGACGAACGAGAAGUUCCACCAGAUCGAGAAGGAGUUCUCCGAGGUCGAGGGC
CGCGUGCAGGACCUGGAGAAGUACGUCGAGGACACCAAGAUCGACCUCUGGAGCUACAAC
GCCGAGCUGCUCGUGGCGCUGGAGAACCAGCACACCAUCGACCUCACGGACUCGGAGAUG
AACAAGCUGUUCGAGAAGACGAAGAAGCAGCUCCGGGAGAACGCCGAGGACAUGGGGAAC
GGCUGCUUCAAGAUCUACCACAAGUGCGACAACGCGUGCAUCGGGUCCAUCCGCAACGAG
ACCUACGACCACAACGUCUACCGGGACGAGGCCCUGAACAACCGCUUCCAGAUCAAGGGC
GUGGAGCUCAAGAGCGGGUACAAGGACUGGAUCCUGUGGAUCUCGUUCGCGAUGUCCUGC
UUCCUCCUGUGCAUCGCCCUCCUGGGCUUCAUCAUGUGGGCGUGCCAGAAGGGGAACAUC
CGGUGCAACAUCUGCAUCUGA
>Influenza B virus/Victoria HAO mutant mRNA
SEQ ID NO: 133
AUGAAGGCCAUCAUCGUGCUGCUGAUGGUGGUGACCAGCAACGCCGACCGGAUCUGCACC
GGCAUCACCAGCUCCAACAGCCCCCACGUGGUGAAGACCGCCACCCAGGGCGAGGUGAAC
GUGACCGGCGUGAUCCCCCUGACCACCACCCCCACCAAGUCCCACUUCGCCAACCUGAAG
GGCACCGAGACCCGGGGCAAGCUGUGCCCCAAGUGCCUGAACUGCACCGACCUGGACGUG
GCCCUGGGCCGCCCCAAGUGCACCGGCAAGAUCCCCAGCGCCCGGGUGUCCAUCCUGCAC
GAGGUGCGCCCCGUGACCAGCGGCUGCUUCCCCAUCAUGCACGACCGCACCAAGAUCCGG
CAGCUGCCCAACCUGCUGCGGGGGUACGAGCACGUCCGCCUGUCCACCCACAACGUGAUC
AACGCCGAGGACGCCCCCGGCCGGCCCUACGAGAUCGGGACCAGCGGCUCCUGCCCCAAC
AUCACCAACGGGAACGGCUUCUUCGCCACCAUGGCCUGGGCCGUCCCCAAGAACAAGACC
GCCACCAACCCCCUGACCAUCGAGGUGCCGUACAUCUGCACCGAGGGGGAGGACCAGAUC
ACCGUCUGGGGCUUCCACAGCGACAACGAGACGCAGAUGGCCAAGCUGUACGGGGACUCC
AAGCCCCAGAAGUUCACGAGCUCCGCCAACGGCGUGACCACCCACUACGUCAGCCAGAUC
GGGGGCUUCCCGAACCAGACGGAGGACGGGGGCCUGCCCCAGUCCGGGCGCAUCGUGGUC
GACUACAUGGUGCAGAAGUCCGGCAAGACGGGGACCAUCACGUACCAGCGGGGCAUCCUG
CUGCCGCAGAAGGUCUGGUGCGCCUCCGGGCGCAGCAAGGUGAUCAAGGGCUCCCUGCCC
CUCAUCGGGGAGGCCGACUGCCUGCACGAGAAGUACGGGGGCUCAACAAGAGCAAGCCGU
ACUACACCGGCGAGCACGCCAAGGCCAUCGGGAACUGCCCCAUCUGGGUCAAGACCCCGC
UGAAGCUCGCCAACGGCACGAAGUACCGGCCCCCGGCCAAGCUGCUCAAGGAGCAGGGGU
UCUUCGGCGCCAUCGCCGGGUUCCUGGAGGGCGGGUGGGAGGGCAUGAUCGCCGGGUGGC
ACGGCUACACGUCCCACGGGGCCCACGGCGUGGCGGUCGCCGCGGACCUCAAGAGCACCC
AGGAGGCCAUCAACAAGAUCACGAAGAACCUGAACUCCCUCAGCGAGCUGGAGGUGAAGA
ACCUCCAGCGCCUGUCCGGGGCGAUGGACGAGCUCCACAACGAGAUCCUGGAGCUCGACG
AGAAGGUCGACGACCUGCGGGCCGACACCAUCAGCUCGCAGAUCGAGCUCGCGGUGCUGC
UCUCCAACGAGGGCAUCAUCAACAGCGAGGACGAGCACCUGCUCGCCCUGGAGCGGAAGC
UCAAGAAGAUGCUGGGGCCCUCGGCGGUCGAGAUCGGCAACGGGUGCUUCGAGACCAAGC
ACAAGUGCAACCAGACGUGCCUCGACCGCAUCGCCGCGGGCACGUUCGACGCCGGGGAGU
UCUCCCUGCCGACCUUCGACAGCCUCAACAUCACGGCGGCCUCGCUGAACGACGACGGCC
UCGACAACCACACCAUCCUGCUCUACUACUCCACCGCGGCCAGCUCGCUGGCGGUGACGC
UCAUGAUCGCCAUCUUCGUCGUGUACAUGGUCUCCCGCGACAACGUGAGCUGCUCGAUCU
GCCUGUGA
>Influenza B virus/Yamagata HAO mutant mRNA
SEQ ID NO: 134
AUGAAGGCCAUCAUCGUGCUGCUGAUGGUGGUGACCAGCAACGCCGACCGGAUCUGCACC
GGCAUCACCAGCUCCAACAGCCCCCACGUGGUGAAGACCGCCACCCAGGGCGAGGUGAAC
GUGACCGGCGUGAUCCCCCUGACCACCACCCCCACCAAGUCCUACUUCGCCAACCUGAAG
GGCACCCGGACCCGCGGCAAGCUGUGCCCCGACUGCCUGAACUGCACCGACCUGGACGUG
GCCCUGGGCCGGCCCAUGUGCGUGGGCACCACCCCCAGCGCCAAGGCCUCCAUCCUGCAC
GAGGUGCGCCCCGUGACCAGCGGCUGCUUCCCCAUCAUGCACGACCGCACCAAGAUCCGG
CAGCUGCCCAACCUGCUGCGGGGGUACGAGAAGAUCCGCCUGUCCACCCAGAACGUCAUC
GACGCCGAGAAGGCCCCCGGCGGGCCCUACCGGCUGGGCACCAGCGGGUCCUGCCCCAAC
GCCACCAGCAAGAUCGGCUUCUUCGCCACCAUGGCCUGGGCCGUGCCCAAGGACAACUAC
AAGAACGCCACCAACCCCCUGACCGUCGAGGUGCCCUACAUCUGCACCGAGGGGGAGGAC
CAGAUCACGGUCUGGGGCUUCCACUCCGACAACAAGACCCAGAUGAAGUCCCUGUACGGG
GACUCCAACCCGCAGAAGUUCACGAGCUCCGCCAACGGCGUGACCACGCACUACGUCAGC
CAGAUCGGGGACUUCCCGGACCAGACCGAGGACGGCGGGCUGCCCCAGUCCGGCCGCAUC
GUGGUCGACUACAUGAUGCAGAAGCCCGGGAAGACGGGCACCAUCGUGUACCAGCGGGGG
GUCCUGCUGCCGCAGAAGGUGUGGUGCGCCAGCGGCCGCUCCAAGGUCAUCAAGGGGAGC
CUGCCCCUCAUCGGCGAGGCCGACUGCCUGCACGAGGAGUACGGGGGCCUCAACAAGUCC
AAGCCGUACUACACGGGGAAGCACGCCAAGGCCAUCGGCAACUGCCCGAUCUGGGUGAAG
ACCCCCCUGAAGCUCGCCAACGGGACGAAGUACCGGCCGCCCGCCAAGCUGCUCAAGGAG
CAGGGCUUCUUCGGGGCCAUCGCCGGCUUCCUGGAGGGGGGCUGGGAGGGGAUGAUCGCC
GGCUGGCACGGGUACACCAGCCACGGCGCCCACGGGGUCGCGGUGGCCGCGGACCUCAAG
UCCACGCAGGAGGCCAUCAACAAGAUCACCAAGAACCUGAACAGCCUCUCCGAGCUGGAG
GUCAAGAACCUCCAGCGCCUGAGCGGCGCGAUGGACGAGCUCCACAACGAGAUCCUGGAG
CUCGACGAGAAGGUGGACGACCUGCGGGCCGACACGAUCUCGUCCCAGAUCGAGCUCGCG
GUCCUGCUCAGCAACGAGGGGAUCAUCAACUCGGAGGACGAGCACCUGCUCGCCCUGGAG
CGGAAGCUCAAGAAGAUGCUGGGCCCGUCCGCGGUGGACAUCGGGAACGGCUGCUUCGAG
ACCAAGCACAAGUGCAACCAGACGUGCCUCGACCGCAUCGCCGCGGGGACCUUCAACGCC
GGCGAGUUCAGCCUGCCCACGUUCGACUCGCUCAACAUCACCGCGGCCUCCCUGAACGAC
GACGGGCUCGACAACCACACGAUCCUGCUCUACUACAGCACCGCGGCCUCGUCCCUGGCG
GUCACGCUCAUGCUGGCCAUCUUCAUCGUGUACAUGGUCAGCCGCGACAACGUGUCGUGC
UCCAUCUGCCUCUGA
>Poly A
SEQ ID NO: 135
AAAAAAAAAAAAAAAAAAAAAAAAAAAAAAAAAAAAAAAAAAAAAAAAAAAAAAAAAAAA
AAAAAAAAAAAAAAAAAAAAAAAAAAAAAAAAAAAAAAAAAAAAAAAAAAAAAAAAAAAA
>SARS-CoV-2 BA.4 spike protein amino acid sequence
SEQ ID NO: 136
MFVFLVLLPLVSSQCVNLITRTQSYTNSFTRGVYYPDKVFRSSVLHSTQDLFLPFFSNVT
WFHAISGTNGTKRFDNPVLPFNDGVYFASTEKSNIIRGWIFGTTLDSKTQSLLIVNNATN
VVIKVCEFQFCNDPFLDVYYHKNNKSWMESEFRVYSSANNCTFEYVSQPFLMDLEGKQGN
FKNLREFVFKNIDGYFKIYSKHTPINLGRDLPQGFSALEPLVDLPIGINITRFQTLLALH
RSYLTPGDXSSGWTAGAAAYYVGYLQPRTFLLKYNENGTITDAVDCALDPLSETKCTLKS
FTVEKGIYQTSNFRVQPTESIVRFPNITNLCPFDEVFNATRFASVYAWNRKRISNCVADY
SVLYNFAPFFAFKCYGVSPTKLNDLCFTNVYADSFVIRGNEVSQIAPGQTGNIADYNYKL
PDDFTGCVIAWNSNKLDSKVGGNYNYRYRLFRKSNLKPFERDISTEIYQAGNKPCNGVAG
VNCYFPLQSYGFRPTYGVGHQPYRVVVLSFELLHAPATVCGPKKSTNLVKNKCVNFNFNG
LKGTGVLTESNKKFLPFQQFGRDIADTTDAVRDPQTLEILDITPCSFGGVSVITPGTNTS
NQVAVLYQGVNCTEVPVAIHADQLTPTWRVYSTGSNVFQTRAGCLIGAEYVNNSYECDIP
IGAGICASYQTQTKSHRRARSVASQSIIAYTMSLGAENSVAYSNNSIAIPTNFTISVTTE
ILPVXMTKTSVDCTMYICGDSTECSNLLLQYGSFCTQLKRALTGIAVEQDKNTQEVFAQV
KQIYKTPPIKYFGGFNFSQILPDPSKPSKRSFIEDLLFNKVTLADAGFIKQYGDCLGDIA
ARDLICAQKFNGLTVLPPLLTDEMIAQYTSALLAGTITSGWTFGAGAALQIPFAMQMAYR
FNGIGVTQNVLYENQKLIANQFNSAIGKIQDSLSSTASALGKLQDVVNHNAQALNTLVKQ
LSSKFGAISSVLNDILSRLDPPEAEVQIDRLITGRLQSLQTYVTQQLIRAAEIRASANLA
ATKMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK
YEQYIKWPWYIWLGFIAGLIAIVMVTIMLCCMTSCCSCLKGCCSCGSCCKFDEDDSEPVL
KGVKLHYT

1. A nucleic acid construct, comprising:
(a) an open reading frame (ORF), and
(b) a 5′ untranslated region element (5′UTR),
wherein the 5′UTR is derived from an ARHGAP gene;
optionally, the ARHGAP is ARHGAP15;
wherein optionally, the 5′UTR comprises the nucleotide sequence set forth in SEQ ID NO: 44 or a truncation thereof.

2. The nucleic acid construct according to claim 1, wherein the 5′UTR comprises at least one point mutation to mutate A into G, C, or T;
wherein optionally:
the 5′UTR truncation still maintains a function of regulating expression of an ORF-encoded protein; or, the 5′UTR truncation has an enhanced function of regulating expression of an ORF-encoded protein as compared to a natural 5′UTR;
a truncation mode for the 5′UTR truncation comprises: in a sequence direction from the 5′ end to the 3′ end, deleting a sequence of contiguous nucleotides from the 5′ end; and/or, in a sequence direction from the 3′ end to the 5′ end, deleting a sequence of contiguous nucleotides from the 3′ end.

3. (canceled)

4. The nucleic acid construct according to claim 2, wherein the 5′UTR truncation comprises a sequence of at least 5 contiguous nucleotides from the sequence set forth in SEQ ID NO: 44;
wherein optionally, the nucleotide at the 5 end of the 5′UTR truncation is the nucleotide at any one of positions 1-57, according to natural counting, of the sequence set forth in SEQ ID NO: 44, optionally the nucleotide at any one of positions 1-13 or 17-29, according to natural counting, of the sequence set forth in SEQ ID NO: 2;
optionally, the 5′UTR truncation comprises CTATAAT or the nucleotide sequence set forth in SEQ ID NO: 193.

5. The nucleic acid construct according to claim 1, wherein the 5′UTR comprises the nucleotide sequence set forth in any one of SEQ ID NOs: 2, 28-43, 76, and 137-196 or comprises CTATAAT; or a nucleotide sequence having at least 80% identity to any one of the aforementioned sequences.

6. The nucleic acid construct according to claim 1, further comprising:
(c) a 3′ untranslated region element (3′UTR), and/or (d) a polyadenylic acid (poly-A) tail;
wherein optionally, the 3′UTR comprises a 3′UTR derived from any one of the gene HBB, ARHGAP, CORO1A, or HPX;
optionally the poly-A tail is selected from the group consisting of HGH polyA, SV40polyA, BGH polyA, rbGlob polyA, and SV40late polyA;
and optionally:
the 3′UTR comprises the nucleotide sequence set forth in any one of SEQ ID NOs: 7-10, or, the 3′UTR comprises a nucleotide sequence having at least 80% identity to any one of SEQ ID NOs: 7-10;
and the poly-A tail comprises the nucleotide sequence set forth in SEQ ID NO: 16 or SEQ ID NO: 135 or a nucleotide sequence having at least 80% identity thereto.

7-9. (canceled)

10. The nucleic acid construct according to claim 1, wherein the ORF encodes a viral antigen; wherein optionally, the ORF encodes a coronavirus antigen or an influenza virus antigen;
wherein optionally the coronavirus is SARS-COV-2, and the coronavirus antigen is a spike protein selected from a spike protein of any one of the viral strain SARS-COV-2, SARS-COV-2 Alpha, SARS-COV-2 Beta, SARS-COV-2 Gamma, SARS-COV-2 Kappa, SARS-COV-2 Delta, or SARS-COV-2 Omicron;
wherein optionally the influenza virus is influenza A virus or influenza B virus, and the antigen is a hemagglutinin protein (HA) and/or a neuraminidase (NA) of any one of the viral strains influenza A virus H1N1, influenza A virus H3N2, influenza B virus Victoria, and influenza B virus Yamagata.

11. The nucleic acid construct according to claim 1, wherein:
the ORF encodes the amino acid sequence set forth in any one of SEQ ID NOs: 12, 14, 21, 23, 25, 80, 83-86, 98-101, and 136;
wherein optionally, the ORF comprises the nucleotide sequence set forth in any one of SEQ ID NOs: 13, 15, 20, 22, 24, 81, 87-90, 97, and 102-105, or a nucleotide sequence having at least 80%, 85%, 90%, 95%, 96%, 97%, 98%, or 99% identity to the nucleotide sequence set forth in any one of SEQ ID NOs: 13, 15, 20, 22, 24, 81, 87-90, 97, and 102-105.

12. The nucleic acid construct according to claim 1, comprising the nucleotide sequences set forth in SEQ ID NOs: 18-19 or nucleotide sequences having at least 80% identity thereto.

13. An RNA molecule, comprising:
(a) an open reading frame (ORF),
(b) a 5′ untranslated region element (5′UTR), and
(c) a 3′ untranslated region element (3′UTR),
wherein the 5′UTR is selected from the group consisting of a 5′UTR derived from any one of the gene ARHGAP, HSPB1, HBB, or CCL13, and the 3′UTR is selected from the group consisting of a 3′UTR derived from any one of the gene HBB, ARHGAP, CORO1A, or HPX;
the ARHGAP is optionally ARHGAP15, and the 5′UTR optionally comprises the nucleotide sequence set forth in SEQ ID NO: 79 or a truncation thereof.

14. The RNA molecule according to claim 13, wherein the 5′UTR and 3′UTR are selected from the any one of the following combinations:
1) the 5′UTR is derived from a 5′UTR of ARHGAP, and the 3′UTR is derived from a 3′UTR of any one of HBB, ARHGAP, CORO1A, or HPX;
2) the 5′UTR is derived from a 5′UTR of HSPB1, and the 3′UTR is derived from a 3′UTR of any one of HBB, ARHGAP, CORO1A, or HPX;
3) the 5′UTR is derived from a 5′UTR of HBB, and the 3′UTR is derived from a 3′UTR of any one of HBB, ARHGAP, CORO1A, or HPX; and
4) the 5′UTR is derived from a 5′UTR of CCL13, and the 3′UTR is derived from a 3′UTR of any one of HBB, ARHGAP, CORO1A, or HPX;
optionally wherein,
the 5′UTR of ARHGAP comprises the nucleotide sequence set forth in any one of SEQ ID NOs: 45-46, 63-75, and 77-78 and CUAUAAU, or comprises CUAUAAU, or a nucleotide sequence having at least 80% identity to any one of the aforementioned sequences;
the 5′UTR derived from HSPB1 comprises the nucleotide sequence set forth in SEQ ID NO: 47 or a nucleotide sequence having at least 80% identity thereto;
the 5′UTR derived from HBB comprises the nucleotide sequence set forth in SEQ ID NO: 48 or a nucleotide sequence having at least 80% identity thereto;
the 5′UTR derived from CCL13 comprises the nucleotide sequence set forth in SEQ ID NO: 49 or a nucleotide sequence having at least 80% identity thereto;
the 3′UTR derived from HBB comprises the nucleotide sequence set forth in SEQ ID NO: 50 or a nucleotide sequence having at least 80% identity thereto;
the 3′UTR derived from ARHGAP comprises the nucleotide sequence set forth in SEQ ID NO: 51 or a nucleotide sequence having at least 80% identity thereto;
the 3′UTR derived from CORO1A comprises the nucleotide sequence set forth in SEQ ID NO: 52 or a nucleotide sequence having at least 80% identity thereto;
the 3′UTR derived from HPX comprises the nucleotide sequence set forth in SEQ ID NO: 53 or a nucleotide sequence having at least 80% identity thereto.

15. The RNA molecule according to claim 13, wherein the ORF encodes a viral antigen; optionally, the ORF encodes a coronavirus antigen or an influenza virus antigen;
the coronavirus is optionally SARS-COV-2, and the coronavirus antigen is optionally a spike protein;
optionally, the spike protein is selected from the group consisting of a spike protein of any one of the viral strain SARS-COV-2, SARS-COV-2 Alpha, SARS-COV-2 Beta, SARS-COV-2 Gamma, SARS-COV-2 Kappa, SARS-COV-2 Delta, or SARS-COV-2 Omicron; or, the influenza virus is selected from the group consisting of influenza A virus and influenza B virus, and the influenza virus antigen is a hemagglutinin protein (HA) and/or a neuraminidase (NA) of any one of the viral strains influenza A virus H1N1, influenza A virus H3N2, influenza B virus Victoria, and influenza B virus Yamagata.

16. The RNA molecule according to claim 15, wherein:
the ORF encodes a polypeptide comprising the amino acid sequence set forth in any one of SEQ ID NOs: 12, 14, 21, 23, 25, 80, 83-86, 98-101, and 136;
optionally, the nucleotide sequence of the ORF comprises the nucleotide sequence set forth in any one of SEQ ID NOs: 54-55, 59-61, 82, and 126-134, or a nucleotide sequence having at least 80%, 85%, 90%, 95%, 96%, 97%, 98%, or 99% identity to the nucleotide sequence set forth in any one of SEQ ID NOs: 54-55, 59-61, 82, and 126-134.

17. The RNA molecule according to claim 13, further comprising:
(d) a polyadenylic acid (poly-A) tail and/or (e) a 5′ cap structure (5′Cap);
wherein optionally:
the poly-A tail is selected from the group consisting of HGH polyA, SV40polyA, BGH polyA, rbGlob polyA, and SV40late polyA; and
the 5′Cap is selected from the group consisting of Cap0, Cap1, Cap2, Cap3, Cap4, ARCA, modified ARCA, inosine, N1-methyl-guanosine, 2′-fluoro-guanosine, 7-deaza-guanosine, 8-oxo-guanosine, 2-amino-guanosine, LNA-guanosine, and 2-azido-guanosine, 3′OMe-m7G(5′)ppp(5′)G, m7G(5′)ppp(5′)(2′OMeA)pU, m7Gppp(A2′O-MOE)pG, m7G(5′)ppp(5′)(2′OMeA)pG, m7G(5′)ppp(5′)(2′OMeG)pG, m7(3′OMeG)(5′)ppp(5′)(2′OMeG)pG, and m7(3′OMeG)(5′)ppp(5′)(2′OMeA)pG;
optionally wherein the poly-A tail comprises the nucleotide sequence set forth in SEQ ID NO: 56 or SEQ ID NO: 135 or a nucleotide sequence having at least 80% identity thereto;
optionally wherein, the RNA molecule comprises the nucleotide sequence set forth in any one of SEQ ID NOs: 57-58 and 106-125.

18.-21. (canceled)

22. The RNA molecule according to claim 13, wherein the RNA molecule is an mRNA;
wherein optionally the RNA further comprises one or more modifications,
wherein optionally, the modifications include a backbone modification, a sugar modification, a base modification, and/or a lipid modification;
and optionally, the base modification is a pseudouridine modification.

23. (canceled)

24. An RNA molecule, comprising the nucleotide sequence set forth in any one of SEQ ID NOs: 54-55, 59-61, 82, and 126-134 or a nucleotide sequence having at least 80% identity to the nucleotide sequence set forth in any one of SEQ ID NOs: 54-55, 59-61, 82, and 126-134,
wherein optionally, the RNA molecule further comprises one or more modifications; optionally, the modifications are pseudouridine modifications.

25. (canceled)

26. A vaccine, comprising the RNA molecule according to claim 13.

27. A vector, comprising the nucleic acid construct according to claim 1, or the RNA molecule according to claim 13.

28. A host cell, comprising the vector according to claim 27.

29-33. (canceled)

34. A method for treating and/or preventing a disease, comprising administering to a subject in need thereof an effective amount of the RNA molecule according to claim 13, wherein the disease is a viral infectious disease or a respiratory disease associated with viral infection;
optionally wherein, the virus is a coronavirus or an influenza virus; optionally, the virus is SARS-CoV-2, influenza A virus, or influenza B virus;
and optionally, the respiratory disease associated with viral infection includes simple infection, fever, cough, sore throat, rhinitis, headache, pneumonia, acute respiratory infection, severe acute respiratory infection (SARI), hypoxemic respiratory failure, acute respiratory distress syndrome, sepsis, septic shock, and severe acute respiratory syndrome (SARS).

35. A method for inducing a neutralizing antibody reaction and/or a T cell immune response in a subject, comprising administering to a subject in need thereof an effective amount of the RNA molecule according to claim 13,
wherein optionally, the neutralizing antibody reaction is a neutralizing antibody reaction against a viral antigen, and the T cell immune response includes a CD4+ and/or CD8+ T cell immune reaction;
the virus is a coronavirus or an influenza virus; optionally, the virus is SARS-CoV-2, influenza A virus, or influenza B virus.


SEQUENCE LISTING (196 sequences)

SEQ ID NO: 1 - DNA, 63 bp, synthetic construct
  source 1..63: mol_type = other DNA; note = 5'UTR I
gagttaggagttgatggcagtttcaataacaggtcattgccgagaaaaggatagcactataat

SEQ ID NO: 2 - DNA, 63 bp, synthetic construct
  source 1..63: mol_type = other DNA; note = 5'UTR I'
gagttaggagttggtggcagtttcaataacaggtcattgccgagaaaaggatagcactataat

SEQ ID NO: 3 - DNA, 43 bp, synthetic construct
  source 1..43: mol_type = other DNA; note = 5'UTR G
gcacttttctgagcagacgtccagagcagagtcagccgccacc

SEQ ID NO: 4 - DNA, 52 bp, synthetic construct
  source 1..52: mol_type = other DNA; note = 5'UTR A
acatttgcttctgacacaactgtgttcactagcaacctcaaacgccgccacc

SEQ ID NO: 5 - DNA, 55 bp, synthetic construct
  source 1..55: mol_type = other DNA; note = 5'UTR J
agagaggcaaagaaacattgtgaaatctccaactcttaaccttcaagccgccacc

SEQ ID NO: 6 - DNA, 52 bp, synthetic construct
  source 1..52: mol_type = other DNA; note = 5'UTR BNT162b2
gaataaactagtattcttctggtccccacagactcagagagaacccgccacc

SEQ ID NO: 7 - DNA, 134 bp, synthetic construct
  source 1..134: mol_type = other DNA; note = 3'UTR B
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 8 - DNA, 180 bp, synthetic construct
  source 1..180: mol_type = other DNA; note = 3'UTR F
cagacaagacaagctactgaatacgttcacatctgtcttgatgcctaatatttttacatttctgtaaacatatttctgaaatattttttgcctttcaagcgacagatgcctcattttgtgaaaacttaatgatgattttgtgtttaagttccaaacatttgaataaaataattgacaata

SEQ ID NO: 9 - DNA, 388 bp, synthetic construct
  source 1..388: mol_type = other DNA; note = 3'UTR E
cctgaggcctcagaacacaggtttcagattgataggcctgcaggtctccaggcagcaaccagctgagcgactaaagggcccaaggccagggctctagggatggggctcagcagaggctggggtaaggggagccagggaggagctgggcctaatgcagcaccgggtccccaggatgccgtgtctcggctggaggaggagatgcggaagctccaggccacggtgcaggagctccagaagcgcttggacaggctggaggagacagtccaggccaagtagagccccgcagggcctccagcagggtcagccattcacacccatccactcacctcccattcccagccacatggcagagaaaaaaatcataataaaatggctttattttctggta

SEQ ID NO: 10 - DNA, 296 bp, synthetic construct
  source 1..296: mol_type = other DNA; note = 3'UTR D
ggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 11 - DNA, 289 bp, synthetic construct
  source 1..289: mol_type = other DNA; note = 3'UTR BNT162b2
ctcgagctggtactgcatgcacgcaatgctagctgcccctttcccgtcctgggtaccccgagtctcccccgacctcgggtcccaggtatgctcccacctccacctgccccactcaccacctctgctagttccagacacctcccaagcacgcagcaatgcagctcaaaacgcttagcctagccacacccccacgggaaacagcagtgattaacctttagcaataaacgaaagtttaactaagctatactaaccccagggttggtcaatttcgtgccagccacaccctgga

SEQ ID NO: 12 - protein, 1271 aa, synthetic construct
  source 1..1271: note = B.1.617.2(Delta)Spike
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - DNA, 3819 bp, synthetic construct
  source 1..3819: mol_type = other DNA; note = B.1.617.2(Delta)Spike CDS
atgttcgtgttcctggtgctgctgcccctggtgagctctcagtgcgtgaacctgaggacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcggggtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactaccggtacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccgccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaaaacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 14 - protein, 1270 aa, synthetic construct
  source 1..1270: note = B.1.1.529(Omicron) Spike
MFVFLVLLPLVSSQCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLRSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3816 bp, synthetic construct
  source 1..3816: mol_type = other DNA; note = B.1.1.529(Omicron)Spike CDS
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgtgatcagcggcaccaacggcaccaagagattcgacaaccccgtgctgcccttcaacgacggcgtgtacttcgctagcatcgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggaccacaagaacaacaagagctggatggagagcgagttcagagtgtacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccaccagatttgctagcgtgtacgcctggaaccggaagagaatcagcaactgcgtggccgactacagcgtgctgtacaacctggcccccttcttcaccttcaagtgctatggcgtcagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctggatagcaaggtgagcggcaactacaattacctgtacagactgttcagaaagagcaacctgaagcccttcgagagagacatcagcaccgagatctaccaagccggcaacaagccctgcaacggcgtggcgggcttcaactgctacttccccctcaggagctacagcttcagacccacctacggcgtgggccatcagccctacagagtggtcgtgctgagcttcgagctgctgcacgcccctgccacagtgtgcggccccaaaaagagcaccaacctggtgaagaacaagtgcgtgaacttcaacttcaacggcctgaagggcaccggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtgtatagcaccggcagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaagggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaatgacatcttctctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 16 - DNA, 112 bp, synthetic construct
  source 1..112: mol_type = other DNA; note = poly A
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 17 - DNA, 20 bp, synthetic construct
  source 1..20: mol_type = other DNA; note = T7 promoter
taatacgactcactataagg

SEQ ID NO: 18 - DNA, 4134 bp, synthetic construct
  source 1..4134: mol_type = other DNA; note = I'-Delta24-B
gagttaggagttggtggcagtttcaataacaggtcattgccgagaaaaggatagcactataatatgttcgtgttcctggtgctgctgcccctggtgagctctcagtgcgtgaacctgaggacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcggggtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactaccggtacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccgccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaaaacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 19 - DNA, 4131 bp, synthetic construct
  source 1..4131: mol_type = other DNA; note = I'-OC24-B
gagttaggagttggtggcagtttcaataacaggtcattgccgagaaaaggatagcactataatatgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgtgatcagcggcaccaacggcaccaagagattcgacaaccccgtgctgcccttcaacgacggcgtgtacttcgctagcatcgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggaccacaagaacaacaagagctggatggagagcgagttcagagtgtacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccaccagatttgctagcgtgtacgcctggaaccggaagagaatcagcaactgcgtggccgactacagcgtgctgtacaacctggcccccttcttcaccttcaagtgctatggcgtcagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctggatagcaaggtgagcggcaactacaattacctgtacagactgttcagaaagagcaacctgaagcccttcgagagagacatcagcaccgagatctaccaagccggcaacaagccctgcaacggcgtggcgggcttcaactgctacttccccctcaggagctacagcttcagacccacctacggcgtgggccatcagccctacagagtggtcgtgctgagcttcgagctgctgcacgcccctgccacagtgtgcggccccaaaaagagcaccaacctggtgaagaacaagtgcgtgaacttcaacttcaacggcctgaagggcaccggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtgtatagcaccggcagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaagggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaatgacatcttctctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 20 - DNA, 3816 bp, synthetic construct
  source 1..3816: mol_type = other DNA; note = B.1.351 Spike CDS
atgttcgtgttcctggtgctgctgcccctggtgagctctcagtgcgtgaacttcacgacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtgagcggcaccaacggcaccaagagattcgccaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtactaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagaggcctgccccaaggcttcagcgccctggagcccctggtggacctgcccatcggcatcaacatcacaagatttcagaccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaacatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtgaagggcttcaactgctacttccccctgcagagctacggctttcagcccacctacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagcccccggagagctagaagcgtggcttctcagagcatcatcgcctacaccatgagcctgggcgtcgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 21 - protein, 1270 aa, synthetic construct
  source 1..1270: note = B.1.351 Spike
MFVFLVLLPLVSSQCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 22 - DNA, 3816 bp, synthetic construct
  source 1..3816: mol_type = other DNA; note = B.1.1.7 Spike CDS
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtggagggcttcaactgctacttccccctgcagagctacggctttcagcccacctacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgacgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccaccggagagctagaagcgtggcttctcagagcatcatcgcctacaccatgagcctgggcgccgagaacagcgtggcctacagcaacaacagcatcgccatccccatcaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctggctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccacccacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 23 - protein, 1270 aa, synthetic construct
  source 1..1270: note = B.1.1.7 Spike
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 24 - DNA, 3825 bp, synthetic construct
  source 1..3825: mol_type = other DNA; note = SARS-COV-2 Spike CDS
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtgagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtactaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtggagggcttcaactgctacttccccctgcagagctacggctttcagcccaccaatggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaagacgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccctcggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctgagcagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 25 - protein, 1273 aa, synthetic construct
  source 1..1273: note = SARS-COV-2 Spike
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 26 - DNA, 1653 bp, synthetic construct
  source 1..1653: mol_type = other DNA; note = Luciferase CDS
atggaggacgccaagaacatcaagaaaggccccgcccccttctaccccctggaggacggcaccgccggcgagcagctgcacaaagccatgaaaagatacgctctggtgcccggcaccatcgccttcaccgacgcccacatcgaggtgaacatcacctacgccgagtacttcgagatgagcgtgagactggccgaggctatgaagagatacggcttgaacaccaaccacagaatcgtggtgtgcagcgagaacagcctgcagttcttcatgcccgtgctgggcgccctgttcatcggcgtggccgttgcccccgccaacgacatctacaacgagagagagctgctgaacagcatgaacatctctcagcccaccgtggttttcgtgtccaaaaagggcctgcagaagatcctgaacgtgcagaagaagctgcccatcattcagaagatcatcatcatggacagcaagaccgactaccaaggctttcagagcatgtacaccttcgtgacaagccacctgccccccggcttcaacgagtacgacttcgtgcccgagagcttcgacagagacaagaccatcgccctgatcatgaacagcagcggcagcaccggcctgcccaagggcgtggccctgccccacagaaccgcctgcgtgagattcagccacgctagagaccccatcttcggcaatcagatcatccccgacaccgccatcctgagcgtagtgcctttccatcatggcttcggcatgttcaccaccctgggctacctgatctgcggcttcagagtggtgctgatgtacagattcgaggaggagctgttcctgagaagcctgcaagactacaagattcagagcgccctgctggtgcccaccctgttcagcttcttcgccaagagcaccctgatcgacaagtatgacctgagcaacctgcacgaaattgcgagcggcggagcgccgctcagcaaagaggtgggcgaggccgtggccaagagattccacctgcccggcatcagacaaggctacggcctgaccgagaccacaagcgccatcctgatcacccccgagggcgacgataagcccggcgccgtgggcaaggtggtaccattcttcgaggccaaggtggtggacctggacaccggcaagaccctgggcgtgaatcagagaggcgagctgtgcgtgagaggccccatgatcatgagcggctacgtgaacaaccccgaggccaccaacgccctcatcgataaggatgggtggctgcacagcggcgacatcgcctactgggacgaggacgagcacttcttcatcgtggacagactcaagagcctgatcaagtacaagggctaccaagtggcccccgccgagttggagagcatcctgctgcagcaccccaatatcttcgacgctggcgtggccggactacccgacgacgacgccggcgagttacccgccgccgtggtggtgctggagcacggcaagaccatgaccgagaaggagatcgtggactacgtggctagccaagtgaccaccgccaagaagctgagaggcggcgtggtgttcgttgacgaagtgcccaagggcctgaccggcaagctggacgctagaaagatcagagagatcctgatcaaggccaagaagggcggcaagagcaagctgtga

SEQ ID NO: 27 - protein, 550 aa, synthetic construct
  source 1..550: note = Luciferase
MEDAKNIKKGPAPFYPLEDGTAGEQLHKAMKRYALVPGTIAFTDAHIEVNITYAEYFEMSVRLAEAMKRYGLNTNHRIVVCSENSLQFFMPVLGALFIGVAVAPANDIYNERELLNSMNISQPTVVFVSKKGLQKILNVQKKLPIIQKIIIMDSKTDYQGFQSMYTFVTSHLPPGFNEYDFVPESFDRDKTIALIMNSSGSTGLPKGVALPHRTACVRFSHARDPIFGNQIIPDTAILSVVPFHHGFGMFTTLGYLICGFRVVLMYRFEEELFLRSLQDYKIQSALLVPTLFSFFAKSTLIDKYDLSNLHEIASGGAPLSKEVGEAVAKRFHLPGIRQGYGLTETTSAILITPEGDDKPGAVGKVVPFFEAKVVDLDTGKTLGVNQRGELCVRGPMIMSGYVNNPEATNALIDKDGWLHSGDIAYWDEDEHFFIVDRLKSLIKYKGYQVAPAELESILLQHPNIFDAGVAGLPDDDAGELPAAVVVLEHGKTMTEKEIVDYVASQVTTAKKLRGGVVFVDEVPKGLTGKLDARKIREILIKAKKGGKSKL

SEQ ID NO: 28 - DNA, 59 bp, synthetic construct
  source 1..59: mol_type = other DNA; note = LUC1- I' truncation
taggagttggtggcagtttcaataacaggtcattgccgagaaaaggatagcactataat

SEQ ID NO: 29 - DNA, 55 bp, synthetic construct
  source 1..55: mol_type = other DNA; note = LUC2- I' truncation
agttggtggcagtttcaataacaggtcattgccgagaaaaggatagcactataat

SEQ ID NO: 30 - DNA, 51 bp, synthetic construct
  source 1..51: mol_type = other DNA; note = LUC3- I' truncation
ggtggcagtttcaataacaggtcattgccgagaaaaggatagcactataat

SEQ ID NO: 31 - DNA, 47 bp, synthetic construct
  source 1..47: mol_type = other DNA; note = LUC4- I' truncation
gcagtttcaataacaggtcattgccgagaaaaggatagcactataat

SEQ ID NO: 32 - DNA, 43 bp, synthetic construct
  source 1..43: mol_type = other DNA; note = LUC5- I' truncation
tttcaataacaggtcattgccgagaaaaggatagcactataat

SEQ ID NO: 33 - DNA, 39 bp, synthetic construct
  source 1..39: mol_type = other DNA; note = LUC6- I' truncation
aataacaggtcattgccgagaaaaggatagcactataat

SEQ ID NO: 34 - DNA, 35 bp, synthetic construct
  source 1..35: mol_type = other DNA; note = LUC7- I' truncation
acaggtcattgccgagaaaaggatagcactataat

SEQ ID NO: 35 - DNA, 31 bp, synthetic construct
  source 1..31: mol_type = other DNA; note = LUC8- I' truncation
gtcattgccgagaaaaggatagcactataat

SEQ ID NO: 36 - DNA, 27 bp, synthetic construct
  source 1..27: mol_type = other DNA; note = LUC9- I' truncation
ttgccgagaaaaggatagcactataat

SEQ ID NO: 37 - DNA, 23 bp, synthetic construct
  source 1..23: mol_type = other DNA; note = LUC10- I' truncation
cgagaaaaggatagcactataat

SEQ ID NO: 38 - DNA, 19 bp, synthetic construct
  source 1..19: mol_type = other DNA; note = LUC11- I' truncation
aaaaggatagcactataat

SEQ ID NO: 39 - DNA, 15 bp, synthetic construct
  source 1..15: mol_type = other DNA; note = LUC12- I' truncation
ggatagcactataat

SEQ ID NO: 40 - DNA, 11 bp, synthetic construct
  source 1..11: mol_type = other DNA; note = LUC13- I' truncation
agcactataat

SEQ ID NO: 41 - DNA, 21 bp, synthetic construct
  source 1..21: mol_type = other DNA; note = 5'UTR truncation
tcaataacaggtcattgccga

SEQ ID NO: 42 - DNA, 63 bp, synthetic construct
  source 1..63: mol_type = other DNA; note = LUC15- I' mutant
gagttaggagttgctggcagtttcaataacaggtcattgccgagaaaaggatagcactataat

SEQ ID NO: 43 - DNA, 63 bp, synthetic construct
  source 1..63: mol_type = other DNA; note = LUC16- I' mutant
gagttaggagttgttggcagtttcaataacaggtcattgccgagaaaaggatagcactataat

SEQ ID NO: 44 - DNA, 63 bp, synthetic construct
  source 1..63: mol_type = other DNA; note = 5'UTR I general formula
  misc_difference 14: note = N is selected from the group consisting of A, G, C, and T
gagttaggagttgntggcagtttcaataacaggtcattgccgagaaaaggatagcactataat

SEQ ID NO: 45 - RNA, 63 nt, synthetic construct
  source 1..63: mol_type = other RNA; note = 5'UTR I
gagttaggagttgatggcagtttcaataacaggtcattgccgagaaaaggatagcactataat

SEQ ID NO: 46 - RNA, 63 nt, synthetic construct
  source 1..63: mol_type = other RNA; note = 5'UTR I'
gagttaggagttggtggcagtttcaataacaggtcattgccgagaaaaggatagcactataat

SEQ ID NO: 47 - RNA, 43 nt, synthetic construct
  source 1..43: mol_type = other RNA; note = 5'UTR G
gcacttttctgagcagacgtccagagcagagtcagccgccacc

SEQ ID NO: 48 - RNA, 52 nt, synthetic construct
  source 1..52: mol_type = other RNA; note = 5'UTR A
acatttgcttctgacacaactgtgttcactagcaacctcaaacgccgccacc

SEQ ID NO: 49 - RNA, 55 nt, synthetic construct
  source 1..55: mol_type = other RNA; note = 5'UTR J
agagaggcaaagaaacattgtgaaatctccaactcttaaccttcaagccgccacc

SEQ ID NO: 50 - RNA, 134 nt, synthetic construct
  source 1..134: mol_type = other RNA; note = 3'UTR B
gctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 51 - RNA, 180 nt, synthetic construct
  source 1..180: mol_type = other RNA; note = 3'UTR F
cagacaagacaagctactgaatacgttcacatctgtcttgatgcctaatatttttacatttctgtaaacatatttctgaaatattttttgcctttcaagcgacagatgcctcattttgtgaaaacttaatgatgattttgtgtttaagttccaaacatttgaataaaataattgacaata

SEQ ID NO: 52 - RNA, 388 nt, synthetic construct
  source 1..388: mol_type = other RNA; note = 3'UTR E
cctgaggcctcagaacacaggtttcagattgataggcctgcaggtctccaggcagcaaccagctgagcgactaaagggcccaaggccagggctctagggatggggctcagcagaggctggggtaaggggagccagggaggagctgggcctaatgcagcaccgggtccccaggatgccgtgtctcggctggaggaggagatgcggaagctccaggccacggtgcaggagctccagaagcgcttggacaggctggaggagacagtccaggccaagtagagccccgcagggcctccagcagggtcagccattcacacccatccactcacctcccattcccagccacatggcagagaaaaaaatcataataaaatggctttattttctggta

SEQ ID NO: 53 - RNA, 296 nt, synthetic construct
  source 1..296: mol_type = other RNA; note = 3'UTR D
ggggccttctgacatgagtctggcctggccccacctcctagttcctcataataaagacagattgcttcttcgcttctcactgaggggccttctgacatgagtctggcctggccccacctccccagtttctcataataaagacagattgcttcttcacttgaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 54 - RNA, 3819 nt, synthetic construct
  source 1..3819: mol_type = other RNA; note = B.1.617.2(Delta) spike mRNA
atgttcgtgttcctggtgctgctgcccctggtgagctctcagtgcgtgaacctgaggacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcggggtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactaccggtacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccgccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaaaacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 55 - RNA, 3816 nt, synthetic construct
  source 1..3816: mol_type = other RNA; note = B.1.1.529(Omicron) spike mRNA
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgtgatcagcggcaccaacggcaccaagagattcgacaaccccgtgctgcccttcaacgacggcgtgtacttcgctagcatcgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggaccacaagaacaacaagagctggatggagagcgagttcagagtgtacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccaccagatttgctagcgtgtacgcctggaaccggaagagaatcagcaactgcgtggccgactacagcgtgctgtacaacctggcccccttcttcaccttcaagtgctatggcgtcagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctggatagcaaggtgagcggcaactacaattacctgtacagactgttcagaaagagcaacctgaagcccttcgagagagacatcagcaccgagatctaccaagccggcaacaagccctgcaacggcgtggcgggcttcaactgctacttccccctcaggagctacagcttcagacccacctacggcgtgggccatcagccctacagagtggtcgtgctgagcttcgagctgctgcacgcccctgccacagtgtgcggccccaaaaagagcaccaacctggtgaagaacaagtgcgtgaacttcaacttcaacggcctgaagggcaccggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtgtatagcaccggcagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaagggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaatgacatcttctctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 56 - RNA, 112 nt, synthetic construct
  source 1..112: mol_type = other RNA; note = poly A
aaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 57 - RNA, 4134 nt, synthetic construct
  source 1..4134: mol_type = other RNA; note = I'-Delta24-B mRNA
gagttaggagttggtggcagtttcaataacaggtcattgccgagaaaaggatagcactataatatgttcgtgttcctggtgctgctgcccctggtgagctctcagtgcgtgaacctgaggacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcggggtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactaccggtacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccgccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaaaacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 58 - RNA, 4131 nt, synthetic construct
  source 1..4131: mol_type = other RNA; note = I'-OC24-B mRNA
gagttaggagttggtggcagtttcaataacaggtcattgccgagaaaaggatagcactataatatgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgtgatcagcggcaccaacggcaccaagagattcgacaaccccgtgctgcccttcaacgacggcgtgtacttcgctagcatcgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggaccacaagaacaacaagagctggatggagagcgagttcagagtgtacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccaccagatttgctagcgtgtacgcctggaaccggaagagaatcagcaactgcgtggccgactacagcgtgctgtacaacctggcccccttcttcaccttcaagtgctatggcgtcagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctggatagcaaggtgagcggcaactacaattacctgtacagactgttcagaaagagcaacctgaagcccttcgagagagacatcagcaccgagatctaccaagccggcaacaagccctgcaacggcgtggcgggcttcaactgctacttccccctcaggagctacagcttcagacccacctacggcgtgggccatcagccctacagagtggtcgtgctgagcttcgagctgctgcacgcccctgccacagtgtgcggccccaaaaagagcaccaacctggtgaagaacaagtgcgtgaacttcaacttcaacggcctgaagggcaccggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtgtatagcaccggcagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaagggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaatgacatcttctctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 59 - RNA, 3816 nt, synthetic construct
  source 1..3816: mol_type = other RNA; note = B.1.351 Spike mRNA
atgttcgtgttcctggtgctgctgcccctggtgagctctcagtgcgtgaacttcacgacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtgagcggcaccaacggcaccaagagattcgccaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtactaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagaggcctgccccaaggcttcagcgccctggagcccctggtggacctgcccatcggcatcaacatcacaagatttcagaccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaacatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtgaagggcttcaactgctacttccccctgcagagctacggctttcagcccacctacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagcccccggagagctagaagcgtggcttctcagagcatcatcgcctacaccatgagcctgggcgtcgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 60 - RNA, 3816 nt, synthetic construct
  source 1..3816: mol_type = other RNA; note = B.1.1.7 Spike mRNA
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtggagggcttcaactgctacttccccctgcagagctacggctttcagcccacctacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgacgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccaccggagagctagaagcgtggcttctcagagcatcatcgcctacaccatgagcctgggcgccgagaacagcgtggcctacagcaacaacagcatcgccatccccatcaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctggctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccacccacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 61 - RNA, 3825 nt, synthetic construct
  source 1..3825: mol_type = other RNA; note = SARS-COV-2 Spike mRNA
atgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtgagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctgggcgtgtactaccacaagaacaacaagagctggatggagagcgagttccgtgtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactacctgtacagactgttcagaaagagcaacctgaagcccttcgagagagacatcagcaccgagatctaccaagccggcagcaccccctgcaacggcgtggagggcttcaactgctacttccccctgcagagctacggctttcagcccaccaatggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaagacgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccctcggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcagaacgcccaagccctgaacaccctggtgaagcagctgagcagcaacttcggcgccatcagcagcgtattgaatgacatcctgagcagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 62 - RNA, 1653 nt, synthetic construct
  source 1..1653: mol_type = other RNA; note = Luciferase mRNA
atggaggacgccaagaacatcaagaaaggccccgcccccttctaccccctggaggacggcaccgccggcgagcagctgcacaaagccatgaaaagatacgctctggtgcccggcaccatcgccttcaccgacgcccacatcgaggtgaacatcacctacgccgagtacttcgagatgagcgtgagactggccgaggctatgaagagatacggcttgaacaccaaccacagaatcgtggtgtgcagcgagaacagcctgcagttcttcatgcccgtgctgggcgccctgttcatcggcgtggccgttgcccccgccaacgacatctacaacgagagagagctgctgaacagcatgaacatctctcagcccaccgtggttttcgtgtccaaaaagggcctgcagaagatcctgaacgtgcagaagaagctgcccatcattcagaagatcatcatcatggacagcaagaccgactaccaaggctttcagagcatgtacaccttcgtgacaagccacctgccccccggcttcaacgagtacgacttcgtgcccgagagcttcgacagagacaagaccatcgccctgatcatgaacagcagcggcagcaccggcctgcccaagggcgtggccctgccccacagaaccgcctgcgtgagattcagccacgctagagaccccatcttcggcaatcagatcatccccgacaccgccatcctgagcgtagtgcctttccatcatggcttcggcatgttcaccaccctgggctacctgatctgcggcttcagagtggtgctgatgtacagattcgaggaggagctgttcctgagaagcctgcaagactacaagattcagagcgccctgctggtgcccaccctgttcagcttcttcgccaagagcaccctgatcgacaagtatgacctgagcaacctgcacgaaattgcgagcggcggagcgccgctcagcaaagaggtgggcgaggccgtggccaagagattccacctgcccggcatcagacaaggctacggcctgaccgagaccacaagcgccatcctgatcacccccgagggcgacgataagcccggcgccgtgggcaaggtggtaccattcttcgaggccaaggtggtggacctggacaccggcaagaccctgggcgtgaatcagagaggcgagctgtgcgtgagaggccccatgatcatgagcggctacgtgaacaaccccgaggccaccaacgccctcatcgataaggatgggtggctgcacagcggcgacatcgcctactgggacgaggacgagcacttcttcatcgtggacagactcaagagcctgatcaagtacaagggctaccaagtggcccccgccgagttggagagcatcctgctgcagcaccccaatatcttcgacgctggcgtggccggactacccgacgacgacgccggcgagttacccgccgccgtggtggtgctggagcacggcaagaccatgaccgagaaggagatcgtggactacgtggctagccaagtgaccaccgccaagaagctgagaggcggcgtggtgttcgttgacgaagtgcccaagggcctgaccggcaagctggacgctagaaagatcagagagatcctgatcaaggccaagaagggcggcaagagcaagctgtga

SEQ ID NO: 63 - RNA, 59 nt, synthetic construct
  source 1..59: mol_type = other RNA; note = LUC1- I' truncation
taggagttggtggcagtttcaataacaggtcattgccgagaaaaggatagcactataat

SEQ ID NO: 64 - RNA, 55 nt, synthetic construct
  source 1..55: mol_type = other RNA; note = LUC2- I' truncation
agttggtggcagtttcaataacaggtcattgccgagaaaaggatagcactataat

SEQ ID NO: 65 - RNA, 51 nt, synthetic construct
  source 1..51: mol_type = other RNA; note = LUC3- I' truncation
ggtggcagtttcaataacaggtcattgccgagaaaaggatagcactataat

SEQ ID NO: 66 - RNA, 47 nt, synthetic construct
  source 1..47: mol_type = other RNA; note = LUC4- I' truncation
gcagtttcaataacaggtcattgccgagaaaaggatagcactataat

SEQ ID NO: 67 - RNA, 43 nt, synthetic construct
  source 1..43: mol_type = other RNA; note = LUC5- I' truncation
tttcaataacaggtcattgccgagaaaaggatagcactataat

SEQ ID NO: 68 - RNA, 39 nt, synthetic construct
  source 1..39: mol_type = other RNA; note = LUC6- I' truncation
aataacaggtcattgccgagaaaaggatagcactataat

SEQ ID NO: 69 - RNA, 35 nt, synthetic construct
  source 1..35: mol_type = other RNA; note = LUC7- I' truncation
acaggtcattgccgagaaaaggatagcactataat

SEQ ID NO: 70 - RNA, 31 nt, synthetic construct
  source 1..31: mol_type = other RNA; note = LUC8- I' truncation
gtcattgccgagaaaaggatagcactataat

SEQ ID NO: 71 - RNA, 27 nt, synthetic construct
  source 1..27: mol_type = other RNA; note = LUC9- I' truncation
ttgccgagaaaaggatagcactataat

SEQ ID NO: 72 - RNA, 23 nt, synthetic construct
  source 1..23: mol_type = other RNA; note = LUC10- I' truncation
cgagaaaaggatagcactataat

SEQ ID NO: 73 - RNA, 19 nt, synthetic construct
  source 1..19: mol_type = other RNA; note = LUC11- I' truncation
aaaaggatagcactataat

SEQ ID NO: 74 - RNA, 15 nt, synthetic construct
  source 1..15: mol_type = other RNA; note = LUC12- I' truncation
ggatagcactataat

SEQ ID NO: 75 - RNA, 11 nt, synthetic construct
  source 1..11: mol_type = other RNA; note = LUC13- I' truncation
agcactataat

SEQ ID NO: 76 - DNA, 18 bp, synthetic construct
  source 1..18: mol_type = other DNA; note = 5'UTR truncation
tcaataacaggtcattgc

SEQ ID NO: 77 - RNA, 63 nt, synthetic construct
  source 1..63: mol_type = other RNA; note = LUC15- I' mutant
gagttaggagttgctggcagtttcaataacaggtcattgccgagaaaaggatagcactataat

SEQ ID NO: 78 - RNA, 63 nt, synthetic construct
  source 1..63: mol_type = other RNA; note = LUC16- I' mutant
gagttaggagttgttggcagtttcaataacaggtcattgccgagaaaaggatagcactataat

SEQ ID NO: 79 - RNA, 63 nt, synthetic construct
  source 1..63: mol_type = other RNA; note = 5'UTR I general formula
  misc_difference 14: note = N is selected from the group consisting of A, G, C, and U
gagttaggagttgntggcagtttcaataacaggtcattgccgagaaaaggatagcactataat

SEQ ID NO: 80 - protein, 1273 aa, Severe acute respiratory syndrome coronavirus2
  source 1..1273: note = Wild SARS-COV-2 Spike
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 81 - DNA, 3822 bp, synthetic construct
  source 1..3822: mol_type = other DNA; note = Wild SARS-COV-2 Spike CDS
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 82 - RNA, 3822 nt, synthetic construct
  source 1..3822: mol_type = other RNA; note = Wild SARS-COV-2 Spike mRNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 83 - protein, 566 aa, Influenza A H1N1
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 84 - protein, 566 aa, Influenza A H3N2 [sequence omitted]

SEQ ID NO: 85 - protein, 582 aa, Influenza B Victoria lineage
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 86 - protein, 584 aa, Influenza B Yamagata lineage
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 87 - DNA, 1701 bp, synthetic construct
  source 1..1701: mol_type = other DNA; note = Influenza A H1N1
atgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccggggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctga

SEQ ID NO: 88 - DNA, 1701 bp, synthetic construct
  source 1..1701: mol_type = other DNA; note = Influenza A H3N2
atgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccgcgggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctga

SEQ ID NO: 89 - DNA, 1749 bp, synthetic construct
  source 1..1749: mol_type = other DNA; note = Influenza B Victoria lineage ( B/Washington/02/2019) HA CDS
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcgcgggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtga

SEQ ID NO: 90 - RNA, 1755 nt, synthetic construct
  source 1..1755: mol_type = other RNA; note = Influenza B Yamagata lineage
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcgcggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctga

SEQ ID NO: 91 - DNA, 57 bp, synthetic construct
  source 1..57: mol_type = other DNA; note = 5'UTR-Moderna
gggaaataagagagaaaagaagagtaagaagaaatataagaccccggcgccgccacc

SEQ ID NO: 92 - DNA, 110 bp, synthetic construct
  source 1..110: mol_type = other DNA; note = 3'UTR-Moderna
gctggagcctcggtggcctagcttcttgccccttgggcctccccccagcccctcctccccttcctgcacccgtacccccgtggtctttgaataaagtctgagtgggcggc

SEQ ID NO: 93 - DNA, 1698 bp, synthetic construct
  source 1..1698: mol_type = other DNA; note = Influenza A H1N1 (A/Wisconsin/588/2019) HA Moderna
atgaaggccatcctggtcgtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgaggggagtggcacccctgcacctgggcaagtgcaacatcgccggctggatcctgggcaaccccgagtgcgagagcctgagcacagcccggagctggagctacatcgtggagaccagcaacagcgacaacggcacctgttaccccggcgacttcatcaactacgaggagctgcgggagcagctgagcagcgtgagcagcttcgagcggttcgagatcttccccaagaccagcagctggcccaaccacgacagcgacaacggcgtgacagcagcctgtccacacgccggagccaagagcttctacaagaacctgatctggctggtgaagaagggcaagagctaccccaagatcaaccagacctacatcaacgacaagggcaaggaggtgctggtgctgtggggcatccaccacccacctaccatcgccgaccagcagagcctgtaccagaacgccgacgcctacgtgttcgtgggcaccagccggtacagcaagaagttcaagccagagatcgccacccggcccaaggtgagagaccaggagggccggatgaactactactggaccctggtggagcccggagacaagattaccttcgaggccaccggcaacctggtggcccctcggtacgccttcaccatggaacgggacgctggcagcggcatcatcatcagcgacactcccgtgcacgactgcaacaccacctgccagactcccgagggcgctatcaacaccagcctgcccttccagaacgtgcaccccatcaccatcggcaagtgccccaagtacgtaaagagcaccaaattgcggctggccaccggactcaggaacgtgcccagcatccaaagccggggcctgtttggcgcaatcgccggcttcatcgagggcggctggactggcatggtggacggctggtacggctaccaccaccagaacgaacaggggagcggctacgcagctgacctgaagagcacccagaacgccatcgacaagatcaccaacaaggtgaacagcgtgatcgagaagatgaacacccagttcaccgccgtgggcaaggagttcaaccacctggagaagcggatcgagaacctgaacaagaaggtggacgacggcttcctggacatctggacctacaacgccgagctgctggttctgctggagaacgagcggaccctggactatcacgacagcaacgtgaagaacctgtacgagaaggtgcggaaccagctgaagaacaacgccaaggagatcggcaacggctgcttcgagttctaccacaagtgcgacaacacctgcatggagagcgtgaagaacggcacctacgactaccccaagtacagcgaggaggccaagctgaaccgggagaagatcgacggcgtgaagctggacagcacccggatctaccagatcctggccatctacagcaccgtggccagcagcctggtgctggtggtgagcctgggcgccatcagcttctggatgtgcagcaacggcagcctgcagtgccggatctgcatc

SEQ ID NO: 94 - DNA, 1698 bp, synthetic construct
  source 1..1698: mol_type = other DNA; note = Influenza A H3N2 (A/Cambodia/e0826360/2020) HA Moderna
atgaagaccatcatcgccctgagctacatcctgtgcctggtgttcgcccagaagatccccggcaacgataacagcaccgccaccctgtgtctgggacaccacgccgtgcccaacggcaccatcgtgaagactatcaccaacgaccggatcgaggtgaccaacgccaccgagctggtgcagaacagcagcatcggcgagatctgcgacagccctcaccagatcctggacggcggcaactgcaccctgatcgacgcactgctgggcgaccctcagtgcgacggctttcagaacaaggagtgggacctgttcgtggagagatcgcgggccaacagcaactgctacccctacgacgtccccgactacgcaagcctgagaagcctcgtggcctcaagcggcaccctggagttcaagaacgagagcttcaactggaccggcgtgaagcagaacggcacctcaagcgcctgcatccggggctccagcagcagcttcttctcacggctgaactggctgacccacctgaactacacctaccccgccctgaacgtgaccatgcccaacaacgagcagttcgacaagctgtacatctggggagtgcaccatcccagcaccgacaaggaccagattagcctgttcgcccagcccagcggccggatcaccgtgagcaccaagcggagccagcaggccgtgatccccaacatcggctctcggcccagaatccgggacatccccagccggatcagcatctactggaccattgtgaagcccggcgacatcctgctgatcaactccaccggcaacctgatcgcccctcggggctatttcaagatccggagcggcaagagcagcatcatgcggagcgacgcccctatcggcaagtgcaagagcgagtgcatcacacccaacggaagcatccccaacgacaagcccttccagaacgtgaaccggataacctacggcgcctgccctagatacgtgaagcagagcaccctgaagctggccaccggcatgcggaacgtgcccgagaagcagactcggggcatcttcggcgccatcgccggcttcatcgagaacggctgggagggcatggtggacggctggtacggcttccggcaccagaactctgagggcagaggacaggccgcagacctgaagagcacccaggccgccatcgaccagatcaacggcaagctgaaccggctgatcggcaagaccaacgagaagttccaccagatcgagaaggagttcagcgaggtggagggcagggtacaggacctggagaagtacgtggaggacaccaagatcgacctgtggagctacaacgccgagctgctggtagccctggagaaccagcacaccatcgacctgaccgacagcgagatgaacaagctgttcgagaagaccaagaagcagctgcgggagaacgccgaggacatgggcaacggctgcttcaagatctaccacaagtgcgacaacgcctgcatcggcagcatccggaacgagacctacgaccacaacgtgtaccgggacgaggccctgaacaaccggttccagatcaagggcgtggagctgaagagcggctacaaggactggatcctgtggatcagcttcgccatgtcctgcttcctgctgtgcatcgccctgctgggtttcatcatgtgggcctgccagaagggcaacatccggtgcaacatctgcatc

SEQ ID NO: 95 - DNA, 1746 bp, synthetic construct
  source 1..1746: mol_type = other DNA; note = Influenza B Victoria lineage (B/Washington/02/2019) HA CDS Moderna
atgaaggccatcatcgtgctgttaatggtggtgaccagcaacgccgaccggatctgcaccggcatcacctctagcaacagccctcacgtggtgaagaccgccacacagggcgaggtgaacgtgaccggcgtgattcccctgaccaccacccctaccaagagccacttcgccaacctgaagggaaccgagacccggggcaagctgtgtcccaagtgcctgaactgcaccgacctggacgtggccctgggcagacccaagtgcaccggcaagatccccagcgcccgggtgtctatcctgcacgaagtgcggcccgtgactagcggctgcttccccatcatgcacgaccggaccaagatccggcagctgcccaacctgctgcggggctacgagcacgtgcggctgagcacccacaacgtgatcaacgccgaagacgcacccgggagaccatacgagatcggcaccagcggctcttgccccaacatcaccaacggcaacggcttcttcgctaccatggcctgggccgtgccaaagaacaagactgccaccaaccctctgaccatcgaggtgccctacatctgcaccgagggcgaggaccagatcaccgtgtggggcttccacagcgacagcgagacccagatggccaagctgtacggcgacagcaagccccagaagttcaccagcagcgccaacggcgtgaccacccactacgtgagccagatcggcggcttccccaaccagaccgaggacggcggcttaccccagagcggccggatcgtggtggactacatggtgcagaagagcggcaagaccggcaccatcacctaccagcggggcatcctgctgccacagaaggtgtggtgcgcctcagggcggtcaaaggtgatcaagggcagcctgccactgattggcgaggccgactgcctgcacgagaagtacggcggcctgaacaagagcaagccctactacaccggcgagcacgccaaggcaatcggcaactgccccatctgggtgaagacacccctgaagctggccaacggcaccaagtaccggccacccgccaaactgctgaaggagcggggcttcttcggcgccattgccggcttcctcgaaggcggttgggagggcatgatcgccggctggcacggctacactagccacggcgcacacggagtagcagtggccgccgacctgaagagcacccaggaggccatcaacaagatcaccaagaacctgaacagcctgagcgagctggaggtgaagaatctgcagcggctgtctggcgctatggacgagctgcacaacgagatcctggagctggacgagaaggtggacgacttacgggccgacaccatcagcagccagatcgagctggccgtgctgctctccaacgagggcatcatcaacagcgaggacgagcacctgctcgccctggagcggaagctgaagaagatgctgggcccttctgccgtggagatcggtaacggctgcttcgagaccaagcacaagtgcaaccagacctgcctggatcggatcgcagccggcacctttgacgccggggagttcagcctgcccaccttcgacagcctgaacatcaccgccgccagcctgaacgacgacggcctggacaaccacaccatcctgctgtactactctacagccgctagcagcctggccgtgaccctgatgatcgccatcttcgtggtgtacatggtgagccgggacaacgtgagctgcagcatctgcctg

SEQ ID NO: 96 - DNA, 1752 bp, synthetic construct
  source 1..1752: mol_type = other DNA; note = Influenza B Yamagata lineage (B/Phuket/3073/2013) HA CDS Moderna
atgaaggccatcatcgtgctactgatggtggtgaccagcaacgccgaccggatctgcaccggcatcaccagcagcaacagcccgcacgtggtgaagaccgccacccaaggcgaggtgaacgtgaccggcgtgatcccactgaccaccactcccaccaagagctacttcgccaacctgaagggcacacggactcggggcaagctgtgccccgactgcctgaactgcaccgacctggacgtggccctgggcagacccatgtgcgtgggcaccaccccttctgccaaggccagcatcctgcacgaggtgagacccgtgaccagcgggtgcttccccatcatgcacgaccggaccaagatccggcagctgcccaacctgctgcggggctacgagaagatccggctgagcacccagaacgtgatcgacgccgagaaggcccctggaggtccctaccggctgggcaccagcggaagctgccccaacgccaccagcaagatcggcttcttcgccaccatggcctgggctgtgcccaaggacaactacaagaacgccaccaatcccctgaccgtggaggtgccctacatctgcaccgagggcgaggaccagatcaccgtgtggggcttccacagcgacaacaagacccagatgaagagcctgtacggcgacagcaatccccagaagttcacaagcagcgccaacggcgtgaccacccactacgtgagccagatcggcgacttccccgaccagaccgaggacggagggctgcctcagagtggccggatcgtggtggactacatgatgcagaagcccggcaagaccggcaccatcgtgtaccagcggggcgtgctgttgcctcagaaagtttggtgtgccagcggcaggagcaaggtgatcaagggcagcctgcccctgatcggcgaggcagactgcctccacgaggagtacggcggcctgaacaagagcaagccctactacaccggcaagcacgccaaggccatcggcaactgccccatctgggtgaagacccctctgaagctggccaacggcaccaagtaccggccaccagccaagctgctgaaggagcggggcttctttggcgccattgccggcttcctcgagggaggctgggagggcatgatcgccggctggcacggctacacaagccacggcgcacacggagtggctgtggctgccgacctgaagagcacccaggaggccatcaacaagatcaccaagaacctgaacagcctgagcgagctggaggtgaagaacctgcagcggctgtcaggcgccatggacgagctgcacaacgagatcctggagctggacgagaaggtggacgacctgcgtgccgacaccatcagcagccagatcgagctggccgtgctgctgagcaacgagggcatcatcaacagcgaggacgagcacctgctggccctggagcggaaactgaagaagatgctgggaccctctgccgtggacatcggcaacggctgcttcgagaccaagcacaagtgcaaccagacctgcctggatcggatcgccgccggaaccttcaacgccggcgagttcagcctgcccaccttcgacagcctgaacatcaccgccgccagcctgaacgacgacggcctggacaaccacaccatcctgctgtactacagcactgccgcctcaagcctggccgtgaccctgatgctggccatcttcatcgtgtacatggtgagccgggacaacgtgagctgcagcatctgcctg

SEQ ID NO: 97 - DNA, 3810 bp, synthetic construct
  source 1..3810: mol_type = other DNA; note = SARS-COV-2 BA.4 Spike CDS
atgttcgtgttcctggtgctgctgcccctggtgagctctcagtgcgtgaacctgatcacaagaacacagtcgtacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcgagttcagagtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggggagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcgtggaacagaaagagaatcagcaactgcgtggccgactacagcgtgctgtacaacttcgctcccttcttcgccttcaagtgctatggcgtcagccccaccaagctgaacgacctgtgcttcaccaacgtgtacgccgacagcttcgtgatcagaggcaacgaggtgagccagattgcccccgggcaaaccggcaacatcgccgactacaactacaagctgcccgacgacttcaccggctgcgtgatcgcgtggaacagcaataagctggattccaaggtgggcggcaactacaactaccggtacagactgttcagaaagagcaacctgaagcccttcgagagagacatcagcaccgagatctaccaagccggcaacaagccctgcaacggcgtggcgggcgtcaactgctacttccccctgcagagctacggctttaggcccacctacggcgtgggccatcagccctacagagtggtggtgctgagcttcgagctgctgcacgcccccgcaaccgtgtgcggccccaaaaagagcaccaacctggtgaagaacaagtgcgtgaacttcaactttaatggccttaagggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcacaacgcccaagccctgaacaccctggtgaagcagctgagcagcaagttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 98 - protein, 566 aa, Influenza A H1N1
MKAILVVMLYTFTTANADTLCIGYHANNSTDTVDTVLEKNVTVTHSVNLLEDKHNGKLCKLRGVAPLHLGKCNIAGWILGNPECESLSTARSWSYIVETSNSDNGTCYPGDFINYEELREQLSSVSSFERFEIFPKTSSWPNHDSDNGVTAACPHAGAKSFYKNLIWLVKKGKSYPKINQTYINDKGKEVLVLWGIHHPPTIADQQSLYQNADAYVFVGTSRYSKKFKPEIATRPKVRDQEGRMNYYWTLVEPGDKITFEATGNLVAPRYAFTMERDAGSGIIISDTPVHDCNTTCQTPEGAINTSLPFQNVHPITIGKCPKYVKSTKLRLATGLRNVPSIQSQGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVKLDSTRIYQILAIYSTVASSLVLVVSLGAISFWMCSNGSLQCRICI

SEQ ID NO: 99 - protein, 566 aa, Influenza A H3N2
MKTIIALSYILCLVFAQKIPGNDNSTATLCLGHHAVPNGTIVKTITNDRIEVTNATELVQNSSIGEICDSPHQILDGGNCTLIDALLGDPQCDGFQNKEWDLFVERSRANSNCYPYDVPDYASLRSLVASSGTLEFKNESFNWTGVKQNGTSSACIRGSSSSFFSRLNWLTHLNYTYPALNVTMPNNEQFDKLYIWGVHHPSTDKDQISLFAQPSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQSTLKLATGMRNVPEKQTQGIFGAIAGFIENGWEGMVDGWYGFRHQNSEGRGQAADLKSTQAAIDQINGKLNRLIGKTNEKFHQIEKEFSEVEGRVQDLEKYVEDTKIDLWSYNAELLVALENQHTIDLTDSEMNKLFEKTKKQLRENAEDMGNGCFKIYHKCDNACIGSIRNETYDHNVYRDEALNNRFQIKGVELKSGYKDWILWISFAMSCFLLCIALLGFIMWACQKGNIRCNICI

SEQ ID NO: 100 - protein, 582 aa, Influenza B Victoria lineage
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGRPYEIGTSGSCPNITNGNGFFATMAWAVPKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKEQGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 101 - protein, 584 aa, Influenza B Yamagata lineage
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPLTVEVPYICTEGEDQITVWGFHSDNKTQMKSLYGDSNPQKFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKEQGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 102 - DNA, 1701 bp, synthetic construct
  source 1..1701: mol_type = other DNA; note = Influenza A H1N1 HA0 mutant CDS
atgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccagggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctga

SEQ ID NO: 103 - DNA, 1701 bp, synthetic construct
  source 1..1701: mol_type = other DNA; note = Influenza A H3N2 HA0 mutant CDS
atgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccaggggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctga

SEQ ID NO: 104 - DNA, 1749 bp, synthetic construct
  source 1..1749: mol_type = other DNA; note = Influenza B Victoria HA0 mutant CDS
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcaggggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtga

SEQ ID NO: 105 - DNA, 1755 bp, synthetic construct
  source 1..1755: mol_type = other DNA; note = Influenza B Yamagata HA0 mutant CDS
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcagggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctga

SEQ ID NO: 106 - RNA, 4142 nt, synthetic construct
  source 1..4142: mol_type = other RNA; note = I'-Delta24-B-120A mRNA
gagttaggagttggtggcagtttcaataacaggtcattgccgagaaaaggatagcactataatatgttcgtgttcctggtgctgctgcccctggtgagctctcagtgcgtgaacctgaggacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgccatccacgtcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcggggtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggtgagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcggcgaggtgttcaacgccacaagattcgctagcgtgtacgcgtggaacagaaagagaatcagcaactgcgtggccgactacagcgtgctgtacaacagcgctagcttcagcaccttcaagtgctatggcgtcagccccaccaagctgaacgacctgtgcttcaccaacgtgtacgccgacagcttcgtgatcagaggcgacgaggtgagacagattgcccccgggcaaaccggcaagatcgccgactacaactacaagctgcccgacgacttcaccggctgcgtgatcgcgtggaacagcaataacctggattccaaggtgggcggcaactacaactaccggtacagactgttcagaaagagcaacctgaagcccttcgagagagacatcagcaccgagatctaccaagccggcagcaagccctgcaacggcgtggagggcttcaactgctacttccccctgcagagctacggctttcagcccaccaacggcgtgggctatcagccctacagagtggtggtgctgagcttcgagctgctgcacgcccccgcaaccgtgtgcggccccaaaaagagcaccaacctggtgaagaacaagtgcgtgaacttcaactttaatggccttaccggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagcacgtgaacaacagctacgagtgcgacatccccatcggcgccggcatctgcgctagctatcagacacagaccaacagccgccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaacagagccctgaccggcatcgccgtggagcaagacaagaacacccaagaggtgttcgcccaagtgaagcagatctacaagaccccccccatcaaggacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaaaacgtggtgaatcagaacgcccaagccctgaacaccctggtgaagcagctgagcagcaacttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 107 - RNA, 4139 nt, synthetic construct
  source 1..4139: mol_type = other RNA; note = I'-OC24-B-120A mRNA
gagttaggagttggtggcagtttcaataacaggtcattgccgagaaaaggatagcactataatatgttcgtgttcctggtgctgctgcccctggtgagctctcagtgcgtgaacctgaccacaagaacacagctgccccccgcctacaccaacagcttcacaagaggcgtgtactaccccgacaaggtgttcagaagcagcgtgctgcactccacccaagacctgtttctgcccttcttcagcaacgtgacctggttccacgtgatcagcggcaccaacggcaccaagagattcgacaaccccgtgctgcccttcaacgacggcgtgtacttcgctagcatcgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggaccacaagaacaacaagagctggatggagagcgagttcagagtgtacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcatcgtgagagagcccgaggacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccaccagatttgctagcgtgtacgcctggaaccggaagagaatcagcaactgcgtggccgactacagcgtgctgtacaacctggcccccttcttcaccttcaagtgctatggcgtcagccccaccaagctgaacgacctgtgcttcaccaacgtgtacgccgacagcttcgtgatcagaggcgacgaggtgagacagatcgcccccgggcagaccggcaacatcgccgactacaactacaagctgcccgacgacttcaccggctgcgtgatcgcctggaacagcaacaagctggatagcaaggtgagcggcaactacaattacctgtacagactgttcagaaagagcaacctgaagcccttcgagagagacatcagcaccgagatctaccaagccggcaacaagccctgcaacggcgtggcgggcttcaactgctacttccccctcaggagctacagcttcagacccacctacggcgtgggccatcagccctacagagtggtcgtgctgagcttcgagctgctgcacgcccctgccacagtgtgcggccccaaaaagagcaccaacctggtgaagaacaagtgcgtgaacttcaacttcaacggcctgaagggcaccggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtgtatagcaccggcagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggctagccaaagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctcctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaagggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaaccacaacgcccaagccctgaacaccctggtgaagcagctgagcagcaagttcggcgccatcagcagcgtgctgaatgacatcttctctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 108 - RNA, 4133 nt, synthetic construct
  source 1..4133: mol_type = other RNA; note = I'-BA4-B-120A mRNA
gagttaggagttggtggcagtttcaataacaggtcattgccgagaaaaggatagcactataatatgttcgtgttcctggtgctgctgcccctggtgagctctcagtgcgtgaacctgatcacaagaacacagtcgtacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcgagttcagagtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggggagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcgtggaacagaaagagaatcagcaactgcgtggccgactacagcgtgctgtacaacttcgctcccttcttcgccttcaagtgctatggcgtcagccccaccaagctgaacgacctgtgcttcaccaacgtgtacgccgacagcttcgtgatcagaggcaacgaggtgagccagattgcccccgggcaaaccggcaacatcgccgactacaactacaagctgcccgacgacttcaccggctgcgtgatcgcgtggaacagcaataagctggattccaaggtgggcggcaactacaactaccggtacagactgttcagaaagagcaacctgaagcccttcgagagagacatcagcaccgagatctaccaagccggcaacaagccctgcaacggcgtggcgggcgtcaactgctacttccccctgcagagctacggctttaggcccacctacggcgtgggccatcagccctacagagtggtggtgctgagcttcgagctgctgcacgcccccgcaaccgtgtgcggccccaaaaagagcaccaacctggtgaagaacaagtgcgtgaacttcaactttaatggccttaagggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcacaacgcccaagccctgaacaccctggtgaagcagctgagcagcaagttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 109 - RNA, 4125 nt, synthetic construct
  source 1..4125: mol_type = other RNA; note = I'-BA4-B mRNA
gagttaggagttggtggcagtttcaataacaggtcattgccgagaaaaggatagcactataatatgttcgtgttcctggtgctgctgcccctggtgagctctcagtgcgtgaacctgatcacaagaacacagtcgtacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcgagttcagagtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggggagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcgtggaacagaaagagaatcagcaactgcgtggccgactacagcgtgctgtacaacttcgctcccttcttcgccttcaagtgctatggcgtcagccccaccaagctgaacgacctgtgcttcaccaacgtgtacgccgacagcttcgtgatcagaggcaacgaggtgagccagattgcccccgggcaaaccggcaacatcgccgactacaactacaagctgcccgacgacttcaccggctgcgtgatcgcgtggaacagcaataagctggattccaaggtgggcggcaactacaactaccggtacagactgttcagaaagagcaacctgaagcccttcgagagagacatcagcaccgagatctaccaagccggcaacaagccctgcaacggcgtggcgggcgtcaactgctacttccccctgcagagctacggctttaggcccacctacggcgtgggccatcagccctacagagtggtggtgctgagcttcgagctgctgcacgcccccgcaaccgtgtgcggccccaaaaagagcaccaacctggtgaagaacaagtgcgtgaacttcaactttaatggccttaagggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcacaacgcccaagccctgaacaccctggtgaagcagctgagcagcaagttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 110 - RNA, 2019 nt, synthetic construct
  source 1..2019: mol_type = other RNA; note = I'-H1N1-B mRNA
gagttaggagttggtggcagtttcaataacaggtcattgccgagaaaaggatagcactataatatgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccggggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 111 - RNA, 2019 nt, synthetic construct
  source 1..2019: mol_type = other RNA; note = I'-H3N2-B mRNA
gagttaggagttggtggcagtttcaataacaggtcattgccgagaaaaggatagcactataatatgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccgcgggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 112 - RNA, 2067 nt, synthetic construct
  source 1..2067: mol_type = other RNA; note = I'-B-Victoria-B mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcgcgggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 113 - RNA, 2073 nt, synthetic construct
  source 1..2073: mol_type = other RNA; note = I'-B-Yamagata-B mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcgcggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 114 - RNA, 2019 nt, synthetic construct
  source 1..2019: mol_type = other RNA; note = I'-H1N1-HA0-mut-B mRNA
gagttaggagttggtggcagtttcaataacaggtcattgccgagaaaaggatagcactataatatgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccagggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 115 - RNA, 2019 nt, synthetic construct
  source 1..2019: mol_type = other RNA; note = I'-H3N2-HA0-mut-B mRNA
gagttaggagttggtggcagtttcaataacaggtcattgccgagaaaaggatagcactataatatgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccaggggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 116 - RNA, 2067 nt, synthetic construct
  source 1..2067: mol_type = other RNA; note = I'-B-Victoria-HA0-mut-B mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcaggggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 117 - RNA, 2073 nt, synthetic construct
  source 1..2073: mol_type = other RNA; note = I'-B-Yamagata-HA0-mut-B mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcagggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaaaa

SEQ ID NO: 118 - RNA, 2027 nt, synthetic construct
  source 1..2027: mol_type = other RNA; note = I'-H1N1-B-120A mRNA
gagttaggagttggtggcagtttcaataacaggtcattgccgagaaaaggatagcactataatatgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccggggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 119 - RNA, 2027 nt, synthetic construct
  source 1..2027: mol_type = other RNA; note = I'-H3N2-B-120A mRNA
gagttaggagttggtggcagtttcaataacaggtcattgccgagaaaaggatagcactataatatgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccgcgggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 120 - RNA, 2075 nt, synthetic construct
  source 1..2075: mol_type = other RNA; note = I'-B-Victoria-B-120A mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcgcgggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 121 - RNA, 2081 nt, synthetic construct
  source 1..2081: mol_type = other RNA; note = I'-B-Yamagata-B-120A mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcgcggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 122 - RNA, 2027 nt, synthetic construct
  source 1..2027: mol_type = other RNA; note = I'-H1N1-HA0-mut-B-120A mRNA
gagttaggagttggtggcagtttcaataacaggtcattgccgagaaaaggatagcactataatatgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccagggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 123 - RNA, 2027 nt, synthetic construct
  source 1..2027: mol_type = other RNA; note = I'-H3N2-HA0-mut-B-120A mRNA
gagttaggagttggtggcagtttcaataacaggtcattgccgagaaaaggatagcactataatatgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccaggggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 124 - RNA, 2075 nt, synthetic construct
  source 1..2075: mol_type = other RNA; note = I'-B-Victoria-HA0-mut-B-120A mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcaggggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 125 - RNA, 2081 nt, synthetic construct
  source 1..2081: mol_type = other RNA; note = I'-B-Yamagata-HA0-mut-B-120A mRNA
gagttaggagttggtggcagtttcaataacaggtcattgccgagaaaaggatagcactataatatgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcagggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctgatgactcgaggctcgctttcttgctgtccaatttctattaaaggttcctttgttccctaagtccaactactaaactgggggatattatgaagggccttgagcatctggattctgcctaataaaaaacatttattttcattgcaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 126 - RNA, 1701 nt, synthetic construct
  source 1..1701: mol_type = other RNA; note = Influenza A H1N1 (A/Wisconsin/588/2019) HA mRNA
atgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccggggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctga

SEQ ID NO: 127 - RNA, 1701 nt, synthetic construct
  source 1..1701: mol_type = other RNA; note = Influenza A H3N2 (A/Cambodia/e0826360/2020) HA mRNA
atgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccgcgggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctga

SEQ ID NO: 128 - RNA, 1749 nt, synthetic construct
  source 1..1749: mol_type = other RNA; note = Influenza B Victoria lineage ( B/Washington/02/2019) HA mRNA
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcgcgggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtga

SEQ ID NO: 129 - RNA, 1755 nt, synthetic construct; identical to SEQ ID NO: 90
  source 1..1755: mol_type = other RNA; note = Influenza B Yamagata lineage (B/Phuket/3073/2013) HA mRNA

SEQ ID NO: 130 - RNA, 3810 nt, synthetic construct
  source 1..3810: mol_type = other RNA; note = SARS-COV-2 BA.4 Spike mRNA
atgttcgtgttcctggtgctgctgcccctggtgagctctcagtgcgtgaacctgatcacaagaacacagtcgtacaccaacagcttcacaagaggcgtgtactaccccgacaaggtgttcagaagcagcgtgctgcactccacccaagacctgtttctgcccttcttcagcaacgtgacctggttccacgccatcagcggcaccaacggcaccaagagattcgacaaccccgtgctgcccttcaacgacggcgtgtacttcgctagcaccgaaaagagcaacatcatcagaggctggatcttcggcaccaccctagactccaagacacaaagcctgctgatcgttaacaacgcgaccaacgtggtgatcaaggtgtgcgagtttcagttctgcaacgaccccttcctggacgtgtactaccacaagaacaacaagagctggatggagagcgagttcagagtctacagcagcgccaacaactgcaccttcgagtacgtgtctcagcccttcctgatggacctggagggcaagcaaggcaatttcaagaacctaagagagttcgtgttcaagaacatcgacggctacttcaagatctacagcaagcacacccccatcaacctggggagagacctgccccaaggcttcagcgccctggagcccctggtggacctgcccatcggcatcaacatcacaagatttcagaccctgctggccctgcaccgtagctacctaacccccggcgatagttcctctggctggacggccggcgctgccgcctactacgtgggctacctgcagcctagaaccttcctgctgaagtacaacgagaacggcactatcactgatgccgtggactgtgccctggaccccctgagcgagaccaagtgcaccctcaagagcttcaccgtggagaagggcatctatcagacaagcaacttcagagtgcagcccaccgagagcatcgtgagattccccaacatcaccaacctgtgccccttcgacgaggtgttcaacgccacaagattcgctagcgtgtacgcgtggaacagaaagagaatcagcaactgcgtggccgactacagcgtgctgtacaacttcgctcccttcttcgccttcaagtgctatggcgtcagccccaccaagctgaacgacctgtgcttcaccaacgtgtacgccgacagcttcgtgatcagaggcaacgaggtgagccagattgcccccgggcaaaccggcaacatcgccgactacaactacaagctgcccgacgacttcaccggctgcgtgatcgcgtggaacagcaataagctggattccaaggtgggcggcaactacaactaccggtacagactgttcagaaagagcaacctgaagcccttcgagagagacatcagcaccgagatctaccaagccggcaacaagccctgcaacggcgtggcgggcgtcaactgctacttccccctgcagagctacggctttaggcccacctacggcgtgggccatcagccctacagagtggtggtgctgagcttcgagctgctgcacgcccccgcaaccgtgtgcggccccaaaaagagcaccaacctggtgaagaacaagtgcgtgaacttcaactttaatggccttaagggcacgggcgtgctgaccgagagcaacaagaagttcctgccatttcagcaattcggcagagacatcgccgacaccaccgacgcggtcagagaccctcagaccctggagatcctggacatcaccccctgtagcttcggcggcgtgagcgtgatcacccccggcactaacacaagcaaccaagtggccgtgctgtaccaaggcgtgaactgcaccgaggtgcccgtggccatccacgccgatcagctgacccccacctggagagtatacagcaccggaagcaacgtgtttcagacaagagccggctgcctgatcggcgccgagtacgtgaacaacagctacgagtgcgacatccccatcggcgccggcatctgcgctagctatcagacacagaccaagagccaccggagagctagaagcgtggcttctcagagcatcatcgcctacaccatgagcctgggcgccgagaacagcgtggcctacagcaacaacagcatcgccatccccaccaacttcaccatcagcgtgaccaccgagatcctgcccgtaagtatgaccaagacaagcgtggactgcaccatgtacatctgcggcgacagcaccgagtgcagcaacctgctgctgcagtacggcagcttctgcacacagctgaagagagccctgaccggcatcgccgtggagcaagacaagaacacccaagaggtgttcgcccaagtgaagcagatctacaagaccccccccatcaagtacttcggcggcttcaacttcagccaaatcctgcccgaccctagcaagcctagcaagcggagcttcatcgaggacctgctgttcaacaaggtgaccctggccgacgccggcttcatcaagcagtacggcgactgcctaggcgacatcgcagctagagacctgatctgcgctcagaagttcaatggactcaccgtgctcccccccctgctgaccgacgagatgatcgctcagtacacctctgccctgctggcgggaaccataacaagcggttggacatttggcgccggggccgcactgcaaatccccttcgccatgcagatggcctacagattcaacggcatcggcgtgacacagaacgtgctgtacgagaatcagaagctgatcgccaatcagttcaacagcgccatcggcaagatccaagacagcctgagcagcaccgctagcgccctgggcaagctgcaagacgtggtgaatcacaacgcccaagccctgaacaccctggtgaagcagctgagcagcaagttcggcgccatcagcagcgtattgaatgacatcctgtctagactggacccccccgaggccgaggtgcagatcgacagactgatcaccggcagactgcagagcctgcagacctacgtgacacagcagctgatcagagccgccgagatcagagctagcgccaacctggccgccaccaagatgagcgagtgcgtgctggggcagagcaagagagtggacttctgcggcaagggctaccacctgatgagcttccctcagagcgccccccacggcgtggtgttcctgcacgtgacctacgtgcccgcccaagagaagaacttcaccaccgctcccgccatctgccacgacggcaaggcccacttccctagagagggcgtgttcgtgagcaacggcacccactggttcgtgacacagagaaacttctacgagcctcagatcatcaccaccgacaacaccttcgtgagcggcaactgcgacgtggtgatcggcatagtgaacaacaccgtatacgaccccctgcagcccgagctggacagcttcaaggaggagctggacaagtacttcaaaaaccatactagccccgacgtggacctgggagacatctctggcatcaacgctagcgtggtgaacattcagaaggagatcgaccggctgaatgaggtggccaagaacctgaacgagagcctgatcgacctgcaagagctgggcaagtacgagcagtacatcaagtggccctggtacatctggctgggcttcatcgccggcctgatcgccatcgtgatggtgaccatcatgctgtgctgcatgacaagctgttgcagctgcctgaaaggttgctgcagctgtggctcgtgctgcaagttcgacgaggacgacagcgagcccgtgctgaagggcgtgaagctgcactacacctgatga

SEQ ID NO: 131 - RNA, 1701 nt, synthetic construct
  source 1..1701: mol_type = other RNA; note = Influenza A H1N1 HA0 mutant mRNA
atgaaggccatcctggtggtgatgctgtacaccttcaccaccgccaacgccgacaccctgtgcatcggctaccacgccaacaacagcaccgacaccgtggacaccgtgctggagaagaacgtgaccgtgacccacagcgtgaacctgctggaggacaagcacaacggcaagctgtgcaagctgcggggcgtggcccccctgcacctgggcaagtgcaacatcgccggctggatcctgggcaaccccgagtgcgagtccctgagcaccgcccgctcctggagctacatcgtggagacctccaacagcgacaacggcacctgctaccccggggacttcatcaactacgaggagctgcgggagcagctgtccagcgtgtccagcttcgagcgcttcgagatcttccccaagacctccagctggcccaaccacgactccgacaacggcgtgaccgccgcctgcccccacgccggggccaagagcttctacaagaacctgatctggctggtgaagaagggcaagtcctaccccaagatcaaccagacctacatcaacgacaaggggaaggaggtcctcgtgctgtggggcatccaccacccccccaccatcgccgaccagcagagcctctaccagaacgccgacgcctacgtcttcgtggggacctcccggtacagcaagaagttcaagcccgagatcgccacccgccccaaggtccgggaccaggagggccgcatgaactactactggaccctggtggagccgggggacaagatcaccttcgaggccaccggcaacctcgtcgccccgcggtacgccttcacgatggagcgcgacgccgggtccggcatcatcatcagcgacacccccgtgcacgactgcaacacgacctgccagacgcccgagggggcgatcaacacctccctgccgttccagaacgtccacccgatcacgatcggcaagtgccccaagtacgtgaagtccaccaagctccggctggccacggggctccgcaacgtcccctccatccagagccagggcctgttcggggcgatcgccggcttcatcgaggggggctggaccgggatggtggacggctggtacgggtaccaccaccagaacgagcagggctccgggtacgcggccgacctcaagtcgacgcagaacgcgatcgacaagatcaccaacaaggtcaacagcgtgatcgagaagatgaacacgcagttcaccgccgtcggcaaggagttcaaccacctggagaagcgcatcgagaacctcaacaagaaggtggacgacgggttcctggacatctggacgtacaacgcggagctcctggtcctcctggagaacgagcggaccctcgactaccacgactccaacgtgaagaacctgtacgagaaggtccgcaaccagctcaagaacaacgccaaggagatcggcaacgggtgcttcgagttctaccacaagtgcgacaacacgtgcatggagtcggtgaagaacggcacctacgactacccgaagtacagcgaggaggcgaagctgaaccgggagaagatcgacggggtcaagctcgactccacgcgcatctaccagatcctggccatctactcgaccgtggcgagctccctcgtcctggtggtctcgctcggcgccatcagcttctggatgtgctccaacgggtcgctgcagtgccggatctgcatctga

SEQ ID NO: 132 - RNA, 1701 nt, synthetic construct
  source 1..1701: mol_type = other RNA; note = Influenza A H3N2 HA0 mutant mRNA
atgaagaccatcatcgccctgagctacatcctgtgcctggtgttcgcccagaagatccccggcaacgacaacagcaccgccaccctgtgcctgggccaccacgccgtgcccaacggcaccatcgtgaagaccatcaccaacgaccggatcgaggtgaccaacgccaccgagctggtgcagaactccagcatcggcgagatctgcgactccccccaccagatcctggacggcggcaactgcaccctgatcgacgccctgctgggcgacccccagtgcgacgggttccagaacaaggagtgggacctgttcgtggagcggagccgcgccaactccaactgctacccctacgacgtgcccgactacgccagcctgcggtccctggtggccagctccggcaccctggagttcaagaacgagagcttcaactggaccggggtcaagcagaacggcacctccagcgcctgcatccgcgggtccagctccagcttcttctcccggctgaactggctcacccacctgaactacacctaccccgccctcaacgtgaccatgcccaacaacgagcagttcgacaagctgtacatctggggcgtccaccaccccagcaccgacaaggaccagatctccctcttcgcccagcccagcgggcgcatcaccgtgtccacgaagcggagccagcaggccgtcatccccaacatcggctcccgcccgcggatccgcgacatccccagccggatctccatctactggacgatcgtgaagccgggggacatcctgctcatcaacagcaccggcaacctgatcgccccccgcgggtacttcaagatccggtccggcaagagctcgatcatgcgctccgacgccccgatcgggaagtgcaagagcgagtgcatcacccccaacggctcgatcccgaacgacaagcccttccagaacgtcaaccggatcacgtacggggcctgcccgcgctacgtgaagcagtccacgctcaagctggccaccggcatgcggaacgtccccgagaagcagacccaggggatcttcggcgccatcgcggggttcatcgagaacggctgggaggggatggtggacggctggtacgggttccggcaccagaacagcgagggccgcgggcaggccgcggacctcaagtcgacgcaggccgcgatcgaccagatcaacggcaagctgaaccggctcatcgggaagacgaacgagaagttccaccagatcgagaaggagttctccgaggtcgagggccgcgtgcaggacctggagaagtacgtcgaggacaccaagatcgacctctggagctacaacgccgagctgctcgtggcgctggagaaccagcacaccatcgacctcacggactcggagatgaacaagctgttcgagaagacgaagaagcagctccgggagaacgccgaggacatggggaacggctgcttcaagatctaccacaagtgcgacaacgcgtgcatcgggtccatccgcaacgagacctacgaccacaacgtctaccgggacgaggccctgaacaaccgcttccagatcaagggcgtggagctcaagagcgggtacaaggactggatcctgtggatctcgttcgcgatgtcctgcttcctcctgtgcatcgccctcctgggcttcatcatgtgggcgtgccagaaggggaacatccggtgcaacatctgcatctga

SEQ ID NO: 133 - RNA, 1749 nt, synthetic construct
  source 1..1749: mol_type = other RNA; note = Influenza B Victoria HA0 mutant mRNA
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcccacttcgccaacctgaagggcaccgagacccggggcaagctgtgccccaagtgcctgaactgcaccgacctggacgtggccctgggccgccccaagtgcaccggcaagatccccagcgcccgggtgtccatcctgcacgaggtgcgccccgtgaccagcggctgcttccccatcatgcacgaccgcaccaagatccggcagctgcccaacctgctgcgggggtacgagcacgtccgcctgtccacccacaacgtgatcaacgccgaggacgcccccggccggccctacgagatcgggaccagcggctcctgccccaacatcaccaacgggaacggcttcttcgccaccatggcctgggccgtccccaagaacaagaccgccaccaaccccctgaccatcgaggtgccgtacatctgcaccgagggggaggaccagatcaccgtctggggcttccacagcgacaacgagacgcagatggccaagctgtacggggactccaagccccagaagttcacgagctccgccaacggcgtgaccacccactacgtcagccagatcgggggcttcccgaaccagacggaggacgggggcctgccccagtccgggcgcatcgtggtcgactacatggtgcagaagtccggcaagacggggaccatcacgtaccagcggggcatcctgctgccgcagaaggtctggtgcgcctccgggcgcagcaaggtgatcaagggctccctgcccctcatcggggaggccgactgcctgcacgagaagtacggcgggctcaacaagagcaagccgtactacaccggcgagcacgccaaggccatcgggaactgccccatctgggtcaagaccccgctgaagctcgccaacggcacgaagtaccggcccccggccaagctgctcaaggagcaggggttcttcggcgccatcgccgggttcctggagggcgggtgggagggcatgatcgccgggtggcacggctacacgtcccacggggcccacggcgtggcggtcgccgcggacctcaagagcacccaggaggccatcaacaagatcacgaagaacctgaactccctcagcgagctggaggtgaagaacctccagcgcctgtccggggcgatggacgagctccacaacgagatcctggagctcgacgagaaggtcgacgacctgcgggccgacaccatcagctcgcagatcgagctcgcggtgctgctctccaacgagggcatcatcaacagcgaggacgagcacctgctcgccctggagcggaagctcaagaagatgctggggccctcggcggtcgagatcggcaacgggtgcttcgagaccaagcacaagtgcaaccagacgtgcctcgaccgcatcgccgcgggcacgttcgacgccggggagttctccctgccgaccttcgacagcctcaacatcacggcggcctcgctgaacgacgacggcctcgacaaccacaccatcctgctctactactccaccgcggccagctcgctggcggtgacgctcatgatcgccatcttcgtcgtgtacatggtctcccgcgacaacgtgagctgctcgatctgcctgtga

SEQ ID NO: 134 - RNA, 1755 nt, synthetic construct
  source 1..1755: mol_type = other RNA; note = Influenza B Yamagata HA0 mutant mRNA
atgaaggccatcatcgtgctgctgatggtggtgaccagcaacgccgaccggatctgcaccggcatcaccagctccaacagcccccacgtggtgaagaccgccacccagggcgaggtgaacgtgaccggcgtgatccccctgaccaccacccccaccaagtcctacttcgccaacctgaagggcacccggacccgcggcaagctgtgccccgactgcctgaactgcaccgacctggacgtggccctgggccggcccatgtgcgtgggcaccacccccagcgccaaggcctccatcctgcacgaggtgcgccccgtgaccagcggctgcttccccatcatgcacgaccgcaccaagatccggcagctgcccaacctgctgcgggggtacgagaagatccgcctgtccacccagaacgtcatcgacgccgagaaggcccccggcgggccctaccggctgggcaccagcgggtcctgccccaacgccaccagcaagatcggcttcttcgccaccatggcctgggccgtgcccaaggacaactacaagaacgccaccaaccccctgaccgtcgaggtgccctacatctgcaccgagggggaggaccagatcacggtctggggcttccactccgacaacaagacccagatgaagtccctgtacggggactccaacccgcagaagttcacgagctccgccaacggcgtgaccacgcactacgtcagccagatcggggacttcccggaccagaccgaggacggcgggctgccccagtccggccgcatcgtggtcgactacatgatgcagaagcccgggaagacgggcaccatcgtgtaccagcggggggtcctgctgccgcagaaggtgtggtgcgccagcggccgctccaaggtcatcaaggggagcctgcccctcatcggcgaggccgactgcctgcacgaggagtacgggggcctcaacaagtccaagccgtactacacggggaagcacgccaaggccatcggcaactgcccgatctgggtgaagacccccctgaagctcgccaacgggacgaagtaccggccgcccgccaagctgctcaaggagcagggcttcttcggggccatcgccggcttcctggaggggggctgggaggggatgatcgccggctggcacgggtacaccagccacggcgcccacggggtcgcggtggccgcggacctcaagtccacgcaggaggccatcaacaagatcaccaagaacctgaacagcctctccgagctggaggtcaagaacctccagcgcctgagcggcgcgatggacgagctccacaacgagatcctggagctcgacgagaaggtggacgacctgcgggccgacacgatctcgtcccagatcgagctcgcggtcctgctcagcaacgaggggatcatcaactcggaggacgagcacctgctcgccctggagcggaagctcaagaagatgctgggcccgtccgcggtggacatcgggaacggctgcttcgagaccaagcacaagtgcaaccagacgtgcctcgaccgcatcgccgcggggaccttcaacgccggcgagttcagcctgcccacgttcgactcgctcaacatcaccgcggcctccctgaacgacgacgggctcgacaaccacacgatcctgctctactacagcaccgcggcctcgtccctggcggtcacgctcatgctggccatcttcatcgtgtacatggtcagccgcgacaacgtgtcgtgctccatctgcctctga

SEQ ID NO: 135 - DNA, 120 bp, synthetic construct
  source 1..120: mol_type = other DNA; note = poly A
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 136 - protein, 1268 aa, Severe acute respiratory syndrome coronavirus 2
  source 1..1268: note = SARS-COV-2 BA.4 Spike
MFVFLVLLPLVSSQCVNLITRTQSYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLDVYYHKNNKSWMESEFRVYSSANNCTFEYVSQPFLMDLEGKQGNFKNLREFVFKNIDGYFKIYSKHTPINLGRDLPQGFSALEPLVDLPIGINITRFQTLLALHRSYLTPGDXSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNFAPFFAFKCYGVSPTKLNDLCFTNVYADSFVIRGNEVSQIAPGQTGNIADYNYKLPDDFTGCVIAWNSNKLDSKVGGNYNYRYRLFRKSNLKPFERDISTEIYQAGNKPCNGVAGVNCYFPLQSYGFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRRARSVASQSIIAYTMSLGAENSVAYSNNSIAIPTNFTISVTTEILPVXMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 137 - DNA, 62 bp, synthetic construct
  source 1..62: mol_type = other DNA; note = 5'UTR I' truncation
agttaggagttggtggcagtttcaataacaggtcattgccgagaaaaggatagcactataat

SEQ ID NO: 138 - DNA, 61 bp, synthetic construct
  source 1..61: mol_type = other DNA; note = 5'UTR I' truncation
gttaggagttggtggcagtttcaataacaggtcattgccgagaaaaggatagcactataat

SEQ ID NO: 139 - DNA, 60 bp, synthetic construct
  source 1..60: mol_type = other DNA; note = 5'UTR I' truncation
ttaggagttggtggcagtttcaataacaggtcattgccgagaaaaggatagcactataat

SEQ ID NO: 140 - DNA, 58 bp, synthetic construct
  source 1..58: mol_type = other DNA; note = 5'UTR I' truncation
aggagttggtggcagtttcaataacaggtcattgccgagaaaaggatagcactataat

SEQ ID NO: 141 - DNA, 57 bp, synthetic construct
  source 1..57: mol_type = other DNA; note = 5'UTR I' truncation
ggagttggtggcagtttcaataacaggtcattgccgagaaaaggatagcactataat

SEQ ID NO: 142 - DNA, 56 bp, synthetic construct
  source 1..56: mol_type = other DNA; note = 5'UTR I' truncation
gagttggtggcagtttcaataacaggtcattgccgagaaaaggatagcactataat

SEQ ID NO: 143 - DNA, 54 bp, synthetic construct
  source 1..54: mol_type = other DNA; note = 5'UTR I' truncation
gttggtggcagtttcaataacaggtcattgccgagaaaaggatagcactataat

SEQ ID NO: 144 - DNA, 53 bp, synthetic construct
  source 1..53: mol_type = other DNA; note = 5'UTR I' truncation
ttggtggcagtttcaataacaggtcattgccgagaaaaggatagcactataat

SEQ ID NO: 145 - DNA, 52 bp, synthetic construct
  source 1..52: mol_type = other DNA; note = 5'UTR I' truncation
tggtggcagtttcaataacaggtcattgccgagaaaaggatagcactataat

SEQ ID NO: 146 - DNA, 50 bp, synthetic construct
  source 1..50: mol_type = other DNA; note = 5'UTR I' truncation
gtggcagtttcaataacaggtcattgccgagaaaaggatagcactataat

SEQ ID NO: 147 - DNA, 49 bp, synthetic construct
  source 1..49: mol_type = other DNA; note = 5'UTR I' truncation
tggcagtttcaataacaggtcattgccgagaaaaggatagcactataat

SEQ ID NO: 148 - DNA, 48 bp, synthetic construct
  source 1..48: mol_type = other DNA; note = 5'UTR I' truncation
ggcagtttcaataacaggtcattgccgagaaaaggatagcactataat

SEQ ID NO: 149 - DNA, 46 bp, synthetic construct
  source 1..46: mol_type = other DNA; note = 5'UTR I' truncation
cagtttcaataacaggtcattgccgagaaaaggatagcactataat

SEQ ID NO: 150 - DNA, 45 bp, synthetic construct
  source 1..45: mol_type = other DNA; note = 5'UTR I' truncation
agtttcaataacaggtcattgccgagaaaaggatagcactataat

SEQ ID NO: 151 - DNA, 44 bp, synthetic construct
  source 1..44: mol_type = other DNA; note = 5'UTR I' truncation
gtttcaataacaggtcattgccgagaaaaggatagcactataat

SEQ ID NO: 152 - DNA, 42 bp, synthetic construct
  source 1..42: mol_type = other DNA; note = 5'UTR I' truncation
ttcaataacaggtcattgccgagaaaaggatagcactataat

SEQ ID NO: 153 - DNA, 41 bp, synthetic construct
  source 1..41: mol_type = other DNA; note = 5'UTR I' truncation
tcaataacaggtcattgccgagaaaaggatagcactataat

SEQ ID NO: 154 - DNA, 40 bp, synthetic construct
  source 1..40: mol_type = other DNA; note = 5'UTR I' truncation
caataacaggtcattgccgagaaaaggatagcactataat

SEQ ID NO: 155 - DNA, 38 bp, synthetic construct
  source 1..38: mol_type = other DNA; note = 5'UTR I' truncation
ataacaggtcattgccgagaaaaggatagcactataat

SEQ ID NO: 156 - DNA, 37 bp, synthetic construct
  source 1..37: mol_type = other DNA; note = 5'UTR I' truncation
taacaggtcattgccgagaaaaggatagcactataat

SEQ ID NO: 157 - DNA, 36 bp, synthetic construct
  source 1..36: mol_type = other DNA; note = 5'UTR I' truncation
aacaggtcattgccgagaaaaggatagcactataat

SEQ ID NO: 158 - DNA, 34 bp, synthetic construct
  source 1..34: mol_type = other DNA; note = 5'UTR I' truncation
caggtcattgccgagaaaaggatagcactataat

SEQ ID NO: 159 - DNA, 33 bp, synthetic construct
  source 1..33: mol_type = other DNA; note = 5'UTR I' truncation
aggtcattgccgagaaaaggatagcactataat

SEQ ID NO: 160 - DNA, 32 bp, synthetic construct
  source 1..32: mol_type = other DNA; note = 5'UTR I' truncation
ggtcattgccgagaaaaggatagcactataat

SEQ ID NO: 161 - DNA, 30 bp, synthetic construct
  source 1..30: mol_type = other DNA; note = 5'UTR I' truncation
tcattgccgagaaaaggatagcactataat

SEQ ID NO: 162 - DNA, 29 bp, synthetic construct
  source 1..29: mol_type = other DNA; note = 5'UTR I' truncation
cattgccgagaaaaggatagcactataat

SEQ ID NO: 163 - DNA, 28 bp, synthetic construct
  source 1..28: mol_type = other DNA; note = 5'UTR I' truncation
attgccgagaaaaggatagcactataat

SEQ ID NO: 164 - DNA, 26 bp, synthetic construct
  source 1..26: mol_type = other DNA; note = 5'UTR I' truncation
tgccgagaaaaggatagcactataat

SEQ ID NO: 165 - DNA, 25 bp, synthetic construct
  source 1..25: mol_type = other DNA; note = 5'UTR I' truncation
gccgagaaaaggatagcactataat

SEQ ID NO: 166 - DNA, 24 bp, synthetic construct
  source 1..24: mol_type = other DNA; note = 5'UTR I' truncation
ccgagaaaaggatagcactataat

SEQ ID NO: 167 - DNA, 22 bp, synthetic construct
  source 1..22: mol_type = other DNA; note = 5'UTR I' truncation
gagaaaaggatagcactataat

SEQ ID NO: 168 - DNA, 21 bp, synthetic construct
  source 1..21: mol_type = other DNA; note = 5'UTR I' truncation
agaaaaggatagcactataat

SEQ ID NO: 169 - DNA, 20 bp, synthetic construct
  source 1..20: mol_type = other DNA; note = 5'UTR I' truncation
gaaaaggatagcactataat

SEQ ID NO: 170 - DNA, 18 bp, synthetic construct
  source 1..18: mol_type = other DNA; note = 5'UTR I' truncation
aaaggatagcactataat

SEQ ID NO: 171 - DNA, 17 bp, synthetic construct
  source 1..17: mol_type = other DNA; note = 5'UTR I' truncation
aaggatagcactataat

SEQ ID NO: 172 - DNA, 16 bp, synthetic construct
  source 1..16: mol_type = other DNA; note = 5'UTR I' truncation
aggatagcactataat

SEQ ID NO: 173 - DNA, 34 bp, synthetic construct
  source 1..34: mol_type = other DNA; note = 5'UTR I' truncation
tcaataacaggtcattgccgagaaaaggatagca

SEQ ID NO: 174 - DNA, 37 bp, synthetic construct
  source 1..37: mol_type = other DNA; note = 5'UTR I' truncation
tcaataacaggtcattgccgagaaaaggatagcacta

SEQ ID NO: 175 - DNA, 36 bp, synthetic construct
  source 1..36: mol_type = other DNA; note = 5'UTR I' truncation
tcaataacaggtcattgccgagaaaaggatagcact

SEQ ID NO: 176 - DNA, 39 bp, synthetic construct
  source 1..39: mol_type = other DNA; note = 5'UTR I' truncation
tcaataacaggtcattgccgagaaaaggatagcactata

SEQ ID NO: 177 - DNA, 27 bp, synthetic construct
  source 1..27: mol_type = other DNA; note = 5'UTR I' truncation
tcaataacaggtcattgccgagaaaag

SEQ ID NO: 178 - DNA, 31 bp, synthetic construct
  source 1..31: mol_type = other DNA; note = 5'UTR I' truncation
tcaataacaggtcattgccgagaaaaggata

SEQ ID NO: 179 - DNA, 30 bp, synthetic construct
  source 1..30: mol_type = other DNA; note = 5'UTR I' truncation
tcaataacaggtcattgccgagaaaaggat

SEQ ID NO: 180 - DNA, 33 bp, synthetic construct
  source 1..33: mol_type = other DNA; note = 5'UTR I' truncation
tcaataacaggtcattgccgagaaaaggatagc

SEQ ID NO: 181 - DNA, 26 bp, synthetic construct
  source 1..26: mol_type = other DNA; note = 5'UTR I' truncation
tcaataacaggtcattgccgagaaaa

SEQ ID NO: 182 - DNA, 28 bp, synthetic construct
  source 1..28: mol_type = other DNA; note = 5'UTR I' truncation
tcaataacaggtcattgccgagaaaagg

SEQ ID NO: 183 - DNA, 25 bp, synthetic construct
  source 1..25: mol_type = other DNA; note = 5'UTR I' truncation
tcaataacaggtcattgccgagaaa

SEQ ID NO: 184 - DNA, 16 bp, synthetic construct
  source 1..16: mol_type = other DNA; note = 5'UTR I' truncation
tcaataacaggtcatt

SEQ ID NO: 185 - DNA, 40 bp, synthetic construct
  source 1..40: mol_type = other DNA; note = 5'UTR I' truncation
tcaataacaggtcattgccgagaaaaggatagcactataa

SEQ ID NO: 186 - DNA, 29 bp, synthetic construct
  source 1..29: mol_type = other DNA; note = 5'UTR I' truncation
tcaataacaggtcattgccgagaaaagga

SEQ ID NO: 187 - DNA, 22 bp, synthetic construct
  source 1..22: mol_type = other DNA; note = 5'UTR I' truncation
tcaataacaggtcattgccgag

SEQ ID NO: 188 - DNA, 38 bp, synthetic construct
  source 1..38: mol_type = other DNA; note = 5'UTR I' truncation
tcaataacaggtcattgccgagaaaaggatagcactat

SEQ ID NO: 189 - DNA, 24 bp, synthetic construct
  source 1..24: mol_type = other DNA; note = 5'UTR I' truncation
tcaataacaggtcattgccgagaa

SEQ ID NO: 190 - DNA, 17 bp, synthetic construct
  source 1..17: mol_type = other DNA; note = 5'UTR I' truncation
tcaataacaggtcattg

SEQ ID NO: 191 - DNA, 32 bp, synthetic construct
  source 1..32: mol_type = other DNA; note = 5'UTR I' truncation
tcaataacaggtcattgccgagaaaaggatag

SEQ ID NO: 192 - DNA, 23 bp, synthetic construct
  source 1..23: mol_type = other DNA; note = 5'UTR I' truncation
tcaataacaggtcattgccgaga

SEQ ID NO: 193 - DNA, 15 bp, synthetic construct
  source 1..15: mol_type = other DNA; note = 5'UTR I' truncation
tcaataacaggtcat

SEQ ID NO: 194 - DNA, 19 bp, synthetic construct
  source 1..19: mol_type = other DNA; note = 5'UTR I' truncation
tcaataacaggtcattgcc

SEQ ID NO: 195 - DNA, 35 bp, synthetic construct
  source 1..35: mol_type = other DNA; note = 5'UTR I' truncation
tcaataacaggtcattgccgagaaaaggatagcac

SEQ ID NO: 196 - DNA, 20 bp, synthetic construct
  source 1..20: mol_type = other DNA; note = 5'UTR I' truncation
tcaataacaggtcattgccg